SEQUENCE LISTING (245 sequences)

SEQ ID NO: 1 - RNA, 15 nt, Artificial Sequence
  Description of Artificial Sequence Synthetic oligonucleotide
agcuagaaauagcaa

SEQ ID NO: 2 - RNA, 22 nt, Artificial Sequence
  Description of Artificial Sequence Synthetic oligonucleotide
agcuagaaauagcaaguuaaaa

SEQ ID NO: 3 - RNA, 30 nt, Artificial Sequence
  Description of Artificial Sequence Synthetic oligonucleotide
agcuagaaauagcaaguuaaaauaaggcua

SEQ ID NO: 4 - RNA, 34 nt, Artificial Sequence
  Description of Artificial Sequence Synthetic oligonucleotide
agcuagaaauagcaaguuaaaauaaggcuagucc

SEQ ID NO: 5 - RNA, 22 nt, Artificial Sequence
  Description of Artificial Sequence Synthetic oligonucleotide
guuuuagagcuagaaauagcaa

SEQ ID NO: 6 - RNA, 29 nt, Artificial Sequence
  Description of Artificial Sequence Synthetic oligonucleotide
guuuuagagcuagaaauagcaaguuaaaa

SEQ ID NO: 7 - RNA, 37 nt, Artificial Sequence
  Description of Artificial Sequence Synthetic oligonucleotide
guuuuagagcuagaaauagcaaguuaaaauaaggcua

SEQ ID NO: 8 - RNA, 41 nt, Artificial Sequence
  Description of Artificial Sequence Synthetic oligonucleotide
guuuuagagcuagaaauagcaaguuaaaauaaggcuagucc

SEQ ID NO: 9 - RNA, 40 nt, Artificial Sequence
  Description of Artificial Sequence Synthetic oligonucleotide
guuaucaacuugaaaaaguggcaccgagucggugcuuuuu

SEQ ID NO: 10 - RNA, 10 nt, Artificial Sequence
  Description of Artificial Sequence Synthetic oligonucleotide
cuugaaaaag

SEQ ID NO: 11 - RNA, 16 nt, Artificial Sequence
  Description of Artificial Sequence Synthetic oligonucleotide
uggcaccgagucggug

SEQ ID NO: 12 - RNA, 6 nt, Artificial Sequence
  Description of Artificial Sequence Synthetic oligonucleotide
cuuuuu

SEQ ID NO: 13 - RNA, 8 nt, Artificial Sequence
  Description of Artificial Sequence Synthetic oligonucleotide
guuaucaa

SEQ ID NO: 14 - RNA, 41 nt, Artificial Sequence
  Description of Artificial Sequence Synthetic oligonucleotide
uuaucaacuugaaaaaguggcaccgagucggugcuuuuuuu

SEQ ID NO: 15 - protein, 7 aa, Influenza virus
PKQKKRK

SEQ ID NO: 16 - protein, 10 aa, Hepatitis delta virus
RKLKKKIKKL

SEQ ID NO: 17 - protein, 10 aa, Mus musculus
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

SEQ ID NO: 20 - DNA, 23 bp, Artificial Sequence
  Description of Artificial Sequence Synthetic oligonucleotide
  modified_base (1)..(20): a, c, t or g
  modified_base (21)..(21): a, c, t, g, unknown or other
nnnnnnnnnnnnnnnnnnnnngg

SEQ ID NO: 21 - DNA, 15 bp, Artificial Sequence
  Description of Artificial Sequence Synthetic oligonucleotide
  modified_base (1)..(12): a, c, t or g
  modified_base (13)..(13): a, c, t, g, unknown or other
nnnnnnnnnnnnngg

SEQ ID NO: 22 - DNA, 23 bp, Artificial Sequence
  Description of Artificial Sequence Synthetic oligonucleotide
  modified_base (1)..(20): a, c, t or g
  modified_base (21)..(21): a, c, t, g, unknown or other
nnnnnnnnnnnnnnnnnnnnngg

SEQ ID NO: 23 - DNA, 27 bp, Artificial Sequence
  Description of Artificial Sequence Synthetic oligonucleotide
  modified_base (1)..(20): a, c, t or g
  modified_base (21)..(22): a, c, t, g, unknown or other
nnnnnnnnnnnnnnnnnnnnnnagaaw

SEQ ID NO: 24 - DNA, 19 bp, Artificial Sequence
  Description of Artificial Sequence Synthetic oligonucleotide
  modified_base (1)..(12): a, c, t or g
  modified_base (13)..(14): a, c, t, g, unknown or other
nnnnnnnnnnnnnnagaaw

SEQ ID NO: 25 - DNA, 27 bp, Artificial Sequence
  Description of Artificial Sequence Synthetic oligonucleotide
  modified_base (1)..(20): a, c, t or g
  modified_base (21)..(22): a, c, t, g, unknown or other
nnnnnnnnnnnnnnnnnnnnnnagaaw

SEQ ID NO: 26 - DNA, 18 bp, Artificial Sequence
  Description of Artificial Sequence Synthetic oligonucleotide
  modified_base (1)..(11): a, c, t or g
  modified_base (12)..(13): a, c, t, g, unknown or other
nnnnnnnnnnnnnagaaw

SEQ ID NO: 27 - DNA, 25 bp, Artificial Sequence
  Description of Artificial Sequence Synthetic oligonucleotide
  modified_base (1)..(20): a, c, t or g
  modified_base (21)..(21): a, c, t, g, unknown or other
  modified_base (24)..(24): a, c, t, g, unknown or other
nnnnnnnnnnnnnnnnnnnnnggng

SEQ ID NO: 28 - DNA, 17 bp, Artificial Sequence
  Description of Artificial Sequence Synthetic oligonucleotide
  modified_base (1)..(12): a, c, t or g
  modified_base (13)..(13): a, c, t, g, unknown or other
  modified_base (16)..(16): a, c, t, g, unknown or other
nnnnnnnnnnnnnggng

SEQ ID NO: 29 - DNA, 25 bp, Artificial Sequence
  Description of Artificial Sequence Synthetic oligonucleotide
  modified_base (1)..(20): a, c, t or g
  modified_base (21)..(21): a, c, t, g, unknown or other
  modified_base (24)..(24): a, c, t, g, unknown or other
nnnnnnnnnnnnnnnnnnnnnggng

SEQ ID NO: 30 - DNA, 16 bp, Artificial Sequence
  Description of Artificial Sequence Synthetic oligonucleotide
  modified_base (1)..(11): a, c, t or g
  modified_base (12)..(12): a, c, t, g, unknown or other
  modified_base (15)..(15): a, c, t, g, unknown or other
nnnnnnnnnnnnggng

SEQ ID NO: 31 - RNA, 42 nt, Artificial Sequence
  Description of Artificial Sequence Synthetic oligonucleotide
guuuuagagcuagaaauagcaaguuaaaauaaggcuaguccg

SEQ ID NO: 32 - RNA, 20 nt, Artificial Sequence
  Description of Artificial Sequence Synthetic oligonucleotide
gggcgaggagcuguucaccg

SEQ ID NO: 33 - protein, 5 aa, Influenza virus
DRLRR

SEQ ID NO: 34 - protein, 1368 aa, Artificial Sequence [sequence omitted]

SEQ ID NO: 35 - RNA, 19 nt, Artificial Sequence
  Description of Artificial Sequence Synthetic oligonucleotide
ggcgaggagcuguucaccg

SEQ ID NO: 36 - RNA, 18 nt, Artificial Sequence
  Description of Artificial Sequence Synthetic oligonucleotide
gcgaggagcuguucaccg

SEQ ID NO: 37 - RNA, 17 nt, Artificial Sequence
  Description of Artificial Sequence Synthetic oligonucleotide
cgaggagcuguucaccg

SEQ ID NO: 38 - RNA, 16 nt, Artificial Sequence
  Description of Artificial Sequence Synthetic oligonucleotide
gaggagcuguucaccg

SEQ ID NO: 39 - protein, 328 aa, Artificial Sequence
  Description of Artificial Sequence Synthetic polypeptide
GSFTMDDISPSELKTILHSKRANLYYLQHCRVLVNGGRVEYVTDEGRHSHYWNIPIANTTSLLLGTGTSITQAAMRELARAGVLVGFCGGGGTPLFSANEVDVEVSWLTPQSEYRPTEYLQRWVGFWFDEEKRLVAARHFQRARLERIRHSWLEDRVLRDAGFAVDATALAVAVEDSARALEQAPNHEHLLTEEARLSKRLFKLAAQATRYGEFVRAKRGSGGDPANRFLDHGNYLAYGLAATATWVLGIPHGLAVLHGKTRRGGLVFDVADLIKDSLILPQAFLSAMRGDEEQDFRQACLDNLSRAQALDFMIDTLKDVAQRSTVSA

SEQ ID NO: 40 - protein, 101 aa, Artificial Sequence
  Description of Artificial Sequence Synthetic polypeptide
MAMLYLIFYDITDDNLRNRVAEFLKKKGLDRIQYSVFMGDLNSSRLKDVEAGLKIIGNRKKLQEDERFFILIVPITENQFRERIVIGYSGSEREEKSNVVW

SEQ ID NO: 41 - protein, 273 aa, Artificial Sequence
  Description of Artificial Sequence Synthetic polypeptide
MAHHHHHHGSRFLIRLVPEDKDRAFKVPYNHQYYLQGLIYNAIKSSNPKLATYLHEVKGPKLFTYSLFMAEKREHPKGLPYFLGYKKGFFYFSTCVPEIAEALVNGLLMNPEVRLWDERFYLHEIKVLREPKKFNGSTFVTLSPIAVTVVRKGKSYDVPPMEKEFYSIIKDDLQDKYVMAYGDKPPSEFEMEVLIAKPKRFRIKPGIYQTAWHLVFRAYGNDDLLKVGYEVGFGEKNSLGFGMVKVEGNKTTKEAEEQEKITFNSREELKTGV

SEQ ID NO: 42 - protein, 211 aa, Artificial Sequence
  Description of Artificial Sequence Synthetic polypeptide
MWLTKLVLNPASRAARRDLANPYEMHRTLSKAVSRALEEGRERLLWRLEPARGLEPPVVLVQTLTEPDWSVLDEGYAQVFPPKPFHPALKPGQRLRFRLRANPAKRLAATGKRVALKTPAEKVAWLERRLEEGGFRLLEGERGPWVQILQDTFLEVRRKKDGEEAGKLLQVQAVLFEGRLEVVDPERALATLRRGVGPGKALGLGLLSVAP

SEQ ID NO: 43 - protein, 244 aa, Artificial Sequence
  Description of Artificial Sequence Synthetic polypeptide
MHHHHHHMKSYFVTMGFNETFLLRLLNETSAQKEDSLVIVVPSPIVSGTRAAIESLRAQISRLNYPPPRIYEIEITDFNLALSKILDIILTLPEPIISDLTMGMRMINTLILLGIIVSRKRFTVYVRDEGGGSRVISFNDNTIRALMRDYSREEMKLLNVLYETKGTGITELAKMLDKSEKTLINKIAELKKFGILTQKGKDRKVELNELGLNVIKLNKSVIESSKSSEELVKENKGKEVNIPY

SEQ ID NO: 44 - RNA, 101 nt, Artificial Sequence
  Description of Artificial Sequence Synthetic polynucleotide
gggcgaggagcuguucaccgguuuuagagcuagaaauagcaaguuaaaauaaggcuaguccguuaucaacuugaaaaaguggcaccgagucggugcuuuuu

SEQ ID NO: 45 - RNA, 101 nt, Artificial Sequence; identical to SEQ ID NO: 44
  Description of Artificial Sequence Synthetic polynucleotide

SEQ ID NO: 46 - RNA, 101 nt, Artificial Sequence; identical to SEQ ID NO: 44
  Description of Artificial Sequence Synthetic polynucleotide

SEQ ID NO: 47 - RNA, 101 nt, Artificial Sequence; identical to SEQ ID NO: 44
  Description of Artificial Sequence Synthetic polynucleotide

SEQ ID NO: 48 - RNA, 101 nt, Artificial Sequence; identical to SEQ ID NO: 44
  Description of Artificial Sequence Synthetic polynucleotide

SEQ ID NO: 49 - RNA, 101 nt, Artificial Sequence; identical to SEQ ID NO: 44
  Description of Artificial Sequence Synthetic polynucleotide

SEQ ID NO: 50 - RNA, 101 nt, Artificial Sequence; identical to SEQ ID NO: 44
  Description of Artificial Sequence Synthetic polynucleotide

SEQ ID NO: 51 - RNA, 101 nt, Artificial Sequence; identical to SEQ ID NO: 44
  Description of Artificial Sequence Synthetic polynucleotide

SEQ ID NO: 52 - RNA, 101 nt, Artificial Sequence; identical to SEQ ID NO: 44
  Description of Artificial Sequence Synthetic polynucleotide

SEQ ID NO: 53 - RNA, 101 nt, Artificial Sequence; identical to SEQ ID NO: 44
  Description of Artificial Sequence Synthetic polynucleotide

SEQ ID NO: 54 - RNA, 101 nt, Artificial Sequence; identical to SEQ ID NO: 44
  Description of Artificial Sequence Synthetic polynucleotide

SEQ ID NO: 55 - RNA, 42 nt, Artificial Sequence
  Description of Artificial Sequence Synthetic oligonucleotide
gggcgaggagcuguucaccgguuuuagagcuaugcuguuuug

SEQ ID NO: 56 - RNA, 42 nt, Artificial Sequence; identical to SEQ ID NO: 55
  Description of Artificial Sequence Synthetic oligonucleotide

SEQ ID NO: 57 - RNA, 42 nt, Artificial Sequence; identical to SEQ ID NO: 55
  Description of Artificial Sequence Synthetic oligonucleotide

SEQ ID NO: 58 - RNA, 42 nt, Artificial Sequence; identical to SEQ ID NO: 55
  Description of Artificial Sequence Synthetic oligonucleotide

SEQ ID NO: 59 - RNA, 42 nt, Artificial Sequence; identical to SEQ ID NO: 55
  Description of Artificial Sequence Synthetic oligonucleotide

SEQ ID NO: 60 - RNA, 42 nt, Artificial Sequence; identical to SEQ ID NO: 55
  Description of Artificial Sequence Synthetic oligonucleotide

SEQ ID NO: 61 - RNA, 42 nt, Artificial Sequence; identical to SEQ ID NO: 55
  Description of Artificial Sequence Synthetic oligonucleotide

SEQ ID NO: 62 - RNA, 42 nt, Artificial Sequence; identical to SEQ ID NO: 55
  Description of Artificial Sequence Synthetic oligonucleotide

SEQ ID NO: 63 - RNA, 42 nt, Artificial Sequence; identical to SEQ ID NO: 55
  Description of Artificial Sequence Synthetic oligonucleotide

SEQ ID NO: 64 - RNA, 42 nt, Artificial Sequence; identical to SEQ ID NO: 55
  Description of Artificial Sequence Synthetic oligonucleotide

SEQ ID NO: 65 - RNA, 42 nt, Artificial Sequence; identical to SEQ ID NO: 55
  Description of Artificial Sequence Synthetic oligonucleotide

SEQ ID NO: 66 - RNA, 42 nt, Artificial Sequence; identical to SEQ ID NO: 55
  Description of Artificial Sequence Synthetic oligonucleotide

SEQ ID NO: 67 - RNA, 42 nt, Artificial Sequence; identical to SEQ ID NO: 55
  Description of Artificial Sequence Synthetic oligonucleotide

SEQ ID NO: 68 - RNA, 42 nt, Artificial Sequence; identical to SEQ ID NO: 55
  Description of Artificial Sequence Synthetic oligonucleotide

SEQ ID NO: 69 - RNA, 42 nt, Artificial Sequence; identical to SEQ ID NO: 55
  Description of Artificial Sequence Synthetic oligonucleotide

SEQ ID NO: 70 - RNA, 42 nt, Artificial Sequence; identical to SEQ ID NO: 55
  Description of Artificial Sequence Synthetic oligonucleotide

SEQ ID NO: 71 - RNA, 42 nt, Artificial Sequence; identical to SEQ ID NO: 55
  Description of Artificial Sequence Synthetic oligonucleotide

SEQ ID NO: 72 - RNA, 42 nt, Artificial Sequence
  Description of Artificial Sequence Synthetic oligonucleotide
cuugccccacagggcaguaaguuuuagagcuaugcuguuuug

SEQ ID NO: 73 - RNA, 42 nt, Artificial Sequence; identical to SEQ ID NO: 72
  Description of Artificial Sequence Synthetic oligonucleotide

SEQ ID NO: 74 - RNA, 42 nt, Artificial Sequence
  Description of Artificial Sequence Synthetic oligonucleotide
gaguccgagcagaagaagaaguuuuagagcuaugcuguuuug

SEQ ID NO: 75 - protein, 7 aa, Simian virus 40
PKKKRKV

SEQ ID NO: 76 - protein, 16 aa, Unknown
  Description of Unknown Nucleoplasmin bipartite NLS sequence
KRPAATKKAGQAKKKK

SEQ ID NO: 77 - protein, 9 aa, Unknown
  Description of Unknown C-myc NLS sequence
PAAKRVKLD

SEQ ID NO: 78 - protein, 11 aa, Unknown
  Description of Unknown C-myc NLS sequence
RQRRNELKRSP

SEQ ID NO: 79 - protein, 38 aa, Homo sapiens
NQSSNFGPMKGGNFGGRSSGPYGGGGQYFAKPRNQGGY

SEQ ID NO: 80 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 81 - protein, 8 aa, Unknown
  Description of Unknown Myoma T protein sequence
VSRKRPRP

SEQ ID NO: 82 - protein, 8 aa, Unknown
  Description of Unknown Myoma T protein sequence
PPKKARED

SEQ ID NO: 83 - protein, 8 aa, Homo sapiens
PQPKKKPL

SEQ ID NO: 84 - protein, 12 aa, Mus musculus
SALIKKKKKMAP

SEQ ID NO: 85 - DNA, 20 bp, Artificial Sequence
  Description of Artificial Sequence Synthetic oligonucleotide
gcacccataccttggagcaa

SEQ ID NO: 86 - DNA, 20 bp, Artificial Sequence
  Description of Artificial Sequence Synthetic oligonucleotide
cccataccttggagcaacgg

SEQ ID NO: 87 - DNA, 20 bp, Artificial Sequence
  Description of Artificial Sequence Synthetic oligonucleotide
gctcgccctcccgtcccagg

SEQ ID NO: 88 - DNA, 20 bp, Artificial Sequence
  Description of Artificial Sequence Synthetic oligonucleotide
gttggtccccaaagtcccca

SEQ ID NO: 89 - DNA, 20 bp, Artificial Sequence
  Description of Artificial Sequence Synthetic oligonucleotide
gctccggcagcagatggcaa

SEQ ID NO: 90 - DNA, 20 bp, Artificial Sequence
  Description of Artificial Sequence Synthetic oligonucleotide
tctttgactctaaggcccaa

SEQ ID NO: 91 - DNA, 20 bp, Artificial Sequence
  Description of Artificial Sequence Synthetic oligonucleotide
catgtggcctggtcaacaag

SEQ ID NO: 92 - DNA, 20 bp, Artificial Sequence
  Description of Artificial Sequence Synthetic oligonucleotide
tgtgctggcttccatgaagg

SEQ ID NO: 93 - DNA, 20 bp, Artificial Sequence
  Description of Artificial Sequence Synthetic oligonucleotide
tagatcgaagacatgtggct

SEQ ID NO: 94 - DNA, 20 bp, Artificial Sequence
  Description of Artificial Sequence Synthetic oligonucleotide
tgaaaacaagagcaaggccg

SEQ ID NO: 95 - DNA, 20 bp, Artificial Sequence
  Description of Artificial Sequence Synthetic oligonucleotide
gcgccgtagcctcagcctga

SEQ ID NO: 96 - DNA, 20 bp, Artificial Sequence
  Description of Artificial Sequence Synthetic oligonucleotide
ggcgcatgtgaactccctgg

SEQ ID NO: 97 - DNA, 20 bp, Artificial Sequence
  Description of Artificial Sequence Synthetic oligonucleotide
ggtcagctactgggacaccg

SEQ ID NO: 98 - DNA, 20 bp, Artificial Sequence
  Description of Artificial Sequence Synthetic oligonucleotide
agtgatgttgaggaagagga

SEQ ID NO: 99 - DNA, 20 bp, Artificial Sequence
  Description of Artificial Sequence Synthetic oligonucleotide
gagcttcctgaattaaactt

SEQ ID NO: 100 - DNA, 20 bp, Artificial Sequence
  Description of Artificial Sequence Synthetic oligonucleotide
catagacccctgttgtaaga

SEQ ID NO: 101 - DNA, 20 bp, Artificial Sequence
  Description of Artificial Sequence Synthetic oligonucleotide
aggaagtcagaatctgggca

SEQ ID NO: 102 - DNA, 20 bp, Artificial Sequence
  Description of Artificial Sequence Synthetic oligonucleotide
tggcttgccttggatttcag

SEQ ID NO: 103 - DNA, 20 bp, Artificial Sequence
  Description of Artificial Sequence Synthetic oligonucleotide
ggtaggggaagaccaccgag

SEQ ID NO: 104 - DNA, 20 bp, Artificial Sequence
  Description of Artificial Sequence Synthetic oligonucleotide
gtatttctactgcgacgagg

SEQ ID NO: 105 - DNA, 20 bp, Artificial Sequence
  Description of Artificial Sequence Synthetic oligonucleotide
cttcggggagacaacgacgg

SEQ ID NO: 106 - DNA, 20 bp, Artificial Sequence
  Description of Artificial Sequence Synthetic oligonucleotide
tcccttctcaggattcctac

SEQ ID NO: 107 - DNA, 20 bp, Artificial Sequence
  Description of Artificial Sequence Synthetic oligonucleotide
agatattcaccattataggt

SEQ ID NO: 108 - DNA, 20 bp, Artificial Sequence
  Description of Artificial Sequence Synthetic oligonucleotide
aattactacttgcttcctgt

SEQ ID NO: 109 - DNA, 20 bp, Artificial Sequence
  Description of Artificial Sequence Synthetic oligonucleotide
ggtcggtctccgagtccccg

SEQ ID NO: 110 - DNA, 20 bp, Artificial Sequence
  Description of Artificial Sequence Synthetic oligonucleotide
gatgatgtacctctgccagc

SEQ ID NO: 111 - DNA, 20 bp, Artificial Sequence
  Description of Artificial Sequence Synthetic oligonucleotide
tcagatcgtgcacgtccgcg

SEQ ID NO: 112 - RNA, 42 nt, Artificial Sequence; identical to SEQ ID NO: 31
  Description of Artificial Sequence Synthetic oligonucleotide

SEQ ID NO: 113 - RNA, 42 nt, Artificial Sequence
  Description of Artificial Sequence Synthetic oligonucleotide
  modified_base (1)..(1): a, c, u, g, unknown or other
  modified_base (5)..(22): a, c, u, g, unknown or other
  modified_base (26)..(26): a, c, u, g, unknown or other
  modified_base (28)..(30): a, c, u, g, unknown or other
  modified_base (32)..(37): a, c, u, g, unknown or other
  modified_base (39)..(41): a, c, u, g, unknown or other
nuuunnnnnnnnnnnnnnnnnnguunannnannnnnngnnng

SEQ ID NO: 114 - RNA, 39 nt, Artificial Sequence
  Description of Artificial Sequence Synthetic oligonucleotide
uuaucaacuugaaaaaguggcaccgagucggugcuuuuu

SEQ ID NO: 115 - RNA, 39 nt, Artificial Sequence
  Description of Artificial Sequence Synthetic oligonucleotide
  modified_base (4)..(5): a, c, u, g, unknown or other
  modified_base (7)..(39): a, c, u, g, unknown or other
uuannannnnnnnnnnnnnnnnnnnnnnnnnnnnnnnnn

SEQ ID NO: 116 - RNA, 42 nt, Artificial Sequence; identical to SEQ ID NO: 72
  Description of Artificial Sequence Synthetic oligonucleotide

SEQ ID NO: 117 - RNA, 101 nt, Artificial Sequence; identical to SEQ ID NO: 44
  Description of Artificial Sequence Synthetic polynucleotide

SEQ ID NO: 118 - RNA, 101 nt, Artificial Sequence; identical to SEQ ID NO: 44
  Description of Artificial Sequence Synthetic polynucleotide

SEQ ID NO: 119 - RNA, 101 nt, Artificial Sequence
  Description of Artificial Sequence Synthetic polynucleotide
cccauaccuuggagcaacggguuuuagagcuagaaauagcaaguuaaaauaaggcuaguccguuaucaacuugaaaaaguggcaccgagucggugcuuuuu

SEQ ID NO: 120 - RNA, 101 nt, Artificial Sequence
  Description of Artificial Sequence Synthetic polynucleotide
gcucgcccucccgucccaggguuuuagagcuagaaauagcaaguuaaaauaaggcuaguccguuaucaacuugaaaaaguggcaccgagucggugcuuuuu

SEQ ID NO: 121 - DNA, 21 bp, Artificial Sequence
  Description of Artificial Sequence Synthetic primer
tcaagcctcagacagtggttc

SEQ ID NO: 122 - DNA, 20 bp, Artificial Sequence
  Description of Artificial Sequence Synthetic primer
tccttgaagtcgatgccctt

SEQ ID NO: 123 - DNA, 27 bp, Artificial Sequence
  Description of Artificial Sequence Synthetic primer
ccaactcctaagccagtgccagaagag

SEQ ID NO: 124 - DNA, 27 bp, Artificial Sequence
  Description of Artificial Sequence Synthetic primer
actcagtgcctatcagaaacccaagag

SEQ ID NO: 125 - DNA, 20 bp, Artificial Sequence
  Description of Artificial Sequence Synthetic primer
tcccgttctccacccaatag

SEQ ID NO: 126 - DNA, 22 bp, Artificial Sequence
  Description of Artificial Sequence Synthetic primer
tgatttccaggctatgcttcca

SEQ ID NO: 127 - DNA, 20 bp, Artificial Sequence
  Description of Artificial Sequence Synthetic primer
gttggcagggagacttagca

SEQ ID NO: 128 - DNA, 21 bp, Artificial Sequence
  Description of Artificial Sequence Synthetic primer
cccatggtacgactgttctca

SEQ ID NO: 129 - DNA, 20 bp, Artificial Sequence
  Description of Artificial Sequence Synthetic primer
tggggccttcaagtgttctt

SEQ ID NO: 130 - DNA, 20 bp, Artificial Sequence
  Description of Artificial Sequence Synthetic primer
gtgtgctcctatgcctggtt

SEQ ID NO: 131 - DNA, 21 bp, Artificial Sequence
  Description of Artificial Sequence Synthetic primer
aaaaccacccttctctctggc

SEQ ID NO: 132 - DNA, 21 bp, Artificial Sequence
  Description of Artificial Sequence Synthetic primer
ggagattggagacacggagag

SEQ ID NO: 133 - DNA, 43 bp, Artificial Sequence
  Description of Artificial Sequence Synthetic primer
ctacacgacgctcttccgatctctagatgagcagagaagaccc

SEQ ID NO: 134 - DNA, 41 bp, Artificial Sequence
  Description of Artificial Sequence Synthetic primer
agacgtgtgctcttccgatcttggtgcccaggacgaggatg

SEQ ID NO: 135 - DNA, 20 bp, Artificial Sequence
  Description of Artificial Sequence Synthetic oligonucleotide
gcaaacttgatggcaaaccg

SEQ ID NO: 136 - DNA, 20 bp, Artificial Sequence
  Description of Artificial Sequence Synthetic oligonucleotide
gcacctgaagagtatcacag

SEQ ID NO: 137 - DNA, 20 bp, Artificial Sequence
  Description of Artificial Sequence Synthetic oligonucleotide
tgtcttgtaaaagaacccag

SEQ ID NO: 138 - RNA, 101 nt, Artificial Sequence
  Description of Artificial Sequence Synthetic polynucleotide
gggcgaggagcuguucaccgguuuuagagcuagaaauagcaaguuaaaauaaggcuaguccguuaucaacuugaaaaagugggaccgagucggugcuuuuu

SEQ ID NO: 139 - RNA, 81 nt, Artificial Sequence
  Description of Artificial Sequence Synthetic oligonucleotide
guuuuagagcuagaaauagcaaguuaaaauaaggcuaguccguuaucaacuugaaaaaguggcaccgagucggugcuuuuu

SEQ ID NO: 140 - RNA, 23 nt, Artificial Sequence
  Description of Artificial Sequence Synthetic oligonucleotide
caaaacagcauagcaaguuaaaa

SEQ ID NO: 141 - RNA, 42 nt, Artificial Sequence; identical to SEQ ID NO: 55
  Description of Artificial Sequence Synthetic oligonucleotide

SEQ ID NO: 142 - DNA, 23 bp, Artificial Sequence
  Description of Artificial Sequence Synthetic oligonucleotide
gggcggtaaacagctcctcgccc

SEQ ID NO: 143 - DNA, 23 bp, Artificial Sequence
  Description of Artificial Sequence Synthetic oligonucleotide
gggcgaggagctgttcaccgccc

SEQ ID NO: 144 - RNA, 20 nt, Artificial Sequence
  Description of Artificial Sequence Synthetic oligonucleotide
gggcgaggagcuguucaccg

SEQ ID NO: 145 - RNA, 101 nt, Artificial Sequence; identical to SEQ ID NO: 44
  Description of Artificial Sequence Synthetic polynucleotide

SEQ ID NO: 146 - DNA, 23 bp, Artificial Sequence
  Synthetic Oligonucleotide
gggcgaggagctgttcaccgggg

SEQ ID NO: 147 - DNA, 23 bp, Artificial Sequence
  Synthetic Oligonucleotide
ccccggtaaacagctcctcgccc

SEQ ID NO: 148 - DNA, 20 bp, Artificial Sequence
  Synthetic oligonucleotide
gggcgaggagctguucaccg

SEQ ID NO: 149 - DNA, 20 bp, Artificial Sequence
  Synthetic oligonucleotide
gggcgaggagctgtucaccg

SEQ ID NO: 150 - DNA, 20 bp, Artificial Sequence
  Synthetic oligonucleotide
gggcgaggagctgttcaccg

SEQ ID NO: 151 - DNA, 20 bp, Artificial Sequence
  Synthetic oligonucleotide
gggcgaggagctgttcaccg

SEQ ID NO: 152 - DNA, 20 bp, Artificial Sequence
  Synthetic oligonucleotide
cccggaggagcuguucaccg

SEQ ID NO: 153 - DNA, 20 bp, Artificial Sequence
  Synthetic oligonucleotide
gggcgaggucgaguucaccg

SEQ ID NO: 154 - DNA, 20 bp, Artificial Sequence
  Synthetic oligonucleotide
gacccccuccaccccgccuc

SEQ ID NO: 155 - DNA, 20 bp, Artificial Sequence
  Synthetic oligonucleotide
gaccccctccaccccgccuc

SEQ ID NO: 156 - DNA, 20 bp, Artificial Sequence
  Synthetic oligonucleotide
gggcccctccaccccgcctc

SEQ ID NO: 157 - DNA, 20 bp, Artificial Sequence
  Synthetic oligonucleotide
gcccccacccaccccgcctc

SEQ ID NO: 158 - DNA, 20 bp, Artificial Sequence
  Synthetic oligonucleotide
cgccctccccaccccgcctc

SEQ ID NO: 159 - DNA, 20 bp, Artificial Sequence
  Synthetic oligonucleotide
gggcacgggcagcuugccgg

SEQ ID NO: 160 - DNA, 20 bp, Artificial Sequence
  Synthetic oligonucleotide
gggcacgggcagcuuggcgg

SEQ ID NO: 161 - DNA, 20 bp, Artificial Sequence
  Synthetic oligonucleotide
gggcacggccagcuugccgg

SEQ ID NO: 162 - DNA, 42 bp, Artificial Sequence
  Synthetic oligonucleotide
gggcgaggagcuguucaccgguuuuugagcuaugcuguuuug

SEQ ID NO: 163 - DNA, 42 bp, Artificial Sequence
  Synthetic oligonucleotide
gggcgaggagcuguucaccggtttttgagctatgctgttttg

SEQ ID NO: 164 - DNA, 42 bp, Artificial Sequence
  Synthetic oligonucleotide
gggcgaggagcuguucaccgguuuuugagctatgctgttttg

SEQ ID NO: 165 - DNA, 20 bp, Artificial Sequence
  Synthetic oligonucleotide
gggcgaggagcuguucaccg

SEQ ID NO: 166 - DNA, 20 bp, Artificial Sequence
  Synthetic oligonucleotide
gggcgtccagcuguucaccg

SEQ ID NO: 167 - DNA, 20 bp, Artificial Sequence
  Synthetic oligonucleotide
gggcguccagcuguucaccg

SEQ ID NO: 168 - DNA, 20 bp, Artificial Sequence
  Synthetic oligonucleotide
gggcgaccagcuguucaccg

SEQ ID NO: 169 - DNA, 20 bp, Artificial Sequence
  Synthetic oligonucleotide
gggcgagcagcuguucaccg

SEQ ID NO: 170 - DNA, 32 bp, Artificial Sequence
  Synthetic oligonucleotide
gggcgaggagcuguucaccgguuuuagagcua

SEQ ID NO: 171 - DNA, 42 bp, Artificial Sequence
  Synthetic oligonucleotide
gggcgaggagcuguucaccgguuuuagagcuaugcuguuuug

SEQ ID NO: 172 - DNA, 42 bp, Artificial Sequence; identical to SEQ ID NO: 171
  Synthetic oligonucleotide

SEQ ID NO: 173 - DNA, 42 bp, Artificial Sequence; identical to SEQ ID NO: 171
  Synthetic oligonucleotide

SEQ ID NO: 174 - DNA, 42 bp, Artificial Sequence; identical to SEQ ID NO: 171
  Synthetic oligonucleotide

SEQ ID NO: 175 - DNA, 42 bp, Artificial Sequence; identical to SEQ ID NO: 171
  Synthetic oligonucleotide

SEQ ID NO: 176 - DNA, 42 bp, Artificial Sequence
  Synthetic oligonucleotide
gggcgaggagctguucaccgguuuuagagcuaugcuguuuug

SEQ ID NO: 177 - DNA, 41 bp, Artificial Sequence
  Synthetic oligonucleotide
gggcgaggagctgtucaccguuuuagagcuaugcuguuuug

SEQ ID NO: 178 - DNA, 42 bp, Artificial Sequence
  Synthetic oligonucleotide
gggcgaggagctgttcaccgguuuuagagcuaugcuguuuug

SEQ ID NO: 179 - DNA, 42 bp, Artificial Sequence; identical to SEQ ID NO: 178
  Synthetic olignucleotide

SEQ ID NO: 180 - DNA, 42 bp, Artificial Sequence; identical to SEQ ID NO: 178
  Synthetic oligonucleotide

SEQ ID NO: 181 - DNA, 42 bp, Artificial Sequence
  Synthetic oligonucleotide
gaguccgagcagaagaagaaguuuuagagcuaugcuguuuug

SEQ ID NO: 182 - DNA, 42 bp, Artificial Sequence; identical to SEQ ID NO: 181
  Synthetic oligonucleotide

SEQ ID NO: 183 - DNA, 42 bp, Artificial Sequence
  Synthetic oligonucleotide
gagtccgagcagaagaagaaguuuuagagcuaugcuguuuug

SEQ ID NO: 184 - DNA, 42 bp, Artificial Sequence; identical to SEQ ID NO: 183
  Synthetic oligonucleotide

SEQ ID NO: 185 - DNA, 42 bp, Artificial Sequence; identical to SEQ ID NO: 183
  Synthetic oligonucleotide

SEQ ID NO: 186 - DNA, 42 bp, Artificial Sequence; identical to SEQ ID NO: 183
  Synthetic oligonucleotide

SEQ ID NO: 187 - DNA, 42 bp, Artificial Sequence; identical to SEQ ID NO: 183
  Synthetic oligonucleotide

SEQ ID NO: 188 - DNA, 42 bp, Artificial Sequence; identical to SEQ ID NO: 183
  Synthetic oligonucleotide

SEQ ID NO: 189 - DNA, 42 bp, Artificial Sequence; identical to SEQ ID NO: 183
  Synthetic oligonucleotide

SEQ ID NO: 190 - DNA, 42 bp, Artificial Sequence; identical to SEQ ID NO: 183
  Synthetic oligonucleotide

SEQ ID NO: 191 - DNA, 42 bp, Artificial Sequence; identical to SEQ ID NO: 183
  Synthetic oligonucleotide

SEQ ID NO: 192 - DNA, 75 bp, Artificial Sequence
  Synthetic oligonucleotide
aaacagcauagcaaguuaaaauaaggcuaguccguuaucaacuugaaaaaguggcaccgagucggugcuuuuuuu

SEQ ID NO: 193 - DNA, 42 bp, Artificial Sequence; identical to SEQ ID NO: 171
  Synthetic oligonuceotide

SEQ ID NO: 194 - DNA, 42 bp, Artificial Sequence
  Synthetic oligonucleotide
gacccccuccaccccgccucguuuuagagcuaugcuguuuug

SEQ ID NO: 195 - DNA, 42 bp, Artificial Sequence
  Synthetic oligonucleotide
gaccccctccaccccgccucguuuuagagcuaugcuguuuug

SEQ ID NO: 196 - DNA, 101 bp, Artificial Sequence
  Synthetic oligonucelotide
gggcgaggagcuguucaccgguuuuagagcuagaaauagcaaguuaaaauaaggcuaguccguuaucaacuugaaaaaguggcaccgagucggugcuuuuu

SEQ ID NO: 197 - DNA, 102 bp, Artificial Sequence
  Synthetic oligonucleotide
gggcgaggragcuguucaccgguuuuagagcuagaaauagcaaguuaaaauaaggcuaguccguuaucaacuugaaaaaguggcaccgagucggugcuuuuu

SEQ ID NO: 198 - DNA, 101 bp, Artificial Sequence
  Synthetic oligonucleotide
gaguccgagcagaagaagaaguuuuagagcuagaaauagcaaguuaaaauaaggcuaguccguuaucaacuugaaaaaguggcaccgagucggugcuuuuu

SEQ ID NO: 199 - DNA, 101 bp, Artificial Sequence
  Synthetic oligonucelotide
gagtccgagcagaagaagaaguuuuagagcuagaaauagcaaguuaaaauaaggcuaguccguuaucaacuugaaaaaguggcaccgagucggugcuuuuu

SEQ ID NO: 200 - DNA, 101 bp, Artificial Sequence
  Synthetic oligonucleotide
gacccccuccaccccgccucguuuuagagcuagaaauagcaaguuaaaauaaggcuaguccguuaucaacuugaaaaaguggcaccgagucggugcuuuuu

SEQ ID NO: 201 - DNA, 20 bp, Artificial Sequence
  Synthetic oligonucelotide
gaccccctccaccccgccuc

SEQ ID NO: 202 - DNA, 81 bp, Artificial Sequence
  Synthetic oligonucleotide
guuuuagagcuagaaauagcaaguuaaaauaaggcuaguccguuaucaacuugaaaaaguggcaccgagucggugcuuuuu

SEQ ID NO: 203 - DNA, 40 bp, Artificial Sequence
  Synthetic oligonucleotide
gggcgaggucgaguucacguuuuagagcuaugcuguuuug

SEQ ID NO: 204 - DNA, 42 bp, Artificial Sequence
  Synthetic oligonucleotide
gggcctccagcuguucaccgguuuuagagcuaugcuguuuug

SEQ ID NO: 205 - DNA, 42 bp, Artificial Sequence
  Synthetic oligonucelotide
cccggaggagcuguucaccgguuuuagagcuaugcuguuuug

SEQ ID NO: 206 - DNA, 42 bp, Artificial Sequence
  Synthetic oligonucelotide
gggcacgggcagcuugccggguuuuagagcuaugcuguuuug

SEQ ID NO: 207 - DNA, 42 bp, Artificial Sequence
  Synthetic oligonucleotide
gggcacgggcagcuuggcggguuuuagagcuaugcuguuuug

SEQ ID NO: 208 - DNA, 42 bp, Artificial Sequence
  Synthetic oligonucleotide
gggcacggccagcuugccggguuuuagagcuaugcuguuuug

SEQ ID NO: 209 - DNA, 42 bp, Artificial Sequence; identical to SEQ ID NO: 206
  Synthetic oligonucleotide

SEQ ID NO: 210 - DNA, 42 bp, Artificial Sequence; identical to SEQ ID NO: 207
  Synthetic oligonucleotide

SEQ ID NO: 211 - DNA, 42 bp, Artificial Sequence; identical to SEQ ID NO: 208
  Synthetic oligonucleotide

SEQ ID NO: 212 - DNA, 42 bp, Artificial Sequence
  Synthetic oligonucleotide
gcucgcccucccgucccaggguuuuagagcuaugcuguuuug

SEQ ID NO: 213 - DNA, 42 bp, Artificial Sequence
  Synthetic oligonucleotide
gctcgccctcccgucccaggguuuuagagcuaugcuguuuug

SEQ ID NO: 214 - DNA, 42 bp, Artificial Sequence
  Synthetic oligonucleotide
ggcacugcggcuggagguggguuuuagagcuaugcuguuuug

SEQ ID NO: 215 - DNA, 42 bp, Artificial Sequence
  Synthetic oligonucleotide
ggcactgcggcuggagguggguuuuagagcuaugcuguuuug

SEQ ID NO: 216 - DNA, 42 bp, Artificial Sequence
  Synthetic oligonucleotide
gggcgtccagcuguucaccgguuuuagagcuaugcuguuuug

SEQ ID NO: 217 - DNA, 42 bp, Artificial Sequence
  Synthetic oligonucleotide
gggcguccagcuguucaccgguuuuagagcuaugcuguuuug

SEQ ID NO: 218 - DNA, 42 bp, Artificial Sequence
  Synthetic oligonucleotide
gggcgaccagcuguucaccgguuuuagagcuaugcuguuuug

SEQ ID NO: 219 - DNA, 42 bp, Artificial Sequence; identical to SEQ ID NO: 218
  Synthetic oligonucleotide

SEQ ID NO: 220 - DNA, 42 bp, Artificial Sequence
  Synthetic oligonucleotide
gggcgagcagcuguucaccgguuuuagagcuaugcuguuuug

SEQ ID NO: 221 - DNA, 44 bp, Artificial Sequence
  Synthetic oligonucleotide
gggcgagcagcuguucaccgguuuuagagcuaugcuguuuugug

SEQ ID NO: 222 - DNA, 101 bp, Artificial Sequence
  Synthetic oligonucleotide
gggcgaggagcuguucaccgguuuuagagcuagaaauagcaaguuaaaauaaggcuaguccguuaucaacuugaaaaaguggcaccgagucggugcuauuu

SEQ ID NO: 223 - DNA, 101 bp, Artificial Sequence; identical to SEQ ID NO: 199
  Synthetic oligonucleotide

SEQ ID NO: 224 - DNA, 102 bp, Artificial Sequence
  Synthetic oligonucleotide
gaccccctccaccccgccucguuuuagagcuagaaauagcaaguuaaaauaaggcuaguccguuaucaacuugaaaaaguggcaccgagucggugrcuuuuu

SEQ ID NO: 225 - DNA, 42 bp, Artificial Sequence
  Synthetic oligonucleotide
gggcgaggagcuguucaccggttttagagctatgctgttttg

SEQ ID NO: 226 - DNA, 42 bp, Artificial Sequence
  Synthetic oligonucleotide
gggcgaggagcuguucaccgguuuuagagctatgctgttttg

SEQ ID NO: 227 - DNA, 42 bp, Artificial Sequence; identical to SEQ ID NO: 226
  Synthetic oligonucleotide

SEQ ID NO: 228 - DNA, 43 bp, Artificial Sequence
  Synthetic oligonucleotide
uaauuucuacucuuguagaucugaugguccaugucuguuacuc

SEQ ID NO: 229 - DNA, 43 bp, Artificial Sequence
  Synthetic oligonucleotide
uaauuucuacucuuguagaucugaugguccauguctgttactc

SEQ ID NO: 230 - DNA, 22 bp, Artificial Sequence
  primer
agagaagtcgaggaagagagag

SEQ ID NO: 231 - DNA, 22 bp, Artificial Sequence
  primer
cagcagaaagttcatggtttcg

SEQ ID NO: 232 - DNA, 20 bp, Artificial Sequence
  Synthetic oligonucleotide
caagatgtgcacttgggcta

SEQ ID NO: 233 - DNA, 20 bp, Artificial Sequence
  primer
gcagcctattgtctcctggt

SEQ ID NO: 234 - DNA, 20 bp, Artificial Sequence
  primer
ccaggtggtgtcagcggagg

SEQ ID NO: 235 - DNA, 21 bp, Artificial Sequence
  primer
tgcctggccctctctgagtct

SEQ ID NO: 236 - DNA, 20 bp, Artificial Sequence
  primer
tgctgcaggtggttccgcag

SEQ ID NO: 237 - DNA, 20 bp, Artificial Sequence
  primer
ctggaaccgcatcctccgca

SEQ ID NO: 238 - DNA, 20 bp, Artificial Sequence
  primer
ccatccccttctgtgaatgt

SEQ ID NO: 239 - DNA, 20 bp, Artificial Sequence
  primer
ggagattggagacacggaga

SEQ ID NO: 240 - DNA, 26 bp, Artificial Sequence
  primer
gagacctgctgagggcggcttctccc

SEQ ID NO: 241 - DNA, 23 bp, Artificial Sequence
  primer
agtcagtccatgcctgcagggtc

SEQ ID NO: 242 - DNA, 26 bp, Artificial Sequence
  primer
cccactgtagtcacacagcaccagag

SEQ ID NO: 243 - DNA, 20 bp, Artificial Sequence
  primer
atcaccccaaccccaaacca

SEQ ID NO: 244 - DNA, 20 bp, Artificial Sequence
  primer
ccaggcgtccatgtccttcc

SEQ ID NO: 245 - DNA, 41 bp, Artificial Sequence
  Synthetic oligonucleotide
aauuucuacuguuguagaugagagauuuaggagagaaugac

(19) United States
(12) Patent Application Publication
(10) Pub. No.: US 2019/0233816 A1
(43) Pub. Date: Aug. 1, 2019

(54) STRUCTURE-GUIDED CHEMICAL MODIFICATION OF GUIDE RNA AND ITS APPLICATIONS

(71) Applicant: Massachusetts Institute of Technology, Cambridge, MA (US)

(72) Inventors: Robert Samuel LANGER, Newton, MA (US); Hao Yin, Cambridge, MA (US); Daniel G. Anderson, Framingham, MA (US); Wen Xue, Natick, MA (US); Chun-Qing Song, Shrewsbury, MA (US)

(21) Appl. No.: 16/256,003

(22) Filed: Jan. 24, 2019

Related U.S. Application Data
(60) Provisional application No. 62/622,696, filed on Jan. 26, 2018.

(51) Int. Cl. C12N 15/113; A61K 31/7105; C12N 15/85; C12N 15/90; C12N 9/22

(52) U.S. Cl. CPC C12N 15/113; A61K 31/7105; C12N 15/85; C12N 2310/20; C12N 9/22; C12Y 301/11002; C12N 15/907

(57) ABSTRACT

The disclosure relates to compositions comprising and methods for chemical modification of single guide RNA (sgRNA), tracrRNA and/or crRNA used individually or in combination with one another or Cas system components. Compositions comprising modified ribonucleic acids have been designed with chemical modification for even higher efficiency as unmodified native strand of sgRNA. Administration of modified ribonucleic acids will allow decreased immune response when administered to a subject, increased stability, increased editing efficiency and facilitated in vivo delivery of sgRNA via various delivery platforms. The disclosure also relates to methods of decreasing off-target effect of CRISPR and a CRISPR complex.

### RELATED APPLICATIONS

This application claims priority to U.S. Provisional Patent Application No. 62/622,696 filed on Jan. 26, 2018, the contents of which are incorporated herein in their entirety.

### SEQUENCE LISTING

The instant application contains a Sequence Listing which has been submitted electronically in ASCII format and is hereby incorporated by reference in its entirety. Said ASCII copy, created on Jan. 23, 2019, is named 127299_00702_Sequence_Listing.txt and is 78 KB in size.

### FIELD OF DISCLOSURE

The disclosure relates to compositions comprising modified nucleic acid sequence and methods of making, using and administering such modified nucleic acid sequences to, among other things, stabilize components of the CRISPR/Cas system.

### BACKGROUND

Therapeutic genome editing has great potential to benefit a range of diseases. A key challenge is the efficient and clinically suitable delivery of genome editing biomacromolecules. The CRISPR (clustered regularly interspersed short palindromic repeats)/Cas9 system is a transforming and powerful genome editing tool. CRISPR/Cas9 consists of a short guide RNA (sgRNA) and an RNA-guided nuclease (Cas9). Cas9-sgRNA complex recognizes the protospacer-adjacent motif (PAM) and a 20 nucleotide sequence in the genome by Watson-Crick base pairing. Site specific double-stranded DNA breaks (DSB) generated by Cas9 are repaired by endogenous cellular mechanisms, including homology-directed repair (HDR) or nonhomologous end-joining (NHEJ). In principle, CRISPR/Cas9 can be applied to treat many genetic diseases. However, its therapeutic potential requires safe and efficient delivery.

An ideal genome editing delivery system would limit the duration of exposure to editing machinery, in order to minimize potential side effects. Cas9-sgRNA ribonucleoprotein (RNP)-based delivery of CRISPR has been tested for cell culture or local delivery in mouse inner ear cells⁷, but these methods are not amenable for systemic in vivo delivery to target major organs such as the liver. Viral vehicles including the adeno-associated virus (AAV) have been used as the delivery agents for long-term CRISPR expression^{8, 9}. However, spCas9, as the most commonly used form of Cas9, is difficult to fit in typical AAV constructs with strong promoters. A smaller form of Cas9 was shown the capability of packing into a single AAV construct. However, concerns regarding potential off-target effects remain if Cas9 is stably expressed by AAV delivery. Moreover, the T cell responses to AAV capsid can limit repeat dosing in patients. The long-term presence of Cas9, a protein from bacteria, in human tissue also increases the risk of immunogenicity. These limitations can be substantively addressed using non-viral delivery system. Previously, we have shown lipid nanoparticles (LNP) encapsulated Cas9 mRNA in combination with a AAV carrying a sgRNA and a repair template inducing efficient genome editing in the mouse liver. However, to date a fully non-viral, systemic Cas9 genome editing system allowing efficient in vivo gene modification has not been described.

### SUMMARY

The present disclosure relates to a nucleic acid sequence comprising at least one or a combination of domains from a 5′ to 3′ orientation: a DNA-binding domain, a Cas protein-binding domain, and a transcription terminator domain. The present disclosure relates to a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1 to about 20 ribonucleotides or deoxyribonucleotides complementary to a DNA target sequence. The present disclosure relates to a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1 to about 20 ribonucleotides or deoxyribonucleotides complementary to a DNA target sequence, wherein the bonds between the first position through fourth position nucleotides are phosphorothioate bonds and the bond between the sixth through 11^{th} position of nucleotides are phosphorothioate bonds.

The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 40% to about 80% modified ribonucleotides and/or the transcription terminator domain comprises from about 40% to about 80% modified ribonucleotides. The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 1% to about 100% modified ribonucleotides and/or the transcription terminator domain comprises from about 1% to about 100% modified ribonucleotides; and the Cas-protein binding domain comprises about 41 nucleotides and at least one or a combination of nucleotides are conserved at positions according to the sequence of FIG. 3a. The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 1% to about 100% modified ribonucleotides and/or the transcription terminator domain comprises from about 1% to about 100% modified ribonucleotides; and the Cas-protein binding domain comprises from about 1 to about 150 nucleotides and at least one or a combination of nucleotides are conserved at the C1 or C2 of the nucleic acid sugar position of positions according to the sequence of FIG. 3a. The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 1% to about 100% modified ribonucleotides and/or the transcription terminator domain comprises from about 1% to about 100% modified ribonucleotides; and the Cas-protein binding domain comprises from about 1 to about 150 nucleotides and at least one or a combination of nucleotides are conserved at the C2 position of the nucleic acid sugar positions according to the sequence of FIG. 3a. The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 1% to about 100% modified ribonucleotides and/or the transcription terminator domain comprises from about 1% to about 100% modified ribonucleotides; and the Cas-protein binding domain comprises from about 1 to about 150 nucleotides and at least one or a combination of nucleotides are conserved at the C3 position of the nucleic acid sugar of positions: 2, 3, 4, 23, 24, 25, 27, 31, 38, 42, 43, 44, 45, 48 according to the sequence of FIG. 1. The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 1% to about 100% modified ribonucleotides and/or the transcription terminator domain comprises from about 1% to about 100% modified ribonucleotides; and the Cas-protein binding domain comprises from about 1 to about 150 nucleotides and at least one or a combination of nucleotides are conserved at the C4 position of the nucleic acid sugar of positions: 2, 3, 4, 23, 24, 25, 27, 31, 38, 42, 43, 44, 45, 48 according to the sequence of FIG. 1. The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 1% to about 100% modified ribonucleotides and/or the transcription terminator domain comprises from about 1% to about 100% modified ribonucleotides; and the Cas-protein binding domain comprises from about 1 to about 150 nucleotides and at least one or a combination of nucleotides are conserved at the C5 position of the nucleic acid sugar of positions: 2, 3, 4, 23, 24, 25, 27, 31, 38, 42, 43, 44, 45, 48 according to the sequence of FIG. 1.

The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises and/or the transcription terminator domain comprises from about 1% to about 99% modified ribonucleotides except the first 3 nucleotides of the 5′ end of the DNA-binding domain if there are modifications in the DNA-binding domain and the last 3 nucleotides of the 3′ end of the transcription terminator domain; if there are modifications in the transcription terminator domain; and the Cas-protein binding domain comprises from about 1 to about 150 nucleotides and at least one or a combination of nucleotides are conserved at positions: 2, 3, 4, 23, 24, 25, 27, 31, 38, 42, 43, 44, 45, 48 according to the sequence of FIG. 1. The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 40% to about 60% fluorinated ribonucleotides at the 2′carbon position of a pentose sugar and/or the transcription terminator domain comprises from about 40% to about 60% fluorinated ribonucleotides at the 2′carbon position of a pentose sugar.

The present disclosure also relates to a nucleic acid sequence comprising at least one or a combination of domains from a 5′ to 3′ orientation: a DNA-binding domain, a Cas-protein binding domain, and a transcription terminator domain, wherein the DNA-binding domain comprises from about 1% to about 99% modified ribonucleotides and/or deoxyribonucleotides and/or the transcription terminator domain comprises from about 1% to about 99% modified ribonucleotides and/or deoxyribonucleotides; and the Cas-protein binding domain comprises from about 1 to about 150 nucleotides. The present disclosure also relates to a nucleic acid sequence comprising at least one or a combination of domains from a 5′ to 3′ orientation: a DNA-binding domain, a Cas protein-binding domain, and a transcription terminator domain; wherein the DNA-binding domain comprises from about 1% to about 99% modified ribonucleotides and/or deoxyribonucleotides and/or the transcription terminator domain comprises from about 1% to about 99% modified ribonucleotides and/or deoxyribonucleotides; and the Cas-protein binding domain comprises from about 1 to about 150 nucleotides and at least one or a combination of 2′-oxygen within the nucleotides which are conserved at positions according to the sequence of FIG. 3a. In some embodiments, while one or a combination of 2′ carbon substituents in the ribose or deoxyribose sugar may be conserved at a given nucleotide of FIG. 1, other functional groups of the nucleotide may be modified in some embodiments. For instance, any of the modifications listed in this application may be incorporated into a modified nucleotide, either a deoxyribonucleotide or ribonucleotide. In one non-limiting example, any of the nucleotides identified at positions according to the sequence of FIG. 3a comprise a conserved 2′ carbon substituent (oxygen atom or hydroxyl, or hydrogen in the case of a deoxyribonucleic acid) but may contain a modified functional group at the 3′ position.

In some embodiments, the disclosure relates to composition comprising a nucleic acid sequence, wherein the nucleic acid sequence comprises a modified Cas-binding domain with between 1%-99% sequence homology with SEQ ID NO: 11, wherein one or any combination of nucleic acids at position 2, 3, 4, 23, 24, 25, 27, 31, 38, 42, 43, 44, 45, 48 are unmodified. In some embodiments, the disclosure relates to composition comprising a nucleic acid sequence, wherein the nucleic acid sequence comprises a modified Cas-binding domain with between 1%-99% sequence homology with SEQ ID NO: 11, wherein one or any combination of functional groups with nucleic acids at position 2, 3, 4, 23, 24, 25, 27, 31, 38, 42, 43, 44, 45, 48 are unmodified. In some embodiments, the functional group left unmodified is the 2′-oxygen or the hydroxyl group at the 2′ carbon of any such positions.

In some embodiments, the disclosure relates to composition comprising a nucleic acid sequence, wherein the nucleic acid sequence comprises a modified Cas-binding domain with between 1%-99% sequence homology with SEQ ID NO: 11, wherein one or any combination of nucleic acids at position 2, 3, 4, 23, 24, 25, 27, 31, 38, 42, 43, 44, 45, 48 are unmodified in the 2′ OH position. In some embodiments, the disclosure relates to composition comprising a nucleic acid sequence, wherein the nucleic acid sequence comprises a modified Cas-binding domain with between 1%-99% sequence homology with SEQ ID NO: 11, wherein one or any combination of functional groups with nucleic acids at position 2, 3, 4, 23, 24, 25, 27, 31, 38, 42, 43, 44, 45, 48 are unmodified. In some embodiments, the functional group left unmodified is the oxygen at the 2′ carbon position.

In some embodiments, the disclosure relates to a composition comprising a nucleic acid sequence, wherein the nucleic acid sequence comprises a DNA-binding domain comprising at least one fluorinated nucleic acid. In some embodiments, the Cas-protein binding domain comprises at least one fluorinated nucleic acid. In some embodiments, the transcription terminator domain comprises at least one fluorinated nucleic acid. In some embodiments, the nucleic acid sequence consists of from about 25 to about 250 ribonucleotides. In some embodiments, the nucleic acid sequence consists of from about 25 to about 200 ribonucleotides. In some embodiments the nucleic acid consists of from about 25 to about 150 nucleotides, wherein at least one or pluralities of nucleotides are modified. In some embodiments, the nucleic acid sequence consists of from about 25 to about 140 ribonucleotides. In some embodiments, the nucleic acid sequence consists of from about 25 to about 130 ribonucleotides. In some embodiments, the nucleic acid sequence consists of from about 25 to about 120 ribonucleotides. In some embodiments, the nucleic acid sequence consists of from about 25 to about 110 ribonucleotides. In some embodiments, the nucleic acid sequence consists of from about 25 to about 100 ribonucleotides.

The present disclosure relates to a composition comprising one or plurality of sgRNA molecules. In some embodiments, the composition comprises at least one sgRNA molecule comprising GUUUUAGAGCUAGAAAUAGCAAGUUAAAAUAAGGCUAGUC CG (SEQ ID NO:31). In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:31 or a nucleotide sequence in which position 2 of SEQ ID NO:31 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:31 or a nucleotide sequence in which position 3 of SEQ ID NO:31 is a uracil.

In some embodiments, all or a portion of the nucleic acid sequence comprises or consists of synthetically assembled nucleotides. In some embodiments, the nucleic acid sequence is an sgRNA molecule free of recombinantly assembled nucleotides. In some embodiments, the nucleic acid sequence is an sgRNA molecule comprising one or a plurality of nucleotides manufactured by polymerase or by synthesizing.

In some embodiments, the DNA-binding domain consists of from about 20 to about 25 contiguous nucleotides; wherein the Cas-protein binding domain consists of from about 38 to about 42 contiguous nucleotides; wherein the transcription terminator domain consists of from about 38 to about 42 contiguous nucleotides. In some embodiments, the DNA-binding domain consists of from about 20 to about 25 contiguous ribonucleotides; wherein the Cas-protein binding domain consists of from about 38 to about 42 contiguous ribonucleotides; wherein the transcription terminator domain consists of from about 38 to about 42 contiguous ribonucleotides. In some embodiments, the DNA-binding domain consists of from about 20 to about 250 contiguous nucleotides; wherein the Cas-protein binding domain consists of from about 38 to about 250 contiguous nucleotides; wherein the transcription terminator domain consists of from about 38 to about 250 contiguous nucleotides. In some embodiments, the DNA-binding domain consists of from about 20 to about 250 contiguous ribonucleotides; wherein the Cas-protein binding domain consists of from about 38 to about 250 contiguous ribonucleotides; wherein the transcription terminator domain consists of from about 38 to about 200 contiguous ribonucleotides.

The disclosure relates to a nucleic acid sequence comprising a Cas-protein binding domain with at least 70%, 80 homology to SEQ ID NO:1 and binds a target sequence of a DNA molecule in the presence of a Cas protein. In some embodiments, the Cas-binding domain is at least 70% homologous to SEQ ID NO:1 and binds a target sequence of a DNA molecule in the presence of a concentration of Cas protein sufficient to cause hybridization of the DNA-binding domain. In some embodiments, the Cas-protein binding domain is at least 70% homologous to SEQ ID NO:1 and binds a target sequence of a DNA molecule in the presence of a Cas9 protein. In some embodiments, the Cas9 binding domain is at least 70%, 80%, 85%, 90%, 91%, 92%, 93%, 94%, 95%, 96%, 97%, 98%, 99%, or 100% homologous to SEQ ID NO:1 and binds a target sequence of a DNA molecule in the presence of a concentration of Cas protein sufficient to cause hybridization of the DNA-binding domain.

The disclosure relates to a Cas-protein binding domain with at least 70% homology to SEQ ID NO:2 and binds a target sequence of a DNA molecule in the presence of a Cas protein. In some embodiments, the Cas-binding domain is at least 70% homologous to SEQ ID NO:2 and binds a target sequence of a DNA molecule in the presence of a concentration of Cas protein sufficient to cause hybridization of the DNA-binding domain. In some embodiments, the Cas-protein binding domain is at least 70% homologous to SEQ ID NO:2 and binds a target sequence of a DNA molecule in the presence of a Cas9 protein. In some embodiments, the Cas9 binding domain is at least 70%, 80%, 85%, 90%, 91%, 92%, 93%, 94%, 95%, 96%, 97%, 98%, 99%, or 100% homologous to SEQ ID NO:2 and binds a target sequence of a DNA molecule in the presence of a concentration of Cas protein sufficient to cause hybridization of the DNA-binding domain.

The disclosure relates to a Cas-protein binding domain with at least 70% homology to SEQ ID NO:3 and binds a target sequence of a DNA molecule in the presence of a Cas protein. In some embodiments, the Cas-binding domain is at least 70% homologous to SEQ ID NO:3 and binds a target sequence of a DNA molecule in the presence of a concentration of Cas protein sufficient to cause hybridization of the DNA-binding domain. In some embodiments, the transcription terminator domain is at least 70% homologous to SEQ ID NO:3 and wherein the nucleic acid sequence binds a target sequence of a DNA molecule in the presence of a Cas9 protein. In some embodiments, the Cas9 binding domain is at least 70% homologous to SEQ ID NO:3 and binds a target sequence of a DNA molecule in the presence of a concentration of Cas9 protein sufficient to cause hybridization of the DNA-binding domain to the target sequence.

In some embodiments, the transcription terminator domain is at least 70% homologous to SEQ ID NO:3 and wherein the nucleic acid sequence binds a target sequence of a DNA molecule in the presence of a Cas protein. In some embodiments, the Cas binding domain is at least 70%, 80%, 85%, 90%, 91%, 92%, 93%, 94%, 95%, 96%, 97%, 98%, 99%, or 100% homologous to SEQ ID NO:3 and binds a target sequence of a DNA molecule in the presence of a concentration of Cas protein sufficient to cause hybridization of the DNA-binding domain to the target sequence.

In some embodiments, the nucleic acid sequence is at least 70% homologous to SEQ ID NO:4 and wherein the nucleic acid sequence binds a target sequence of a DNA molecule in the presence of a Cas9 protein. In some embodiments, the Cas9 binding domain is at least 70% homologous to SEQ ID NO:4 and binds a target sequence of a DNA molecule in the presence of a concentration of Cas9 protein sufficient to cause hybridization of the DNA-binding domain to the target sequence.

In some embodiments, the nucleic acid sequence is at least 70% homologous to SEQ ID NO:4 and wherein the nucleic acid sequence binds a target sequence of a DNA molecule in the presence of a Cas protein. In some embodiments, the Cas binding domain is at least 70%, 80%, 85%, 90%, 91%, 92%, 93%, 94%, 95%, 96%, 97%, 98%, 99%, or 100% homologous to SEQ ID NO:4 and binds a target sequence of a DNA molecule in the presence of a concentration of Cas protein sufficient to cause hybridization of the DNA-binding domain to the target sequence.

In some embodiments, the nucleic acid sequence is at least 70% homologous to SEQ ID NO:5 and wherein the nucleic acid sequence binds a target sequence of a DNA molecule in the presence of a Cas9 protein. In some embodiments, the Cas9 binding domain is at least 70% homologous to SEQ ID NO:5 and binds a target sequence of a DNA molecule in the presence of a concentration of Cas9 protein sufficient to cause hybridization of the DNA-binding domain to the target sequence. In some embodiments, the nucleic acid sequence is at least 70% homologous to SEQ ID NO:5 and wherein the nucleic acid sequence binds a target sequence of a DNA molecule in the presence of a Cas protein. In some embodiments, the Cas binding domain is at least 70%, 80%, 85%, 90%, 91%, 92%, 93%, 94%, 95%, 96%, 97%, 98%, 99%, or 100% homologous to SEQ ID NO:5 and binds a target sequence of a DNA molecule in the presence of a concentration of Cas protein sufficient to cause hybridization of the DNA-binding domain to the target sequence.

In some embodiments, the nucleic acid sequence is at least 70% homologous to SEQ ID NO:6 and wherein the nucleic acid sequence binds a target sequence of a DNA molecule in the presence of a Cas9 protein. In some embodiments, the Cas9 binding domain is at least 70% homologous to SEQ ID NO:6 and binds a target sequence of a DNA molecule in the presence of a concentration of Cas9 protein sufficient to cause hybridization of the DNA-binding domain to the target sequence. In some embodiments, the nucleic acid sequence is at least 70% homologous to SEQ ID NO:6 and wherein the nucleic acid sequence binds a target sequence of a DNA molecule in the presence of a Cas protein. In some embodiments, the Cas binding domain is at least 70%, 80%, 85%, 90%, 91%, 92%, 93%, 94%, 95%, 96%, 97%, 98%, 99%, or 100% homologous to SEQ ID NO:6 and binds a target sequence of a DNA molecule in the presence of a concentration of Cas protein sufficient to cause hybridization of the DNA-binding domain to the target sequence.

In some embodiments, the nucleic acid sequence comprises a sequence at least 70% homologous to SEQ ID NO:8 and wherein the nucleic acid sequence binds a target sequence of a DNA molecule in the presence of a Cas9 protein. In some embodiments, the Cas9 binding domain is at least 70% homologous to SEQ ID NO:8 and binds a target sequence of a DNA molecule in the presence of a concentration of Cas9 protein sufficient to cause hybridization of the DNA-binding domain to the target sequence. In some embodiments, the nucleic acid sequence is at least 70% homologous to SEQ ID NO:8 and wherein the nucleic acid sequence binds a target sequence of a DNA molecule in the presence of a Cas protein. In some embodiments, the Cas binding domain is at least 70%, 80%, 85%, 90%, 91%, 92%, 93%, 94%, 95%, 96%, 97%, 98%, 99%, or 100% homologous to SEQ ID NO:8 and binds a target sequence of a DNA molecule in the presence of a concentration of Cas protein sufficient to cause hybridization of the DNA-binding domain to the target sequence.

In some embodiments, the DNA-binding domain comprises from about 15% to about 85% fluorinated ribonucleotides at the 2′carbon position of a pentose sugar. In some embodiments, the transcription terminator comprises from about 60% to about 85% fluorinated ribonucleotides at the 2′carbon position of a pentose sugar. In some embodiments, the transcription terminator comprises from about 70% to about 85% fluorinated ribonucleotides at the 2′carbon position of a pentose sugar. In some embodiments, the transcription terminator comprises from about 85% to about 95% fluorinated ribonucleotides at the 2′carbon position of a pentose sugar.

In some embodiments, the DNA-binding domain consists of a sequence a RNA sequence at least 80%, 85%, 90%, 91%, 92%, 93%, 94%, 95%, 96%, 97%, 98%, 99%, or 100% complementary to a DNA target sequence and contiguous with SEQ ID NO:6.

The present disclosure also relates to a composition comprising: (a) a nucleic acid sequence comprising a regulatory element operable in a eukaryotic cell operably linked to at least one nucleotide sequence encoding a Cas protein or functional fragment thereof; and (b) any guide sequence disclosed herein, wherein the DNA-domain hybridizes with a target sequence of a DNA sequence in a eukaryotic cell that contains the DNA sequence, wherein the DNA sequence encodes and the eukaryotic cell expresses at least one gene product. In some embodiments, the nucleic acid sequence comprising a regulatory element operable in a eukaryotic cell operably linked to at least one nucleotide sequence encoding a Cas protein or functional fragment thereof is on a first nucleic acid molecule and the guide sequence is a component of a second nucleic acid molecule, optionally comprising one or a plurality of regulatory elements operable in a eukaryotic cell. Pharmaceutical compositions comprising any one or more nucleic acid sequences or pharmaceutically acceptable salts thereof and a pharmaceutically acceptable carrier are contemplated by this disclosure.

The present disclosure also relates to a composition comprising: (a) a nucleic acid sequence comprising a regulatory element operable in a eukaryotic cell operably linked to at least one nucleotide sequence encoding a deactivated Cas protein; and (b) any one or plurality of guide sequences or nucleic acid sequences disclosed herein wherein the DNA-domain hybridizes with a target sequence of a DNA sequence in a eukaryotic cell that contains the DNA sequence, wherein the DNA sequence encodes and the eukaryotic cell expresses at least one gene product.

The present disclosure also relates to a composition comprising: (a) a nucleic acid sequence comprising a regulatory element operable in a eukaryotic cell operably linked to at least one nucleotide sequence encoding a Cas protein; and (b) a nucleic acid molecule comprising any DNA-binding domain described herein, wherein the DNA-binding domain is capable of hybridizing with a target sequence within a DNA sequence in a eukaryotic cell that contains the DNA sequence, wherein the DNA sequence encodes and the eukaryotic cell expresses at least one gene product. In some embodiments, the composition further comprises a lipid or polymer that encapsulates any of the nucleic acids disclosed herein, including any ribonucleotide described herein. In some embodiments, the composition further comprises a pharmaceutically acceptable carrier.

The present disclosure also relates to a composition comprising: (a) a nucleic acid sequence comprising a regulatory element operable in a eukaryotic cell operably linked to at least one nucleotide sequence encoding a Type-II Cas9 protein; and (b) a ribonucleotide of any of nucleotide sequences disclosed herein wherein the DNA-domain hybridizes with a target sequence of a DNA sequence in a eukaryotic cell that contains the DNA sequence, wherein the DNA sequence encodes and the eukaryotic cell expresses at least one gene product. In some embodiments, the composition further comprises a lipid or polymer that encapsulates the ribonucleotide described herein. In some embodiments, the composition further comprises a pharmaceutically acceptable carrier.

The present disclosure also relates to a kit comprising: (a) one or more vectors comprising: a first regulatory element operable in a eukaryotic cell operably linked to a nucleotide sequence encoding a Cas protein; and (b) any nucleic acid sequence described herein. In some embodiments, the one or more vectors and any nucleic acid sequence described herein are lyophilized or desiccated. The present disclosure also relates to a kit comprising: (a) one or more vectors comprising: a first regulatory element operable in a eukaryotic cell operably linked to a nucleotide sequence encoding a Type-II Cas9 protein; and (b) any nucleic acid sequence described herein. In some embodiments, the one or more vectors and any nucleic acid sequence described herein are lyophilized or desiccated. In some embodiments, the kit further comprises at least one container comprising a reconstitution fluid. In some embodiments, the vectors are free of viral sequences. In some embodiments, the compositions are free of viral protein or polypeptides, but may comprise viral nucleic acid sequence. In some embodiments, the compositions are free of viral nucleic acid or viral polypeptide vectors.

The present disclosure also relates to a method of chemically synthesizing a small guide ribonucleic acid molecule comprising integrating a modification into a nucleic acid. The present disclosure also relates to a method of chemically synthesizing a small guide ribonucleic acid molecule comprising integrating a modification into a ribonucleic acid or a deoxyribonucleic acid. The present disclosure also relates to a method of chemically synthesizing a small guide ribonucleic acid molecule comprising integrating a fluorine atom into or reacting compound comprising a fluorine atom with a nucleic acid sequence.

The present disclosure also relates to a method of altering expression of at least one gene product in a cell comprising introducing into a cell an engineered, non-naturally occurring Clustered Regularly Interspaced Short Palindromic Repeats (CRISPR)-CRISPR associated (Cas) (CRISPR-Cas) system comprising: (a) a vector comprising a nucleotide sequence encoding a Type-II Cas9 protein; and (b) a nucleic acid described herein, wherein components (a) and (b) are located on same or different vectors of the system; wherein the cell contains and expresses a DNA molecule having a target sequence and encoding the gene product; and wherein the guide RNA targets and, at concentration sufficient to hybridize the DNA target sequence, hybridizes with a DNA target sequence and the Cas9 protein cleaves the DNA molecule, whereby expression of the at least one gene product is altered. The present disclosure also relates to a method of altering expression of at least one gene product in a cell comprising introducing into a cell an engineered, non-naturally occurring CRISPR-Cas system comprising: (a) a vector comprising a nucleotide sequence encoding a Cas protein; and (b) a nucleic acid described herein, wherein components (a) and (b) are located on same or different vectors of the system; wherein the cell contains and expresses a DNA molecule having a target sequence and encoding the gene product; and wherein the guide RNA targets and, at concentration sufficient to hybridize the DNA target sequence, hybridizes with a DNA target sequence and the Cas protein cleaves the DNA molecule, whereby expression of the at least one gene product is altered.

In some embodiments, the DNA-binding domain comprises from about 40% to about 60% fluorinated ribonucleotides at the 2′carbon position of a pentose sugar. In some embodiments, the transcription terminator domain comprises from about 40% to about 60% fluorinated ribonucleotides at the 2′carbon position of a pentose sugar. In some embodiments, the DNA-binding domain consists of a sequence a RNA sequence at least 90% complementary to a DNA target sequence and contiguous with SEQ ID NO:6. In some embodiments, the Cas-binding domain of the nucleic acid sequence of the disclosure consists of bases 100% homolgous to any one or more of: SEQ ID NO:1, SEQ ID NO:2, SEQ ID NO:3, SEQ ID NO:4, SEQ ID NO:5, SEQ ID NO:6, SEQ ID NO:7, and SEQ ID NO:8, wherein any one or plurality of nucleotides at or between any one or plurality of positions comprises a modification. In some embodiments, if the nucleotide sequence or sequences comprise bases 100% SEQ ID NO:1, the nucleotide comprises a 2-O-methyl modification at the 2′carbon position at each of the positions. In some embodiments, the Cas-binding domain is free of 2′ fluorine or 2′ halogen modification at the 2′ carbons of each position. In some embodiments, the Cas-binding domain is free of phosphorothioate modifications at the bonds between nucleotides.

The present disclosure also relates to a method of improving the enzymatic efficiency of a Cas protein comprising: exposing the Cas protein to a chemically modified nucleic acid sequence comprising at least one fluorinated nucleotide. In some embodiments, the enzymatic efficiency is increased by no less than from about 5% to about 10%. In some embodiments, the transcription terminator domain is at least 70% homologous to SEQ ID NO:8. In some embodiments, the Cas-binding domain of the nucleic acid sequence of the disclosure consists of bases 100% homolgous to SEQ ID NO:8, wherein nucleotides at positions 1, 8 through 22, 26, 28, 32-37, 40 and 41 are modified and the other nucleotides in the sequence are unmodified ribonucleic acid or deoxyribonucleic acid. In some embodiments, the Cas-binding domain of the nucleic acid sequence of the disclosure consists of bases 100% homolgous to SEQ ID NO:8, wherein nucleotides at positions 1, 8 through 22, 26, 28, 32-37, 40 and 41 are modified.

The present disclosure also relates to a method of reducing off-target enzyme activity of a Cas protein comprising: exposing the Cas protein to a chemically modified nucleic acid sequence comprising at least one fluorinated nucleotide. In some embodiments, the off-target enzyme activity is reduced no less than about 5%.

The present disclosure also relates to a method of introducing a mutation in the genomic DNA of a eukaryotic cell comprising contacting said cell with a nucleic acid sequence or guide sequence described herein or any composition described herein. In some embodiments, the step of contacting is performed in vitro, ex vivo, or in vivo. In some embodiments, the eukaryotic cell is a stem cell or cancer cell. In some embodiments, the step of contacting is performed in vivo. In some embodiments, the cell is a lymphocyte isolated from a subject. In some embodiments, the cell is a cultured T-cell or CAR T cell. In some embodiments, the cell is a cell from the liver, lung, neuron, skin, intestine, stomach, breast, or colon.

### BRIEF DESCRIPTION OF THE DRAWINGS

FIG. 1A-1D shows partial DNA replacement at the guide region of a GFP crRNA induced gene editing in human cells. (A) Diagram of the CRISPR system. (B) HEK293T cells stably expressing both EFs promoter-SpCas9 and EF1a promoter-GFP were transfected with a crRNA targeting GFP and the tracrRNA. Cas9-mediated frame shift NHEJ yields GFP negative cells. When replacement of DNA nucleotides in crRNA is tolerated by Cas9, the % of GFP negative cells will be retained. (C) Illustration of DNA replacement at the guide sequence of GFP crRNAs. The 20 nt guide region is shown. RNA and DNA are shown in black and red, respectively. Red asterisk denotes 10 nt DNA replacement which retains genome editing activity. (D) HEK293T cells described above were incubated with the tracrRNA and a GFP-targeting crRNA illustrated. TIDE analysis was performed to calculate the % of indels at GFP locus at day 3. n=9 biologically independent samples. *, P<0.01 by One-Way ANOVA with Tukey post hoc test. Sequences shown in FIG. 1A are: GFP crRNA (42 bp) (SEQ ID NO: 55); tracrRNA (SEQ ID NO: 140); Genomic Target DNA 5′ (SEQ ID NO: 146); Genomic Target DNA 3′ (SEQ ID NO: 147). Sequences shown in FIG. 1C are: Native crRNA, 2 DNA, 4 DNA, 6 DNA, 8 DNA, 10 DNA (SEQ ID NO: 32); 12 DNA (SEQ ID NO: 148); 14 DNA (SEQ ID NO: 149); 16 DNA, 18 DNA, 20 DNA (SEQ ID NO: 150).

FIG. 2A-2F shows that partial DNA replacement at the guide region of crRNA or sgRNA induced efficient gene editing in human cells. (A) Partial DNA replacement at the guide region of a crRNA targeting EMX1 induced indels in human cells. HEK293T cells described above were incubated with the tracrRNA and an EMX1-targeting crRNA. TIDE analysis was performed to determine indels at EMX1 locus. n=3 biologically independent samples. *, P<0.01 by One-Way ANOVA with Tukey post hoc test. (B) sgRNAs targeting GFP, EMX1 or VEGFA with 8 nt DNA and 10 nt replacement at 5′ end (sgRNA-8D and sgRNA-10D) induced indels in HEK293T cell. HEK293T cells described above were incubated with a native sgRNA or a sgRNA with 8 DNA nucleotides at 5′ end. TIDE analysis was performed to determine indels. n=3 biologically independent samples. (C) DNA-RNA chimeric crRNAs or sgRNA can mediate efficient genome editing in a RNP setting. VEGFA crRNA+tracrRNA or sgRNA were incubated with SpCas9 protein to form RNP complexes and electroporated into Jurkat T cells by Neon transfection. Genomic DNA was harvested at day 3 and indel % was measured by TIDE. n=3 biologically independent samples. (D) A DNA-RNA chimeric crRNA guides AsCpf1 for efficient genome editing. HEK293T cells were co-transfected with a plasmid expressing AsCpf1 and a native crRNA or crRNA with 8 nt DNA replacement at the 3′ end targeting DNMT1. TIDE analysis was performed to determine indels at DNMT1 locus 3 days after transfection. n=3 biologically independent samples. (e-f) 4 nt DNA replacement at the 3′ end of guide sequence, 4 nt mutation of DNA (DNA mut-1 or 2, in yellow) at 5′ end, or 4 nt mutation of RNA (RNA mut, in blue) abolished activity of CRISPR in human cells. (E) Illustration of DNA replacement at 3′ end or mutations. (f) HEK293T cells described above were incubated with the tracrRNA and a GFP-targeting crRNA as in e. *, P<0.01. n=6 biologically independent samples. Error bars, mean±s.d. Sequences shown in FIG. 2E are Native crRNA, 8 DNA (5′), 4 DNA (3′) (SEQ ID NO: 32); DNA mut-1 (SEQ ID NO: 151); DNA mut-2 (SEQ ID NO: 152); RNA mut (SEQ ID NO: 153).

FIG. 3A-3G shows that partial DNA replacement at the guide region reduced off-target effect in human cells. (A) Illustration of DNA replacement at the guide sequence of VEGFA crRNA. Arrows denote mismatches between target and off-target sites. (B-C) HEK293T cells described in FIG. 1 were incubated with the tracrRNA and a VEGFA-targeting crRNA with 10 DNA nucleotides replacement at the 5′ end of the guide sequence (10 DNA) or native crRNA. Surveyor assay were performed to determine indels at b) VEGFA locus and (C) 3 top off-target sites of VEGFA guide sequence. n=3 biologically independent samples. *, P<0.01 by One-Way ANOVA with Tukey post hoc test. (D-E) Partial DNA replacement in GFP crRNA reduced off-target activity. (D) Illustration of mismatch mutations of GFP2 sequences. Arrows denote point mutations. (E) TIDE analysis was performed to determine indels. n=3 biologically independent samples. *, P<0.05 by student's t-tests. (F) GUIDE-Seq genome-wide off-target analysis of native and 10 DNA crRNAs of three endogenous genes. The chart indicates the number of off-target peaks detected by GUIDE-Seq for each type of crRNA. 6 total mismatches are allowed in the guide and PAM. (G) Number of GUIDE-Seq reads of 293 site 4. Target is the crRNA target site. OT1-OT6 are top off-target sites in the native crRNA dataset. Error bars, mean±s.d. Sequences shown in FIG. 3A are: Native crRNA (SEQ ID NO: 154); 10 DNA (SEQ ID NO: 155); OT1 (SEQ ID NO: 156); OT2 (SEQ ID NO: 157); OT3 (SEQ ID NO: 158). Sequences shown in FIG. 3D are: Native crRNA, 10 DNA (SEQ ID NO: 159); RNA mut-1, 10 DNA-mut 1 (SEQ ID NO: 160); RNA mut-2, 10 DNA-mut 2 (SEQ ID NO: 161).

FIG. 4A-4B shows that an optimized DNA-RNA chimeric crRNA enables efficient genome editing in human cells. (A) Illustration of DNA substitution of GFP targeting crRNAs. RNA and DNA are shown in black and red, respectively. Cas9 binding region is shown in blue box. (B) U2OS-GFP-PEST cells stably expressing Cas9 were transfected with GFP crRNAs and the tracrRNA. GFP negative cells caused by Cas9-mediated frame shift NHEJ were measured by FACS at day 3. 8DNA16DNA design (8 nt DNA in 5′ and 16 nt DNA in 3′, avoiding Cas9 binding region) mediates efficient genome editing. 3′ all DNA (22DNA) abolished genome editing. n=3 biologically independent samples. Error bars, mean±s.d. Sequences shown in FIG. 4A are Native crRNA, 8 DNA (SEQ ID NO: 162); 22 DNA-3′ (SEQ ID NO: 162); 16 DNA-3′, 8DNA16DNA (SEQ ID NO: 164).

FIG. 5A-5B shows that partial DNA replacement at the 5′ end of guide sequence of a crRNA induced gene editing in human cells. HEK293T cells stably expressing both EFs promoter-SpCas9 and EF1a promoter-GFP were transfected with a crRNA targeting GFP and tracrRNA. Percentage of GFP negative cells was determined by FACS analysis. (A) Percentage of GFP negative cells determined by FACS analysis. n=3 biologically independent samples. Error bars, mean±s.d. (B) Representative FACS plots.

FIG. 6 shows that partial DNA replacement at the 5′ end of guide sequence of a crRNA targeting EMX1 induced indels in human cells. HEK293T cells stably expressing SpCas9 were transfected with tracrRNA and an EMX1-targeting crRNA. Surveyor assay were performed to determine indels at EMX1 locus. Arrowheads indicate surveyor nuclease cleaved fragments of the EMX1 PCR product. These experiments were repeated three times independently with similar results.

FIG. 7A-7B shows that partial DNA replacement at the 5′ end of guide sequence of a crRNA targeting VEGFA efficiently reduced off-target activity in human cells. Surveyor assay were performed to determine indels at (A) VEGFA locus and (B) 3 top off-target sites of VEGFA guide sequence. Red arrowheads indicate surveyor nuclease cleaved fragments of PCR products of the VEGFA or off-target sites. These experiments were repeated three times independently with similar results.

FIG. 8 shows that partial DNA replacement at the 5′ end of guide sequence of a crRNA and truncated crRNA targeting VEGFA efficiently reduced off-target activity in human cells. Surveyor assay were performed to determine indels at 2 top off-target sites of VEGFA guide sequence. Red arrowheads indicate surveyor nuclease cleaved fragments of 2 PCR products of the off-target sites. These experiments were repeated three times independently with similar results.

FIG. 9A-9B shows that native crRNA, but not the 8 DNA crRNA, tolerates single nucleotide mismatch. (A) Illustration of DNA replacement and mismatches at the 20 nt guide region of GFP crRNAs. RNA, DNA, and mismatch are shown in black, red and underlined, respectively. (B) HEK293T-GFP-SpCas9 cells were transfected with tracrRNA and crRNAs in (A). TIDE analysis was performed at day 3 to calculate the % of indel at GFP locus. n=3 biologically independent samples. Error bars, mean±s.d. Sequences shown in FIG. 9A are: GFP 8D crRNA (SEQ ID NO: 165); 3 nt DNA mismatch (SEQ ID NO: 166); 3 nt RNA (SEQ ID NO: 167); 2 nt DNA, 2 nt RNA (SEQ ID NO: 168); 1 nt DNA, 1 nt RNA (SEQ ID NO: 169).

FIG. 10A-10B shows that an optimized DNA-RNA chimeric crRNA enables efficient genome editing in human cells and significantly reduces cost. (A) DNA % in crRNA design in FIG. 4A. (B) 8DNA16DNA significantly reduces synthesis cost. Cost shown is for 100 nMole custom RNA or DNA-RNA chimeric oligos ordered from IDT.

### DETAILED DESCRIPTION

The disclosure relates to the Clustered Regularly Interspaced Short Palindromic Repeats (CRISPR) and CRISPR associated (Cas) proteins (CRISPR/Cas) system to drive both non-homologous end joining (NHEJ) based gene disruption and homology directed repair (HDR) based precise gene editing to achieve highly efficient and simultaneous targeting of multiple nucleic acid sequences in cells and nonhuman mammals.

International applications WO2017/004279 and WO2017/136794 are incorporated by reference herein in their entirety.

The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” one of,” “only one of,” or “exactly one of” “Consisting essentially of,” when used in the claims, shall have its ordinary meaning as used in the field of patent law.

As used herein, “activity” in the context of CRISPR/Cas activity, Cas protein activity, Cas9 activity, sgRNA activity, sgRNA:nuclease activity and the like refers to the ability of a nucleic acid and/or protein to bind to a target sequence and/or label or cleave the target sequence. Such activity can be measured in a variety of ways as known in the art. For example, expression, activity, or level of a reporter gene can be measured, and sgRNA:nucleases targeting the reporter gene sequence can be assayed for their ability to reduce the expression, activity, or level of the reporter gene. For example, a cell can be transfected with an expression cassette encoding a green fluorescent protein under the control of a constitutive promoter. The fluorescence intensity can be measured and compared to the intensity of the cell after transfection with Cas9 and candidate sgRNAs to identify optimized sgRNAs.

The term “analog” as used herein refers to compounds that are similar but not identical in chemical formula and share the same or substantial function of the compound with the similar chemical formula.

The terms “biophysically effective amount” refers to an amount of nucleic acid in a system under physiological conditions (such as temperature, pH, exposure to percent oxygen, etc.) sufficient to associate to or bind a Cas protein or functional fragment thereof in the presence of a Cas protein or functional fragment thereof. In some embodiments, the nucleic acid is a sgRNA, or a crRNA/tracr RNA duplex. In some embodiments, the Cas protein or functional fragment thereof is chosen from any of the sequences of Tables D or E or functional fragments thereof.

As used herein, “conservative” amino acid substitutions may be defined as set out in Tables A, B, or C below. The polypeptides of the disclosure include those wherein conservative substitutions (from either nucleic acid or amino acid sequences) have been introduced by modification of polynucleotides encoding polypeptides. In some embodiments, these polypeptides comprise or consist or enzymes (such as those enzymes capable to forming a complex with one or a plurality of sgRNA sequences) or functional fragments thereof. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has a binding affinity to a target or binding partner similar to the binding affinity of the sequence upon which the conservative substitution is based. Exemplary conservative substitutions are set out in Table 1A.

TABLE 1A
Conservative Substitutions I

| Side Chain Characteristics | Amino Acid |
|---|---|
| Aliphatic | |
| Non-polar | G A P I L V F |
| Polar - uncharged | C S T M N Q |
| Polar - charged | D E K R |
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 1B.

TABLE 1B
Conservative Substitutions II

| Side Chain Characteristic | Amino Acid |
|---|---|
| Non-polar (hydrophobic) | |
| Aliphatic: | A L I V P |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

Alternately, exemplary conservative substitutions are set out in Table 2.

TABLE 2
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the enzymes (such as the Cas9 enzyme) or any functional fragments thereof described herein are intended to include amino acid sequences comprising polypeptides bearing one or more insertions, deletions, or substitutions, or any combination thereof, of amino acid residues as well as modifications other than insertions, deletions, or substitutions of amino acid residues, such as but not limited to conservative amino acid substitutions.

“Cas binding domain” refers to a nucleic acid element or domain within a nucleic acid sequence or polynucleotide sequence that, in a biophysically effective amount, will bind or have an affinity for one or a plurality of proteins (or functional fragments thereof) encoded by one or a plurality of CRISPR-associated genes. In some embodiments, in the presence of a the one or a plurality of proteins (or functional fragments thereof) and a target sequence, the one or plurality of proteins and the nucleic acid element forms a biologically active CRISPR complex and/or can be enzymatically active on a target sequence. The terms “CRISPR-associated genes” refer to any nucleic acid that encodes a regulatory or expressible gene that regulates a component or encodes a component of the CRISPR system. In some embodiments, the terms “CRISPR-associated genes” refer to any nucleic acid sequence that encodes any of the proteins in Table 3 or Table 13 (or functional fragments or variants thereof that are at least about 70, 75, 80, 85, 90, 95, 96, 97, 98, or 99% homologous to the sequences disclosed in either Table). In some embodiments, the terms “Cas-binding domain” or “Cas protein-binding domain” refers to a nucleic acid element or domain within a nucleic acid sequence or polynucleotide sequence that, in a biophysically effective amount, will bind to or have an affinity for one or a plurality of proteins in Table 3 or Table 13 (or functional fragments or variants thereof that are at least about 70, 75, 80, 85, 90, 95, 96, 97, 98, or 99% homologous to the sequences disclosed in either Table). In some embodiments, the Cas binding domain consists of no more than about 10, 11, 12, 13, 14, 15, 16, 17 18, 19, 20, 25, 30, 35, 40, 41, 42, 43, 44, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active CRISPR system at a concentration and within microenvironment suitable for CRISPR system formation. In some embodiments, the composition or pharmaceutical compositions comprises one or a combination of sgRNA, crRNA, and/or tracrRNA that consists of no more than about 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially associates or binds to a biologically active amino acid sequence (or functional fragment disclosed herein) disclosed in Table 13 at a concentration and within microenvironment suitable for CRISPR system formation and CRISPR enzymatic activity on a target sequence. In some embodiments, the Cas protein derived from the Cas9 family of Cas proteins or a functional fragment thereof.

The terms “transcription terminator domain” refers to a nucleic acid element or domain within a nucleic acid sequence (or polynucleotide sequence) that, in a biophysically effective amount, prevents bacterial transcription when the CRISPR complex is in a bacterial species and/or creates a secondary structure that stabilizes the association of the nucleic acid sequence to one or a plurality of Cas proteins (or functional fragments thereof) encoded by one or a plurality of CRISPR-associated genes such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of no more than about 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

The terms “DNA-binding domain” refer to an element or refers to a nucleic acid element or domain within a nucleic acid sequence or sgRNA that is complementary to a target sequence. In some embodiments, in a biophysically effective amount upstream from a Cas-binding domain, the DNA-binding domain will bind or have an affinity for one or a plurality of target nucleic acid sequences such that, in the presence of a biologically active CRISPR complex, one or plurality of Cas proteins can be enzymatically active on the target sequence. In some embodiments, the DNA binding domain consists of no more than about 20, 25, 30, 35, 40, 45, 50, 60, 70, 80, 90, 100, 110, 120, 130, 140, 150, 160, 170, 180, 190, 200, 210, 220, 230, 240, 250 or more nucleotides in length and comprises at least one sequence that is capable of forming Watson Crick basepairs with a target sequence as part of a biologically active CRISPR system at a concentration and microenvironment suitable for CRISPR system formation.

“CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is an guanine and N is any naturally occurring nucleic acid. In some embodiments the target DNA comprises any one or combination of NNG, NNA, GAA, NNAGAAW and NGGNG, where G is an guanine, A is adenine, and N is any naturally occurring nucleic acid

In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the disclosure, an exogenous template polynucleotide may be referred to as an editing template. In an aspect of the disclosure the recombination is homologous recombination. In some embodiments, a composition disclosed herein comprises a recombination template. A recombination template may be a component of another vector as described herein, contained in a separate vector, or provided as a separate polynucleotide. In some embodiments, a recombination template is designed to serve as a template in homologous recombination, such as within or near a target sequence nicked or cleaved by a CRISPR enzyme (or equivalently a “Cas protein”) as a part of a CRISPR complex. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a type II Cas9 protein. In some embodiments, the chemically synthesized guide sequence is used in conjunction with one or more vectors, wherein each vector comprises a coding sequence that encodes a CRISPR enzyme, such as a type II Cas9 protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, one or more additional guide sequence, tracr mate sequence, and/or tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid form a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

One or a plurality of vectors may also be components in any system or composition provided herein. In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression, the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple, different guide sequences are used, a single expression construct may be used to target CRISPR activity to multiple, different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell. The disclosure relates to any composition comprising any of the aforementioned elements and one or more artificially synthesized guide sgRNA described herein.

Another aspect of the disclosure relates to a CRISPR system comprising a modified CRISPR enzyme (or “Cas protein”) or a nucleotide sequence encoding one or more Cas proteins. Any protein capable of enzymatic activity in cooperation with a guide sequence is a Cas protein. In some embodiments, the disclosure relates to a system comprises a vector comprising a regulatory element operably linked to an enzyme-coding sequence encoding a CRISPR enzyme, such as a Cas protein from the Cas family of enzymes. In some embodiments, the disclosure relates to a system, composition, or pharmaceutical composition comprising any one or plurality of Cas proteins either individually or in combination with one or a plurality of guide sequences. Compositions of one or a plurality of Cas proteins may be administered to a subject with any of the disclosed guide sequences sequentially or contemporaneously. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, type V CRISPR-Cas systems, variants and fragments thereof, or modified versions thereof having at least 70% homology to the sequences of Table 13, wherein are incorporated by reference in their entireties. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme or Cas protein that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase may be used in combination with guide sequenc(es), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

In some embodiments, the composition of the disclosure comprise an amino acid sequence at least about 70%, 80%, 90%, 95%, 96%, 97%, 98%, 99%, or 100% homolgous to Cas9 below:

(SEQ ID NO: 34)

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In some embodiments, an enzyme coding sequence encoding a CRISPR enzyme is codon optimized for expression in particular cells, such as eukaryotic cells. The eukaryotic cells may be those of or derived from a particular organism or a particular subject, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

TABLE 3
CRISPR enzymes (Cas proteins or Cas-like proteins) organized by Family

| | | | | Structure of | | |
|---|---|---|---|---|---|---|
| | | | Name | encoded | Families (and | |
| | | Name | from | protein | superfamily) | |
| Proposed | System type or | from Haft | Brouns | (PDB | of encoded | |
| gene name‡ | subtype | et al.^{§} | et al.^{\|\|} | accessions)^{¶} | protein^{#}** | Representatives |
| cas1 | Type I | cas1 | cas1 | 3GOD, 3LFX | COG1518 | SERP2463, |
| | Type II | | | and 2YZS | | SPy1047 and |
| | Type III | | | | | ygbT |
| cas2 | Type I | cas2 | cas1 | 2IVY, 2I8E | COG1343 | SERP2462, |
| | Type II | | | and 3EXC | and | SPy1048, |
| | Type III | | | | COG3512 | SPy1723 (N- |
| | | | | | | terminal |
| | | | | | | domain) and |
| | | | | | | ygbF |
| cas3′ | Type I^{‡‡} | cas3 | cas3 | NA | COG1203 | APE1232 and |
| | | | | | | ygcB |
| cas3″ | Subtype I-A | NA | NA | NA | COG2254 | APE1231 and |
| | Subtype I-B | | | | | BH0336 |
| cas4 | Subtype I-A | cas4 and | NA | NA | COG1468 | APE1239 and |
| | Subtype I-B | csa1 | | | | BH0340 |
| | Subtype I-C | | | | | |
| | Subtype I-D | | | | | |
| | Subtype II-B | | | | | |
| cas5 | Subtype I-A | cas5a, | casD | 3KG4 | COG1688 | APE1234, |
| | Subtype I-B | cas5d, | | | (RAMP) | BH0337, devS |
| | Subtype I-C | cas5e, | | | | and ygcl |
| | Subtype I-E | cas5h, | | | | |
| | | cas5p, | | | | |
| | | cas5t and | | | | |
| | | cmx5 | | | | |
| cas6 | Subtype I-A | cas6 and | NA | 3I4H | COG1583 | PF1131 and |
| | Subtype I-B | cmx6 | | | and | slr7014 |
| | Subtype I-D | | | | COG5551 | |
| | Subtype III-A | | | | (RAMP) | |
| | Subtype III-B | | | | | |
| cas6e | Subtype I-E | cse3 | casE | 1WJ9 | (RAMP) | ygcH |
| cas6f | Subtype I-F | csy4 | NA | 2XLI | (RAMP) | y1727 |
| cas7 | Subtype I-A | csa2, | casC | NA | COG1857 and | devR and ygcJ |
| | Subtype I-B | csd2, | | | COG3649 | |
| | Subtype I-C | cse4, | | | (RAMP) | |
| | Subtype I-E | csh2, csp1 | | | | |
| | | and cst2 | | | | |
| cas8a1 | Subtype I-A^{‡‡} | cmx1, | NA | NA | BH0338-like | LA3191^{§§} and |
| | | cst1, csx8, | | | | PG2018^{§§} |
| | | csx13 and | | | | |
| | | CXXC- | | | | |
| | | CXXC | | | | |
| cas8a2 | Subtype I-A^{‡‡} | csa4 and | NA | NA | PH0918 | AF0070, AF1873, |
| | | csx9 | | | | MJ0385, PF0637, |
| | | | | | | PH0918 and |
| | | | | | | SSO1401 |
| cas8b | Subtype I-B^{‡‡} | csh1 and | NA | NA | BH0338-like | MTH1090 and |
| | | TM1802 | | | | TM1802 |
| cas8c | Subtype I-C^{‡‡} | csd1 and | NA | NA | BH0338-like | BH0338 |
| | | csp2 | | | | |
| cas9 | Type II^{‡‡} | csn1 and | NA | NA | COG3513 | FTN_0757 and |
| | | csx12 | | | | SPy1046 |
| cas10 | Type III^{‡‡} | cmr2, | NA | NA | COG1353 | MTH326, |
| | | csm1 and | | | | Rv2823c§§ and |
| | | csx11 | | | | TM1794§§ |
| cas10d | Subtype I-D^{‡‡} | csc3 | NA | NA | COG1353 | slr7011 |
| csy1 | Subtype I-F^{‡‡} | csy1 | NA | NA | y1724-like | y1724 |
| csy2 | Subtype I-F | csy2 | NA | NA | (RAMP) | y1725 |
| csy3 | Subtype I-F | csy3 | NA | NA | (RAMP) | y1726 |
| cse1 | Subtype I-E^{‡‡} | cse1 | casA | NA | YgcL-like | ygcL |
| cse2 | Subtype I-E | cse2 | casB | 2ZCA | YgcK-like | ygcK |
| csc1 | Subtype I-D | csc1 | NA | NA | alr1563-like | alr1563 |
| | | | | | (RAMP) | |
| csc2 | Subtype I-D | csc1 and | NA | NA | COG1337 | slr7012 |
| | | csc2 | | | (RAMP) | |
| csa5 | Subtype I-A | csa5 | NA | NA | AF1870 | AF1870, |
| | | | | | | MJ0380, PF0643 |
| | | | | | | and SSO1398 |
| csn2 | Subtype II-A | csn2 | NA | NA | SPy1049-like | SPy1049 |
| csm2 | Subtype III-A^{‡‡} | csm2 | NA | NA | COG1421 | MTH1081 and |
| | | | | | | SERP2460 |
| csm3 | Subtype III-A | csc2 and | NA | NA | COG1337 | MTH1080 and |
| | | csm3 | | | (RAMP) | SERP2459 |
| csm4 | Subtype III-A | csm4 | NA | NA | COG1567 | MTH1079 and |
| | | | | | (RAMP) | SERP2458 |
| csm5 | Subtype III-A | csm5 | NA | NA | COG1332 | MTH1078 and |
| | | | | | (RAMP) | SERP2457 |
| csm6 | Subtype III-A | APE2256 | NA | 2WTE | COG1517 | APE2256 and |
| | | and csm6 | | | | SSO1445 |
| cmr1 | Subtype III-B | cmr1 | NA | NA | COG1367 | PF1130 |
| | | | | | (RAMP) | |
| cmr3 | Subtype III-B | cmr3 | NA | NA | COG1769 | PF1128 |
| | | | | | (RAMP) | |
| cmr4 | Subtype III-B | cmr4 | NA | NA | COG1336 | PF1126 |
| | | | | | (RAMP) | |
| cmr5 | Subtype III-B^{‡‡} | cmr5 | NA | 2ZOP and | COG3337 | MTH324 and |
| | | | | 2OEB | | PF1125 |
| cmr6 | Subtype III-B | cmr6 | NA | NA | COG1604 | PF1124 |
| | | | | | (RAMP) | |
| csb1 | Subtype I-U | GSU0053 | NA | NA | (RAMP) | Balac_1306 and |
| | | | | | | GSU0053 |
| csb2 | Subtype I-U^{§§} | NA | NA | NA | (RAMP) | Balac_1305 and |
| | | | | | | GSU0054 |
| csb3 | Subtype I-U | NA | NA | NA | (RAMP) | Balac_1303^{§§} |
| csx17 | Subtype I-U | NA | NA | NA | NA | Btus_2683 |
| csx14 | Subtype I-U | NA | NA | NA | NA | GSU0052 |
| csx10 | Subtype I-U | csx10 | NA | NA | (RAMP) | Caur_2274 |
| csx16 | Subtype III-U | WA1548 | NA | NA | NA | VVA1548 |
| csaX | Subtype III-U | csaX | NA | NA | NA | SSO1438 |
| csx3 | Subtype III-U | csx3 | NA | NA | NA | AF1864 |
| csx1 | Subtype III-U | csa3, | NA | 1XMX and | COG1517 | MJ1666, |
| | | csx1, csx2, | | 2I71 | and | NE0113, PF1127 |
| | | DXTHG, | | | COG4006 | and TM1812 |
| | | NE0113 | | | | |
| | | and | | | | |
| | | TIGR02710 | | | | |
| csx15 | Unknown | NA | NA | NA | TTE2665 | TTE2665 |

In some embodiments, a vector encodes a CRISPR enzyme comprising one or more nuclear localization sequences (NLSs), such as about (or more than about) 1, 2, 3, 4, 5, 6, 7, 8, 9, 10, or more NLSs. In some embodiments, the CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In a preferred embodiment of the disclosure, the CRISPR enzyme comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Typically, an NLS consists of one or more short sequences of positively charged lysines or arginines exposed on the protein surface, but other types of NLS are known. Non-limiting examples of NLSs include an NLS sequence derived from: the NLS of the SV40 virus large T-antigen, having the amino acid sequence PKKKRKV (SEQ ID NO: 75); the NLS from nucleoplasmin (e.g. the nucleoplasmin bipartite NLS with the sequence KRPAATKKAGQAKKKK (SEQ ID NO:76)); the c-myc NLS having the amino acid sequence PAAKRVKLD (SEQ ID NO:77) or RQRRNELKRSP (SEQ ID NO:78); the hRNPAI M9 NLS having the sequence NQSSNFGPMKGGNFGGRSSGPYGGGGQYFAKPRNQGGY (SEQ ID NO:79); the sequence RMRIZFKNKGKDTAELRRRRVEVSVELRKAKKDEQILKRRNV (SEQ ID NO:80) of the IBB domain from importin-alpha; the sequences VSRKRPRP (SEQ ID NO:81) and PPKKARED (SEQ ID NO:82) of the myoma T protein; the sequence PQPKKKPL (SEQ ID NO:83) of human p53; the sequence SALIKKKKKMAP (SEQ ID NO:84) of mouse c-abl IV; the sequences DRLRR (SEQ ID NO:33) and PKQKKRK (SEQ ID NO:15) of the influenza virus NS1; the sequence RKLKKKIKKL (SEQ ID NO:16) of the Hepatitis virus delta antigen; the sequence REKKKFLKRR (SEQ ID NO:17) of the mouse Mx protein; the sequence KRKGDEVDGVDEVAKKKSKK (SEQ ID NO:18) of the human poly(ADP-ribose) polymerase; and the sequence RKCLQAGMNLEARKTKK (SEQ ID NO:19) of the steroid hormone receptors (human) glucocorticoid. In general, the one or more NLSs are of sufficient strength to drive accumulation of the CRISPR enzyme in a detectable amount in the nucleus of a eukaryotic cell. Strength of nuclear localization activity may derive from the number of NLSs in the CRISPR enzyme, the particular NLS(s) used, or a combination of these factors. Detection of accumulation in the nucleus may be performed by any suitable technique. For example, a detectable marker may be fused to the CRISPR enzyme, such that location within a cell may be visualized, such as in combination with a means for detecting the location of the nucleus (e.g. a stain specific for the nucleus such as DAPI). Examples of detectable markers include fluorescent proteins (such as Green fluorescent proteins, or GFP; RFP; CFP), and epitope tags (HA tag, flag tag, SNAP tag). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence homology to the wild-type sequence upon which the sequence is derived.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The present disclosure also relates to isotopically-enriched compounds, which are structurally similar to the nucleic acid sequences disclosed herein, but for the fact that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorous, fluorine and chlorine, such as ²H, ³H, ¹³C, ¹⁴C, ¹⁵N, ¹⁶O, ¹⁷O, ³¹P, ³²P, ³⁵S, ¹⁸F, and ³⁶Cl. Nucleic acids of the present disclosures that contain the aforementioned isotopes and/or other isotopes of other atoms are within the scope of this disclosure. Certain isotopically-labelled compounds of the present disclosure, for example those into which radioactive isotopes such as 3H and 14C are incorporated, are useful in drug and/or substrate tissue distribution assays. Tritiated, i.e., 3H, and carbon-14, i.e., 14C, isotopes are particularly preferred for their ease of preparation and detection. Further, substitution with heavier isotopes such as deuterium, i.e., 2H, can afford certain therapeutic advantages resulting from greater metabolic stability, for example increased in vivo half-life or reduced dosage requirements and, hence, may be preferred in some circumstances. Isotopically enriched compounds of this disclosure can generally be prepared by substituting a readily available isotopically labeled reagent for a non-isotopically enriched reagent. The disclosure relates to nucleic acids disclosed herein unsolvated forms as well as solvated forms, including hydrated forms. The compounds of the disclosure also are capable of forming both pharmaceutically acceptable salts, including but not limited to acid addition and/or base addition salts. Furthermore, compounds of the present disclosure may exist in various solid states including an amorphous form (noncrystalline form), and in the form of clathrates, prodrugs, polymorphs, bio-hydrolyzable esters, racemic mixtures, non-racemic mixtures, or as purified stereoisomers including, but not limited to, optically pure enantiomers and diastereomers. In general, all of these forms can be used as an alternative form to the free base or free acid forms of the compounds, as described above and are intended to be encompassed within the scope of the present disclosure.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside. In some embodiments, the nucleotide is characterized as being modified if the 3′ phosphate group is covalently linked to a contiguous nucleotide by any linkage other than a phosphodiester bond.

“Compound comprising a modified oligonucleotide consisting of a number of linked nucleosides means a compound that includes a modified oligonucleotide having the specified number of linked nucleosides. Thus, the compound may include additional substituents or conjugates. Unless otherwise indicated, the compound does not include any additional nucleosides beyond those of the modified oligonucleotide.

“Modified oligonucleotide” means an oligonucleotide having one or more modifications relative to a naturally occurring terminus, sugar, nucleobase, and/or internucleoside linkage. A modified oligonucleotide may comprise unmodified nucleosides.

“Single-stranded modified oligonucleotide” means a modified oligonucleotide which is not hybridized to a complementary strand.

“Modified nucleoside” means a nucleoside having any change from a naturally occurring nucleoside. A modified nucleoside may have a modified sugar, and an unmodified nucleobase. A modified nucleoside may have a modified sugar and a modified nucleobase. A modified nucleoside may have a natural sugar and a modified nucleobase. In certain embodiments, a modified nucleoside is a bicyclic nucleoside. In certain embodiments, a modified nucleoside is a non-bicyclic nucleoside.

A “polymorph” refers to solid crystalline forms of a compound. In some embodiments, one or more nucleic acids disclosed herein are in polymorph form. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Different physical properties of polymorphs can affect their processing.

The guide sequences, nucleic acid sequences, proteins or other agents of the present disclosure can be administered, inter alia, as pharmaceutically acceptable salts, esters, or amides. The term “salts” refers to inorganic and organic salts of compounds of the present disclosure. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977).

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after synthesis or polymerization, such as by conjugation with a labeling component.

The oligonucleotides of the disclosure also include those nucleic acid sequences disclosed herein that comprise nucleosides connected by charged linkages, and/or whose sequences are divided into at least two subsequences. In some embodiments, a first, second, and third subsequence or domains include a nucleotide binding domain (or DNA-binding domain), a Cas-binding domain, and a transcription terminator domain. In some embodiments, a first, second, and third subsequence or domains include a nucleotide binding domain, a Cas-binding domain, and a transcription terminator sequence, but, if any two domains are present the they must be oriented such that the nucleotide binding domain precedes the Cas-binding domain which, in turn precedes the transcription terminator domain in a 5′ to 3′ orientation. Any of the nucleosides within any of the domains may be 2′-substituted-nucleosides linked by a first type of linkage. The second subsequence includes nucleosides linked by a second type of linkage. In some embodiments, there exists a third subsequence whose nucleosides are selected from those selectable for the first subsequence, and the second subsequence is positioned between the first and the third subsequences. Such oligonucleotides of the disclosure are known as “chimeras,” or “chimeric” or “gapped” oligonucleotides.

In the context of this disclosure, the term “oligonucleotide” also refers to a plurality of nucleotides joined together in a specific sequence from naturally and non-naturally occurring nucleobases. Nucleobases of the disclosure are joined through a sugar moiety via phosphorus linkages, and include any one or combination of adenine, guanine, cytosine, uracil, thymine, xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, other aza and deaza thymidines, other aza and deaza cytosines, other aza and deaza adenines, other aza and deaza guanines, 5-trifluoromethyl uracil and 5-trifluoro cytosine. The sugar moiety may be deoxyribose or ribose. The sugar moiety may be a modified deoxyribose or ribose with one or more modifications on the C₁, C₂, C₃, C₄, and/or C₅ carbons. The oligonucleotides of the disclosure may also comprise modified nucleobases or nucleobases having other modifications consistent with the spirit of this disclosure, and in particular modifications that increase their nuclease resistance in order to facilitate their use as therapeutic, diagnostic or research reagents.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, “more than one” or “two or more” 2, 3, 4, 5, 6, 7, 8, 9, or 10 or more. In some embodiments, “more than one” means 2, 3, 4, or 5 of the amino acids or nucleic acids or mutations described herein. In some embodiments, “more than one” means 2, 3, or 4 of the amino acids or nucleic acids or mutations described herein. In some embodiments, “more than one” means 2 or 3 of the amino acids or nucleic acids or mutations described herein. In some embodiments, “more than one” means 2 of the amino acids or nucleic acids or mutations described herein.

“Sugar moiety” means a naturally occurring furanosyl or a modified sugar moiety.

“Modified sugar moiety” means a substituted sugar moiety or a sugar surrogate.

“Substituted sugar moiety” means a furanosyl that is not a naturally occurring furanosyl. Substituted sugar moieties include, but are not limited to sugar moieties comprising modifications at the 2′-position, the 5′-position and/or the 4′-position of a naturally occurring furanosyl. Certain substituted sugar moieties are bicyclic sugar moieties.

“Sugar surrogate” means a structure that does not comprise a furanosyl and that is capable of replacing the naturally occurring furanosyl of a nucleoside, such that the resulting nucleoside is capable of (1) incorporation into an oligonucleotide and (2) hybridization to a complementary nucleoside. Such structures include relatively simple changes to the furanosyl, such as rings comprising a different number of atoms (e.g., 4, 6, or 7-membered rings); replacement of the oxygen of the furanosyl with a non-oxygen atom (e.g., carbon, sulfur, or nitrogen); or both a change in the number of atoms and a replacement of the oxygen. Such structures may also comprise substitutions corresponding with those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

The terms “therapeutically effective amount” mean a quantity sufficient to achieve a desired therapeutic and/or prophylactic effect, for example, an amount which results in the prevention or amelioration of or a decrease in the symptoms associated with a disease that is being treated. The amount of composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The regimen of administration can affect what constitutes an effective amount. The compound of the disclosure can be administered to the subject either prior to or after the onset of disease or disorder. Further, several divided dosages, as well as staggered dosages, can be administered daily or sequentially, or the dose can be continuously infused, or can be a bolus injection. Further, the dosages of the compound(s) of the disclosure can be proportionally increased or decreased as indicated by the exigencies of the therapeutic or prophylactic situation. Typically, an effective amount of the compounds of the present disclosure, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. A therapeutically effective amount of a pharmaceutical composition comprising any one or a plurality of any of the guide sequences disclosed herein (and, optionally, any nucleic acid sequence encoding a Cas protein of the present disclosure) can also be administered in combination with each other, or with one or more additional therapeutic compounds. Those skilled in the art will recognize and determine a therapeutically effective amount of any of the guide sequences disclosed herein whether calculated when administered alone or part of a therapeutic regimen that includes one or more other beta-catenin nuclear translocation inhibitors and/or one or more one or more other therapeutic agents and/or one or more other therapeutic treatments or interventions. Generally, therapeutically effective amount refers to an amount of a guide sequence (such as an sgRNA) that, in combination with one or a plurality of CRISPR system components causes a mutation in a target sequence sufficient to ameliorate symptoms, or reverse, prevent or reduce the rate of progress of disease, or extend life span of a subject when administered alone or in combination with other therapeutic agents or treatments as compared to the symptoms, rate of progress of disease, or life span of an individual not receiving a therapeutically effective amount an sgRNA disclosed herein. In some embodiments, the therapeutically effective amount thereof is the amount of sgRNA needed to form a CRISPR complex with any disclosed Cas protein and cause the Cas protein within the complex to adequately perform its enzymatic function at or proximate to the target sequence.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include di- and multivalent radicals, having the number of carbon atoms designated (i.e., C₁-C₁₀ means one to ten carbons). Alkyl is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds (e.g. alkene, alkyne). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂—. Typically, an alkyl (or alkylene) group will have from about 1 to about 24 carbon atoms, with those groups having 10 or fewer carbon atoms being preferred in the present disclosure. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. Heteroalkyl is not cyclized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Cycloalkyl and heterocycloalkyl are non-aromatic. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, monophosphate (or derivatives thereof), diphosphate (or derivatives thereof), triphosphate (or derivatives thereof), in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the disclosure includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″″, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the disclosure includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In embodiments, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)q-U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)r-B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)s-X′— (C″R″R′″)d-, where s and d are independently integers of from 0 to 3, and X′ is —O—, —NR—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), and silicon (Si).

A “substituent group,” as used herein, means a group selected from the following moieties: (A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, monophosphate (or derivatives thereof), diphosphate (or derivatives thereof), or triphosphate (or derivatives thereof), and (B) alkyl, heteroalkyl, cycloalkyl, heterocycloalkyl, aryl, heteroaryl, substituted with at least one substituent selected from:

(i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, monophosphate (or derivatives thereof), diphosphate (or derivatives thereof), or triphosphate (or derivatives thereof), substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, —NHSO₂CH₃, —N₃, unsubstituted alkyl, unsubstituted heteroalkyl, unsubstituted cycloalkyl, unsubstituted heterocycloalkyl, unsubstituted aryl, unsubstituted heteroaryl,

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. More specifically, In embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds, compositions and pharmaceutical compositions disclosed herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In embodiments herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In embodiments, the compound is a chemical species set forth in the Examples section below.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that the compositions of this disclosure comprise nucleic acid sequences or molecules with nucleic acids that may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by 13C- or 14C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (125I), or carbon-14 (14C) including the radioisotopes of Table 7. All isotopic variations of the compounds of the present disclosure, whether radioactive or not, are encompassed within the scope of the present disclosure.

The symbol “-” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The symbol “ [symbol] ” denotes the point of attachment of a chemical moiety to the remainder of a molecule or chemical formula. The symbol “ [symbol] ” denotes one or more than one modified or unmodified contiguous nucleotide.

A “base,” as used herein, means a group selected from the following: adenine, guanine, cytosine, uracil, thymine, uridine, pyrimidine, purine, pseudouridine, inosine, hypoxanthine, rhodamine, fluroscein, 2-aminopurine, cytidine, 2′-deoxycytidine, 1,3-Diaza-2-oxophenothiazine, dihydrouridine, queuosine, wyosine, cyanophage S-2L diaminopurine, isoguanine, isocytosine, diaminopyrimidine, 2,4-difluorotoluene, 4-methylbenzimidazole, isoquinoline, pyrrolo[2,3-b]pyridine, 2-amino-6-(2-thienyl)purine, pyrrole-2-carbaldehyde, 2,6-bis(ethylthiomethyl)pyridine, pyridine-2,6-dicarboxamide, 2′-deoxyinosine, 2-amino-8-(2-thienyl)purine, pyridine-2-one, 7-(2-thienyl)imidazo[4,5-b]pyridine, pyrrole-2-carbaldehyde, 4-[3-(6-aminohexanamido)-1-propynyl]-2-nitropyrrole, or modified derivative thereof.

The term “phosphodiester,” by itself or as part of another substituent, means, unless otherwise stated, —O—P(O)₂—O—, wherein the phosphate atom is doubly bonded to one oxygen atom and bound to other substituents through the adjacent oxygen atoms.

The term “LNA,” as used herein, means any nucleic acid analog disclosed herein comprising a cyclic structure between the C2 and C4 carbon of the sugar moiety of a nucleic acid. In some embodiments, the LNA has the structure below:

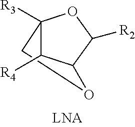

wherein R₂ is independently selected from: any base or nucleobase, adenine, guanine, cytosine, uracil, thymine, uridine, pyrimidine, purine, pseudouridine, inosine, or hypoxanthine;

wherein R₃ is independently selected from a: phosphodiester, phosphorothioate, aldehyde, carboxyl, carbonyl, ether, ester, or amino; wherein R₄ is independently selected from a: phosphodiester, phosphorothioate, aldehyde, carboxyl, carbonyl, ether, ester, or amino;

or a pharmaceutically active salt thereof.

In some embodiments, the present disclosure provides a composition comprising a nucleic acid sequence comprising at least one nucleic acid having Formula W:

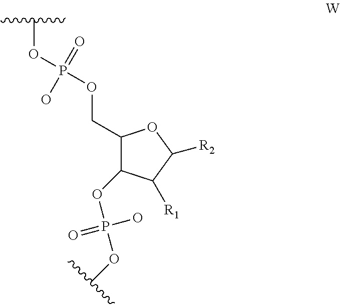

wherein R₁ is independently selected from: hydrogen, hydroxyl, halogen, alkyl, alkenyl, alkynyl, acyl, ester, sulfonyl, amide, amine, alkyloxy, methoxyethyl, or DNP (2,4′-dinitrophenol);

wherein R₂ is independently selected from: hydrogen, hydroxyl, halogen, alkyl, alkenyl, alkynyl, acyl, a base or nucleobase, adenine, guanine, cytosine, uracil, thymine, uridine, pyrimidine, purine, pseudouridine, inosine, or hypoxanthine;

or a pharmaceutically active salt thereof.

In some embodiments, the present disclosure provides a composition comprising a compound having Formula X:

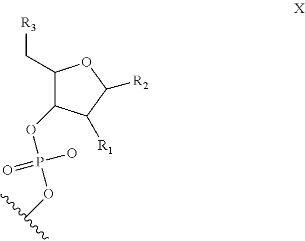

wherein R₁ is independently selected from: hydrogen, hydroxyl, halogen, alkyl, alkenyl, alkynyl, acyl, ester, sulfonyl, amide, amine, alkyloxy, methoxyethyl, or DNP (2,4′-dinitrophenol);

wherein R₂ is independently selected from: hydrogen, hydroxyl, halogen, alkyl, alkenyl, alkynyl, acyl, adenine, guanine, cytosine, uracil, thymine, uridine, pyrimidine, purine, pseudouridine, inosine, or hypoxanthine;

wherein R₃ is independently selected from a: aikylphosphonate, phosphotriester, phosphorodithioate, phosphoramidate, ketone, sulfone, carbonate thioamidate, phosphodiester, phosphorothioate, aldehyde, carboxyl, carbonyl, ether, ester, or amine; wherein, in some embodiments, the alkylphosphonate, phosphotriester, phosphorodithioate, phosphoramidate, ketone, sulfone, carbonate thioamidate, phosphodiester, aldehyde, carboxyl, carbonyl, ether, ester, or amine is bonded to a contiguous nucleic acid or nucleoside, such that the formula reads

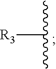

or a pharmaceutically active salt thereof.

In some embodiments, the present disclosure provides a composition comprising a compound having Formula Y:

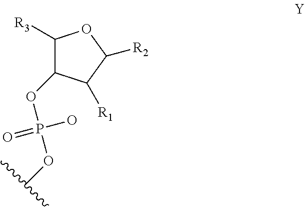

wherein R₁ is independently selected from: hydrogen, hydroxyl, halogen, alkyl, alkenyl, alkynyl, acyl, ester, sulfonyl, amide, amine, alkyloxy, methoxyethyl, or DNP (2,4′-dinitrophenol);

wherein R₂ is independently selected from: hydrogen, hydroxyl, halogen, alkyl, alkenyl, alkynyl, acyl, adenine, guanine, cytosine, uracil, thymine, uridine, pyrimidine, purine, pseudouridine, inosine, or hypoxanthine;

wherein R₃ is independently selected from a: phosphorothioate, phosphodiester, aldehyde, carboxyl, carbonyl, ether, ester, or amine; and

wherein, in some optional embodiments, the phosphodiester, aldehyde, carboxyl, carbonyl, ether, ester, or amine is bonded to a contiguous nucleic acid, such that R₃reads

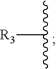

or a pharmaceutically active salt thereof.

In some embodiments, the present disclosure provides a composition comprising a compound having Formula Z:

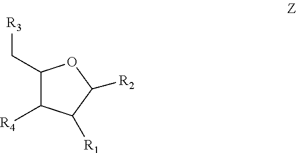

wherein R₁ is independently selected from: hydrogen, hydroxyl, halogen, alkyl, alkenyl, alkynyl, acyl, ester, sulfonyl, amide, amine, alkyloxy, methoxyethyl, or DNP (2,4′-dinitrophenol);

wherein R₂ is independently selected from: hydrogen, hydroxyl, halogen, alkyl, alkenyl, alkynyl, acyl, adenine, guanine, cytosine, uracil, thymine, uridine, any pyrimidine, any purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphodiester, phosphorothioate, aldehyde, carboxyl, carbonyl, ether, ester, or amine phosphodiester, aldehyde, carboxyl, carbonyl, ether, ester, or amine; and, in some optional embodiments, the alkylphosphonate, phosphotriester, phosphorodithioate, phosphoramidate, ketone, sulfone, carbonate thioamidate, phosphodiester, aldehyde, carboxyl, carbonyl, ether, ester, or amine is bonded to a contiguous nucleic acid, such that R₃ reads

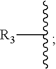

wherein R₄ is independently selected from a: alkylphosphonate, phosphotriester, phosphorodithioate, phosphoramidate, ketone, sulfone, carbonate thioamidate. phosphodiester, phosphorothioate, aldehyde, carboxyl, carbonyl, ether, ester, or amine; in some optional embodiments, the alkylphosphonate, phosphotriester, phosphorodithioate, phosphoramidate, ketone, sulfone, carbonate thioamidate, phosphodiester, aldehyde, carboxyl, carbonyl, ether, ester, or amine is bonded to one or a plurality of contiguous nucleic acids, such that R₄ reads

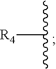

or a pharmaceutically active salt thereof.

In some embodiments, the present disclosure provides a composition comprising a compound having Formula W:

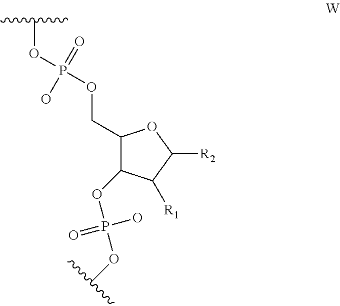

wherein R₁ is independently selected from a halogen, methyl, or methoxy ethyl;

wherein R₂ is independently selected from: hydrogen, hydroxyl, halogen, alkyl, alkenyl, alkynyl, acyl, a base, adenine, guanine, cytosine, uracil, thymine, uridine, pyrimidine, purine, pseudouridine, inosine, or hypoxanthine;

or a pharmaceutically active salt thereof.

In some embodiments, the present disclosure provides a composition comprising a compound having Formula X:

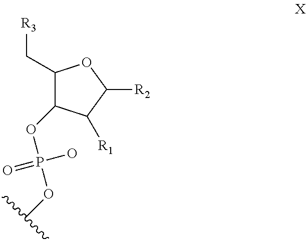

wherein R₁ is independently selected from a halogen, methyl, or methoxy ethyl;

wherein R₂ is independently selected from: any nucleobase, hydrogen, hydroxyl, halogen, alkyl, alkenyl, alkynyl, acyl, adenine, guanine, cytosine, uracil, thymine, uridine, a pyrimidine, a purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphodiester, aldehyde, carboxyl, carbonyl, ether, ester, or amine; in some embodiments, the phosphodiester, alkylphosphonate, phosphotriester, phosphorodithioate, phosphoramidate, ketone, sulfone, carbonate thioamidate, aldehyde, carboxyl, carbonyl, ether, ester, or amine is bonded to a contiguous nucleic acid or nucleoside, such that the R₃ reads

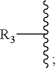

or a pharmaceutically active salt thereof.

In some embodiments, the present disclosure provides a composition comprising a compound having Formula Y:

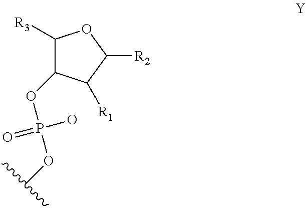

wherein R₁ is independently selected from: hydrogen, hydroxyl, halogen, methyl, or methoxy ethyl;

wherein R₂ is independently selected from: hydrogen, hydroxyl, halogen, alkyl, alkenyl, alkynyl, acyl, any base, adenine, guanine, cytosine, uracil, thymine, uridine, a pyrimidine, a purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, amine or a CH2-bonded to a phosphodiester, aldehyde, carboxyl, carbonyl, ether, ester, amine;

wherein, in some optional, embodiments, the alkylphosphonate, phosphotriester, phosphorodithioate, phosphoramidate, ketone, sulfone, carbonate thioamidate, phosphodiester, aldehyde, carboxyl, carbonyl, ether, ester, or amine is bonded to a contiguous nucleic acid, such that the R₃ reads

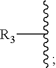

or a pharmaceutically active salt thereof.

In some embodiments, the present disclosure provides a composition comprising a compound having Formula Z:

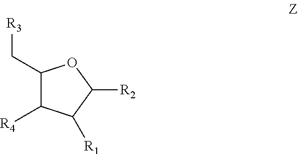

wherein R₁ is independently selected from: a hydrogen, a hydroxyl, a halogen, methyl, or methoxy ethyl;

wherein R₂ is independently selected from: hydrogen, hydroxyl, halogen, alkyl or heteroakyl, alkenyl, alkynyl, acyl, any base, pyrimidine, purine, adenine, guanine, cytosine, uracil, thymine, uridine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine;

wherein R₄ is independently selected from a one or a combination of: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine;

or a pharmaceutically active salt thereof; wherein the compound X is positioned between or bonded to any one or plurality of unmodified or modified nucleotides at R₃ and/or R₄.

In some embodiments, the present disclosure provides a composition comprising a compound having Formula W:

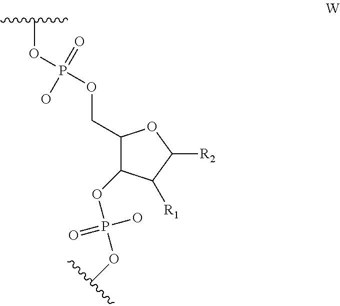

wherein R₁ is a hydrogen;

wherein R₂ is independently selected from: adenine, guanine, cytosine, uracil, thymine, uridine, pyrimidine, purine, pseudouridine, inosine, or hypoxanthine;

or a pharmaceutically active salt thereof.

In some embodiments, the present disclosure provides a composition comprising a compound having Formula X:

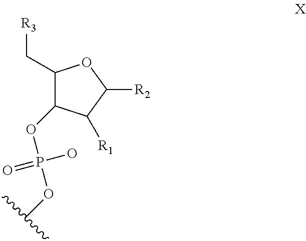

wherein R₁ is a hydrogen;

wherein R₂ is independently selected from: adenine, guanine, cytosine, uracil, thymine, uridine, pyrimidine, purine, pseudouridine, inosine, or hypoxanthine;

wherein R₃ is independently selected from a: phosphodiester, aldehyde, carboxyl, carbonyl, ether, ester, or amine;

or a pharmaceutically active salt thereof.

In some embodiments, the present disclosure provides a composition comprising a compound having Formula Y:

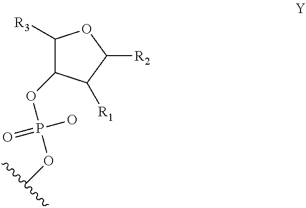

wherein R₁ is a hydrogen;

wherein R₂ is independently selected from: adenine, guanine, cytosine, uracil, thymine, uridine, pyrimidine, purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine;

or a pharmaceutically active salt thereof.

In some embodiments, the present disclosure provides a composition comprising a compound having Formula Z:

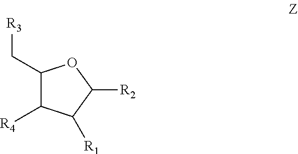

wherein R₁ independently selected from is a hydrogen, heteroakyl, methyl, methoxy ethyl, or halogen;

wherein R₂ is independently selected from: aryl, heteroaryl, cycloalkyl, heterocycloalkyl adenine, guanine, cytosine, uracil, thymine, uridine, pyrimidine, purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine;

wherein R₄ is independently selected from a: alkylphosphonate, phosphotriester, phosphorodithioate, phosphoramidate, ketone, sulfone, carbonate thioamidate, hydrogen, methyl, methoxy ethyl, phosphodiester, phosphorothioate, aldehyde, carboxyl, carbonyl, ether, ester, or amine; or

or a pharmaceutically active salt thereof.

In some embodiments, the present disclosure provides a composition comprising a compound having Formula W:

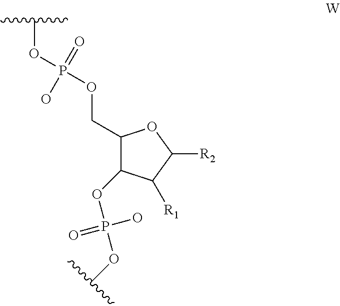

wherein R₁ is a hydroxyl;

wherein R₂ is independently selected from: adenine, guanine, cytosine, uracil, thymine, uridine, pyrimidine, purine, pseudouridine, inosine, or hypoxanthine;

or a pharmaceutically active salt thereof.

In some embodiments, the present disclosure provides a composition comprising a compound having Formula X:

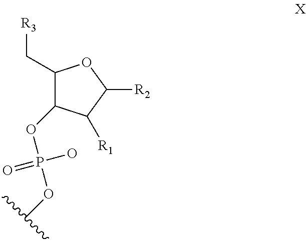

wherein R₁ is a hydroxyl;

wherein R₂ is independently selected from: adenine, guanine, cytosine, uracil, thymine, uridine, pyrimidine, purine, pseudouridine, inosine, or hypoxanthine;

wherein R₃ is independently selected from a: phosphorothioate, phosphodiester, aldehyde, carboxyl, carbonyl, ether, ester, or amine;

or a pharmaceutically active salt thereof.

In some embodiments, the present disclosure provides a composition comprising a compound having Formula Y:

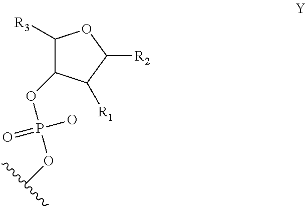

wherein R₁ is a hydroxyl;

wherein R₂ is independently selected from: adenine, guanine, cytosine, uracil, thymine, uridine, pyrimidine, purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine; wherein the groups are optionally further bound to one or a plurality of nucleotides or nucleosides, in deoxyribonucleic acid or ribonucleic acid forms.

or a pharmaceutically active salt thereof.

In some embodiments, the present disclosure provides a composition comprising a compound having Formula Z:

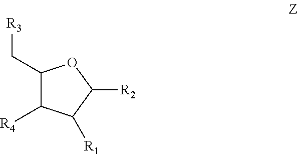

wherein R₁ is a hydroxyl;

wherein R₂ is independently selected from: hydrogen, hydroxyl, halogen, alkyl, alkenyl, alkynyl, acyl, any nucleobase, adenine, guanine, cytosine, uracil, thymine, uridine, pyrimidine, purine, pseudouridine, inosine, or hypoxanthine;

wherein R₃ is independently selected from a: alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine optionally bound to one or a plurality or modified or unmodified nucleotides;

wherein R₄ is independently selected from a: phosphodiester, phosphorothioate aldehyde, carboxyl, carbonyl, ether, ester, or amine optionally bound to one or a plurality or modified or unmodified nucleotides and/or nucleosides; or

a pharmaceutically active salt thereof.

In any of the forgoing formulae, any natural or non-natural nucleic acid may be one of several nucleic acids in a contiguous sequence within any of the disclosed sgRNAs, tracrRNAs, crRNAs, or other nucleic acid sequences disclosed herein, such that R₃ and/or R₄ are optionally comprising a substituent independently selected from one or a combination of: a alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine is bonded to a contiguous nucleic acid, such that the R₃ and/or R₄ reads

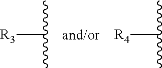

respectively. In any of the forgoing formulae, any natural or non-natural nucleic acid may be one of several nucleic acids in a contiguous sequence within any of the disclosed sgRNAs, tracrRNAs, crRNAs, or other nucleic acid sequences disclosed herein, such that R₁ is free of an O-methyl group at positions within the nucleic acid sequence that bind or are capable of interacting with a Cas protein.

In any of the forgoing formulae, any natural or non-natural nucleic acid may be one of several nucleic acids in a contiguous sequence within any of the disclosed sgRNAs, tracrRNAs, crRNAs, or other nucleic acid sequences disclosed herein, such that R₁ is a halogen at positions within the nucleic acid sequence that bind or are capable of interacting with a Cas protein. In any of the forgoing formulae, any natural or non-natural nucleic acid may be one of several nucleic acids in a contiguous sequence within any of the disclosed sgRNAs, tracrRNAs, crRNAs, or other nucleic acid sequences disclosed herein, such that R₁ is a fluorine at positions within the nucleic acid sequence that bind or are capable of interacting with a Cas protein. In any of the forgoing formulae, any natural or non-natural nucleic acid may be one of several nucleic acids in a contiguous sequence within any of the disclosed sgRNAs, tracrRNAs, crRNAs, or other nucleic acid sequences disclosed herein, such that R₁ is a halogen at positions within the nucleic acid sequence that bind or are capable of interacting with a Cas protein and wherein R₃ and/or R₄ are an internucleotide linkage comprising or selected from the group consisting of: a alkylphosphonate, phosphotriester, phosphorodithioate, phosphoramidate, ketone, sulfone, carbonate thioamidate, phosphorothioate, phosphodiester, aldehyde, carboxyl, carbonyl, ether, ester, or amine. In any of the forgoing formulae, any natural or non-natural nucleic acid may be one of several nucleic acids in a contiguous sequence within any of the disclosed sgRNAs, tracrRNAs, crRNAs, or other nucleic acid sequences disclosed herein, such that R₁ is a halogen at positions within the nucleic acid sequence that bind or are capable of interacting with a Cas protein and wherein R₃ and/or R₄ are an internucleotide linkage free of a phosphodiester bond. In any of the forgoing formulae, any natural or non-natural nucleic acid may be one of several nucleic acids in a contiguous sequence within any of the disclosed sgRNAs, tracrRNAs, crRNAs, or other nucleic acid sequences disclosed herein, such that R₁ is a fluorine at one or a plurality of positions within the nucleic acid sequence that bind or are capable of interacting with a Cas protein and wherein R₃ and/or R₄ are an phosphorothioate internucleotide linkage.

In some embodiments, the disclosure relates to a composition or pharmaceutical composition comprising a nucleic acid sequence comprising formulae W, X, Y, and Z in any contiguous or non-contiguous order or pattern, such that the total number of nucleic acids in the nucleic acid sequence is from about 15 to about 200. In some embodiments, the disclosure relates to a composition or pharmaceutical composition comprising a nucleic acid sequence comprising formulae W, X, Y, and Z in any contiguous or non-contiguous order or pattern, such that the total number of nucleic acids in the nucleic acid sequence is 101. In some embodiments, the nucleic acid molecules of the disclosure comprise any one or combination of formulae W, X, Y, and Z, but wherein R₁ from any or all of the formula is free of a alkyl group and/or O-alkyl group.

In some embodiments, any natural or non-natural nucleic acid formula may be repeated across 1, 2, 3, 4, 5, 6, 7, 8, 9, 10 or more nucleic acids in contiguous nucleic acids or in a non-contiguous pattern across the length of the nucleic acid. In some embodiments, the disclosed nucleic acid sequences comprises 1, 2, 3, 4, 5, 6, 7, 8, 9, 10, 11, 12, 13, 14, 15, 16, 17, 18, 19, 20 or more contiguous or non-contiguous nucleic acids across a length of the nucleic acid.

In some embodiments, the composition or pharmaceutical composition disclosed herein comprises a nucleic acid disclosed herein that comprises ribonucleic acid and about 10%, 11%, 12%, 13%, 14%, 15%, 16%, 17%, 18%, 19%, 20%, 22%, 25%, 27%, 30%, 32%, 35%, 37%, 40%, 42%, 45%, 47%, 50%, 52%, 55%, 57%, 60%, 62%, or 65% deoxyribonucleic acid or variants or modified derivatives thereof.

In some embodiments, any of the forgoing formulae may comprise one or a plurality of LNA molecules positioned between or bound to one or a plurality of modified or unmodified nucleotides.

In some embodiments, the composition or pharmaceutical composition disclosed herein comprises a nucleic acid sequence comprising a total of about 50, 60, 70, 80, 90, 100, 150, or 200 nucleotides in length and comprising in 5′ to 3′ orientation: a nucleotide binding domain, a Cas protein binding domain (or Cas binding domain), and, optionally a transcription terminator domain; wherein each of the aforementioned domains independently consists of no more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, or 190 nucleotides; and wherein from about 1% to about 100% of the nucleotides are modified.

In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 10% of the nucleotides are modified at the 2′ carbon position of the sugar moiety. In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 20% of the nucleotides are modified at the 2′ carbon position of the sugar moiety. In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 30% of the nucleotides are modified at the 2′ carbon position of the sugar moiety. In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 40% of the nucleotides are modified at the 2′ carbon position of the sugar moiety. In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 50% of the nucleotides are modified at the 2′ carbon position of the sugar moiety. In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 60% of the nucleotides are modified at the 2′ carbon position of the sugar moiety. In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide or DNA binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 70% of the nucleotides are modified at the 2′ carbon position of the sugar moiety. In some embodiments, the composition or pharmaceutical composition disclosed herein comprises a nucleic acid sequence comprising total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 80% of the nucleotides are modified at the 2′ carbon position of the sugar moiety. In some embodiments, the composition or pharmaceutical composition disclosed herein comprises a nucleic acid sequence comprising a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 90% of the nucleotides are modified at the 2′ carbon position of the sugar moiety. In some embodiments, the composition or pharmaceutical composition disclosed herein comprises a nucleic acid sequence comprising a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 95% of the nucleotides are modified at the 2′ carbon position of the sugar moiety. In some embodiments, the composition or pharmaceutical composition disclosed herein comprises a nucleic acid sequence comprising a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 40%, 50%, 60%, 65%, 70%, 75%, 80%, 85%, 90%, or 95% of the nucleotides comprise halogens at the 2′ carbon position of the sugar moiety. In any of the foregoing embodiments, the 2′ carbon position may be a hydroxyl or hydrogen at any one or plurality of positions capable of interacting with or binding to a Cas protein in an active CRISPR complex. In any of the foregoing embodiments, the 2′ carbon position may be a hydroxyl or hydrogen at any one or plurality of conserved positions capable of interacting with or binding to a Cas protein in an active CRISPR complex and identified in the Tables or Figures disclosed herein.

In some embodiments, the composition or pharmaceutical composition disclosed herein comprises a nucleic acid sequence comprising a total of about 50, 60, 70, 80, 90, 100, 150, or 200 nucleotides in length and comprising in 5′ to 3′ orientation: a nucleotide or DNA binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 10% of the nucleotides are modified at the 2′ carbon position of the sugar moiety, but one or a combination of the following positions within the domains comprise a hydroxyl group at the 2′ carbon of the sugar moiety of the nucleotide:

positions 1, 12, 15, 16, and/or 19 of the nucleotide-binding domain;
positions 22, 23, 24, 25, 26, 27, 43, 44, 45, 47, 49, 51, 58, 59, and/or 62 of the Cas-binding domain;
positions 63, 64, 65, 68, 69, and/or 82 of the transcription terminator domain; wherein the position number 1 of the nucleic acid sequence corresponds to the first nucleotide in the nucleotide binding domain.

In some embodiments, the composition or pharmaceutical composition disclosed herein comprises a nucleic acid comprises a total of about 50, 60, 70, 80, 90, 100, 150, or 200 nucleotides in length and comprise in 5′ to 3′ orientation: a nucleotide binding domain, a Cas protein binding domain, and, optionally, a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 10% of the nucleotides are modified at the 2′ carbon position of the sugar moiety, but one or a combination of the following positions within the domains consist of a hydroxyl group at the 2′ carbon of the sugar moiety of the nucleotide: positions 1, 12, 15, 16, and/or 19 of the nucleotide-binding domain;

positions 22, 23, 24, 25, 26, 27, 43, 44, 45, 47, 49, 51, 58, 59, and/or 62 of the Cas-binding domain;
positions 63, 64, 65, 68, 69, and/or 82 of the transcription terminator domain; wherein the position number 1 of the nucleic acid sequence corresponds to the first nucleotide in the nucleotide binding domain.

In some embodiments, the composition or pharmaceutical composition disclosed herein comprises a nucleic acid sequence comprising a total of about 50, 60, 70, 80, 90, 100, 150, or 200 nucleotides in length and comprise in 5′ to 3′ orientation: a nucleotide binding domain, a Cas protein binding domain, and, optionally a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 10% of the nucleotides are modified at the 3′ carbon position carbon position of the sugar moiety.

In some embodiments, the composition or pharmaceutical composition disclosed herein comprises a nucleic acid comprising a total of about 50, 60, 70, 80, 90, 100, 150, or 200 nucleotides in length and comprising, in 5′ to 3′ orientation: a nucleotide binding domain, a Cas protein binding domain, and, optionally a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 10% of the nucleotides are modified at the 4′carbon position of the sugar moiety. In some embodiments, the composition or pharmaceutical composition disclosed herein comprises a nucleic acid sequence comprising a total of about 50, 60, 70, 80, 90, 100, 150, or 200 nucleotides in length and comprise in 5′ to 3′ orientation: a nucleotide binding domain, a Cas protein binding domain, and, optionally a transcription terminator domain; wherein each of the aforementioned domains independently consists of no more than about 20, 30, 40, 50, 60, 70, 80, 90, 100, 110, 120, 130, 140, 150, 160, 170, 180, or 190 nucleotides; and wherein at least 10% of the nucleotides are modified at the 5′carbon position of the sugar moiety.

In some embodiments, the composition or pharmaceutical composition disclosed herein comprises a nucleic acid molecule comprising at least one or a combination of domains from a 5′ to 3′ orientation: a DNA-binding domain, a Cas-protein binding domain, and a transcription terminator domain.

In certain embodiments, the nucleic acid molecule comprising the DNA-binding domain is an sgRNA or a crRNA. The length of the DNA-binding domain may vary depending, for example, on the target sequence. In some embodiments, the DNA-binding domain comprises about 25, 30, 35, 40, 45, 50 or 55 nucleotides. Any of the these values may be used to define a range for the length of the DNA-binding domain. For example, in some embodiments, the DNA-binding domain comprises about 35-45, about 25-45, or about 25-55 nucleotides.

In some embodiments, one or more nucleotides in the DNA-binding domain are modified. For example, in some embodiments, about 5%, 10%, 15%, 25%, 30%, 35%, 40%, 45%, 50% or 55% of the nucleotides in the DNA-binding domain are modified. In some embodiments, less than 5%, 10%, 15%, 20%, 21%, 22%, 23%, 24% 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or 55% of the nucleotides in the DNA-binding domain are modified. Any of these values may be used to define a range for the percentage of nucleotides in the DNA-binding domain that are modified. For example, in some embodiments, 26% to 34%, 26% to 50%, or 21% to 50% of the nucleotides in the DNA-binding domain are modified. In some embodiments, fewer than 2, 3, 4, 5, 6, 7, 8, 9, 10, 11, 12, 13, 14 or 15 of the nucleotides in the DNA-binding domain are modified. Any of these values may be used to define a range for the number of nucleotides that are modified in the DNA-binding domain. For example, in some embodiments, 2 to 15, 7 to 15, or 13 to 15 of the nucleotides in the DNA-binding domain are modified.

In certain embodiments, the modification of the nucleotide in the DNA-binding domain is one or more of 2′-O-methyl, 2′-O-fluoro, or phosphorothioate. In certain embodiments, the nucleotide is modified at the 2′ position of the sugar moiety. In certain embodiments, the modification at the 2′ position of the sugar moiety is 2′-O-methyl or 2′-O-fluoro. In certain embodiments, the nucleotide is modified at the 3′ position of the sugar moiety. In certain embodiments, the modification at the 3′ position of the sugar moiety is phosphorothioate. In certain embodiments, the nucleotide is modified at both the 2′ position of the sugar moiety and at the 3′ position of the sugar moiety. In certain embodiments, the nucleotide is not modified at the 2′ position of the sugar moiety. In certain embodiments, the nucleotide is not modified at the 3′ position of the sugar moiety.

In certain embodiments, the nucleic acid molecule (e.g. an sgRNA or a crRNA) comprises a DNA-binding domain comprising about 25 to about 55 nucleotides, wherein the nucleotides of the nucleic acid sequence are modified at one or more of positions 1, 2, 3, 4, 5, 6, 7, 8, 9, 10, 11, 13, 14, 17 or 18 of the DNA-binding domain. In some embodiments, the nucleic acid molecule is modified at one or more of positions 1, 2, 3, 6, 7, 8, 9, 10, 11, 13, 14, 17 or 18 of the DNA-binding domain. In some embodiments, the nucleotide at one or more of positions 4, 5 and 12 of the DNA-binding domain is not modified. In some embodiments, the nucleotide at one or more of positions 1, 2, 3, 4 and 5 of the DNA-binding domain is not modified.

In a particular embodiment, the nucleic acid molecule (e.g. an sgRNA or a crRNA) comprises a DNA-binding domain comprising about 25 to about 55 nucleotides, wherein the nucleotides of the nucleic acid sequence are modified at one or more of positions 1, 2, 3, 6, 7, 8, 9, 10, 11, 13, 14, 17 or 18 of the DNA-binding domain, and wherein the nucleotide at one or more of positions 4, 5 and 12 of the DNA-binding domain is not modified.

In certain embodiments, the nucleic acid molecule is a crRNA and is combined with a second nucleic acid molecule comprising at least one transcription terminator domain. In certain embodiments, the second nucleic acid molecule is a tracrRNA.

In some embodiments, the nucleic acid molecule comprises a Cas-protein binding domain. In certain embodiments, the Cas-protein binding domain comprises about 30, 35, 40, 41, 42, 43, 44, 45, 46, 47, 48, 49, 50, 51, 52, 53, 54 or 55 nucleotides. Any of these values may be used to define a range for the length of the Cas-protein binding domain. For example, in some embodiments, the Cas-protein binding domain comprises about 30 to 55, about 40 to 45, or about 40 to 50 nucleotides. In a particular embodiment, the Cas-protein binding domain comprises about 41 nucleotides.

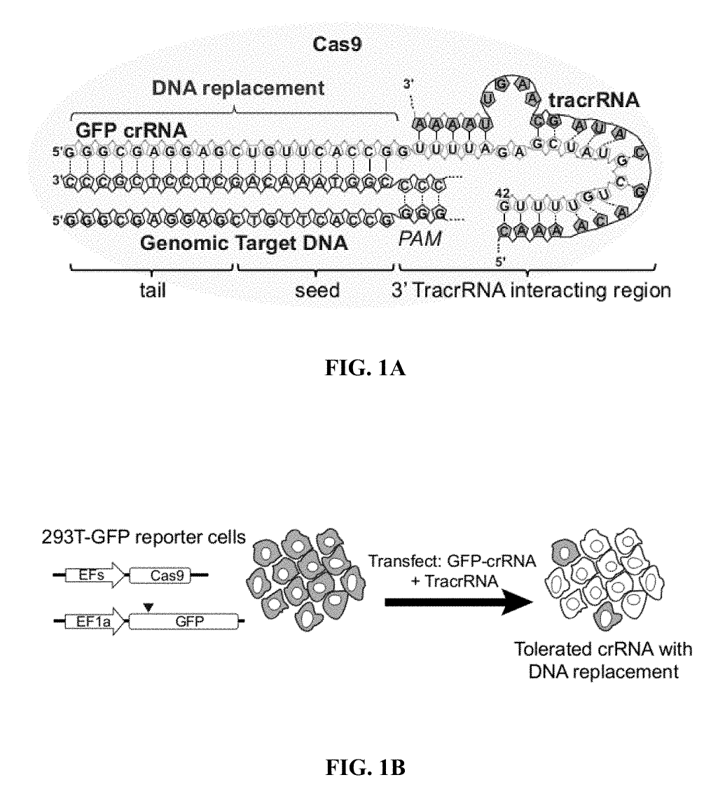

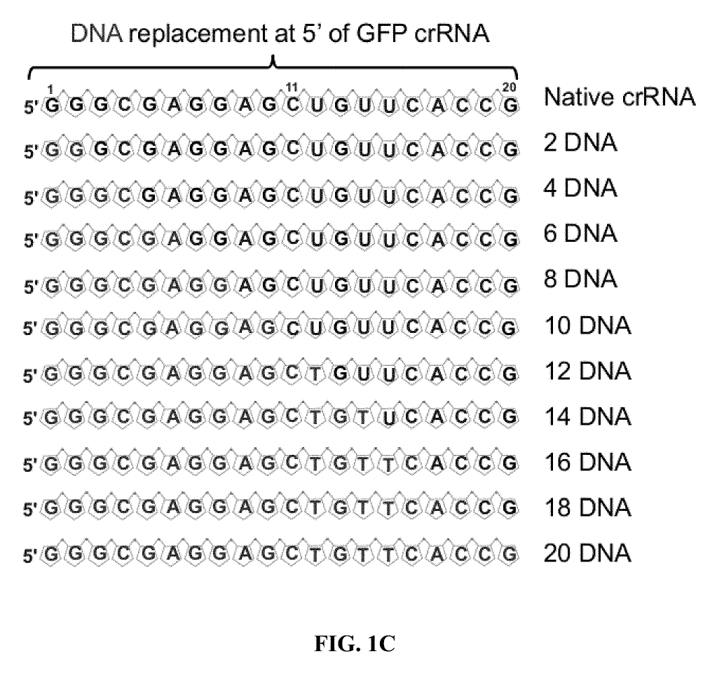

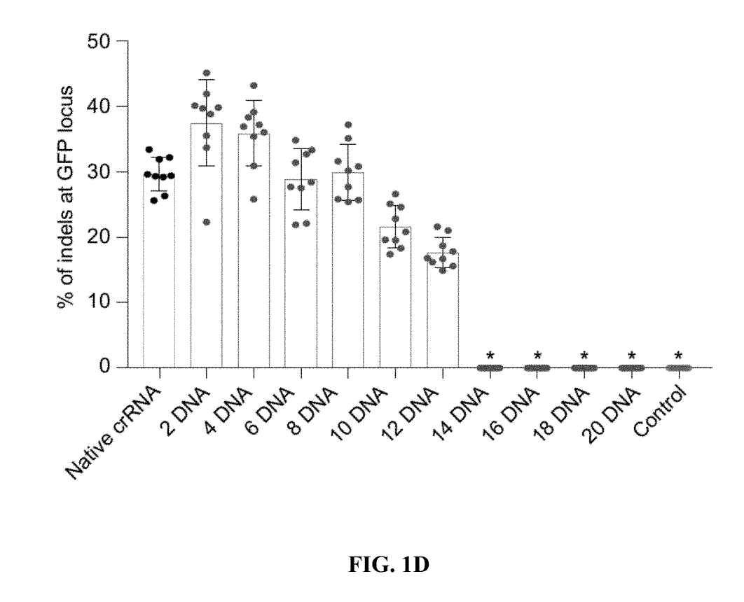

In certain embodiments, the Cas-protein binding domain comprises the nucleic acid sequence of SEQ ID NO: 112: GUUUUAGAGCUAGAAAUAGCAAGUUAAAAUAAGGCUAGUCCG. SEQ ID NO: 112 represents the Cas-protein binding domain shown in FIG. 1. In some embodiments, the Cas-protein binding domain comprises a nucleic acid sequence having at least 10, 20, 30, 40, 50, 60, 70, 75, 80, 85, 90, 95, 96, 97, 98 or 99% sequence identity to the nucleic acid sequence of SEQ ID NO: 112. In certain embodiments, the Cas-protein binding domain comprises a nucleic acid sequence in which at least one or a combination of nucleotides are conserved at positions: 2, 3, 4, 23, 24, 25, 27, 31, 38 and 42 of SEQ ID NO: 112. In certain embodiments, the Cas-protein binding domain comprises the sequence of SEQ ID NO: 113: NUUUNNNNNNNNNNNNNNNNNNGUUNANNNANNNNNNGNNNG (SEQ ID NO: 113), wherein “N” may be any nucleotide.

In certain embodiments about 1, 2, 3, 4, 5, 6, 7, 8, 9, or 10 nucleotides in the Cas-protein binding domain are modified. In certain embodiments, fewer than 2, 3, 4, 5, 6, 7, 8, 9, or 10 nucleotides in the Cas-protein binding domain are modified. In a particular embodiment, 8 nucleotides in the Cas-protein binding domain are modified.

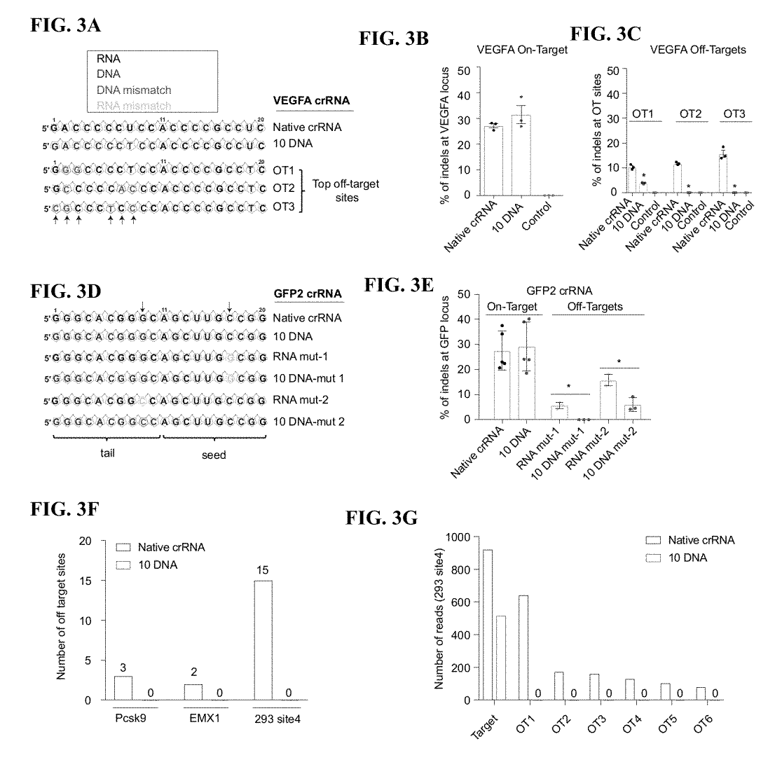

In certain embodiments, the modification of the nucleotide in the Cas-protein binding domain is one or more of 2′-O-methyl, 2′-fluoro, or phosphorothioate. In certain embodiments, the modification of the nucleotide in the Cas-protein binding domain is one or more of 2′-O-methyl, 2′-fluoro, or phosphorothioate according to FIG. 3a. In certain embodiments, the nucleotide is modified at the 2′ position of the sugar moiety. In certain embodiments, the modification at the 2′ position of the sugar moiety is 2′-O-methyl or 2′-fluoro. In certain embodiments, the nucleotide is modified at the 3′ position of the sugar moiety. In certain embodiments, the modification at the 3′ position of the sugar moiety is phosphorothioate. In certain embodiments, the nucleotide is modified at both the 2′ position of the sugar moiety and at the 3′ position of the sugar moiety. In certain embodiments, the nucleotide is not modified at the 2′ position of the sugar moiety. In certain embodiments, the nucleotide is not modified at the 3′ position of the sugar moiety.

In certain embodiments, the Cas-protein binding domain is modified at one or more of positions 10, 11, 12, 14, 15, 17, 18 and 19 of the Cas-protein binding domain (e.g. SEQ ID NO: 112).

In some embodiments, the nucleic acid molecule comprises a transcription terminator domain. In certain embodiments, the transcription terminator domain comprises about 15, 16, 17, 18, 19, 20, 25, 30, 35, 36, 37, 38, 39, 40, 41, 42, 43, 44 or 45 nucleotides. Any of these values may be used to define a range for the length of the transcription terminator domain. For example, in some embodiments, the transcription terminator domain comprises about 35 to 45, about 35 to 40, or about 17 to 45 nucleotides. In a particular embodiment, the transcription terminator domain comprises about 39 nucleotides.

In some embodiments, the transcription terminator domain comprises the nucleic acid sequence of SEQ ID NO: 114:

(SEQ ID NO: 114)

UUAUCAACUUGAAAAAGUGGCACCGAGUCGGUGCUUUUU.

SEQ ID NO: 114 represents the transcription terminator domain shown in FIG. 1. In some embodiments, the transcription terminator domain comprises a nucleic acid sequence having at least 10, 20, 30, 40, 50, 60, 70, 75, 80, 85, 90, 95, 96, 97, 98 or 99% sequence identity to the nucleic acid sequence of SEQ ID NO: 114. In certain embodiments, the transcription terminator domain comprises a nucleic acid sequence in which at least one or a combination of nucleotides are conserved at positions 1, 2, 3 or 6 of the nucleic acid sequence of SEQ ID NO: 114. In certain embodiments, the transcription terminator domain comprises the nucleic acid sequence of SEQ ID NO: 115:

(SEQ ID NO: 115)

UUANNANNNNNNNNNNNNNNNNNNNNNNNNNNNNNNNNN,

wherein “N” may be any nucleotide.

In some embodiments, one or more nucleotides in the transcription terminator domain are modified. For example, in some embodiments, 1, 2, 3, 4, 5, 6, 7, 8, 9, 10, 11, 12, 13, 14, 15, 16, 17 or 18 nucleotides in the transcription terminator domain are modified. In some embodiments, fewer than 2, 3, 4, 5, 6, 7, 8, 9, 10, 11, 12, 13, 14, 15, 16, 17 or 18 nucleotides in the transcription terminator domain are modified. In certain embodiments, one or more nucleotides at positions 4, 5, 8, 9, 10, 20, 23, 25, 26, 30, 31, 34, 36 of the transcription terminator domain (e.g. SEQ ID NO: 114) are modified. In certain embodiment, one or more nucleotides at positions 4, 5, 8, 9, 10, 18, 21, 23, 24, 28, 29, 32, 33, 34, 35, or 36 of the transcription terminator domain (e.g. SEQ ID NO: 114) are modified.

In certain embodiments, the modification of a nucleotide in the transcription terminator domain is one or more of 2′-O-methyl, 2′-O-fluoro, or phosphorothioate. In a particular embodiment, the modification in the transcription terminator domain is 2′-O-fluoro. In certain embodiments, the nucleotide is modified at the 2′ position of the sugar moiety. In certain embodiments, the modification at the 2′ position of the sugar moiety is 2′-O-methyl or 2′-O-fluoro. In certain embodiments, the nucleotide is modified at the 3′ position of the sugar moiety. In certain embodiments, the modification at the 3′ position of the sugar moiety is phosphorothioate. In certain embodiments, the nucleotide is modified at both the 2′ position of the sugar moiety and at the 3′ position of the sugar moiety. In certain embodiments, the nucleotide is not modified at the 2′ position of the sugar moiety. In certain embodiments, the nucleotide is not modified at the 3′ position of the sugar moiety. In some embodiments, the nucleotide at one or more of positions 1, 2, 3, 4 and 5 of the transcription terminator domain (e.g. SEQ ID NO: 114) is not modified.

In a particular embodiment, the nucleic acid molecule comprises a transcription terminator domain comprising from about 17 to 45 nucleotides, wherein the transcription terminator domain has at least 70% sequence homology to the nucleic acid sequence of SEQ ID NO: 114, and wherein one or more of the nucleotides are modified.

In certain embodiments of the aforementioned nucleic acid molecules, only the DNA-binding domain comprises one or more modified nucleotides. In certain embodiments, only the Cas-protein binding domain of the nucleic acid molecule comprises one or more modified nucleotides. In certain embodiments, only the transcription terminator domain of the nucleic acid molecule comprises one or more modified nucleotides. In certain embodiments, both the DNA-binding domain and the Cas-protein binding domain of the nucleic acid molecule comprise one or more modified nucleotides. In certain embodiments, both the DNA-binding domain and the transcription terminator domain comprise one or more modified nucleotides. In certain embodiments, both the Cas-protein binding domain and the transcription terminator domain comprise one or more modified nucleotides. In certain embodiments, the DNA-binding domain, Cas-protein binding domain and transcription terminator domain each comprise one or more modified nucleotides.

In certain aspects, the invention also relates to a pharmaceutical composition comprising any of the aforementioned nucleic acid molecules in a pharmaceutically effective amount. In certain embodiments, the pharmaceutical composition comprises a nanoparticle comprising any of the aforementioned nucleic acid molecules in a pharmaceutically effective amount.

The disclosure relates to a nucleic acid sequence comprising a DNA binding domain of formula V₀, wherein V₀ is about 5 nucleotides with formula N₁N₂N₃N₄N″; wherein N₁N₂N₃N₄ are modified nucleotides with a base complementary to a DNA target sequence; and wherein N″ is an unmodified nucleotide with a base complementary to a DNA target sequence. The disclosure also relates to a nucleic acid sequence comprising a formula V₀, wherein V₀ is about 5 nucleotides with formula N₁N₂N₃N₄N″; wherein N₁N₂N₃ comprise a 2′F with a base complementary to a DNA target sequence; wherein the bond between N₃ and N₄ is a phosphorothioate bond; wherein N″ is an unmodified base complementary to a base from the DNA target sequence. The disclosure also relates to a nucleic acid sequence comprising a DNA binding domain of formula V₀, wherein V₀ is GGGCG.

In some embodiments, the disclosure relates to a nucleic acid sequence comprising a DNA binding domain of formula V₁, wherein V₁ is about 7 nucleotides with formula N₅N₆N₇N₈N₉N₁₀N′; wherein N₅N₆N₇N₈N₉N₁₀ are modified nucleotides with a base complementary to a base from the DNA target sequence; and wherein N″ is an unmodified nucleotide with a base complementary to a base from the DNA target sequence. The disclosure relates to a nucleic acid sequence comprising a formula V₁, wherein V₁ is about 7 nucleotides with formula N₅N₆N₇N₈N₉N₁₀N′; wherein N₅N₆N₇N₈N₉N₁₀ comprise a 2′F with a base complementary to a base from a DNA target sequence; wherein the bond between N₅ and N₆ is a phosphorothioate bond. wherein N″ is an unmodified base complementary to a base from the DNA target sequence. This disclosure relates to a nucleic acid sequence comprising a formula V₁, wherein V₁ is AGGAGCU.

In some embodiments, the disclosure relates to a nucleic acid sequence comprising a DNA binding domain of formula V₂, wherein V₂ is about 8 nucleotides with formula N₁₁N₁₂N₁₃N₁₄N₁₅N₁₆N₁₇N₁₈; wherein N₁₁N₁₂N₁₅N₁₆ are modified nucleotides with a base complementary to a DNA target sequence; and wherein N₁₃N₁₄N₁₇N₁₈ are an unmodified nucleotides with a base complementary to a DNA target sequence. The disclosure relates to a nucleic acid sequence comprising a formula V₂, wherein V₂ is about 8 nucleotides with formula N₁₁N₁₂N₁₃N₁₄N₁₅N₁₆N₁₇N₁₈; wherein N₁₁N₁₂N₁₅N₁₆ comprise a 2′F with a base complementary to a DNA target sequence.

The disclosure also relates to a nucleic acid sequence comprising a formula V₂, wherein V₂ is GUUCACCG.

In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; and wherein the DNA-binding domain comprise a nucleotide sequence at least 70, 80, 85, 90, 91, 92, 93, 94 95, 96, 97, 98, 99, or 100% homologous to V₀ (N₁N₂N₃N₄N″), V₁ (N₅N₆N₇N₈N₉N₁₀N′), V₂ (N₁₁N₁₂N₁₃N₁₄N₁₅N₁₆N₁₇N₁₈), or any combination of those nucleotide sequences with that formula, wherein any position with N₁, N₂, N₃, N₄, N₅, N₆, N₇, N₈, N₉, N₁₀, N₁₁ N₁₂, N₁₃, N₁₄, N₁₅, N₁₆, N₁₇, N₁₈ is a modified nucleotide independently selectable from formula W, X, Y, or Z, and wherein N′ or N″ are unmodified nucleotides.

In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; and wherein the DNA-binding domain comprise a nucleotide sequence at least 70, 80, 85, 90, 91, 92, 93, 94 95, 96, 97, 98, 99, or 100% homologous to V₀ (N₁N₂N₃N₄N″), V₁ (N₅N₆N₇N₈N₉N₁₀N′), V₂ (N₁₁N₁₂N₁₃N₁₄N₁₅N₁₆N₁₇N₁₈), or any combination of those nucleotide sequences with that formula, wherein any position with N₁, N₂, N₃, N₄, N₅, N₆, N₇, N₈, Ng, N₁₀, N₁₁ N₁₂, N₁₃, N₁₄, N₁₅, N₁₆, N₁₇, N₁₈ is a modified nucleotide independently selected from formula W, X, Y, or Z, and wherein N′ or N″ are unmodified nucleotides.

In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; and wherein the DNA-binding domain comprises a nucleotide sequence at least about 70%, 80, 85, 90, 91, 92, 93, 94 95, 96, 97, 98, 99, or about 100% homologous to a nucleotide sequence with formula with contiguous sequences in 5′ to 3′ order of V₀, —V₁-V₂, wherein V₀ is about 5 nucleotides with formula N₁N₂N₃N₄N″; wherein N₁N₂N₃ comprise a 2′F with a base complementary to a DNA target sequence; wherein the bond between N₃ and N₄ is a phosphorothioate bond; wherein N″ is an unmodified base complementary to a base from the DNA target sequence; wherein V₁ is about 7 nucleotides with formula N₅N₆N₇N₈N₉N₁₀N′; wherein N₅N₆N₇N₈N₉N₁₀ comprise a 2′F with a base complementary to a base from a DNA target sequence; wherein the bond between N₅ and N₆ is a phosphorothioate bond. wherein N″ is an unmodified base complementary to a base from the DNA target sequence; wherein V₂ is about 8 nucleotides with formula N₁₁N₁₂N₁₃N₁₄N₁₅N₁₆N₁₇N₁₈; wherein N₁₁N₁₂N₁₅N₁₆ comprise a 2′F with a base complementary to a DNA target sequence.

In some embodiments, the DNA-binding domain comprises the formula V2 and it contiguously flanks the 5′ end of the Cas protein-binding domain.

In some embodiments, the disclosure relates to n some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; wherein the Cas protein-binding domain comprises or consists of a sequence at least about 70%, 80, 85, 90, 91, 92, 93, 94 95, 96, 97, 98, 99, or about 100% homologous to the sequences chosen from: SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, and SEQ. ID NO. 8.

SEQ. ID NO. 1: AGCUAGAAAUAGCAA;
SEQ. ID NO. 2: AGCUAGAAAUAGCAAGUUAAAA;
SEQ. ID NO. 3: AGCUAGAAAUAGCAAGUUAAAAUAAGGCUA;
SEQ. ID NO. 4: AGCUAGAAAUAGCAAGUUAAAAUAAGGCUAGUCC
SEQ. ID NO. 5: GUUUUAGAGCUAGAAAUAGCAA
SEQ. ID NO. 6: GUUUUAGAGCUAGAAAUAGCAAGUUAAAA
SEQ. ID NO. 7: GUUUUAGAGCUAGAAAUAGCAAGUUAAAAUAAGGC
UA
SEQ. ID NO. 8: GUUUUAGAGCUAGAAAUAGCAAGUUAAAAUAAGGC
UAGUCC

In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; wherein the Cas protein-binding domain comprises or consists of a sequence at least about 70%, 80, 85, 90, 91, 92, 93, 94 95, 96, 97, 98, 99, or about 100% homologous to SEQ ID NO:8, wherein positions 1, 8 through 22, 26, 28, 32 through 37, 40, and 41 are modified nucleotides. In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; wherein the Cas protein-binding domain comprises or consists of a sequence about 100% homologous to SEQ ID NO:8, wherein positions 1, 8 through 22, 26, 28, 32 through 37, 40, and 41 are modified nucleotides with the base of SEQ ID NO:8. In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; wherein the Cas protein-binding domain comprises or consists of a sequence about 100% homologous to SEQ ID NO:8, wherein positions 1, 8 through 22, 26, 28, 32 through 37, 40, and 41 are modified nucleotides with the base of SEQ ID NO:8, where, at each position the nucleotide is independently selectable comprising Formula W, X, Y, or Z. In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; wherein the Cas protein-binding domain comprises or consists of a sequence about 100% homologous to SEQ ID NO:8, wherein positions 1, 8 through 22, 26, 28, 32 through 37, 40, and 41 are modified nucleotides with the base of SEQ ID NO:8, where, at each position the nucleotide is independently variable comprising Formula W, X, Y, or Z and wherein there are 2-O-methyl substitutions at the 2′ carbons of positions 1, 8 through 22, 26, 28, 32 through 37, 40, and 41.

In some embodiments, the disclosure relates to a nucleic acid sequence is a ribonucleic sequence or an sgRNA sequence that comprises modified nucleic acids in one, two or three domains which, in the 5′ to 3′ orientation, are: a DNA-binding domain, a Cas protein-binding domain, and a transcription terminator domain; wherein the transcription terminator domain comprises or consists of a sequence at least about 70%, 80, 85, 90, 91, 92, 93, 94 95, 96, 97, 98, 99, or about 100% homologous to the sequences chosen from: SEQ. ID NO. 9, SEQ. ID NO. 10, SEQ. ID NO. 11, SEQ. ID NO. 12, and SEQ. ID NO. 13

SEQ ID NO: 9

GUUAUCAACUUGAAAAAGUGGCACCGAGUCGGUGCUUUUU

SEQ ID NO: 10

CUUGAAAAAG

SEQ ID NO: 11

UGGCACCGAGUCGGUG

SEQ ID NO: 12

CUUUUU

SEQ ID NO: 13

GUUAUCAA;

Wherein, if the sequence comprises SEQ ID NO:9, position 21 comprises an unmodified nucleotide except that the bond between the nucleotide at position 21 and 22 is a phosphorothioate bond, and positions 5, 6, 9 through 20 and 22 through 40 are modified nucleotides with a formula independently selected from W, X, Y, or Z.

In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain, a Cas protein-binding domain, and a transcription terminator domain; wherein the transcription terminator domain comprises or consists of a base sequence at least about 70%, 80, 85, 90, 91, 92, 93, 94 95, 96, 97, 98, 99, or about 100% homologous to the sequences chosen from: SEQ. ID NO. 9, SEQ. ID NO. 10, SEQ. ID NO. 11, SEQ. ID NO. 12, and SEQ. ID NO. 13

SEQ ID NO: 9

GUUAUCAACUUGAAAAAGUGGCACCGAGUCGGUGCUUUUU

SEQ ID NO: 10

CUUGAAAAAG

SEQ ID NO: 11

UGGCACCGAGUCGGUG

SEQ ID NO: 12

CUUUUU

SEQ ID NO: 13

GUUAUCAA;

Wherein, if the sequence comprises SEQ ID NO:9, position 21 comprises an unmodified nucleotide except that the bond between the nucleotide at position 21 and 22 is a phosphorothioate bond, and positions 5, 6, 9 through 20 and 22 through 40 are modified nucleotides with a formula independently selected from formulae W, X, Y, or Z; wherein position 5 and 6, 9 through 20, 22-40 comprise 2-O-methyl groups in their 2′Carbon. In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain, a Cas protein-binding domain, and a transcription terminator domain; wherein the transcription terminator domain comprises or consists of a base sequence at least about 70%, 80, 85, 90, 91, 92, 93, 94 95, 96, 97, 98, 99, or about 100% homologous to the sequences chosen from: SEQ. ID NO. 9, SEQ. ID NO. 10, SEQ. ID NO. 11, SEQ. ID NO. 12, and SEQ. ID NO. 13

SEQ ID NO: 9

GUUAUCAACUUGAAAAAGUGGCACCGAGUCGGUGCUUUUU

SEQ ID NO: 10

CUUGAAAAAG

SEQ ID NO: 11

UGGCACCGAGUCGGUG

SEQ ID NO: 12

CUUUUU

SEQ ID NO: 13

GUUAUCAA;

Wherein, if the sequence comprises SEQ ID NO:9, position 21 comprises an unmodified nucleotide except that the bond between the nucleotide at position 21 and 22 is a phosphorothioate bond, and positions 5, 6, 9 through 20 and 22 through 40 are modified nucleotides with a formula independently selected from formulae W, X, Y, or Z; wherein position 5 and 6, 9 through 20, 22-40 comprise 2-O-methyl groups in their 2′Carbon; and wherein the other positions of SEQ ID NO:9 are unmodified nucleotides with the assigned base.

In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain, a Cas protein-binding domain, and a transcription terminator domain; wherein the transcription terminator domain comprises or consists of a base sequence at least about 70%, 80, 85, 90, 91, 92, 93, 94 95, 96, 97, 98, 99, or about 100% homologous to the sequences chosen from: SEQ. ID NO. 9, SEQ. ID NO. 10, SEQ. ID NO. 11, SEQ. ID NO. 12, and SEQ. ID NO. 13

SEQ ID NO: 9

GUUAUCAACUUGAAAAAGUGGCACCGAGUCGGUGCUUUUU

SEQ ID NO: 10

CUUGAAAAAG

SEQ ID NO: 11

UGGCACCGAGUCGGUG

SEQ ID NO: 12

CUUUUU

SEQ ID NO: 13

GUUAUCAA;

Wherein, if the sequence comprises SEQ ID NO:9, position 21 comprises an unmodified nucleotide except that the bond between the nucleotide at position 21 and 22 is a phosphorothioate bond, and positions 5, 6, 9 through 20 and 22 through 40 are modified nucleotides with a formula independently selected from formulae W, X, Y, or Z; wherein position 5 and 6, 9 through 20, 22-40 comprise 2-O-methyl groups in their 2′Carbon and the bonds between positions 9 through 18 and 23 through 40 are phosphorothioate bonds. In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain, a Cas protein-binding domain, and a transcription terminator domain; wherein the transcription terminator domain comprises or consists of a base sequence at least about 70%, 80, 85, 90, 91, 92, 93, 94 95, 96, 97, 98, 99, or about 100% homologous to the sequences chosen from: SEQ. ID NO. 9, SEQ. ID NO. 10, SEQ. ID NO. 11, SEQ. ID NO. 12, and SEQ. ID NO. 13

SEQ ID NO: 9

GUUAUCAACUUGAAAAAGUGGCACCGAGUCGGUGCUUUUU

SEQ ID NO: 10

CUUGAAAAAG

SEQ ID NO: 11

UGGCACCGAGUCGGUG

SEQ ID NO: 12

CUUUUU

SEQ ID NO: 13

GUUAUCAA;

Wherein, if the sequence comprises SEQ ID NO:9, position 21 comprises an unmodified nucleotide except that the bond between the nucleotide at position 21 and 22 is a phosphorothioate bond, and positions 5, 6, 9 through 20 and 22 through 40 are modified nucleotides with a formula independently selected from formulae W, X, Y, or Z; wherein the bonds between positions 9 through 18 and 23 through 40 are phosphorothioate bonds. In some embodiments, if any of the nucleic acids or guide sequences of the disclosure comprise at least about 70%, 80, 85, 90, 91, 92, 93, 94 95, 96, 97, 98, 99, or about 100% homologous to the sequences chosen from: SEQ. ID NO. 9, SEQ. ID NO. 10, SEQ. ID NO. 11, SEQ. ID NO. 12, and SEQ. ID NO. 13, then the nucleic acid or guide sequence may comprise any one or more mutations disclosed in FIG. 3a individually or combination.

In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain, a Cas protein-binding domain, and a transcription terminator domain; wherein the Cas protein-binding domain comprises or consists of a base sequence at least about 70%, 80, 85, 90, 91, 92, 93, 94 95, 96, 97, 98, 99, or about 100% homologous to the sequences chosen from: SEQ. ID NO. 8; wherein the transcription terminator domain comprises or consists of a base sequence at least about 70%, 80, 85, 90, 91, 92, 93, 94 95, 96, 97, 98, 99, or about 100% homologous to the sequences chosen from: SEQ. ID NO. 9, and wherein the modification or conserved regions are chosen from any one or plurality of positions disclosed herein. In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain, a Cas protein-binding domain, and a transcription terminator domain; wherein the Cas protein-binding domain comprises or consists of a base sequence at least about 70%, 80, 85, 90, 91, 92, 93, 94 95, 96, 97, 98, 99, or about 100% homologous to the sequences chosen from: SEQ. ID NO. 8; wherein the transcription terminator domain comprises or consists of a base sequence at least about 70%, 80, 85, 90, 91, 92, 93, 94 95, 96, 97, 98, 99, or about 100% homologous to the sequences chosen from: SEQ. ID NO. 9; wherein the DNA-binding domain comprises a nucleotide sequence at least 70, 80, 85, 90, 91, 92, 93, 94 95, 96, 97, 98, 99, or 100% homologous to V₀ (N₁N₂N₃N₄N″), V₁ (N₅N₆N₇N₈N9N₁₀N′), V₂ (N₁₁N₁₂N₁₃N₁₄N₁₅N₁₆N₁₇N₁₈), or any combination of those nucleotide sequences with that formula, wherein any position with N₁, N₂, N₃, N₄, N₅, N₆, N₇, N₈, N₉, N₁₀, N₁₁ N₁₂, N₁₃, N₁₄, N₁₅, N₁₆, N₁₇, N₁₈ is a modified nucleotide independently selectable from formula W, X, Y, or Z, and wherein N′ or N″ are unmodified nucleotides. and wherein the modification or conserved regions of SEQ ID NO:8 and/or SEQ ID NO:9 are chosen from any one or plurality of modifications or conserved positions disclosed herein.

In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain of about 20 nucleotides, a Cas protein-binding domain of about 41, and a transcription terminator domain of about 40 nucleotides; wherein the Cas protein-binding domain comprises or consists of a base sequence at least about 70%, 80, 85, 90, 91, 92, 93, 94 95, 96, 97, 98, 99, or about 100% homologous to the base sequence SEQ. ID NO. 8; wherein the transcription terminator domain comprises or consists of a base sequence at least about 70%, 80, 85, 90, 91, 92, 93, 94 95, 96, 97, 98, 99, or about 100% homologous to the base sequences SEQ. ID NO. 9; wherein the DNA-binding domain comprises a nucleotide sequence at least 70, 80, 85, 90, 91, 92, 93, 94 95, 96, 97, 98, 99, or 100% homologous to nucleotide sequences independently selectable from V₀ (N₁N₂N₃N₄N″), V₁ (N₅N₆N₇N₈N₉N₁₀N′), V₂ (N₁₁N₁₂N₁₃N₁₄N₁₅N₁₆N₁₇N₁₈), or any combination of those nucleotide sequences with that formula, wherein any position with N₁, N₂, N₃, N₄, N₅, N₆, N₇, N₈, N₉, N₁₀, N₁₁ N₁₂, N₁₃, N₁₄, N₁₅, N₁₆, N₁₇, N₁₈ is a modified nucleotide independently selectable from formula W, X, Y, or Z, and wherein N′ or N″ are unmodified nucleotides; and wherein the modification or conserved regions of SEQ ID NO:8 and/or SEQ ID NO:9 are chosen from any one or plurality of modifications or conserved positions disclosed herein.

In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain of about 20 nucleotides, a Cas protein-binding domain of about 41, and a transcription terminator domain of about 40 nucleotides; wherein the Cas protein-binding domain comprises or consists of a base sequence at least about 70%, 80, 85, 90, 91, 92, 93, 94 95, 96, 97, 98, 99, or about 100% homologous to the base sequence SEQ. ID NO. 8; wherein the transcription terminator domain comprises or consists of a base sequence at least about 70%, 80, 85, 90, 91, 92, 93, 94 95, 96, 97, 98, 99, or about 100% homologous to the base sequences SEQ. ID NO. 9; wherein the DNA-binding domain comprises a nucleotide sequence at least 70, 80, 85, 90, 91, 92, 93, 94 95, 96, 97, 98, 99, or 100% homologous to nucleotide sequences with contiguous formula V₀ (N₁N₂N₃N₄N″)—V₁ (N₅N₆N₇N₈N₉N₁₀N′)—V₂ (N₁₁N₁₂N₁₃N₁₄N₁₅N₁₆N₁₇N₁₈), wherein any position with N₁, N₂, N₃, N₄, N₅, N₆, N₇, N₈, N₉, N₁₀, N₁₁ N₁₂, N₁₃, N₁₄, N₁₅, N₁₆, N₁₇, N₁₈ is a modified nucleotide independently selectable from formula W, X, Y, or Z, and wherein N′ or N″ are unmodified nucleotides; and wherein the modification or conserved regions of SEQ ID NO:8 and/or SEQ ID NO:9 are chosen from any one or plurality of modifications or conserved positions disclosed herein.

In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain of about 20 nucleotides, a Cas protein-binding domain of about 41, and a transcription terminator domain of about 40 nucleotides; wherein the Cas protein-binding domain comprises or consists of a base sequence at least about 70%, 80, 85, 90, 91, 92, 93, 94 95, 96, 97, 98, 99, or about 100% homologous to the base sequence SEQ. ID NO. 8; wherein the transcription terminator domain comprises or consists of a base sequence at least about 70%, 80, 85, 90, 91, 92, 93, 94 95, 96, 97, 98, 99, or about 100% homologous to the base sequences SEQ. ID NO. 9; wherein the DNA-binding domain comprises a nucleotide sequence at least 70, 80, 85, 90, 91, 92, 93, 94 95, 96, 97, 98, 99, or 100% homologous to nucleotide sequences with contiguous formula V₀ (N₁N₂N₃N₄N″)—V₁ (N₅N₆N₇N₈N₉N₁₀N′)—V₂(N₁ᵢN₁₂N₁₃N₁₄N₁₅N₁₆N₁₇N₁₈), wherein any position with N₁, N₂, N₃, N₄, N₅, N₆, N₇, N₈, N₉, N₁₀, N₁₁ N₁₂, N₁₃, N₁₄, N₁₅, N₁₆, N₁₇, N₁₈ is a modified nucleotide independently selectable from formula W, X, Y, or Z, and wherein N′ or N″ are unmodified nucleotides; and wherein the modification or conserved regions of SEQ ID NO:8 and/or SEQ ID NO:9 are chosen from any one or plurality of modifications or conserved positions disclosed herein.

In some embodiments, the disclosure relates to a nucleic acid sequence is a ribonucleic sequence or an sgRNA sequence that comprises modified nucleic acids in one, two or three contiguous domains which, in the 5′ to 3′ orientation, are: a DNA-binding domain of about 20 nucleotides, a Cas protein-binding domain of about 41, and a transcription terminator domain of about 40 nucleotides; wherein the Cas protein-binding domain comprises or consists of a base sequence at least about 70%, 80, 85, 90, 91, 92, 93, 94 95, 96, 97, 98, 99, or about 100% homologous to the base sequence SEQ. ID NO. 8; wherein the transcription terminator domain comprises or consists of a base sequence at least about 70%, 80, 85, 90, 91, 92, 93, 94 95, 96, 97, 98, 99, or about 100% homologous to the base sequences SEQ. ID NO. 9; wherein the DNA-binding domain comprises a nucleotide sequence at least 70, 80, 85, 90, 91, 92, 93, 94 95, 96, 97, 98, 99, or 100% homologous to nucleotide sequences with contiguous formula V₀ (N₁N₂N₃N₄N″)—V₁ (N₅N₆N₇N₈N₉N₁₀N′)—V₂(N₁₁N₁₂N₁₃N₁₄N₁₅N₁₆N₁₇N₁₈), wherein any position with N₁, N₂, N₃, N₄, N₅, N₆, N₇, N₈, N₉, N₁₀, N₁₁ N₁₂, N₁₃, N₁₄, N₁₅, N₁₆, N₁₇, N₁₈ is a modified nucleotide independently selectable from formula W, X, Y, or Z, and wherein N′ or N″ are unmodified nucleotides; and wherein the modification or conserved regions of SEQ ID NO:8 and/or SEQ ID NO:9 are chosen from any one or plurality of modifications or conserved positions disclosed in FIG. 3a. In any of the disclosed sequences, the 5′ end may be flanked by one or more leader sequences comprising any modified or unmodified nucleotides in number from about 1 to about 100, 125, 150, or about 200 nucleotides in length.

Any nucleotide sequence disclosed herein (whether tracrRNA, tracrmate RNA, sgRNA, without or with DNA modification) may be a component in a pharmaceutical composition. In any such pharmaceutical composition, the composition comprises one or a plurality of disclosed nucleotide sequences in a pharmaceutically effective amount and one or a plurality of pharmaceutically acceptable carriers. In some embodiments, the pharmaceutical compositions comprise nanoparticles comprising one or a plurality of disclosed nucleotide sequences in a pharmaceutically effective amount. In some embodiments, the nanoparticles are lipid-containing nanoparticles in homogenous or heterogenous mixtures, such that, if a mixture is homogenous, the nanoparticles comprise the same or substantially the same modified nucleotide sequences disclosed herein (whether tracrRNA, tracrmate RNA, sgRNA, without or with DNA modification). In a heterogenous mixture, the pharmaceutical composition comprises a plurality of nanoparticles comprising different modified nucleotide sequences disclosed herein (whether tracrRNA, tracrmate RNA, sgRNA, without or with DNA modification) within each particle or among several particles.

The pharmaceutical composition comprising any of the disclosed nucleic acid molecules in pharmaceutically effective amounts may be administered to a subject to modify one or more target sequences. The dosage of the pharmaceutical composition administered to a subject may be optimized to maximize the percentage of target sequences in the subject that are modified by the nucleic acid molecules. In certain embodiments, the pharmaceutical composition is administered at a dosage sufficient to modify at least about 4%, 5%, 6%, 7%, 8%, 9%, 10%, 11%, 12%, 13%, 14%, 15%, 16%, 17%, 18%, 19% or about 20% of the target sequences. In a particular embodiment, the pharmaceutical composition is administered at a dosage sufficient to modify at least 4% of the target sequences. In certain embodiments, the pharmaceutical composition is administered to the subject at a dosage from about 100 μg/kg body weight of the subject to about 10 mg/kg body weight of the subject. In a particular embodiment, the pharmaceutical composition is administered at a dosage of about 1 mg/kg body weight of the subject.

In some embodiments, a small guide RNA (sgRNA) molecule is provided. The disclosure also relates to pharmaceutical compositions comprising any of the sgRNAs provided herein (including those sgRNA with percentages of deoxyribonucleic acids) or pharmaceutically acceptable salts thereof in a pharmaceutically effective amount. sgRNAs contain a nucleotide binding region that determines the sequence specificity of the sgRNA and the sgRNA:nuclease complex, a 5′ stem-loop region that, at least in part, participates in assembly and interaction with a sgRNA-mediated enzyme (such as a Cas protein-binding domain); and a transcription termination sequence. In some embodiments, the sgRNA or guide sequence comprises an intervening sequence between the transcription terminator domain and/or a 3′ stem-loop region in the transcription terminator domain. In some embodiments, the intervening sequence is no more than about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length.

The nucleotide binding region can be from about 5 to about 150 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In some cases, the binding region is from about 15 to about 30 nucleotides in length (e.g., from about 15 to about 29, 15-26, 15-25; 16-30, 16-29, 16-26, 16-25; or about 18-30, 18-29, 18-26, or 18-25 nucleotides in length). Generally, the nucleotide binding region is designed to complement or substantially complement the target nucleic acid sequence or sequences, such as a DNA target sequence. In some cases, the nucleotide binding domain is also called a “DNA-binding region,” and such terms are used equivalently in this application, because of its ability to bind to complementary or partially complementary target DNA sequences. Generally, for purposes of this application the nucleotide binding or DNA-binding domain is split between a seed region and a tail region. In most orientations and embodiments, the seed region is the 5′ most portion of the nucleotide binding domain and the tail region is the 3′ most portion of the nucleotide domain. The seed region can be no more than 6, 7, 8, 9, 10 or more contiguous nucleotides in length which is also contiguous with the tail region. In some embodiments, the tail region is also no more than 6, 7, 8, 9, 10 or more contiguous nucleotides in length. The position number of the nucleotides in the region is important in some embodiments because some positions of the nucleotide-binding portion of the sequences disclosed herein enhance the binding of the Cas protein to the nucleotide sequence and therefore enhance the enzymatic efficiency of the CRISPR complex.

The nucleotide binding domain can incorporate wobble or degenerate bases to bind multiple sequences. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is from about 40% and about 60% (e.g., 40%, 45%, 50%, 55%, 60%). In some cases, the nucleotide binding region can contain modified nucleotides such as, without limitation, methylated, phosphorylated, fluorinated, or hydroxylated nucleotides. In some cases, the nucleotide binding region can contain modified nucleotides such as, without limitation, methylated, phosphorylated, fluorinated, or hydroxylated nucleotides; wherein if the nucleotide is fluorinated, the nucleotide may also be bound to one or more adjacent modified or unmodified nucleotides by a phosphorothioate bond, in either R or S orientation.

In some embodiments, the nucleotide binding region binds or is capable of hybridizing with DNA, RNA, or hybrid RNA/DNA sequences, such as any of those target sequences described herein. In some embodiments, any of the domains or elements comprises DNA, RNA, or hybrid RNA/DNA sequences. In some embodiments, the nucleotide binding region comprises from about 5% to about 100% modified nucleotides based upon the total number of the nucleotides in the element or domain or entire guide sequence. In some embodiments, the nucleotide binding region comprises from about 5% to about 90% modified nucleotides as compared to an unmodified or naturally occurring nucleotide sequence. In some embodiments, the nucleotide binding region comprises from about 5% to about 80% modified nucleotides. In some embodiments, the nucleotide binding region comprises from about 5% to about 70% modified nucleotides. In some embodiments, the nucleotide binding region comprises from about 5% to about 60% modified nucleotides. In some embodiments, the nucleotide binding region comprises from about 5% to about 50% modified nucleotides. In some embodiments, the nucleotide binding region comprises from about 5% to about 40% modified nucleotides. In some embodiments, the nucleotide binding region comprises from about 5% to about 30% modified nucleotides. In some embodiments, the nucleotide binding region comprises from about 5% to about 20% modified nucleotides. In some embodiments, the nucleotide binding region comprises from about 5% to about 10% modified nucleotides.

In some embodiments, any domain comprises hybrid RNA/DNA sequences of either unmodified or modified nucleotides. In some embodiments, the DNA-targeting domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides is a modified or unmodified deoxyribonucleic acid. In some embodiments, the DNA-targeting domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides from the 5′ end of the guide sequence is a modified or unmodified deoxyribonucleic acid. In some embodiments, the Cas-binding domain comprises no less than about 250, 200, 150, 100, 50, 45, 40, 35, 30, 25, or 20 nucleotides, wherein no more than about 50, 45, 40, 35, 30, 25, 20, 15, 14, 13, 12, 11, 10, 9, 8, 7, 6, 5, 4, 3, 2, or 1 nucleotides is a modified or unmodified deoxyribonucleic acid. In some embodiments, the transcription terminator domain comprises no less than about 250, 200, 150, 100, 50, 45, 40, 35, 30, 25, or 20 nucleotides, wherein no more than about 50, 45, 40, 35, 30, 25, 20, 15, 14, 13 12, 11, 10, 9, 8, 7, 6, 5, 4, 3, 2, or 1 nucleotides is a modified or unmodified deoxyribonucleic acid. In some embodiments, the transcription terminator domain is free of modified or unmodified deoxyribonucleic acid. In some embodiments, the Cas-binding domain is free of modified or unmodified deoxyribonucleic acid.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

The terms “subject,” “individual,” and “patient” are used interchangeably herein to refer to a vertebrate, preferably a mammal, more preferably a human. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the animal is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly.

“Internucleotide linkage” refers to any group, molecules or atoms that covalently or noncovalently join two nucleosides. Unmodified internucleotide linkages are phosphodiester bonds. In some embodiments, the nucleic acid sequence or guide sequence comprises at least about 10%, 20%, 30%, 40%, 50%, 60%, 70%, 75%, 80%, 85%, 90%, 91%, 92%, 93%, 94%, 95%, 96%, 97%, 98%, 99% or more modified internucleotide linkages. Modified internucleotide linkages are set forth in the U.S. Pat. No. 8,133,669 and WO1994002499, each of which is incorporated herein in its entirety. Examples of such well known modified linkages, for which conventional synthesis schemes are known, include alkylphosphonate, phosphodiester, phosphotriester, phosphorothioate, phosphorodithioate, phosphoramidate, ketone, sulfone, carbonate and thioamidate linkages.

“2′-O-methyl sugar” or “2′-OMe sugar” means a sugar having a O-methyl modification at the 2′ position.

“2′-O-methoxyethyl sugar” or “2′-MOE sugar” means a sugar having a 0-methoxy ethyl modification at the 2′ position.

“2′-O-fluoro” or “2′-F” means a sugar having a fluoro modification of the 2′ position.

### Compositions

The CRISPR/Cas or the CRISPR-Cas system (both terms are used interchangeably throughout this application) does not require the generation of customized proteins to target specific sequences but rather a single Cas protein (or CRISPR enzyme) can be programmed by a short RNA molecule to recognize a specific DNA target, in other words the Cas enzyme (such as a type II Cas9 protein) can be recruited to a specific DNA target using a short RNA molecule complementary to at least a portion of such specific DNA target. One aspect of the disclosure is a modified guide sequence. Adding the guide sequence to the repertoire of genome sequencing techniques and analysis methods may significantly simplify the methodology and accelerate the ability to catalog, map genetic factors associated with a diverse range of biological functions and diseases and treat disease. To utilize the CRISPR-Cas system effectively for genome editing without deleterious effects, it is critical to understand aspects of engineering and optimization of these genome engineering tools, which are aspects of the disclosure.

In some embodiments, the disclosure relates to a nucleic acid sequence and compositions comprising the same. In another aspect, the disclosure relates to a nucleic acid sequence disclosed herein and compositions comprising the same with or without a vector that comprises a CRISPR enzyme or functional fragment thereof. In some embodiments, the nucleic acid sequence is a ribonucleic sequence or an sgRNA sequence that comprises from about 1% to about 99% modified nucleic acids in one, two or three domains which, in the 5′ to 3′ orientation, are: a DNA-binding domain, a Cas protein-binding domain, and a transcription terminator domain. Any combination or sequence of Formulae W, X, Y and Z are comtemplated in this disclosure. As a non-limiting example, compositions of the disclosure can comprise a guide sequence of N′—[Z]ₙ—N″; wherein N′ is any modified or unmodified 5′ terminal nucleotide; N″ is any modified or unmodified 3′ terminal nucleotide; any n is any positive integer from about 1 to about 250, wherein each position of Z in the formula may have an independently selected positions at their respective R₁, R₂, R₃, and R₄, subgroups; wherein, if a Z is at a position that binds to or interacts with a Cas protein in an active CRISPR complex, then R₁ is a hydroxyl or hydrogen; and R₃ and R₄ are natural or phosphosdiester linkages; and wherein, if a Z is at a position that does bind to or interact with a Cas protein in an active CRISPR complex, then at least 10%, 20%, 30%, 40%, 50%, 60%, 70%, 75%, 80%, 85%, 90%, 91%, 92%, 93%, 94%, 95%, 96%, 97%, 98%, 99% of Z comprise: an R₁ is free of hydroxyl or hydrogen; and R₃ and R₄ are free of natural or phosphosdiester internucleotide linkages. As a non-limiting example, compositions of the disclosure relate to a guide sequence of N′—[Z]ₙ—N″; wherein N′ is any modified or unmodified 5′ terminal nucleotide; N″ is any modified or unmodified 3′ terminal nucleotide; any n is any positive integer from about 1 to about 102, wherein each position of Z in the formula may have an independently selected positions at their respective R₁, R₂, R₃, and R₄, subgroups; wherein, if a Z is at a position that binds to or interacts with a Cas protein in an active CRISPR complex, then R₁ is a hydroxyl or hydrogen; and R₃ and/or R₄ are phosphosdiester linkages; and wherein, if a Z is at a position that does bind to or interact with a Cas protein in an active CRISPR complex, then at least 10%, 20%, 30%, 40%, 50%, 60%, 70%, 75%, 80%, 85%, 90%, 91%, 92%, 93%, 94%, 95%, 96%, 97%, 98%, or 99% of the R₁ is free of hydroxyl or hydrogen; and each R₃ and/or each R₄ are independently selected as an internucleotide linkage chosen from: an alkylphosphonate, phosphotriester, phosphorothioate, phosphorodithioate, phosphoramidate, ketone, sulfone, carbonate, aldehyde, carboxyl, carbonyl, ether, ester except a (phosphoester bond), amine and thioamidate. As a another non-limiting example, compositions of the disclosure may comprise a guide sequence of N′—[Z]ₙ—N″; wherein N′ is any modified or unmodified 5′ terminal nucleotide; N″ is any modified or unmodified 3′ terminal nucleotide; any n is any positive integer from about 1 to about 100, wherein each position of Z in the guide sequence may have an independently selected positions at their respective R₁, R₂, R₃, and R₄, subgroups; wherein, if a Z is at position that sufficiently binds to or interacts with a Cas protein to form an active CRISPR complex, then at least one of the Z has a hydroxyl or hydrogen at R₁; a phosphoester linkage at R₃ and/or R₄; and wherein, if a Z is at a position that does bind to or interact with a Cas protein to form an active CRISPR complex (each a non-binding Z), then at least 10%, 20%, 30%, 40%, 50%, 60%, 70%, 75%, 80%, 85%, 90%, 91%, 92%, 93%, 94%, 95%, 96%, 97%, 98%, or 99% of the non-binding Z is: (i) free of hydroxyl or hydrogen at the R₁ position; and (ii) each R₃ and/or each R₄ are independently selected as an internucleotide linkage chosen from: an alkylphosphonate, phosphotriester, phosphorothioate, phosphorodithioate, phosphoramidate, ketone, sulfone, carbonate, aldehyde, carboxyl, carbonyl, ether, ester except a (phosphoester bond), amine and thioamidate. As a another non-limiting example, compositions of the disclosure may comprise a guide sequence of N′—[Z]ₙ—N″; wherein N′ is any modified or unmodified 5′ terminal nucleotide; N″ is any modified or unmodified 3′ terminal nucleotide; any n is any positive integer from about 1 to about 100, wherein each position of Z in the guide sequence may have an independently selected positions at their respective R₁, R₂, R₃, and R₄, subgroups; wherein, if a Z is at position that binds to or interacts with a Cas protein to form an active CRISPR complex (a “binding-Z”), then at least one of the binding-Z has a hydroxyl or hydrogen at R₁; a phosphosester linkage at R₃ and/or R₄; and wherein, if a Z is at a position that does bind to or interact with a Cas protein to form an active CRISPR complex (each a non-binding Z), then at least 10%, 20%, 30%, 40%, 50%, 60%, 70%, 75%, 80%, 85%, 90%, 91%, 92%, 93%, 94%, 95%, 96%, 97%, 98%, or 99% of the non-binding Z is: (i) a halogen at its R₁; and (ii) each R₃ and/or each R₄ are independently selected as an internucleotide linkage chosen from: an alkylphosphonate, phosphorothioate, phosphorodithioate, phosphoramidite, and thioamidate. In some embodiments, any one or plurality of Z of the guide sequence of N′—[Z]ₙ—N″ may be replaced with one or a plurality of contiguous or noncontiguous, modified or unmodified nucleotides chosen from Formula W, X, and/or Y. In some embodiments, the non-binding Zs are at positions chosen from any position other than one or a plurality of positions on Tables 1, 5 and 6. In some embodiments, the guide sequence comprises one or a plurality of binding Zs at positions chosen from any one or plurality of positions identified on Tables 1, 5 or 6. As a another non-limiting example, compositions of the disclosure may comprise a guide sequence of N′—[Z]ₙ—N″; wherein N′ is any modified or unmodified 5′ terminal nucleotide; N″ is any modified or unmodified 3′ terminal nucleotide; any n is any positive integer from about 1 to about 100, wherein the guide sequence comprises the following domains in the 5′ to 3′ orientation: a nucleotide-binding domain; a Cas-binding domain; and a transcription terminator domain; and wherein each position of Z (Z₁ through Z₁₀₀) in the guide sequence may have an independently selectable substituents at their respective R₁, R₂, R₃, and R₄, subgroups; wherein, if a Z is at position that binds to or interacts with a Cas protein to form an active CRISPR complex (a “binding-Z”), then at least one of the binding-Z has a hydroxyl or hydrogen at R₁; a phosphosester linkage at R₃ and/or R₄; and wherein, if a Z is at a position that does bind to or interact with a Cas protein to form an active CRISPR complex (each a non-binding Z), then at least 10%, 20%, 30%, 40%, 50%, 60%, 70%, 75%, 80%, 85%, 90%, 91%, 92%, 93%, 94%, 95%, 96%, 97%, 98%, or 99% of the non-binding Z is: (i) a halogen at its R₁; and (ii) each R₃ and/or each R₄ are phosphorothioate. In some embodiments, any one or plurality of Z (Z₁ through Zₙ) of the guide sequence of N′—[Z]ₙ—N″ may be replaced with one or a plurality of contiguous or noncontiguous, modified or unmodified nucleotides chosen from Formula W, X, and/or Y. In some embodiments, the non-binding Zs are at positions chosen from any position other than one or a plurality of positions on Tables 1, 5 and 6. In some embodiments, the guide sequence comprises one or a plurality of binding Zs at positions chosen from any one or plurality of positions identified on Tables 1, 5 and/or 6.

In general, a “guide sequence” is any polynucleotide sequence having sufficient complementarity with a target polynucleotide sequence to hybridize with the target sequence and direct sequence-specific binding of a CRISPR complex (comprising any one or combination of Cas proteins) to the target polynucleotide sequence. The terms “guide sequence” includes any one or plurality of nucleic acid molecules consisting of an sgRNA, tracrRNA, crRNA, or tracr/crRNA duplex that hybridize with the target sequence and direct sequence-specific binding of a CRISPR complex to the target sequence. It is generally desirable to select a target sequence of DNA or RNA which is involved in the production of a protein whose synthesis is ultimately to be modulated, cut, altered or inhibited in its entirety by activity of a Cas9 enzyme or functional fragment thereof. The oligonucleotides of the disclosure are conveniently synthesized using solid phase synthesis of known methodology, and is designed at least at the nucleotide-binding domain to be complementary to or specifically hybridizable with the preselected nucleotide sequence of the target RNA or DNA. Nucleic acid synthesizers are commercially available and their use is understood by persons of ordinary skill in the art as being effective in generating any desired oligonucleotide of reasonable length. It is also possible to synthesize the sgRNA by use of T7 RNA polymerase and a DNA template added to a mixture with individual dNTPs at an appropriate concentrations so that each nucleotide (whether it be RNA nucleotide or a DNA nucleotide) of the sgRNA is polymerized sequentially by the T7 polymerase catalyzing a reaction linking each base. Methods of making the guide sequences disclosed herein are contemplated by this application in which such nucleotide sequences may be manufactured by solid phase synthesis, by recombinant expression of one or more nucleotides in an in vitro culture, or a combination of both in which modifications may be introduced at one or more positions across the length of the sequences.

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested, and a control guide sequence different from the test guide sequence, and comparing binding or rate of cleavage at the target sequence between the test and control guide sequence reactions. Other assays are possible, and will occur to those skilled in the art. In other experiments, a cell may be transfected with any one or combination of guide sequences without transfection of a nucleic acid encoding a Cas protein. The transfected cell may be engineered to already express a Cas protein.

A guide sequence may be selected to target any target sequence. In some embodiments, the target sequence is a sequence within a genome of a cell, either in vitro, ex vivo (such as in the generation of CAR T cells), or in vivo such as pharmaceutical compositions comprising any of the disclosed guide sequences being administered directly to a subject. In some embodiments, the compositions disclosed herein comprise a synthetic guide RNA comprising or consisting of any sequence selected to target any target sequence. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 20) where NNNNNNNNNNNNXGG (SEQ ID NO: 21) (N is A, G, T, or C; and X can be anything) has a single occurrence in the genome. A unique target sequence in a genome may include an S. pyogenes Cas9 target site of the form MMMMMMMMMNNNNNNNNNNNXGG (SEQ ID NO: 22) where NNNNNNNNNNNXGG (N is A, G, T, or C; and X can be anything) has a single occurrence in the genome. For the S. thermophilus CRISPR-Cas9, a unique target sequence in a genome may include a Cas9 target site of the form MMMMMMMMNNNNNNNNNNNNXXAGAAW (SEQ ID NO: 23) where NNNNNNNNNNNNXXAGAAW (SEQ ID NO: 24) (N is A, G, T, or C; X can be anything; and W is A or T) has a single occurrence in the genome. A unique target sequence in a genome may include an S. thermophilus CRISPR1 Cas9 target site of the form MMMMMMMMMNNNNNNNNNNNXXAGAAW (SEQ ID NO: 25) where NNNNNNNNNNNXXAGAAW (SEQ ID NO: 26) (N is A, G, T, or C; X can be anything; and W is A or T) has a single occurrence in the genome. For the S. pyogenes Cas9, a unique target sequence in a genome may include a Cas9 target site of the form MMMMMMMMNNNNNNNNNNNNXGGXG (SEQ ID NO: 27) where NNNNNNNNNNNNXGGXG (SEQ ID NO: 28) (N is A, G, T, or C; and X can be anything) has a single occurrence in the genome. A unique target sequence in a genome may include an S. pyogenes Cas9 target site of the form MMMMMMMMMNNNNNNNNNNNXGGXG (SEQ ID NO: 29) where NNNNNNNNNNNXGGXG (SEQ ID NO: 30) (N is A, G, T, or C; and X can be anything) has a single occurrence in the genome. In each of these sequences “M” may be A, G, T, or C, and need not be considered in identifying a sequence as unique.

In some embodiments the disclosure relates to a composition comprising a guide sequence that is an RNA molecule that comprises a DNA-binding sequence that comprises at least one or a combination of the sgRNA sequences of Table 4. In some embodiments, the composition comprises any one or combination of one or a plurality of sgRNA sequences or tracrRNA/crRNA sequences disclosed here comprising at least one DNA-binding domain at least about 70%, about 75%, about 80%, about 85%, about 90%, about 91%, about 92%, about 93%, about 94%, about 95%, about 96%, about 97%, about 98%, about 99% homologous, or about 100% homologous to a nucleotide sequence of Table 4. In some embodiments, the composition comprises any one or combination of one or a plurality of sgRNA sequences or tracrRNA/crRNA sequences disclosed here comprising at least one DNA-binding domain at least about 70%, about 75%, about 80%, about 85%, about 90%, about 91%, about 92%, about 93%, about 94%, about 95%, about 96%, about 97%, about 98%, about 99% homologous, or about 100% homologous to a nucleotide sequence that is complementary to a nucleotide sequence of Table 4.

TABLE 4
Proposed DNA-binding sequences of sgRNAs

| | | | sgRNA | |
|---|---|---|---|---|
| | | | target | |
| | | | sequence | |
| | Reference | | (Spacer | |
| Gene Name | Sequence No. | Species | Sequence) | Sequence |
| PCSK9 | NM_153565.2. | Mouse | sgRNA-1 | GCACCCATACCTTGGAGCAA |
| | | | | (SEQ ID NO: 85) |
| | | | sgRNA-2 | CCCATACCTTGGAGCAACGG |
| | | | | (SEQ ID NO: 86) |
| | | | sgRNA-3 | GCTCGCCCTCCCGTCCCAGG |
| | | | | (SEQ ID NO: 87) |
| PCSK9 | NM_174936.3. | Human | sgRNA-1 | GTTGGTCCCCAAAGTCCCCA |
| | | | | (SEQ ID NO: 88) |
| | | | sgRNA-2 | GCTCCGGCAGCAGATGGCAA |
| | | | | (SEQ ID NO: 89) |
| | | | sgRNA-3 | TCTTTGACTCTAAGGCCCAA |
| | | | | (SEQ ID NO: 90) |
| FIX | NM_000133.3. | Human | sgRNA-1 | CATGTGGCCTGGTCAACAAG |
| | | | | (SEQ ID NO: 91) |
| | | | sgRNA-2 | TGTGCTGGCTTCCATGAAGG |
| | | | | (SEQ ID NO: 92) |
| | | | sgRNA-3 | TAGATCGAAGACATGTGGCT |
| | | | | (SEQ ID NO: 93) |
| IL-10 | NM_000572.2. | Human | sgRNA-1 | TGAAAACAAGAGCAAGGCCG |
| | | | | (SEQ ID NO: 94) |
| | | | sgRNA-2 | GCGCCGTAGCCTCAGCCTGA |
| | | | | (SEQ ID NO: 95) |
| | | | sgRNA-3 | GGCGCATGTGAACTCCCTGG |
| | | | | (SEQ ID NO: 96) |
| VEGFR1 | NM_002019.4 | Human | sgRNA-1 | GGTCAGCTACTGGGACACCG |
| | | | | (SEQ ID NO: 97) |
| | | | sgRNA-2 | AGTGATGTTGAGGAAGAGGA |
| | | | | (SEQ ID NO: 98) |
| | | | sgRNA-3 | GAGCTTCCTGAATTAAACTT |
| | | | | (SEQ ID NO: 99) |
| CTLA-4 | NM_005214.4 | Human | sgRNA-1 | CATAGACCCCTGTTGTAAGA |
| | | | | (SEQ ID NO: 100) |
| | | | sgRNA-2 | AGGAAGTCAGAATCTGGGCA |
| | | | | (SEQ ID NO: 101) |
| | | | sgRNA-3 | TGGCTTGCCTTGGATTTCAG |
| | | | | (SEQ ID NO: 102) |
| cMyc | NM_002467 | Human | sgRNA-1 | GGTAGGGGAAGACCACCGAG |
| | | | | (SEQ ID NO: 103) |
| | | | sgRNA-2 | GTATTTCTACTGCGACGAGG |
| | | | | (SEQ ID NO: 104) |
| | | | sgRNA-3 | CTTCGGGGAGACAACGACGG |
| | | | | (SEQ ID NO: 105) |
| Kras | NM_004985 | Human | sgRNA-1 | TCCCTTCTCAGGATTCCTAC |
| | | | | (SEQ ID NO: 106) |
| | | | sgRNA-2 | AGATATTCACCATTATAGGT |
| | | | | (SEQ ID NO: 107) |
| | | | sgRNA-3 | AATTACTACTTGCTTCCTGT |
| | | | | (SEQ ID NO: 108) |
| Yap1 | NM_006106 | Human | sgRNA-1 | GGTCGGTCTCCGAGTCCCCG |
| | | | | (SEQ ID NO: 109) |
| | | | sgRNA-2 | GATGATGTACCTCTGCCAGC |
| | | | | (SEQ ID NO: 110) |
| | | | sgRNA-3 | TCAGATCGTGCACGTCCGCG |
| | | | | (SEQ ID NO: 111) |

In some embodiments, the disclosure relates to compositions comprising a guide sequence, wherein the guide sequence comprises a nucleotide binding domain or a DNA-binding domain comprising at least one modified nucleotide. In some embodiments, the nucleotide binding domain or a DNA-binding domain consists of from about 15 to about 25 nucleotides; wherein the from 15 to about 25 nucleotides comprises a sequence similarity of about 90, 91, 92, 93, 94, 95, 96, 97, 98, 99%, or 100% sequence homology to any target sequences identified herein or in the table provided above. In some embodiments, the nucleotide binding domain or a DNA-binding domain consists of from about 15 to about 30 nucleotides; wherein the from 15 to about 25 nucleotides comprises a sequence similarity of about 90, 91, 92, 93, 94, 95, 96, 97, 98, 99%, or 100% sequence homology to any target sequence identified herein. In some embodiments, the nucleotide binding domain or a DNA-binding domain consists of from about 15 to about 40 nucleotides; wherein the from 15 to about 25 nucleotides comprises a sequence similarity of about 90, 91, 92, 93, 94, 95, 96, 97, 98, 99%, or 100% sequence homology to any target sequence identified herein. In some embodiments, the nucleotide binding domain or a DNA-binding domain consists of from about 15 to about 25 nucleotides; wherein the from 15 to about 25 nucleotides comprises a sequence similarity of about 90, 91, 92, 93, 94, 95, 96, 97, 98, 99%, or about 100% sequence homology to any target sequence identified herein. For instance, one of ordinary skill in art could identify other DNA-binding domains which may be structurally related to those sequences provided in Table 4 to be used in connection with a CRISPR complex utilizing a Cas enzyme. For instance, it is possible that for modulation of PCSK9 (GCACCCATACCTTGGAGCAA) (SEQ ID NO: 85), the sgRNA sequence used may have about 90, 91, 92, 93, 94, 95, 96, 97, 98, or 99% sequence homology to any of the sgRNA-1, 2, or 3 associated with the DNA-binding domain of PCSK9.

In some embodiments, any of the sequences disclosed herein may have a DNA-binding domain, a Cas-binding domain, a transcription termination domain and an RNA-binding domain Any of the domains of the disclosed oligonucleotides may be in any order from 5′ to 3′ orientation and may be contiguous as to each other or any one or multiple domains or elements may be non-contiguous in relation to one or more of the other domains, such that a different element, amino acid sequence, nucleotide or set of modified nucleotides may precede the 5′ and/or 3′ area of any domain.

In some embodiments, for instance, any one or combination of domains or sequences disclosed herein may comprise a sequence of about 1, 2, 3, 4, 5, 6, 7, 8, 9, 10, 15, 20, 25, 30, 35, 40, 45, or 50 or more modified or unmodified nucleotides flanking the 3′ or 5′ end of each domain. In some embodiments, for instance, any one or combination of domains or sequences disclosed herein may comprise a sequence of about 1, 2, 3, 4, 5, 6, 7, 8, 9, 10, 15, 20, 25, 30, 35, 40, 45, or 50 or more modified or unmodified uracils flanking the 3′ or 5′ end of each domain. In some embodiments, the disclosed nucleic acid sequences has contiguous domains from the 5′ to the 3′ direction including a DNA-targeting domain, a Cas-binding domain, a transcription terminator domain, and, optionally a RNA-binding domain. In some embodiments, the disclosed nucleic acid sequences has contiguous domains from the 5′ to the 3′ direction including a DNA-targeting domain, a Cas-9 binding domain, a transcription terminator domain, and, optionally a RNA-binding domain. Each domain may comprise from about 10 to about 15, 20, 25, 30, 35, 40, 45, 50, 60, 65, 70, 75, 80, 85, 90, 95, 100, 110, 120, 130, 140, 150, 160, 170, 180, 190, 200, 210, 220, 230, 240, or 250 or more modified or unmodified nucleic acids of DNA or RNA.

The disclosure relates, among other things, to the rationale design of sgRNA, tracr/crRNA duplexes, and, generally, guide sequences that activate and/or catalyze the reaction of a CRISPR enzyme with a target nucleic acid sequence. The disclosure relates to the discovery that guide sequences (whether in the form of sgRNA, tracr/crRNA duplexes, or tracr/crRNA single strands) can be heavily modified to enhance on-target enzymatic efficiency as long as certain nucleotides that bind to the CRISPR enzyme, variant or functional fragments thereof are conserved at certain positions and/or, in some cases, conserved in respect to certain substituents on each nucleotide that are capable of binding a Cas protein, variant or functional fragments thereof in the presence of such a the Cas protein, variant or functional fragments thereof. Certain positions of the guide sequence can be more heavily modified based upon their functional association to other components of the CRISPR complex. For instance, in some embodiments, the composition or pharmaceutical composition disclosed herein comprises one or a plurality of nucleic acid sequences on one or plurality of nucleic acid molecules wherein the nucleic acid sequences comprise contiguous domains in the 5′ to 3′ orientation: a DNA-targeting domain, a Cas-binding domain, and a transcription terminator domain.

In some embodiments, the composition or pharmaceutical composition disclosed herein comprise a guide sequence or pharmaceutically acceptable salt thereof, comprising the following domains in 5′ to 3′ orientation: a DNA-targeting domain, a Cas-9 binding domain, a transcription terminator domain; wherein position 1 of the guide sequence is considered the first nucleotide position in the DNA-binding domain and wherein the DNA-binding domain comprises positions 1 through 20, the Cas-9 binding domain comprises positions 21 through 62, and the transcription terminator domain comprises positions 63 through 102.

In some embodiments, the composition or pharmaceutical composition disclosed herein comprise a guide sequence or pharmaceutically acceptable salt thereof, comprise one or a plurality of contiguous domains, in the 5′ to 3′ orientation, selected from: a DNA-targeting domain, a Cas-binding domain, and a transcription terminator domain; wherein position 1 of the guide sequence is considered the first nucleotide in the DNA-binding domain and where the DNA-binding domain comprises positions 1 through 20, the Cas-binding domain comprises positions 21-62, and the transcription terminator domain comprises positions 63 through 102; wherein any modification disclosed herein is at any position within the guide sequence, except that any one or plurality of nucleotides that binds or associates with a Cas protein in any domain is unmodified. In some embodiments, the composition or pharmaceutical composition disclosed herein comprise a guide sequence or pharmaceutically acceptable salt thereof, comprise one or a plurality of contiguous domains, in the 5′ to 3′ orientation, selected from: a DNA-targeting domain, a Cas-binding domain, and a transcription terminator domain; wherein position 1 of the guide sequence is considered the first nucleotide in the DNA-binding domain and where the DNA-binding domain comprises positions 1 through 20, the Cas-binding domain comprises positions 21-62, and the transcription terminator domain comprises positions 63 through 102; wherein any modification disclosed herein is at any position within the guide sequence, except that any one or plurality of nucleotides that binds or associates with a Cas protein in any domain is unmodified at the 2′ carbon position of the sugar moiety. In some embodiments, the guide sequence may have no more than 5%, 6%, 7%, 8%, 9%, 10%, 20%, 30%, 40%, 50%, 60%, 70%, 80%, 90%, 91%, 92%, 93%, 94%, 95%, 96%, 97%, 98%, or 99% modifications at any of the nucleotides in the guide sequence, except that any nucleotide that increases the stability between the guide sequence and a Cas protein (in a CRISPR complex or system) is left unmodified. In some embodiments, the guide sequence may have no more than 5%, 6%, 7%, 8%, 9%, 10%, 20%, 30%, 40%, 50%, 60%, 70%, 80%, 90%, 91%, 92%, 93%, 94%, 95%, 96%, 97%, 98%, or 99% modifications at any of the nucleotides in the guide sequence, except that any nucleotide that increases the stability between the guide sequence and a Cas protein (in a CRISPR complex or system) is left unmodified only at its 2′ carbon position of the sugar moiety.

The disclosure also relates to compositions or pharmaceutical compositions comprising guide sequences (optionally with one or more pharmaceutically acceptable salts at such positions) comprising a conserved hydroxyl group at the 2′ carbon of the ribose sugar or sugar moiety of one or a combination of the following positions of Table 5. In some embodiments, modifications to one or more of the positions in Table 5 may cause a decrease or abolishment of efficiency and/or efficacy of the sgRNA in which they are present.

TABLE 5
Conserved RNA Positions within a Guide Sequence for
Enhanced CRISPR complex activity

| | Position of the nucleotides of | |
|---|---|---|
| | guide sequence with | |
| | sequence in 5′ to 3′ | |
| | orientation of formula: | Nucleotide |
| | V₀-V₁-V₂-SEQ ID NO: 8-SEQ | interaction at |
| | ID NO: 9 | 2′OH |
| guide sequence | 1 | Varies |
| guide sequence | 12 | Varies |
| guide sequence | 15 | Varies |
| guide sequence | 16 | Varies |
| guide sequence | 19 | Varies |
| Fixed region | 22 | U |
| Fixed region | 23 | U |
| Fixed region | 24 | U |
| Fixed region | 25 | U |
| Fixed region | 26 | A |
| Fixed region | 27 | G |
| Fixed region | 43 | G |
| Fixed region | 44 | U |
| Fixed region | 45 | U |
| Fixed region | 47 | A |
| Fixed region | 49 | A |
| Fixed region | 51 | A |
| Fixed region | 58 | G |
| Fixed region | 59 | U |
| Fixed region | 62 | G |
| Fixed region | 63 | U |
| Fixed region | 64 | U |
| Fixed region | 65 | A |
| Fixed region | 68 | A |
| Fixed region | 69 | A |
| Fixed region | 82 | U |

In some embodiments, the composition or pharmaceutical composition comprises a guide sequence, or one or more pharmaceutically acceptable salts thereof, comprising the following domains in 5′ to 3′ orientation: a DNA-binding domain, a Cas-binding domain, and a transcription terminator domain; wherein position 1 of the guide sequence corresponds to the first nucleotide position in the DNA-binding domain and wherein the DNA-binding domain comprises positions from about 1 through about 20, the Cas-binding domain comprises positions from about 21 through about 62, and the transcription terminator domain comprises positions from about 63 through about 102. In some embodiments, position 1 is a uracil, thymine, adenine, or cytosine with a 2′ hydroxyl group on the 2′ carbon of the nucleotide of the sugar moiety and the guide sequence comprises any one or a plurality of modifications at positions 2-102. In some embodiments, position 12 is a uracil, thymine, adenine, or cytosine with a 2′ hydroxyl group on the 2′ carbon of the nucleotide of the sugar moiety and the guide sequence comprises any one or a plurality of modifications at positions 1-11 and 13-102. In some embodiments, position 15 is a uracil, thymine, adenine, or cytosine with a 2′ hydroxyl group on the 2′ carbon of the nucleotide of the sugar moiety and the guide sequence comprises any one or a plurality of modifications at positions 1-14 and 16-102. In some embodiments, position 16 is a uracil, thymine, adenine, or cytosine with a 2′ hydroxyl group on the 2′ carbon of the sugar moiety of the nucleotide and the guide sequence comprises any one or a plurality of modifications at positions 1-15 and 17-102. In some embodiments, position 19 is a uracil, thymine, adenine, or cytosine with a 2′ hydroxyl group on the 2′ carbon of the sugar moiety and the guide sequence comprises any one or a plurality of modifications at positions 1-18 and 20-102. In some embodiments, position 22 is a uracil, thymine, adenine, or cytosine with a 2′ hydroxyl group on the 2′ carbon of the sugar moiety and the guide sequence comprises any one or a plurality of modifications at positions 1-21 and 23-102. In some embodiments, position 23 is a uracil, thymine, adenine, or cytosine with a 2′ hydroxyl group on the 2′ carbon of the sugar moiety and the guide sequence comprises any one or a plurality of modifications at positions 1-22 and 24-102. In some embodiments, position 24 is a uracil, thymine, adenine, or cytosine with a 2′ hydroxyl group on the 2′ carbon of the sugar moiety and the guide sequence comprises any one or a plurality of modifications at positions 1-23 and 25-102. In some embodiments, position 25 is a uracil, thymine, adenine, or cytosine with a 2′ hydroxyl group on the 2′ carbon of the sugar moiety and the guide sequence comprises any one or a plurality of modifications at positions 1-24 and 26-102. In some embodiments, position 26 is a uracil, thymine, adenine, or cytosine with a 2′ hydroxyl group on the 2′ carbon of the sugar moiety and the guide sequence comprises any one or a plurality of modifications at positions 1-25 and 27-102. In some embodiments, position 27 is a uracil, thymine, adenine, or cytosine with a 2′ hydroxyl group on the 2′ carbon of the sugar moiety and the guide sequence comprises any one or a plurality of modifications at positions 1-26 and 28-102. In some embodiments, position 43 is a uracil, thymine, adenine, or cytosine with a 2′ hydroxyl group on the 2′ carbon of the sugar moiety and the guide sequence comprises any one or a plurality of modifications at positions 1-42 and 44-102. In some embodiments, position 44 is a uracil, thymine, adenine, or cytosine with a 2′ hydroxyl group on the 2′ carbon of the sugar moiety and the guide sequence comprises any one or a plurality of modifications at positions 1-43 and 45-102. In some embodiments, position 45 is a uracil, thymine, adenine, or cytosine with a 2′ hydroxyl group on the 2′ carbon of the sugar moiety and the guide sequence comprises any one or a plurality of modifications at positions 1-44 and 46-102. In some embodiments, position 47 is a uracil, thymine, adenine, or cytosine with a 2′ hydroxyl group on the 2′ carbon of the sugar moiety and the guide sequence comprises any one or a plurality of modifications at positions 1-46 and 48-102. In some embodiments, position 49 is a uracil, thymine, adenine, or cytosine with a 2′ hydroxyl group on the 2′ carbon of the sugar moiety and the guide sequence comprises any one or a plurality of modifications at positions 1-48 and 50-102. In some embodiments, position 51 is a uracil, thymine, adenine, or cytosine with a 2′ hydroxyl group on the 2′ carbon of the sugar moiety and the guide sequence comprises any one or a plurality of modifications at positions 1-50 and 52-102. In some embodiments, position 58 is a uracil, thymine, adenine, or cytosine with a 2′ hydroxyl group on the 2′ carbon of the sugar moiety and the guide sequence comprises any one or a plurality of modifications at positions 1-57 and 59-102. In some embodiments, position 59 is a uracil, thymine, adenine, or cytosine with a 2′ hydroxyl group on the 2′ carbon of the sugar moiety and the guide sequence comprises any one or a plurality of modifications at positions 1-58 and 60-102. In some embodiments, position 62 is a uracil, thymine, adenine, or cytosine with a 2′ hydroxyl group on the 2′ carbon of the sugar moiety and the guide sequence comprises any one or a plurality of modifications at positions 1-61 and 63-102. In some embodiments, position 63 is a uracil, thymine, adenine, or cytosine with a 2′ hydroxyl group on the 2′ carbon of the sugar moiety and the guide sequence comprises any one or a plurality of modifications at positions 1-62 and 64-102. In some embodiments, position 64 is a uracil, thymine, adenine, or cytosine with a 2′ hydroxyl group on the 2′ carbon of the sugar moiety and the guide sequence comprises any one or a plurality of modifications at positions 1-63 and 65-102. In some embodiments, position 65 is a uracil, thymine, adenine, or cytosine with a 2′ hydroxyl group on the 2′ carbon of the sugar moiety and the guide sequence comprises any one or a plurality of modifications at positions 1-64 and 66-102. In some embodiments, position 68 is a uracil, thymine, adenine, or cytosine with a 2′ hydroxyl group on the 2′ carbon of the sugar moiety and the guide sequence comprises any one or a plurality of modifications at positions 1-67 and 69-102. In some embodiments, position 69 is a uracil, thymine, adenine, or cytosine with a 2′ hydroxyl group on the 2′ carbon of the sugar moiety and the guide sequence comprises any one or a plurality of modifications at positions 1-68 and 70-102. In some embodiments, position 82 is a uracil, thymine, adenine, or cytosine with a 2′ hydroxyl group on the 2′ carbon of the sugar moiety and the guide sequence comprises any one or a plurality of modifications at positions 1-81 and 83-102.

In some embodiments, the composition or pharmaceutical composition comprises a guide sequence, or one or more pharmaceutically acceptable salts thereof, comprising the following domains in 5′ to 3′ orientation: a DNA-binding domain, a Cas-binding domain, and a transcription terminator domain; wherein position 1 of the guide sequence corresponds to the first nucleotide position in the DNA-binding domain and wherein the DNA-binding domain comprises positions 1 through 20, the Cas-binding domain comprises positions 21 through 62, and the transcription terminator domain comprises positions 63 through 102. In some embodiments, the guide sequence comprises any 1, 2, 3, 4, and/or 5 conserved hydroxyl groups on the 2′ carbon of the sugar moiety at positions 1, 12, 15, 16, and/or 19 of the DNA-binding domain. In some embodiments, the guide sequence comprises any 1, 2, 3, 4, 5, 6, 7, 8, 9, 10, 11, 12, 13, 14, or 15 conserved hydroxyl groups on the 2′ carbon of the sugar moiety at positions 22, 23, 24, 25, 26, 27, 43, 44, 45, 47, 49, 51, 58, 59, and/or 62 of the Cas-binding domain. In some embodiments, the guide sequence comprises any 1, 2, 3, 4, 5, and/or 6 conserved hydroxyl groups on the 2′ carbon of the sugar moiety at positions 63, 64, 65, 68, 69, and/or 82 of the transcription terminator domain. In some embodiments, the guide sequence comprises any 1, 2, 3, 4, and/or 5 conserved hydroxyl groups on the 2′ carbon of the sugar moiety at positions 1, 12, 15, 16, and/or 19 of the DNA-binding domain and any 1, 2, 3, 4, 5, 6, 7, 8, 9, 10, 11, 12, 13, 14, and/or 15 conserved hydroxyl groups on the 2′ carbon of the sugar moiety at positions 22, 23, 24, 25, 26, 27, 43, 44, 45, 47, 49, 51, 58, 59, and/or 62 of the Cas-binding domain. In some embodiments, the guide sequence comprises any 1, 2, 3, 4, and/or 5 conserved hydroxyl groups on the 2′ carbon of the sugar moiety at positions 1, 12, 15, 16, and/or 19 of the DNA-binding domain and any 1, 2, 3, 4, 5, and/or 6 conserved hydroxyl groups on the 2′ carbon of the sugar moiety at positions 63, 64, 65, 68, 69, and/or 82 of the transcription terminator domain. In some embodiments, the guide sequence comprises any 1, 2, 3, 4, 5, 6, 7, 8, 9, 10, 11, 12, 13, 14, and/or 15 conserved hydroxyl groups on the 2′ carbon of the sugar moiety at positions 22, 23, 24, 25, 26, 27, 43, 44, 45, 47, 49, 51, 58, 59, and/or 62 of the Cas-binding domain and any 1, 2, 3, 4, 5, and/or 6 conserved hydroxyl groups on the 2′ carbon of the sugar moiety at positions 63, 64, 65, 68, 69, and/or 82 of the transcription terminator domain. In some embodiments, the guide sequence comprises any 1, 2, 3, 4, and/or 5 conserved hydroxyl groups on the 2′ carbon of the sugar moiety at positions 1, 12, 15, 16, and/or 19 of the DNA-binding domain, any 1, 2, 3, 4, 5, 6, 7, 8, 9, 10, 11, 12, 13, 14, and/or 15 conserved hydroxyl groups on the 2′ carbon of the sugar moiety at positions 22, 23, 24, 25, 26, 27, 43, 44, 45, 47, 49, 51, 58, 59, and/or 62 of the Cas-binding domain, and any 1, 2, 3, 4, 5, and/or 6 conserved hydroxyl groups on the 2′ carbon of the sugar moiety at positions 63, 64, 65, 68, 69, and/or 82 of the transcription terminator domain

In some embodiments, the guide sequence comprises any 1, 2, 3, 4, and/or 5 unmodified nucleic acid molecules at positions 1, 12, 15, 16, and/or 19 of the DNA-binding domain. In some embodiments, the guide sequence comprises any 1, 2, 3, 4, 5, 6, 7, 8, 9, 10, 11, 12, 13, 14, and/or 15 unmodified nucleic acid molecules at positions 22, 23, 24, 25, 26, 27, 43, 44, 45, 47, 49, 51, 58, 59, and/or 62 of the Cas-binding domain. In some embodiments, the guide sequence comprises any 1, 2, 3, 4, 5, and/or 6 unmodified nucleic acid molecules at positions 63, 64, 65, 68, 69, and/or 82 of the transcription terminator domain. In some embodiments, the guide sequence comprises any 1, 2, 3, 4, and/or 5 unmodified nucleic acid molecules at positions 1, 12, 15, 16, and/or 19 of the DNA-binding domain and any 1, 2, 3, 4, 5, 6, 7, 8, 9, 10, 11, 12, 13, 14, and/or 15 unmodified nucleic acid molecules at positions 22, 23, 24, 25, 26, 27, 43, 44, 45, 47, 49, 51, 58, 59, and/or 62 of the Cas-binding domain. In some embodiments, the guide sequence comprises any 1, 2, 3, 4, and/or 5 unmodified nucleic acid molecules at positions 1, 12, 15, 16, and/or 19 of the DNA-binding domain and any 1, 2, 3, 4, 5, and/or 6 unmodified nucleic acid molecules at positions 63, 64, 65, 68, 69, and/or 82 of the transcription terminator domain. In some embodiments, the guide sequence comprises any 1, 2, 3, 4, 5, 6, 7, 8, 9, 10, 11, 12, 13, 14, and/or 15 unmodified nucleic acid molecules at positions 22, 23, 24, 25, 26, 27, 43, 44, 45, 47, 49, 51, 58, 59, and/or 62 of the Cas-binding domain and any 1, 2, 3, 4, 5, and/or 6 unmodified nucleic acid molecules at positions 63, 64, 65, 68, 69, and/or 82 of the transcription terminator domain. In some embodiments, the guide sequence comprises any 1, 2, 3, 4, and/or 5 unmodified nucleic acid molecules at positions 1, 12, 15, 16, and/or 19 of the DNA-binding domain, any 1, 2, 3, 4, 5, 6, 7, 8, 9, 10, 11, 12, 13, 14, and/or 15 unmodified nucleic acid molecules at positions 22, 23, 24, 25, 26, 27, 43, 44, 45, 47, 49, 51, 58, 59, and/or 62 of the Cas-binding domain, and any 1, 2, 3, 4, 5, and/or 6 unmodified nucleic acid molecules at positions 63, 64, 65, 68, 69, and/or 82 of the transcription terminator domain. In some embodiments, the guide sequence is free of a modification at any one or combination of positions set forth in Table 5. In some embodiments, the guide sequence is free of any akyl modification at any one or combination of 2′ carbons in the ribose at the positions set forth in Table 5. In some embodiments, the guide sequence is free of any O-methyl modification at any one or combination of positions set forth in Table 5.

The disclosure also relates to the discovery that certain domains within guide sequences (whether in the form of sgRNA, tracr/crRNA duplexes, or tracr/crRNA single strands) can be heavily modified to enhance on-target enzymatic efficiency as long as certain nucleotides that bind to the Cas protein, variant or functional fragments thereof are conserved at certain positions and/or conserved in respect to certain substituents on each nucleotide that interact with the Cas protein, variant or functional fragments thereof in the presence of such as the Cas protein, variant or functional fragments thereof. In some embodiments, the guide sequences described herein comprise modifications in the DNA-binding domain, or, in some embodiments, in the seed region of the DNA-binding domain. In some embodiments, their sugar moiety such that the 2′ position of the sugar moiety is a hydroxyl group. In some embodiments, the disclosure relates to a composition or pharmaceutical composition comprising a nucleic acid comprising the following domains contiguously oriented in the 5′ to 3′ direction:

X₁ domain-DNA-binding domain-Cas binding domain-transcription terminator domain-X₂ domain;

wherein the X₁ domain is from about 0 to about 100 nucleotides in length, the DNA-binding domain is from about 1 to about 20 nucleotides in length, the Cas-binding domain is from about 30 to about 50 nucleotides in length, the transcription terminator domain is from about 30 to about 70 nucleotides in length, and wherein the X₂ domain is from about 0 to about 200 nucleotides in length, and wherein position 1 corresponds to the first nucleotide in the DNA-binding domain and each position thereafter is a successive positive integer; and each nucleotide in the X₁ domain, if not 0 nucleotides in length, is assigned a position of a negative integer beginning with the position −1 at the nucleotide adjacent to position 1 in the 5′ direction. In some embodiments, the disclosure relates to a composition or pharmaceutical composition comprising a nucleic acid that comprises the following domains contiguously oriented in the 5′ to 3′ direction:

X₁ domain-DNA-binding domain-Cas binding domain-transcription terminator domain-X₂ domain; wherein the X₁ domain and the X₂ domain are 0 nucleotides in length, the DNA-binding domain is about 20 nucleotides in length, the Cas-binding domain is about 40 nucleotides in length, the transcription terminator domain is about 39 nucleotides in length.

In some embodiments, the disclosure relates to a composition or pharmaceutical composition comprising a nucleic acid comprises the following domains contiguously oriented in the 5′ to 3′ direction: X₁ domain-DNA-binding domain-Cas binding domain-transcription terminator domain-X₂ domain;

wherein the X1 domain and the X2 domain are 0 nucleotides in length, the DNA-binding domain is about 20 nucleotides in length, the Cas-binding domain is about 40 nucleotides in length, the transcription terminator domain is about 39 nucleotides in length; and wherein the nucleic acid sequence comprises one or a combination of ribonucleotides at the positions identified in Table 5. In some embodiments, the one or a combination of ribonucleotides at the positions identified in Table 5 comprise 2′ hydroxyl groups within the sugar moieties of the nucleotide.

The disclosure also relates to the combination of one or a plurality of modifications in the guide sequence. Any modifications at any position of the guide sequence or sequences may be made. In some embodiments, however, the modification are free of 2′O-methyl mutations at one or more of the positions identified in this disclosure. In some embodiments, the guide sequence or sequences are free of 2′O-alkyl mutations at one or more of the positions in the Cas-binding domain. In some embodiments, the modifications are free of 2′-fluoro mutations at one or more of the positions in the Cas-binding domain. In some embodiments, the guide sequence or sequences are free of phosphorothioate linkages at one or more of the positions in the Cas-binding domain. In some embodiments, the guide sequence or sequences are free of phosphorothioate linkages at one or more of the nucleotides capable of increasing the stability of the guide sequence association with a Cas protein in a CRISPR complex. In some embodiments, the guide sequence or sequences are free of phosphorothioate linkages at one or more of the nucleotides capable of increasing the stability of the guide sequence association with a Cas protein in a CRISPR complex. n some embodiments, the guide sequence or sequences are free of phosphorothioate linkages at one or more of the nucleotides capable of enhancing the enzymatic efficiency of the guide sequence association with a Cas protein in a CRISPR complex.

The disclosure relates to compositions and pharmaceutical compositions comprising one or a plurality of guide sequences disclosed herein, wherein the one or a plurality of guide sequences comprises from about 1% to about 99% modified nucleotides, wherein each modified nucleotide comprises at least two modification disclosed herein. The disclosure also relates to compositions and pharmaceutical compositions comprising one or a plurality of guide sequences disclosed herein, wherein the one or a plurality of guide sequences comprises from about 1% to about 99% modified nucleotides, wherein each modified nucleotide comprises a 2′ halogen at its 2′ carbon of its sugar moiety and a phosphorothioate linkage between at least one of its adjacent nucleotides. In any embodiment, the one or plurality of guide sequences may comprise one or more nucleotides having Formula W, X, Y, and/or Z positioned in the sequence either contiguously or noncontiguously.

In some embodiments, the disclosure relates to compositions comprising a guide sequence, wherein the guide sequence comprises a transcription terminator domain comprising at least one modified nucleotide. In some embodiments, the disclosure relates to compositions comprising a guide sequence, wherein the guide sequence comprises a transcription terminator domain comprising at least one modified nucleotide comprising a modification at a 2′ carbon in its sugar moiety. In some embodiments, the disclosure relates to compositions comprising a guide sequence, wherein the guide sequence comprises a transcription terminator domain comprising from about 1% to about 100% modified nucleotides. In some embodiments, the disclosure relates to compositions comprising a guide sequence, wherein the guide sequence comprises a transcription terminator domain comprising from about 10% to about 100% modified nucleotides. In some embodiments, the disclosure relates to compositions comprising a guide sequence, wherein the guide sequence comprises a transcription terminator domain comprising from about 20% to about 100% modified nucleotides. In some embodiments, the disclosure relates to compositions comprising a guide sequence, wherein the guide sequence comprises a transcription terminator domain comprising from about 30% to about 100% modified nucleotides. In some embodiments, the disclosure relates to compositions comprising a guide sequence, wherein the guide sequence comprises a transcription terminator domain comprising from about 40% to about 100% modified nucleotides. In some embodiments, the disclosure relates to compositions comprising a guide sequence, wherein the guide sequence comprises a transcription terminator domain comprising from about 50% to about 100% modified nucleotides. In some embodiments, the disclosure relates to compositions comprising a guide sequence, wherein the guide sequence comprises a transcription terminator domain comprising from about 60% to about 100% modified nucleotides. In some embodiments, the disclosure relates to compositions comprising a guide sequence, wherein the guide sequence comprises a transcription terminator domain comprising from about 70% to about 100% modified nucleotides. In some embodiments, the disclosure relates to compositions comprising a guide sequence, wherein the guide sequence comprises a transcription terminator domain comprising from about 80% to about 100% modified nucleotides. In some embodiments, the disclosure relates to compositions comprising a guide sequence, wherein the guide sequence comprises a transcription terminator domain comprising from about 90% to about 100% modified nucleotides. In some embodiments, the disclosure relates to compositions comprising a guide sequence, wherein the guide sequence comprises a transcription terminator domain comprising from about 95% to about 100% modified nucleotides.

The disclosure relates to compositions and pharmaceutical compositions comprising a guide sequence and/or a crRNA-tracrRNA duplex, wherein the guide sequence or crRNA-tracrRNA comprises a DNA-binding domain (the sequence complementary to a target sequence of choice) comprising at least one unmodified nucleotide. In some embodiments, the disclosure relates to compositions comprising a guide sequence and/or a crRNA-tracrRNA duplex, wherein the a guide sequence and/or a crRNA-tracrRNA duplex comprises a DNA-binding domain comprising at least one nucleotide comprising an unmodified hydroxyl or hydrogen substituent at its 2′ carbon in its sugar moiety. In some embodiments, the disclosure relates to compositions comprising a guide sequence and/or a crRNA-tracrRNA duplex, wherein the a guide sequence and/or a crRNA-tracrRNA duplex comprises a DNA-binding domain comprising at least one nucleotide comprising an unmodified hydroxyl group at its 2′ carbon in its sugar moiety. In some embodiments, the disclosure relates to compositions comprising a guide sequence and/or a crRNA-tracrRNA duplex, wherein the a guide sequence and/or a crRNA-tracrRNA duplex comprises a DNA-binding domain comprising one or a combination of unmodified hydroxyl group at its 2′ carbon in its sugar moiety at positions identified in Table 5. In some embodiments, the disclosure relates to compositions comprising a guide sequence and/or a crRNA-tracrRNA duplex, wherein the a guide sequence and/or a crRNA-tracrRNA duplex comprises a DNA-binding domain comprising 1, 2, 3, 4, 5, 6, 7, 8, 9, 10, 11, 12, 13, 14, 15, 16, 17, 18, 19, 20, 21, 22, 23, 24, 25, 26, or 27 of the unmodified hydroxyl groups at the 2′ carbon in its sugar moiety at positions identified in Table 5.

In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 1% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 10% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 20% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 30% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 40% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 50% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 60% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 70% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 80% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 90% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 95% to about 100% modified nucleotides.

In some embodiments, the disclosure relates to compositions comprising a guide sequence and/or a crRNA-tracrRNA duplex, wherein the guide sequence and/or a crRNA-tracrRNA duplex comprises a nucleotide binding domain (such as a DNA-binding domain) comprising at least one modified nucleotide. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising at least one modified nucleotide at its 2′ carbon. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 1% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 10% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 20% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 30% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 40% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 50% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 60% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 70% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 80% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 90% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 95% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 35% to about 75% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 40% to about 60% modified nucleotides.

In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to the RNA sequence:

(SEQ ID NO: 14)

UUAUCAACUUGAAAAAGUGGCACCGAGUCGGUGCUUUUUUU.

In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein the guide sequence comprises at one modified nucleotide. In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein the guide sequence comprises at least one modified nucleotide at its 2′ carbon. In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein the guide sequence comprises from about 1% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 1% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 10% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 20% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 30% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 40% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 50% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 60% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 70% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, 99% sequence identity to SEQ ID NO:12, wherein SEQ ID NO:12 comprises from about 80% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 90% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:12, wherein SEQ ID NO:12 comprises from about 95% to about 100% modified nucleotides.

In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to the RNA sequence: GGGCGAGGAGCUGUUCACCG (SEQ ID NO: 32). In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein the guide sequence comprises at one modified nucleotide. In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein the guide sequence comprises at least one modified nucleotide at its 2′ carbon. In some embodiments, the disclosure relates to compositions comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein the guide sequence comprises from about 1% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 1% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 10% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 20% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 30% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 40% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, 99% sequence identity to SEQ ID NO:32, wherein SEQ ID NO:32 comprises from about 50% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, 99% sequence identity to SEQ ID NO:32, wherein SEQ ID NO:32 comprises from about 60% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 70% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 80% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 90% to about 100% modified nucleotides. In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to SEQ ID NO:32, wherein SEQ ID NO:32 comprises from about 95% to about 100% modified nucleotides.

In some embodiments, the disclosure relates to a composition comprising a guide sequence comprising, consisting essentially of, or consisting of a sequence that has at least 70%, 75%, 80%, 85%, 90%, 91%, 92%, 93%, 94%, 95%, 96%, 97%, 98%, or 99% sequence identity to any nucleic acid or amino acid sequence disclosed herein.

In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to any one or combination of SEQ ID NO: 1, SEQ ID NO: 2, SEQ ID NO: 3, SEQ ID NO: 4. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 1. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 2. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 3. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 4. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 5. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 6. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 7. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 8. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 9. In some embodiments, the disclosure relates to a compositions comprising a guide sequence comprising, consisting essentially of, or consisting of a sequence that is at least 70%, 75%, 80%, 85%, 90%, 91%, 92%, 93%, 94%, 95%, 96%, 97%, 98%, 99% or 100% homologous to SEQ ID NO: 10.

In some embodiments, the disclosure relates to a compositions comprising a guide sequence comprising a Cas-binding domain comprising, consisting essentially of, or consisting of a sequence that is at least 70%, 75%, 80%, 85%, 90%, 91%, 92%, 93%, 94%, 95%, 96%, 97%, 98%, 99%, or 100% homologous to SEQ ID NO: 8 flanking sequence SEQ ID NO:9. In some embodiments, the Cas-binding domain comprises a sequence at least 70%, 75%, 80%, 85%, 90%, 91%, 92%, 93%, 94%, 95%, 96%, 97%, 98%, or 99% homologous to SEQ ID NO:8 flanking sequence SEQ ID NO:9 and comprises between about 42 nucleotides to about 150 nucleotides in length and comprises at least one or a combination of conserved nucleotides disclosed in Table 6 whereby the position number of 1 corresponds to position 1 of SEQ ID NO:8, and wherein, if the Cas-binding domain is more than 42 nucleotides long, position 43 an onward is contiguous with position 42 of SEQ ID NO:11 (such as SEQ ID NO:11-N_{(1-110 nt)}, where N_{(1-110 nt)} can be any modified or unmodified nucleotide (A, U, C, G) in length from 1-110 or more nucleotides. In some embodiments, the N_{(1-110 nt)} can be any modified or unmodified nucleotide (A, U, C, G) in length capable of forming a modified or unmodified loop region as set forth in “Genome-scale transcriptional activation by an engineered CRISPR-Cas9 complex” Nature (Zhang, et al.); 517, 583-588 (29 Jan. 2015), which is herein incorporated by reference in its entirety. The additional nucleotides in the Cas-binding domain may bind other RNAs or proteins as desired while conserving cas-binding to the sgRNA in the Cas-binding domain

In some embodiments, the disclosure relates to a compositions comprising a guide sequence comprising a Cas-binding domain comprising, consisting essentially of, or consisting of SEQ ID NO:8 or a domain sharing a disclosed percent homology with SEQ ID NO:8 optionally comprising from about 5, 10, 20, 30, 40, 50, 60, 70, 80, 90, 100, 110, 120, 130, 140, 150, 160, 170, 180, 190 or more nucleotides on the 3′ end of SEQ ID NO:8. In some embodiments, the disclosure relates to a compositions comprising a guide sequence comprising a DNA-binding domain or nucleotide binding domain comprising, consisting essentially of, or consisting of GGGCGAGGAGCUGUUCACCG (SEQ ID NO: 32) or a domain sharing a disclosed percent homology with GGGCGAGGAGCUGUUCACCG (SEQ ID NO: 32) optionally comprising from 1, 2, 3, 4, 5, or more nucleotides on the 3′ end of GGGCGAGGAGCUGUUCACCG (SEQ ID NO: 32). In some embodiments, the disclosure relates to a compositions comprising a guide sequence comprising a DNA-binding domain or nucleotide binding domain comprising, consisting essentially of, or consisting of GGCGAGGAGCUGUUCACCG (SEQ ID NO: 35), GCGAGGAGCUGUUCACCG (SEQ ID NO: 36), CGAGGAGCUGUUCACCG (SEQ ID NO: 37), or GAGGAGCUGUUCACCG (SEQ ID NO: 38) or any functional fragment thereof capable of binding a nucleotide sequence encoding a functional fragment of GFP. In some embodiments, the In some embodiments, the disclosure relates to a compositions comprising a guide sequence comprising a DNA-binding domain or nucleotide binding domain comprising, consisting essentially of, or consisting of the sequences set forth in Table 4 or disclosed herein.

In some embodiments, the disclosure relates to a compositions comprising a guide sequence comprising a Cas-binding domain comprising, consisting essentially of, or consisting of a sequence that is 70%, 75%, 80%, 85%, 90%, 91%, 92%, 93%, 94%, 95%, 96%, 97%, 98%, or 99%, homologous to SEQ ID NO:8 flanking SEQ ID NO:9 and comprising at least one or combination of conserved nucleotides identified in Table 6.

TABLE 6
sgRNA conserved nucleotides. sgRNA comprising one or
a combination of the following nucleotides of the Cas9
binding domain and/or the transcription terminator
region maintain or enhance Cas9 binding. sgRNA has been
modified at 2O′ position in one or a combination of
the following nucleotides has reduced Cas9 binding.

| | nucleotide |
|---|---|
| Cas9 binding domain # | |
| Based upon position of | |
| SEQ ID NO: 8 | |
| 2 | U |
| 3 | U |
| 4 | U |
| 23 | G |
| 24 | U |
| 25 | U |
| 27 | A |
| 31 | A |
| 38 | G |
| Terminator region # | |
| (based upon position | |
| number of SEQ ID | |
| NO: 9) | nucleotide |
| 2 | U |
| 3 | U |
| 4 | A |
| 7 | A |

In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 2 of SEQ ID NO:11 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 3 of SEQ ID NO:11 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 4 of SEQ ID NO:11 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 23 of SEQ ID NO:11 is a guanine. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 24 of SEQ ID NO:11 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 25 of SEQ ID NO:11 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 27 of SEQ ID NO:11 is an adenine. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides or deoxyribonucleotides; and the Cas-protein binding domain comprises from about 42 to about 150 nucleotides comprising SEQ ID NO:11 or a nucleotide sequence in which position 31 of SEQ ID NO:11 is an adenine. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the transcription terminator domain comprises from about 1% to about 99% modified ribonucleotides; and the Cas-protein binding domain comprises from about 42 to about 150 nucleotides comprising SEQ ID NO:11 or a nucleotide sequence in which position 38 of SEQ ID NO:11 is a guanine. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% ribonucleotides or deoxyribonucleotides and/or the transcription terminator domain comprises from about 1% to about 99% modified ribonucleotides or deoxyribonucleotides; and the Cas-protein binding domain comprises from about 42 to about 150 nucleotides comprising SEQ ID NO:11 or a nucleotide sequence in which position 42 of SEQ ID NO:11 is a guanine.

In some embodiments, the composition comprises any sgRNA or tracr/mate sequences disclosed herein, wherein the sgRNA or tracr/mate sequence comprises a plurality of contiguous domains from a 5′ to 3′ orientation: a DNA-binding domain, a Cas-protein binding domain, and a transcription terminator domain; wherein the DNA binding comprises a sequence at least 60, 70, 80, 90 or 100% complementary to a target sequence and is from about 15 to about 30 nucleotides long; wherein the Cas-protein binding domain comprises a nucleotide sequence that has 75, 80, 85, 90, 95, 96, 97, 98, 99, or 100% sequence homology to SEQ ID NO:8 and is from about 42 to about 200 nucleotides long; and wherein the transcription terminator domain comprises a sequence at least 60, 70, 80, 90 or 100% complementary to SEQ ID NO:9 and is from about 35 to about 200 nucleotides long. In some embodiments, the composition comprises any sgRNA disclosed herein, wherein the sgRNA comprises a plurality of contiguous domains from a 5′ to 3′ orientation: a DNA-binding domain, a Cas-protein binding domain, and a transcription terminator domain; wherein the DNA binding comprises a sequence at least 60, 70, 80, 90 or 100% complementary to a target sequence and is from about 25 to about 30 nucleotides long; wherein the Cas-protein binding domain comprises a nucleotide sequence that has 75, 80, 85, 90, 95, 96, 97, 98, 99, or 100% sequence homology to the bases of SEQ ID NO:8 and is from about 42 to about 200 nucleotides long; and wherein the transcription terminator domain comprises a sequence at least 60, 70, 80, 90 or 100% homolgous to the bases of SEQ ID NO:9 and is from about 35 to about 200 nucleotides long. In some embodiment, the transcription terminator region is free of 2′ fluorines on any 2′ C within its sequence.

In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the Cas-protein binding domain comprises from about 1% to about 99% modified ribonucleotides; and the transcription terminator domain comprises from about 35 to about 200 or more nucleotides comprising SEQ ID NO:9 or a nucleotide sequence in which position 2 of SEQ ID NO:9 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the Cas-protein binding domain comprises from about 1% to about 99% modified ribonucleotides; and the transcription terminator domain comprises from about 35 to about 200 or more nucleotides comprising SEQ ID NO:9 or a nucleotide sequence in which position 3 of SEQ ID NO:9 is a uracil. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the Cas-protein binding domain comprises from about 1% to about 99% modified ribonucleotides; and the transcription terminator domain comprises from about 35 to about 200 or more nucleotides comprising SEQ ID NO:9 or a nucleotide sequence in which position 4 of SEQ ID NO:9 is an adenine. In some embodiments, the composition comprises a plurality of domains from a 5′ to 3′ orientation: a DNA-binding domain, a Cas-protein binding domain, and a transcription terminator domain, wherein the DNA-binding domain comprises from about 1% to about 99% modified ribonucleotides and/or the Cas-protein binding domain comprises from about 1% to about 99% modified ribonucleotides; and the transcription terminator domain comprises from about 35 to about 200 or more nucleotides comprising SEQ ID NO:9 or a nucleotide sequence in which position 7 of SEQ ID NO:12 is an adenine.

In some embodiments, the disclosure relates to a compositions comprising a guide sequence comprising, consisting essentially of, or consisting of a sequence that is 70%, 75%, 80%, 85%, 90%, 91%, 92%, 93%, 94%, 95%, 96%, 97%, 98%, 99% homologous to any one or combination of sequences disclosed herein, wherein the guide sequence comprises a fragment or variant of the sequences disclosed herein but possesses the same or substantially the same function as the full-length sequence disclosed herein. For example, in the case of a fragment or variant of a guide RNA disclosed herein that comprises modified nucleotides in the DNA-binding domain, in some embodiments, the variant or fragment would be functional insomuch as it would exceed or retain some or all of its capacity to bind DNA at that domain as compared to the full-length sequence.

In some embodiments, the DNA-binding domain is free of modifications in any one of its first 2, 3, 4, 5 or more nucleotides on its 5′ end. In some embodiments the transcription terminator domain is free of modifications on any of its last 2, 3, 4, 5 or more nucleotides on its 3′ end.

The disclosure relates to a nucleic acid sequence comprising at least one or a combination of domains from a 5′ to 3′ orientation: a DNA-binding domain, a Cas protein-binding domain, and a transcription terminator domain, wherein the DNA-binding domain comprises from about 1% to about 99% modified nucleotides and/or the transcription terminator domain comprises from about 1% to about 99% modified nucleotides. The disclosure relates to a nucleic acid sequence consisting of a series of contiguous domains from a 5′ to 3′ orientation: a DNA-binding domain, a Cas protein-binding domain, and a transcription terminator domain, wherein the DNA-binding domain comprises from about 1% to about 99% modified nucleotides and/or the transcription terminator domain comprises from about 1% to about 99% modified nucleotides; and wherein the Cas protein-binding domain comprises from about 1% to about 99% modified nucleotides comprising one or a combination of the nucleotides in Table 6.

Any of the disclosed nucleic acid sequences may comprise any one or combination or set of modifications disclosed herein. In some embodiments, the guide nucleic acid, crRNA and/or tracer comprises RNA, DNA, or combinations of both RNA and DNA. In some embodiments, the either as a part of a modified nucleobase or a modified sugar.

Modifications to nucleotides are known in the art but include any of the disclosed modifications disclosed in the present application. Oligonucleotides particularly suited for the practice of one or more embodiments of the present disclosure comprise 2′-sugar modified oligonucleotides wherein one or more of the 2′-deoxy ribofuranosyl moieties of the nucleoside is modified with a halo, alkoxy, aminoalkoxy, alkyl, azido, or amino group. For example, the substitutions which may be independently selected from F, CN, CF₃, OCF₃, OCN, O-alkyl, S-alkyl, SMe, SO₂Me, ONO₂, NO₂, NH₃, NH₂, NH-alkyl, OCH₃═CH₂ and OCCH. In each of these, alkyl is a straight or branched chain of C₁ to C₂₀, having unsaturation within the carbon chain. A preferred alkyl group is C₁-C₉ alkyl. A further preferred alkyl group is C₅-C₂₀ alkyl.

A first group of substituents include 2′-deoxy-2′-fluoro substituents. A further preferred group of substituents include C₁ through C₂₀ alkoxyl substituents. An additional group of substituents include cyano, fluoromethyl, thioalkoxyl, fluoroalkoxyl, alkylsulfinyl, alkylsulfonyl, allyloxy or alkeneoxy substituents.

In further embodiments of the present disclosure, the individual nucleotides of the oligonucleotides of the disclosure are connected via phosphorus linkages. Phosphorus linkages include phosphodiester, phosphorothioate and phosphorodithioate linkages. In one preferred embodiment of this disclosure, nuclease resistance is conferred on the oligonucleotides by utilizing phosphorothioate internucleoside linkages.

In further embodiments of the disclosure, nucleosides can be joined via linkages that substitute for the internucleoside phosphate linkage. Macromolecules of this type have been identified as oligonucleosides. The term “oligonucleoside” thus refers to a plurality of nucleoside units joined by non-phosphorus linkages. In such oligonucleosides the linkages include an —O—CH₂—CH₂—O-linkage (i.e., an ethylene glycol linkage) as well as other novel linkages disclosed in U.S. Pat. No. 5,223,618, issued Jun. 29, 1993, U.S. Pat. No. 5,378,825, issued Jan. 3, 1995 and U.S. patent application Ser. No. 08/395,168, filed Feb. 27, 1995. Other modifications can be made to the sugar, to the base, or to the phosphate group of the nucleotide. Representative modifications are disclosed in International Publication Numbers WO 91/10671, published Jul. 25, 1991, WO 92/02258, published Feb. 20, 1992, WO 92/03568, published Mar. 5, 1992, and U.S. Pat. No. 5,138,045, issued Aug. 11, 1992, all of which are herein incorporated by reference in their entireties.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62). Further algorithms may be found in U.S. application Ser. No. 61/836,080 filed Jun. 17, 2013 (attorney docket 44790.11.2022; Broad Reference BI-2013/004A); incorporated herein by reference.

In some embodiments, the disclosure relates to modifications of the guide sequence that include positions of the sequences disclosed herein replaced by modified nucleotides or guide sequences that include additions of long non-coding RNAs (lncRNAs).

lncRNA has attracted much attention due to their large number and biological significance. Many lncRNAs have been identified as mapping to regulatory elements including gene promoters and enhancers, ultraconserved regions and intergenic regions of protein-coding genes. Yet, the biological function and molecular mechanisms of lncRNA in human diseases in Data from the literature suggest that lncRNA, often via interaction with proteins, functions in specific genomic loci or use their own transcription loci for regulatory activity. In some embodiments, the guide sequence of the disclosure comprises a length of contiguous lncRNA from about 150 nucleotides to about 250, 300, 350, 400, 450, or 500 nucleotides. In some embodiments, the guide sequence comprises a nucleotide domain that is at least 70%, 75%, 80%, 85%, 90%, 95%, 96%, 97%, 98%, 99% complementary to a known lncRNA sequence. The guide sequence may comprise an RNA binding domain that comprises such a complementary sequence or may comprise one or a plurality of RNA binding domains that comprises a at least 70%, 75%, 80%, 85%, 90%, 95%, 96%, 97%, 98%, 99% complementary to a known lncRNA sequence.

In another embodiment, the disclosure provides a cell or a vector comprising one of the sgRNAs of the disclosure or functional fragments thereof. The cell may be an animal cell or a plant cell. In some embodiments, the cell is a mammalian cell, such as a human cell.

In one aspect, the disclosure provides a vector system comprising one or more vectors. In some embodiments, the system comprises: (a) a synthetic guide sequence comprising at least one of the nucleic acid sequences disclosed herein, wherein the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and, optionally (2) a tracr mate sequence that is hybridized to a tracr sequence; and (b) a first regulatory element operably linked to an enzyme-coding sequence encoding said CRISPR enzyme comprising a nuclear localization sequence; wherein expressible components (the enzyme-coding sequence and the tracr sequences) are located on the same or different vectors of the system. In some embodiments, component (a) further comprises the tracr sequence downstream of the tracr mate sequence under the control of a tracr regulatory element. In some embodiments, component (a) further comprises one or more additional guide sequences operably linked to the tracr regulatory element, wherein when expressed, each the additional guide sequences direct sequence specific binding of a CRISPR complex to a different target sequence in a eukaryotic cell. In some embodiments, the system comprises the tracr sequence under the control of its own, second regulatory element, such as a polymerase III promoter. In some embodiments, the tracr sequence exhibits at least 50%, 60%, 70%, 80%, 90%, 95%, or 99% of sequence complementarity along the length of the tracr mate sequence when optimally aligned. Determining optimal alignment is within the purview of one of skill in the art. For example, there are publically and commercially available alignment algorithms and programs such as, but not limited to, ClustalW, Smith-Waterman in matlab, Bowtie, Geneious, Biopython and SeqMan. In some embodiments, the CRISPR complex comprises one or more nuclear localization sequences of sufficient strength to drive accumulation of said CRISPR complex in a detectable amount in the nucleus of a eukaryotic cell. Without wishing to be bound by theory, it is believed that a nuclear localization sequence is not necessary for CRISPR complex activity in eukaryotes, but that including such sequences enhances activity of the system, especially as to targeting nucleic acid molecules in the nucleus. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes, or S. thermophilus Cas9, and may include mutated Cas9 derived from these organisms. The enzyme may be a Cas9 homolog or ortholog. In some embodiments, the CRISPR enzyme is codon-optimized for expression in a eukaryotic cell. In some embodiments, the CRISPR enzyme directs cleavage of one or two strands at the location of the target sequence. In some embodiments, the CRISPR enzyme lacks DNA strand cleavage activity. In some embodiments, the first regulatory element is a polymerase III promoter. In some embodiments, the second regulatory element is a polymerase II promoter.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Another aspect of the disclosure relates to a composition comprising a nucleic acid disclosed herein and one or a plurality of recombinant expression vectors. Generally, the disclosure relates to composition comprising a synthetic guide sequence and one or a plurality of recombinant expression vectors. Recombinant expression vectors can comprise a nucleic acid of the disclosure in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol Ill promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the (3-actin promoter, the phosphoglycerol kinase (PGK) promoter, and the EF1a promoter. Also encompassed by the term “regulatory element” are enhancer elements, such as WPRE; CMV enhancers; the R-U5′ segment in LTR of HTLV-1 (Mol. Cell. Biol., Vol. 8(1), p. 466-472, 1988); SV40 enhancer; and the intron sequence between exons 2 and 3 of rabbit 3-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. One or more nucleic acid sequences and one or more vectors can be introduced into host cells to thereby form complexes with other cellular or non-natural compounds, produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

The disclosure also relates to pharmaceutical compositions comprising: (i) one or guide sequences disclosed herein or one or more pharmaceutically acceptable salts thereof; and (ii) a pharmaceutically acceptable carrier. The term “pharmaceutically acceptable salts” refers to physiologically and pharmaceutically acceptable salts of the nucleic acid sequences of the disclosure: i. e., salts that retain the desired biological activity of the nucleic acid sequences and do not impart undesired toxicological effects thereto.

Pharmaceutically acceptable base addition salts are formed with metals or amines, such as alkali and alkaline earth metals or organic amines Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharnut Sci., 1977, 66:1). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure. As used herein, a “pharmaceutical addition salt” includes a pharmaceutically acceptable salt of an acid form of one of the components of the compositions of the disclosure. These include organic or inorganic acid salts of the amines. In some embodiments, a pharmaceutically acceptable salt is selected from one or a combination of hydrochlorides, acetates, salicylates, nitrates and phosphates.

Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids; for example acetic acid, propionic acid, glycolic acid, succinic acid, malefic acid, hydroxymaleic acid, methylmaleic acid, fiunaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, malefic acid, fumaric acid, glucoruc acid, citric acid, malic acid, ascorbic acid, benzoic acid, tannic acid, palimitic acid, alginic acid, polyglutamic acid, naphthalenesulfonic acid, methanesulfonic acid, p-toluenesulfonic acid, naphthalenedisulfonic acid, polygaiacturonic acid, and the like; and (d) salts formed from elemental anions such as chlorine, bromine, and iodine.

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)), all of which are incorporated by reference in their entireties.

The term radioactive moiety means a substituent or component of a compound that comprises at least one radioisotope. Any radioisotope may be used. In some embodiments, the radioisotope is selected from Table 7. In some embodiments, the substituent or component of a compound of the present invention may incorporate any one, two, three, or more radioisotopes disclosed in Table 7. In some pharmaceutical compositions or methods disclosed herein, the compositions comprises a chemotherapeutic agent or method comprising administering a chemotherapeutic agent before, simultaneously with or after administration of the pharmaceutical compositions disclosed herein. In some embodiments the chemotherapeutic agents are chosen from one or a combination of those in Table 8.

TABLE 7
Radioisotopes that may be incorporated into
pharmaceutical compositions

²H, ³H, ¹³C, ¹⁴C, ¹⁵N, ¹⁶O, ¹⁷O, ³¹P, ³²P, ³⁵S, ¹⁸F, ³⁶Cl, ²²⁵Ac,
²²⁷Ac, ²¹²Bi, ²¹³Bi, ¹⁰⁹Cd, ⁶⁰Co, ⁶⁴Cu, ⁶⁷Cu, ¹⁶⁶Dy, ¹⁶⁹Er, ¹⁵²Eu, ¹⁵⁴Eu,
¹⁵³Gd, ¹⁹⁸Au, ¹⁶⁶Ho, ¹²⁵I, ¹³¹I, ¹⁹²Ir, ¹⁷⁷Lu, ⁹⁹Mo, ¹⁹⁴Os, ¹⁰³Pd, ^{195m}Pt,
³²P, ³³P, ²²³Ra, ¹⁸⁶Re, ¹⁸⁸Re, ¹⁰⁵Rh, ¹⁴⁵Sm, ¹⁵³Sm, ⁴⁷Sc, ⁷⁵Se, ⁸⁵Sr, ⁸⁹Sr,
^{99m}Tc, ²²⁸Th, ²²⁹Th, ¹⁷⁰Tm, ^{117m}Sn, ¹⁸⁸W, ¹²⁷Xe, ¹⁷⁵Yb, ⁹⁰Y, ⁹¹Y

TABLE 8
Table of Chemotherapeutic Agents

Alkylating agents
Cyclophosphamide
Mechlorethamine
Chlorambucil
Melphalan
Anthracyclines
Daunorubicin
Doxorubicin
Epirubicin
Idarubicin
Mitoxantrone
Valrubicin
Cytoskeletal disruptors (Taxanes)
Paclitaxel
Docetaxel
Epothilones
Histone Deacetylase Inhibitors
Vorinostat
Romidepsin
Inhibitors of Topoisomerase I
Irinotecan
Topotecan
Inhibitors of Topoisomerase II
Etoposide
Teniposide
Tafluposide
Kinase inhibitors
Bortezomib
Erlotinib
Gefitinib
Imatinib
Vemurafenib
Vismodegib
Monoclonal antibodies
Bevacizumab
Cetuximab
Ipilimumab
Ofatumumab
Ocrelizumab
Panitumab
Rituximab
Nucleotide analogs and precursor analogs
Azacitidine
Azathioprine
Capecitabine
Cytarabine
Doxifluridine
Fluorouracil
Gemcitabine
Hydroxyurea
Mercaptopurine
Methotrexate
Tioguanine (formerly Thioguanine)
Peptide antibiotics
Bleomycin
Actinomycin
Platinum-based agents
Carboplatin
Cisplatin
Oxaliplatin
Retinoids
Tretinoin
Alitretinoin
Bexarotene
Vinca alkaloids and derivatives
Vinblastine
Vincristine
Vindesine
Vinorelbine
Actinomycin
All-trans retinoic acid
Azacitidine
Azathioprine
Bleomycin
Bortezomib
Carboplatin
Capecitabine
Cisplatin
Chlorambucil
Cyclophosphamide
Cytarabine
Daunorubicin
Docetaxel
Doxifluridine
Doxorubicin
Epirubicin
Epothilone
Etoposide
Fluorouracil
Gemcitabine
Hydroxyurea
Idarubicin
Imatinib
Irinotecan
Mechlorethamine
Mercaptopurine
Methotrexate
Mitoxantrone
Oxaliplatin
Paclitaxel
Pemetrexed
Teniposide
Tioguanine
Topotecan
Valrubicin
Vinblastine
Vincristine
Vindesine
Vinorelbine

Compositions of the disclosure include pharmaceutical compositions comprising: a particle comprising any of the guides sequences or nucleic acid sequences disclosed herein, or pharmaceutically acceptable salts thereof: and a pharmaceutically acceptable carrier.

As used herein, a “particle” refers to any entity having a diameter of less than 100 microns (μm). Typically, particles have a longest dimension (e.g. diameter) of 1000 nm or less. In some embodiments, particles have a diameter of 300 nm or less. In some embodiments, nanoparticles have a diameter of 200 nm or less. In some embodiments, nanoparticles have a diameter of 100 nm or less. In general, particles are greater in size than the renal excretion limit, but are small enough to avoid accumulation in the liver. In some embodiments, a population of particles may be relatively uniform in terms of size, shape, and/or composition. In general, inventive particles are biodegradable and/or biocompatible. Inventive particles can be solid or hollow and can comprise one or more layers. In some embodiments, particles are spheres, spheroids, flat, plate-shaped, cubes, cuboids, ovals, ellipses, cylinders, cones, or pyramids. In some embodiments, particles can be a matrix of polymers. In some embodiments, the matrix is cross-linked. In some embodiments, formation of the matrix involves a cross-linking step. In some embodiments, the matrix is not substantially cross-linked. In some embodiments, formation of the matrix does not involve a cross-linking step. In some embodiments, particles can be a non-polymeric particle (e.g. a metal particle, quantum dot, ceramic, inorganic material, bone, etc.). Components of the pharmaceutical compositions disclosed herein may comprise particles or may be microparticles, nanoparticles, liposomes, and/or micelles comprising one ore more disclosed nucleic acid sequences. As used herein, the term “nanoparticle” refers to any particle having a diameter of less than 1000 nm. Examples of nanoparticles are disclosed in Nature Biotechnology 31, 638-646, which is herein incorporated by reference in its entirety.

Pharmaceutical “carrier” or “excipient”, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient or carrier is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

### Methods of Making Compositions and Modifications

Modified oligonucleotides and guide sequence of the disclosure may be made with automated, solid phase synthesis methods known in the art. During solid phase synthesis, phosphoramidite monomers are sequentially coupled to a nucleoside that is covalently linked to a solid support. This nucleoside is the 3′ terminal nucleoside of the modified oligonucleotide. Typically, the coupling cycle comprises four steps: detritylation (removal of a 5′-hydroxyl protecting group with acid), coupling (attachment of an activated phosphoroamidite to the support bound nucleoside or oligonucleotide), oxidation or sulfurization (conversion of a newly formed phosphite trimester with an oxidizing or sulfurizing agent), and capping (acetylation of unreacted 5′-hydroxy 1 groups). After the final coupling cycle, the solid support-bound oligonucleotide is subjected to a detritylation step, followed by a cleavage and deprotection step that simultaneously releases the oligonucleotide from the solid support and removes the protecting groups from the bases. The solid support is removed by filtration, the filtrate is concentrated and the resulting solution is tested for identity and purity. The oligonucleotide is then purified, for example using a column packed with anion-exchange resin.

This term includes oligonucleotides composed of naturally-occurring nucleobases, sugars and covalent internucleoside (backbone) linkages as well as oligonucleotides having non-naturally-occurring portions which function similarly Such modified or substituted oligonucleotides are often preferred over native forms because of desirable properties such as, for example, enhanced cellular uptake, enhanced affinity for nucleic acid target and increased stability in the presence of nucleases.

In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference in its entirety.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Any of the olignucleotide backbone modifications here may replace any one of the internucleotide linkages set forth in Formula W, X, Y, and/or Z.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference in its entirety.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Some embodiments of the disclosure are oligonucleotides with phosphorothioate backbones and oligonucleosides with heteroatom backbones, and in particular —CH2-NH—O—CH2-, —CH2-N(CH3)-O—CH2- [known as a methylene (methylimino) or MMI backbone], —CH2-O—N(CH3)-CH2-, —CH2-N(CH3)-N(CH3)-CH2- and —O—N(CH3)-CH2-CH2- [wherein the native phosphodiester backbone is represented as —O—P—O—CH2-] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. In some embodiments, oligonucleotides of the disclosure comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)nO]mCH₃, O(CH₂)nOCH₃, O(CH₂)nNH₂, O(CH₂)nCH₃, O(CH₂)nONH₂, and O(CH₂)nON[(CH₂)nCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, acetamide, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH2CH2OCH3, also known as 2′-O-(2-methoxyethyl) or 2′-MOE) (Martin et al., Helv. Chim Acta, 1995, 78, 486-504) i.e., an alkoxyalkoxy group. Another modification includes 2′-dimethylaminooxyethoxy, i.e., a O(CH₂)₂O N(CH₃)₂ group, also known as 2′-DMAOE, and 2′-dimethylamino-ethoxyethoxy (2′-DMAEOE), i.e., 2′-O—CH₂—O—CH2-N(CH₂)₂.

Other modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides may also include a modified thioester group on the 2′, 3′ and/or 5′ nucleoside. Such modifications in the 5′ carbon of the ribose sugar also for formation of single 5′-S-thioester linkages between nucleotides in a synthetic nucleotide sequence. In any 3′ or 5′ linkage between nucleotides any one or both positions may create a series of linkages between nucleotides in one or a plurality of synthetic guide nucleic acids disclosed herein. The linkages at the 2′ or 3′ can create thioester bond, phosphorothioriate linkages between two or a plurality of nucleosides in the oligonucleotide. In some embodiments, the guide nucleic acid comprises at least two contiguous nucleosides linked by a phosphate containing group as shown in the following formula:

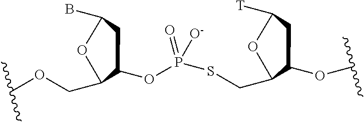

wherein B and T are independently selected as any natural or non-natural (modified) nucleobase, O is oxygen, P is phosphorous, and S is sulphur. In some embodiments, the naturally occurring 3′ and/or 2′ linkage in the nucleotide is replaced or supplemented with one or a plurality of linkers atoms. Such linkages are disclosed in US Publication WO/2002/061110, which is incorporated by reference in its entirety, but any chemical linker to bridge a 3′ or 2′ bond between two nucleotides is contemplated herein. Strategically placed sulfur atoms in the backbone of nucleic acids have found widespread utility in probing of specific interactions of proteins, enzymes and metals. Sulfur replacement for oxygen may be carried out at the 2′-position of RNA and in the 3′-5′-positions of RNA and of DNA. Polyribonucleotide containing phosphorothioate linkages were obtained as early as 1967 by Eckstein et al. using DNA-dependent RNA polymerase from E. coli (57). DNA-dependent RNA polymerase is a complex enzyme whose essential function is to transcribe the base sequence in a segment of DNA into a complementary base sequence of a messenger RNA molecule. Nucleoside triphosphates are the substrates that serve as the nucleotide units in RNA. In the polymerization of triphosphates, the enzyme requires a DNA segment that serves as a template for the base sequence in the newly synthesized RNA. In the original procedure, Uridine 5′-O-(1-thiotriphosphate), adenosine 5′-O-triphosphate, and only d (AT) as a template was used. As a result, an alternating copolymer is obtained, in which every other phosphate is replaced by a phosphorothioate group. Using the same approach and uridine 5′-O-(1-thiotriphosphate) and adenosine 5′-O-(1-thiotriphosphate), polyribonucleotide containing an all phosphorothioate backbone can also synthesized. In both cases, nucleoside 5′-O-(1-thiotriphosphates) as a mixture of two diastereomers can be used. In some embodiments, alternating phosphorothioate groups link a DNA or RNA or hybrid sequence of predominantly RNA to form alternating phosphorothioate backbones. Optionally, linkers of any cyclic or acyclic hydrocarbon chains of varying length may be incorporated into the guide nucleic acid. In some embodiments, linkers of the disclosure comprise one or a plurality of: branched or non-branched alkyl, hydroakyl, hydroxyl, halogen, metal, nitrogen, or other atoms.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the disclosure. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941, and 5,750,692, each of which is herein incorporated by reference in its entirety.

Another modification of the oligonucleotides of the disclosure involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), palmityl moiety (Mishra et al., Biochim Biophys. Acta, 995, 1264, 229-237), or an octadecylamine or hexylamino-carbonyl-oxycholesterol moiety (Crooke et al., J. Pharmacol. Exp. Ther., 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference in its entirety.

It is not necessary for all positions in a given compound to be uniformly modified, and in fact more than one of the aforementioned modifications may be incorporated in a single sequence or compound or even at a single nucleoside or functional group within one or a plurality of positions within a nucleoside or an oligonucleotide.

Oligonucleotides of the present disclosure also relate to guide sequences comprising a one or a combination of: a DNA-binding domain, a Cas protein-binding domain, and a transcription terminator domain, and one or more targeting domains. As used herein “targeting domains” may be oligonucleotides, amino acid sequences, sugar moieties, lipid moieties or hybrids of any of the foregoing that are responsible for directing transformation or transfection or anchoring of the guide sequence disclosed herein into a cell of choice that comprises a target sequence. Creation of such chimeric molecules can be synthetically manufacture by known chemical arts.

For example, GalNAc-conjugated modification are known to direct oligonucleotides to liver cells. Modifications, such as GalNAc-conjugated modification, may be made to any one or combination of oligonucleotides disclosed herein with automated solid phase synthesis, similar to the solid phase synthesis that produced unconjugated oligonucleotides. During the synthesis of GalNAc-conjugated oligonucleotides, the phosphoramidite monomers are sequentially coupled to a GalNAc conjugate which is covalently linked to a solid support. The synthesis of GalNAc conjugates and GalNAc conjugate solid support is described, for example in U.S. Pat. No. 8,106,022, which is herein incorporated by reference in its entirety for the description of the synthesis of carbohydrate-containing conjugates, including conjugates comprising one or more GalNAc moieties, and of the synthesis of conjugate covalently linked to solid support.

The disclosure also relates to synthesizing one or a plurality of oligonucleotides, such as sgRNA molecules. 2′-deoxy-2′-modified nucleosides of adenine, guanine, cytosine, thymidine and certain analogs of these nucleobases may be prepared and incorporated into oligonucleotides via solid phase nucleic acid synthesis. Novel oligonucleotides can be assayed for their hybridization properties and their ability to resist degradation by nucleases compared to the unmodified oligonucleotides. Initially, small electronegative atoms or groups can be selected because they would not be expected to sterically interfere with required Watson-Crick base pair hydrogen bonding (hybridization). However, electronic changes due to the electronegativity of the atom or group in the 2′-position may profoundly affect the sugar conformation.

2′-Substituted oligonucleotides can be synthesized by standard solid phase nucleic acid synthesis using an automated synthesizer such as Model 380B (Perkin-Elmer/Applied Biosystems) or MilliGen/Biosearch 7500 or 8800. Triester, phosphoramidite, or hydrogen phosphonate coupling chemistries [Oligonucleotides. Antisense Inhibitors of Gene Expression. M. Caruthers, p. 7, J. S. Cohen (Ed.), CRC Press, Boca Raton, Fla., 1989] are used with these synthesizers to provide the desired oligonucleotides. The Beaucage reagent [J. Amer. Chem. Soc., 112, 1253 (1990)] or elemental sulfur [Beaucage et al., Tet. Lett., 22, 1859 (1981)] is used with phosphoramidite or hydrogen phosphonate chemistries to provide 2′-substituted phosphorothioate oligonucleotides.

2′-substituted nucleosides (A, G, C, T(U), and other modified nucleobases) may be prepared by modification of several literature procedures as described below.

Procedure 1. Nucleophilic Displacement of 2′-Leaving Group in Arabino Purine Nucleosides. Nucleophilic displacement of a leaving group in the 2′-up position (2′-deoxy-2′-(leaving group)arabino sugar) of adenine or guanine or their analog nucleosides. General synthetic procedures of this type have been described by Ikehara et al., Tetrahedron, 34, 1133 (1978); ibid., 31, 1369 (1975); Chemistry and Pharmaceutical Bulletin, 26, 2449 (1978); ibid., 26, 240 (1978); Ikehara, Accounts of Chemical Research, 2, 47 (1969); and Ranganathan, Tetrahedron Letters, 15, 1291 (1977).

Procedure 2. Nucleophilic Displacement of 2,2′-Anhydro Pyrimidines. Nucleosides thymine, uracil, cytosine or their analogs are converted to 2′-substituted nucleosides by the intermediacy of 2,2′-cycloanhydro nucleoside as described by Fox et al., Journal of Organic Chemistry, 29, 558 (1964).

Procedure 3. 2′-Coupling Reactions. Appropriately 3′,5′-sugar and base protected purine and pyrimidine nucleosides having a unprotected 2′-hydroxyl group are coupled with electrophilic reagents such as methyl iodide and diazomethane to provide the mixed sequences containing a 2′-OMe group H. Inoue et al., Nucleic Acids Research, 15, 6131.

Procedure 4. 2-Deoxy-2-substituted Ribosylations. 2-Substituted-2-deoxyribosylation of the appropriately protected nucleic acid bases and nucleic acids base analogs has been reported by Jarvi et al., Nucleosides & Nucleotides, 8, 1111-1114 (1989) and Hertel et al., Journal of Organic Chemistry, 53, 2406 (1988).

Procedure 5. Enzymatic Synthesis of 2′-Deoxy-2′-Substituted Nucleosides. The 2-Deoxy-2-substituted glycosyl transfer from one nucleoside to another with the aid of pyrimidine and purine ribo or deoxyribo phosphorolyses has been described by Rideout and Krenitsky, U.S. Pat. No. 4,381,344 (1983).

Procedure 6. Conversion of 2′-Substituents Into New Substituents. 2′-Substituted-2′-deoxynucleosides are converted into new substituents via standard chemical manipulations. For example, Chladek et al. [Journal of Carbohydrates, Nucleosides & Nucleotides, 7, 63 (1980)] describes the conversion of 2′-deoxy-2′-azidoadenosine, prepared from arabinofuranosyladenine, into 2′-deoxy-2′-aminoadenosine.

Procedure 7. Free Radical Reactions. Conversions of halogen substituted nucleosides into 2′-deoxy-2′-substituted nucleosides via free radical reactions has been described by Parkes and Taylor [Tetrahedron Letters, 29, 2995 (1988)].

Procedure 8. Conversion of Ribonucleosides to 2′-Deoxy-2′-Substituted Nucleoside. Appropriately 3′,5′-sugar and base protected purine and pyrimidine nucleosides having a unprotected 2′-hydroxyl group are converted to 2′-deoxy-2′-substituted nucleosides by the process of oxidation to the 2′-keto group, reaction with nucleophilic reagents, and finally 2′-deoxygenation. Procedures of this type have been described by De las Heras, et al. [Tetrahedron Letters, 29, 941 (1988)].

Procedure 9. In one process of the disclosure, 2′-deoxy substituted guanosine compounds are prepared via an (arabinofuranosyl)guanine intermediate obtained via an oxidation-reduction reaction. A leaving group at the 2′ position of the arabinofuranosyl sugar moiety of the intermediate arabino compound is displaced via an SN2 reaction with an appropriate nucleophile. This procedure thus incorporates principles of both Procedure 1 and Procedure 8 above. 2′-Deoxy-2′-fluoroguanosine is preferably prepared via this procedure. The intermediate arabino compound was obtained utilizing a variation of the oxidation-reduction procedure of Hansske et al. [Tetrahedron, 40, 125 (1984)]. According to this disclosure, the reduction was effected starting at −78° C. and allowing the reduction reaction to exothermically warm to about −2° C. This results in a high yield of the intermediate arabino compound.

In conjunction with use of a low temperature reduction, utilization of a tetraisopropyldisiloxane blocking group (a “TPDS” group) for the 3′ and 5′ positions of the starting guanosine compound contributes to an improved ratio of intermediate arabino compound to the ribo compound following oxidation and reduction. Following oxidation and reduction, the N2 guanine amino nitrogen and the 2′-hydroxyl moieties of the intermediate arabino compound are blocked with isobutyryl protecting groups (“Ibu” groups). The tetraisopropyldisiloxane blocking group is removed and the 3′ and 5′ hydroxy groups are further protected with a second blocking group, a tetrahydropyranyl blocking group (“THP” group). The isobutyryl group is selectively removed from 2′-hydroxyl group followed by derivation of the 2′ position with a triflate leaving group. The triflate group was then displaced with inversion about the 2′ position to yield the desired 2′-deoxy-2′-fluoroguanosine compound.

In addition to the triflate leaving group, other leaving groups include, but are not limited to, alkylsulfonyl, substituted alkylsulfonyl, arylsulfonyl, substituted arylsulfonyl, heterocyclosulfonyl or trichloroacetimidate. Representative examples include p-(2,4-dinitroanilino)benzenesulfonyl, benzenesulfonyl, methylsulfonyl, p-methylbenzenesulfonyl, p-bromobenzenesulfonyl, trichloroacetimidate, acyloxy, 2,2,2-trifluoroethanesulfonyl, imidazolesulfonyl and 2,4,6-trichlorophenyl.

The isobutyryl group remaining on the N2 heterocyclic amino moiety of the guanine ring can be removed to yield a completely deblocked nucleoside. However, preferably, for incorporation of the 2′-deoxy-2′-substituted compound into an oligonucleotide, deblocking of the 2 isobutyryl protecting group is deferred until after oligonucleotide synthesis is complete. Normally for use in automated nucleic acid synthesizers, blocking of the N2 guanine moiety with an isobutyryl group is preferred. Thus, advantageously, the N2-isobutyryl-blocked 2′-deoxy-2′-substituted guanosine compounds resulting from the method of the disclosure can be directly used for oligonucleotide synthesis on automated nucleic acid synthesizers.

### Methods

The disclosure relates to a method of reducing off-target enzyme activity of a Cas protein or functional fragment thereof comprising exposing the Cas protein or functional fragment thereof to a chemically modified nucleic acid sequence disclosed herein comprising at least one fluorinated nucleotide.

The disclosure relates to a method of enhancing enzyme activity of a Cas protein or functional fragment thereof comprising exposing the Cas protein or functional fragment thereof to a chemically modified nucleic acid sequence disclosed herein comprising at least one unmodified nucleotide at one or more positions that bind to or interact with to the Cas protein or functional fragment thereof in an enzymatically active CRISPR complex.

The disclosure also relates to a method of altering expression of at least one gene product in a cell comprising introducing into a cell an engineered, non-naturally occurring CRISPR associated (Cas) (CRISPR-Cas) system comprising: (a) a vector comprising a nucleotide sequence encoding any CRISPR enzyme disclosed herein, any mutated CRISPR enzyme having at least 70%, 75%, 80%, 85%, 90%, 91%, 92%, 93%, 94%, 95%, 9%, 97%, 98%, or 99% sequence homology to any CRISPR enzyme disclosed herein (such as Table 13), or functional fragment thereof; and (b) a nucleic acid sequence disclosed herein, wherein components (a) and (b) are located on same or different vectors of the system; wherein the cell contains and expresses a DNA molecule having a target sequence and encoding the gene product; and wherein the guide RNA targets and hybridizes with a DNA target sequence, the CRISPR enzyme or functional fragment thereof cleaves the DNA molecule, whereby expression of the at least one gene product is altered.

The disclosure also relates to a method of altering expression of at least one gene product in a cell comprising introducing into a cell an engineered, non-naturally occurring CRISPR associated (Cas) (CRISPR-Cas) system comprising: (a) a vector comprising a nucleotide sequence encoding a Type I, Type-II, or Type III Cas9 protein or functional fragment thereof; and (b) a nucleic acid sequence disclosed herein, wherein components (a) and (b) are located on same or different vectors of the system; wherein the cell contains and expresses a DNA molecule having a target sequence and encoding the gene product; and wherein the guide RNA targets and hybridizes with a DNA target sequence and the Cas9 protein or functional fragment thereof cleaves the DNA molecule, whereby expression of the at least one gene product is altered.

The disclosure also relates to a method of improving the enzymatic efficiency of a Cas protein or functional fragment thereof comprising: exposing the Cas protein or functional fragment thereof to a chemically a modified nucleic acid sequence disclosed herein. In some embodiments, the modified nucleic acid sequence is a guide sequence comprising ribonucleotides and at least one fluorinated nucleotide in at least one or plurality of any domain disclosed herein. In some embodiments, the step of exposing the Cas protein or functional fragment thereof to a chemically a modified nucleic acid sequence disclosed herein. In some embodiments the enzymatic efficiency is increased by no less than from about 5% to about 10%. In some embodiments the enzymatic efficiency is increased by no less than from about 5% to about 15%. In some embodiments the enzymatic efficiency is increased by no less than from about 5% to about 20%. In some embodiments the enzymatic efficiency is increased by no less than from about 5% to about 25%. In some embodiments the enzymatic efficiency is increased by no less than from about 1% to about 25%. In some embodiments the enzymatic efficiency is increased by no less than from about 1% to about 20%. In some embodiments the enzymatic efficiency is increased by no less than from about 1% to about 15%. In some embodiments the enzymatic efficiency is increased by no less than from about 1% to about 10%. In some embodiments the enzymatic efficiency is increased by no less than from about 2 times to about 10 times the efficiency of the same Cas protein exposed to a unmodified guide sequence.

The disclosure also relates to a method of increasing the sensitivity of a cancer cell to one or more chemotherapeutic agents, the method comprising contacting a cancer cell with one or more pharmaceutical compositions disclosed herein.

The disclosure also relates to a method of increasing the sensitivity of a cancer in a subject in need thereof to one or more chemotherapeutic agents, the method comprising administering to a subject diagnosed with cancer or suspected of having cancer one or more pharmaceutical compositions disclosed herein. In some embodiments, the cancer in the subject is not responsive to chemotherapeutic agents.

The disclosure also relates to a method of destroying a cancer stem cell, the method comprising contacting a cancer stem cell with one or more pharmaceutical compositions disclosed herein. The disclosure also relates to a method of making a chimeric antigen receptor (CAR) positive T cell by exposing one or more T cells, isolated from a subject, to one or a plurality of guide sequences disclosed herein.

The disclosure also relates to a method of treating or preventing growth and/or proliferation of a cancer stem cell in a subject diagnosed with or suspected of having cancer, the method comprising administering to a subject diagnosed with cancer or suspected of cancer one or more pharmaceutical compositions disclosed herein.

The disclosure also relates to a method of treating or preventing liver disease in a subject diagnosed with or suspected of having liver disease, the method comprising administering to a subject diagnosed with liver disease a pharmaceutically effective amount of one or more pharmaceutical compositions disclosed herein.

The disclosure also relates to a method of treating or preventing cardiovascular disease in a subject diagnosed with or suspected of having cardiovascular disease, the method comprising administering to a subject diagnosed with cardiovascular disease one or more pharmaceutical compositions disclosed herein, wherein the composition comprises a concentration of one or plurality of sgRNA molecules comprising a DNA-binding domain capable of binding PSCK9 (such as disclosed in Table 4) sufficient to activate a Cas enzyme in the subject.

According to one aspect of the present disclosure, a eukaryotic cell is transfected with a two component system including one or a plurality of guide sequences complementary to genomic DNA and an enzyme that interacts with the guide sequence when it is duplexed with the target sequence of genomic DNA. The one or a plurality of guide sequences and the enzyme are expressed by the cell. The RNA of the RNA/enzyme complex then binds to complementary genomic DNA. The enzyme then performs a function, such as cleavage of the genomic DNA. In some embodiments, the one or a plurality of guide sequences include from about 10 nucleotides to about 250 nucleotides. In some embodiments, the one or a plurality of guide sequences include from about 20 nucleotides to about 100 nucleotides. According to certain aspects, the enzyme may perform any desired function in a site specific manner for which the enzyme has been engineered. According to one aspect, the eukaryotic cell is a yeast cell, plant cell or mammalian cell. According to one aspect, the enzyme cleaves genomic sequences targeted by one or a plurality of guide sequences, thereby creating a genomically altered eukaryotic cell.

According to one aspect, the present disclosure provides a method of genetically altering a human cell by including: (i) one or a plurality of synthetic guide sequence; and (ii) one or a plurality of nucleic acids encoding an RNA complementary to genomic DNA into the genome of the cell; and (iii) a nucleic acid encoding an enzyme that performs a desired function on genomic DNA into the genome of the cell. According to one aspect, the RNA and the enzyme are expressed, and the RNA hybridizes with complementary genomic DNA. According to one aspect, the enzyme is activated to perform a desired function, such as cleavage or nicking, in a site-specific manner when the RNA is hybridized to the complementary genomic DNA. According to one aspect, the RNA and the enzyme are components of a bacterial Type I, Type II, or Type III CRISPR system.

According to one aspect, the disclosure relates to a method of altering a eukaryotic cell comprising: transfecting the eukaryotic cell with a nucleic acid disclosed herein complementary to genomic DNA of the eukaryotic cell, transfecting the eukaryotic cell with a nucleic acid encoding an enzyme that interacts with the RNA and cleaves the genomic DNA in a site-specific manner, wherein the cell expresses the RNA and the enzyme, the RNA binds to complementary genomic DNA and the enzyme cleaves the genomic DNA in a site specific manner According to one aspect, the enzyme is Cas9 or modified Cas9 or a homolog of Cas9. According to one aspect, the eukaryotic cell is a yeast cell, a plant cell or a mammalian cell. According to one aspect, the a nucleic acid disclosed herein comprises from about 10 to about 250 nucleotides. According to one aspect, the nucleic acid disclosed herein comprises from about 20 to about 100 nucleotides.

According to one aspect, a method of altering a human cell is provided including transfecting the human cell with a nucleic acid encoding RNA complementary to genomic DNA of the eukaryotic cell, transfecting the human cell with a nucleic acid encoding an enzyme that interacts with the RNA and cleaves the genomic DNA in a site specific manner, wherein the human cell expresses the RNA and the enzyme, the RNA binds to complementary genomic DNA and the enzyme cleaves the genomic DNA in a site specific manner According to one aspect, the enzyme is Cas9 or modified Cas9 or a homolog of Cas9. Modified cas9 proteins or homologs of Cas9 are for instance disclosed in U.S. Pat. No. 9,074,199, which is incorporated herein by reference. According to one aspect, the RNA includes between about 10 to about 250 nucleotides. According to one aspect, the RNA includes between about 20 to about 100 nucleotides. The step of transfecting a nucleic acid encoding an RNA may be added to any method disclosed herein so that there is sequential or concurrent transfection of not only synthetic guide sequences such as those disclosed herein but also one or a plurality of vectors comprising

According to one aspect, the disclosure relates to a method of altering a eukaryotic cell at a plurality of genomic DNA sites comprising: transfecting the eukaryotic cell with one or a plurality of nucleic acids complementary to different sites on genomic DNA of the eukaryotic cell, transfecting the eukaryotic cell with a nucleic acid encoding an enzyme that interacts with the nucleic acid complementary to different sites on genomic DNA of the eukaryotic cell, such that the enzyme cleaves the genomic DNA in a site-specific manner, wherein the cell expresses the enzyme, the nucleic acids complementary to different sites on genomic DNA of the eukaryotic cell bind to complementary genomic DNA and the enzyme cleaves the genomic DNA in a site specific manner. According to one aspect, the enzyme is Cas9. According to one aspect, the eukaryotic cell is a yeast cell, a plant cell or a mammalian cell. According to one aspect, the a nucleic acid disclosed herein comprises from about 10 to about 250 nucleotides. According to one aspect, the nucleic acid disclosed herein comprises from about 20 to about 100 nucleotides.

The disclosure relates to a composition comprising a cell with any one or combination of nucleic acid sequences disclosed herein. In some embodiments, the cell is a plant, insect or mammalian cell. In some embodiments, the cell is a eukaryotic cell or a prokaryotic cell. the cell may be isolated from the body, a component of a culture system, or part of an organism. In general, the system and methods described herein include at least two components: (1) the RNAs or DNA/RNA hybrid (guide nucleic acid, a crRNA, tracrRNA, and/or a single cr/tracrRNA hybrid) targeted to a particular sequence in a cell (e.g., either genomic DNA, or in an extrachromosomal plasmid, such as a reporter); and (2) a Cas protein disclosed herein. In some cases, a system also can include a nucleic acid containing a donor sequence targeted to a sequence in the cell. The donor sequence and the guide sequence may be on one or a plurality of nucleic acid molecules. The Cas protein disclosed herein can create targeted DNA double-strand breaks at the desired locus (or loci), and the host cell can repair the double-strand break using the provide donor DNA sequence, thereby incorporating the modification stably into the host genome.

The construct(s) containing the guide RNA or RNA/DNA hybrid molecules, crRNA, tracrRNA, cr/tracrRNA hybrid, Cas protein disclosed herein coding sequence, and, where applicable, donor sequence, can be delivered to a cell using, for example, biolistic bombardment, electrostatic potential or through transformation permeability reagents (reagents known to increase the permeability of the cell wall or cell membrane). Alternatively, the system components can be delivered using Agrobacterium-mediated transformation, insect vectors, grafting, or DNA abrasion, according to methods that are standard in the art, including those described herein. In some embodiments, the system components can be delivered in a viral vector (e.g., a vector from a DNA virus such as, without limitation, geminivirus, AAV, adenovirus, lentiviral strains attenuated for human use, bean yellow dwarf virus, wheat dwarf virus, tomato leaf curl virus, maize streak virus, tobacco leaf curl virus, tomato golden mosaic virus, or Faba bean necrotic yellow virus, or a vector from an RNA virus such as, without limitation, a tobravirus (e.g., tobacco rattle virus, tobacco mosaic virus), potato virus X, or barley stripe mosaic virus.

After an organism is infected, administered or transfected with a sequence encoding a Cas protein disclosed herein or a functional fragment thereof, a crRNA, a trRNA, a crRNA and a tracrRNA, a cr/tracrRNA hybrid, and/or a synthetic guide nucleic acid (and, in some cases, a donor sequence), any suitable method can be used to determine whether targeted mutagenesis has occurred at the target site. In some embodiments, a phenotypic change can indicate that a donor sequence has been integrated into the target site. Such is the case for transgenic plants encoding a defective GUS:NPTII reporter gene, for example PCR-based methods also can be used to ascertain whether a genomic target site contains targeted mutations or donor sequence, and/or whether precise recombination has occurred at the 5′ and 3′ ends of the donor.

### Kits

In some embodiments, kits in accordance with the present disclosure may be used to mutate endogenous genetic material in cell types of interest. In some embodiments, kits for mutating cells comprise the nucleic acids, compositions, or pharmaceutical compositions described herein and, optionally, cell growth medium and a cell type of interest. Any nucleic acid, composition, or component thereof disclosed may be arranged in a kit either individually or in combination with any other Any nucleic acid, composition, or component thereof. The disclosure provides a kit to perform any of the methods described herein. In some embodiments, the kit comprises at least one container comprising one or a plurality of oligonucleotides comprising a DNA-binding domain sequence complementary to genomic DNA inside of a cell. In some embodiments, the kit comprises at least one container comprising any of the polypeptides or functional fragments described herein. In some embodiments, the polypeptides are in solution (such as a buffer with adequate pH and/or other necessary additive to minimize degradation of the polypeptides during prolonged storage). In some embodiments, the polypeptides or oligonucleotides are lyophilized for the purposes of resuspension after prolonged storage. In some embodiments, the kit comprises: at least one container comprising one or a plurality of polypeptides comprising or functional fragments disclosed herein and/or oligonucleotides disclosed herein; and a solid support upon which genomic DNA of a cell may be mutated. In some embodiments, the kit optionally comprises instructions to perform any or all steps of any method described herein.

The kit may contain two or more containers, packs, or dispensers together with instructions for preparation of an array. In some embodiments, the kit comprises at least one container comprising the oligonucleotides described herein and a second container comprising a means for maintenance, use, and/or storage of the oligonucleotides such as storage buffer. In some embodiments, the kit comprises a composition comprising any polypeptide disclosed herein in solution or lyophilized or dried and accompanied by a rehydration mixture. In some embodiments, the polypeptides and rehydration mixture may be in one or more additional containers.

The compositions included in the kit may be supplied in containers of any sort such that the shelf-life of the different components are preserved, and are not adsorbed or altered by the materials of the container. For example, suitable containers include simple bottles that may be fabricated from glass, organic polymers, such as polycarbonate, polystyrene, polypropylene, polyethylene, ceramic, metal or any other material typically employed to hold reagents or food; envelopes, that may consist of foil-lined interiors, such as aluminum or an alloy. Other containers include test tubes, vials, flasks, and syringes. The containers may have two compartments that are separated by a readily removable membrane that upon removal permits the components of the compositions to mix. Removable membranes may be glass, plastic, rubber, or other inert material.

Kits may also be supplied with instructional materials. Instructions may be printed on paper or other substrates, and/or may be supplied as an electronic-readable medium, such as a floppy disc, CD-ROM, DVD-ROM, zip disc, videotape, audio tape, or other readable memory storage device. Detailed instructions may not be physically associated with the kit; instead, a user may be directed to an internet web site specified by the manufacturer or distributor of the kit, or supplied as electronic mail.

The disclosure also provides a kit comprising: an guide sequence disclosed herein; and a vector comprising a nucleic acid sequence encoding a Cas protein or CRISPR enzyme operably linked to a regulatory element active in a eukaryotic cell. In some embodiments, the kit further comprises at least one of the following: one or a plurality of eukaryotic cells comprising regulatory protein capable of trans-activation of the regulatory element, cell growth media, a volume of fluorescent stain or dye, and a set of instructions, optionally accessible remotely through an electronic medium.

Any and all journal articles, patent applications, issued patents, or other cited references disclosed herein are incorporated by reference in their respective entireties.
PCT/US16/40221
U.S. Pat. No. 8,697,359
WO/2015/048690, 60, 72
201410067479
Nature Biotechnology 32, 1262-1267
Nature Biotech, 2014 March; 32(3) 279-84 (K. Juong-MGH).
US Patent Publication 2010/0076057
US Patent Publication 2011/0189776
US Patent Publication 2011/0223638
US Patent Publication 2013/0130248
Makarova et al., “Evolution and classification of the CRISPR-Cas systems” 9(6) Nature Reviews Microbiology 467-477 (1-23) (June 2011).
Wiedenheft et al., “RNA-guided genetic silencing systems in bacteria and archaea” 482 Nature 331-338 (Feb. 16, 2012).
Jinek et al., “A Programmable Dual-RNA-Guided DNA Endonuclease in Adaptive Bacterial Immunity” 337 Science 816-821 (Aug. 17, 2012).
Carroll, “A CRISPR Approach to Gene Targeting” 20(9) Molecular Therapy 1658-1660 (September 2012).
Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289.
Hale et al., Essential Features and Rational Design of CRISPR RNAs That Function With the Cas RAMP Module Complex to Cleave RNAs, Molecular Cell, (2012) vol. 45, Issue 3, 292-302.
Erik Sontheimer, Project 7: Establishing RNA-Directed DNA Targeting in Eukaryotic Cells; Project dates: Nov. 16, 2011 to Dec. 31, 2012 (Feb. 4, 2012).
Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012).
U.S. Pat. Nos. 7,919,277, 8,282,920, 8,361,725, 8,501,405, 8,546,553, 8,557,561, 8,586,709, 8,592,556, 8,609,421, 8,673,568, 8,758,764, 8,771,766, 8,771,945, 8,795,965, 8,809,026, 8,841,260, 8,846,329, 8,846,354, 8,865,406, 8,871,445, 8,889,356, 8,889,418, 8,895,308, 8,906,616, 8,932,814, 8,940,507, 8,945,839, 8,993,233, 8,999,641, 9,023,649, 9,057,071, 9,068,179, 9,074,199, 9,101,100, 9,102,936

TABLE 9
Exemplary CRISPR enzyme sequences

| PDB | |
|---|---|
| Accession | |
| No. | Amino Acid Sequence |
| 3GOD | GSFTMDDISPSELKTILHSKRANLYYLQHCRVLVNGGRVEYVTDEGRHSHYWNIPIANTTSLLLGT |
| (SEQ ID | GTSITQAAMRELARAGVLVGFCGGGGTPLFSANEVDVEVSWLTPQSEYRPTEYLQRWVGFWFDEEK |
| NO: 39) | RLVAARHFQRARLERIRHSWLEDRVLRDAGFAVDATALAVAVEDSARALEQAPNHEHLLTEEARLS |
| | KRLFKLAAQATRYGEFVRAKRGSGGDPANRFLDHGNYLAYGLAATATWVLGIPHGLAVLHGKTRRG |
| | GLVFDVADLIKDSLILPQAFLSAMRGDEEQDFRQACLDNLSRAQALDFMIDTLKDVAQRSTVSA |
| 2I8E (SEQ | MAMLYLIFYDITDDNLRNRVAEFLKKKGLDRIQYSVFMGDLNSSRLKDVEAGLKIIGNRKKLQEDE |
| ID NO: 40) | RFFILIVPITENQFRERIVIGYSGSEREEKSNVVW |
| 3I4H (SEQ | MAHHHHHHGSRFLIRLVPEDKDRAFKVPYNHQYYLQGLIYNAIKSSNPKLATYLHEVKGPKLFTYS |
| ID NO: 41) | LFMAEKREHPKGLPYFLGYKKGFFYFSTCVPEIAEALVNGLLMNPEVRLWDERFYLHEIKVLREPK |
| | KFNGSTFVTLSPIAVTVVRKGKSYDVPPMEKEFYSIIKDDLQDKYVMAYGDKPPSEFEMEVLIAKP |
| | KRFRIKPGIYQTAWHLVFRAYGNDDLLKVGYEVGFGEKNSLGFGMVKVEGNKTTKEAEEQEKITFN |
| | SREELKTGV |
| 1WJ9 | MWLTKLVLNPASRAARRDLANPYEMHRTLSKAVSRALEEGRERLLWRLEPARGLEPPVVLVQTLTE |
| (SEQ ID | PDWSVLDEGYAQVFPPKPFHPALKPGQRLRFRLRANPAKRLAATGKRVALKTPAEKVAWLERRLEE |
| NO: 42) | GGFRLLEGERGPWVQILQDTFLEVRRKKDGEEAGKLLQVQAVLFEGRLEVVDPERALATLRRGVGP |
| | GKALGLGLLSVAP |
| 2WTE | MHHHHHHMKSYFVTMGFNETFLLRLLNETSAQKEDSLVIVVPSPIVSGTRAAIESLRAQISRLNYP |
| (SEQ ID | PPRIYEIEITDFNLALSKILDIILTLPEPIISDLTMGMRMINTLILLGIIVSRKRFTVYVRDEGGG |
| NO: 43) | SRVISFNDNTIRALMRDYSREEMKLLNVLYETKGTGITELAKMLDKSEKTLINKIAELKKFGILTQ |
| | KGKDRKVELNELGLNVIKLNKSVIESSKSSEELVKENKGKEVNIPY |

### Methods of Treating Liver Disease

The disclosure also relates to a method of treating or preventing a liver disease in a subject in need thereof, the method comprising administering to the subject a therapeutically effective amount of a pharmaceutical composition comprising a chemically modified sgRNA, thereby treating or preventing the liver disease in the subject. In certain embodiments, the subject is afflicted with liver disease. In certain embodiments, the subject is diagnosed with liver disease. In some embodiments, the subject is at risk for developing liver disease. In a particular embodiment, the subject is a human. In some embodiments, the pharmaceutical composition comprises one or more polymers. In some embodiments, the pharmaceutical composition comprises a lipid nanoparticle (e.g. a C12-200 particle) comprising the chemically modified sgRNA.

In some embodiments the liver disease is selected from the group consisting of fatty liver disease, nonalcoholic steatohepatitis, cirrhosis of the liver, and hepatocellular carcinoma. Fatty liver disease (FLD, also know as hepatosteatosis) is a prevalent liver condition that occurs when lipids accumulate in liver cells. The lipid accumulation causes cellular injury and sensitizes the liver to further injuries. The accumulated lipids may also impair hepatic microvascular circulation. FLD may arise from a number of sources, including excessive alcohol consumption and metabolic disorders, such as those associated with insulin resistance, obesity, and hypertension. Nonalcoholic fatty liver disease (NAFLD) may also result from metabolic disorders such as, e.g., galactosemia, glycogen storage diseases, homocystinuria, and tyrosemia, as well as dietary conditions such as malnutrition, total parenteral nutrition, starvation, and overnutrition. In certain cases, NAFLD is associated with jejunal bypass surgery. Other causes include exposure to certain chemicals such as, e.g., hydrocarbon solvents, and certain medications, such as, e.g., amiodarone, corticosteroids, estrogens (e.g., synthetic estrogens), tamoxifen, maleate, methotrexate, nucleoside analogs, and perhexiline Acute fatty liver conditions can also arise during pregnancy. Nonalcoholic steatohepatitis (NASH; metabolic steatohepatitis), is a condition characterized by liver inflammation and damage, often accompanied by fibrosis or cirrhosis of the liver. NASH may progress to further liver damage ultimately leading to chronic liver failure and, in some cases, hepatocellular carcinoma. See, for example, U.S. Pat. No. 9,556,155.

A subject in need of treatment may be one who is at increased risk of developing liver disease. For example, a subject having abnormal fat metabolism, alcoholism, advanced age (e.g., greater than 40, 50, 60, or 70 years of age), celiac disease, diabetes mellitus (e.g., type II diabetes mellitus), dyslipidemia, exposure to industrial solvents, galactosemia, glycogen storage diseases, homocystinuria, hyperferritinemia, hyperinsulinemia, hyperlipidemia, hypertension, hypertriglyceridemia, hyperuricemia, hypoxia, impaired fasting glycemia, inborn metabolic disorders (e.g., related to galactose, glycogen, homocysteine, or tyrosine metabolism), insulin resistance, iron overload, jejunal bypass surgery, low levels of high-density lipoprotein, Madelung's lipomatosis, malnutrition, Mauriac syndrome, metabolic syndrome, mitochondrial dysfunction, mitochondrial injury, mitochondrialopathies, niacin deficiency, Niemann-Pick disease, obesity (especially visceral adiposity or central obesity), overnutrition, pantothenic acid deficiency, peroxisomal diseases, polycystic ovarian syndrome, pregnancy, rapid weight loss, riboflavin deficiency, sleep apnea, starvation, tyrosemia, Weber-Christian disease, or Wilson's disease may have, or be at increased risk of developing, a disorder associated with hepatic lipid deposits. NAFLD has also been associated with rapid weight loss. In addition, patients treated with certain medications, such as, e.g., amiodarone, corticosteroids, estrogens (e.g., synthetic estrogens), maleate, methotrexate, perhexyline, salicylate, tamoxifen, tetracycline, and valproic acid may have, or be at increased risk of developing, a disorder associated with hepatic lipid deposits.

A subject in need of treatment may be presumptively diagnosed on the basis of symptoms. However, steatosis, particularly macrovesicular steatosis (in which hepatocytes are filled with large lipid droplets which displace the nuclei to the periphery), is often asymptomatic in adults and children. Alcohol-related fatty liver disease in general, is often asymptomatic. Microvesicular steatosis (in which hepatocytes are filled with small lipid droplets, and nuclei are centrally located) is more commonly symptomatic. NAFLD may also be more likely to be symptomatic in children. Carey et al., eds., 1998, The Washington Manual of Medical Therapeutics, 29th ed. (Lippincott Williams & Williams, Philadelphia).

Symptoms of a disorder associated with hepatic lipid deposits, when present, may be valuable in establishing a presumptive diagnosis. Such symptoms include, e.g., abdominal discomfort (e.g., discomfort in the right upper abdominal quadrant), acanthosis nigricans, bowel dismotility, coma, constipation, disseminated intravascular coagulopathy, epigastric pain, fatigue, hepatomegaly (generally with a smooth, firm surface upon palpation), hypoglycemia, jaundice, lipomatosis, lipoatrophy, lipodystrophy, nausea, neurological defects, Palmer erythema, panniculitis, periumbilical pain, small bowel bacterial overgrowth, spider angiomata, splenomegaly, subacute liver failure, and vomiting.

A subject in need of treatment may also be presumptively diagnosed by serum tests of liver enzymes. For example, steatosis may be indicated by elevated serum levels (often moderately elevated, e.g., elevated approximately 2, 3, 4, 5, 6, 7, 9, 10, 11, or 12-fold above normal levels) of liver enzymes (such as, e.g., alanine aminotransferase, aspartate aminotransferase, .gamma.-glutamyltransferase, alkaline phosphatase) when other causes (such as, e.g., acute hepatitis, autoimmune disease, chronic hepatitis, cirrhosis, fulminant hepatitis, hepatocellular carcinoma, metastatic carcinoma, right heart failure, and viral hepatitis) have been eliminated. For example, alanine aminotransferase (ALT or SGPT) values greater than 32, 24, or 56 units per liter of serum or at least 2, 3, 4, 5, 6, 7, 8, 9, 10, 11, 12, or more times normal values may be indicative of a disorder associated with hepatic lipid deposits, or by aspartate aminotransferase (AST or SGOT) values greater than 40 units per liter of serum or at least 2, 3, 4, 5, 6, 7, 8, 9, 10, 11, 12, or more times normal values. The ratio of AST to ALT is often less than one in NAFLD, but may be greater than one in patients with alcoholic liver disease or advanced liver disease. In addition, .gamma.-glutamyltransferase levels may be significantly elevated, e.g., at least 2, 3, 4, 5, 6, 7, 8, 9, 10, 11, 12, or more times normal values. The mean corpuscular volume (MPV) may be greater than, e.g., 86, 98, 100, or 110 femtoliters.

A subject in need of treatment may also be presumptively diagnosed by noninvasive imaging techniques (e.g., ultrasonography, computed tomography, and magnetic resonance imaging) when steatosis is greater than, e.g., 25% or 30%. In general, it may be difficult to distinguish between NAFLD and NASH to detect fibrosis, or to determine the progression of disease, by such imaging methods. NAFLD may present as a focal or diffuse accumulation of lipid, but in NASH the lipid is generally diffuse. NAFLD may also be detected by magnetic resonance spectroscopy, a technique which may be of value for quantitative determination of hepatic lipid levels. For example, determination of hepatic triglyceride levels by MRI has been demonstrated to correlate with histologic biopsy results. See, e.g., Kawamitsu et al., Magn. Reson. Med. Sci. 2:47-50 (2003).

A subject in need of treatment may be definitively diagnosed by liver biopsy. A liver is considered to be steatotic when a biopsy reveals at least 5-10% w/w fatty deposits (in practice, this is value may be determined microscopically as the fraction of lipid-filled hepatocytes). See, e.g., Clark et al., J. Am. Med. Assoc. 289:3000-3004 (2003) and Adams et al., Can. Med. Assoc. J. 172:899-905 (2005). A liver with fatty deposits comprising up to 25% w/w may be considered mildly steatotic, and a liver with fatty deposits comprising greater than 25% w/w may be considered severely steatotic. Histological findings indicative of NASH include steatosis, hepatocyte ballooning, lobular inflammation, Mallory hyaline bodies, mixed inflammatory infiltrate, pericellular fibrosis, and perisinusoidal fibrosis. Additional information may be found in, e.g., Neuschwander-Tetri et al., Hepatology 37:1202-1219 (2003).

Disease progression in NAFLD/NASH, as assessed by fibrosis in liver histology, has been reported to correlate with the degree of insulin resistance and other features of metabolic syndrome. Ryan et al., Diabetes Care, 28:1222-1224 (2005). Elevated levels of serum immunoglobulin A have also been associated with disease progression. Neuschwander-Tetri et al., Hepatology 37:1202-1219. Other markers proposed to be related to fibrosis in NAFLD patients include laminin, hyaluronan, type IV collagen, and aspartate aminotransferase. DosSantos et al., Braz. J. Med. Biol. Res. 38:747-753 (2005). Female gender is also associated with more rapid disease progression.

Efficacy of treatment may also be determined by detection of a reduction in one or more symptoms or clinical manifestations of a disease as well as any of the test described above for diagnosis.

Administration of a pharmaceutical composition comprising a chemically modified sgRNA to a subject may reduce serum levels of a hepatic enzyme (e.g., alanine aminotransferase, aspartate aminotransferase, γ-glutamyltransferase, or alkaline phosphatase) at least 10%, such as, e.g., at least 15, 20, 30, 40, 50, 60, 62, 64, 66, 68, or 70%, as compared to pre-treatment control.

Administration of a pharmaceutical composition comprising a chemically modified sgRNA to a subject may reduce serum levels of a disease marker (such as, e g, laminin, hyaluronan, type IV collagen, or immunoglobulin A) at least 10%, such as, e.g., at least 15, 20, 30, 40, 50, 60, 62, 64, 66, 68, or 70%, as compared to pre-treatment control. Administration of an inhibitor of a GSL synthesis enzyme, such as, e.g., a compound of Formula I, to a subject may reduce, e.g., hyperlipidemia, hypertriglyceridemia, or insulin resistance at least 10%, such as, e.g., at least 15, 20, 30, 40, 50, 60, 62, 64, 66, 68, or 70%.

Administration of a pharmaceutically effective amount of one or a plurality of pharmaceutical compositions comprising a chemically modified sgRNA to a subject may reduce histological features of a hepatic disorder associated with lipid deposition such as, e.g., cholestasis, fat cysts, fibrosis, granular iron, hepatocellular ballooning, increased numbers of eosinophils, inflammation, lobular disarray, lobular inflammation, macrovesicular steatosis, Mallory bodies, megamitochondria, necrosis, periodic acid-Schiff stained globules, portal inflammation, microvesicular steatosis, or steatosis, as determined by sequential liver biopsies. For example, the fraction of hepatocytes having pathogenic lipid deposits and/or the over-all amount of liver fat (e.g., triglycerides) may be reduced by, e.g., at least 15, 20, 30, 40, 50, 60, 62, 64, 66, 68, or 70%, as compared to pre-treatment control.

The chemically modified sgRNA may target a gene that is known to be involved in the development of liver disease, for example, PCSK9. In some embodiments, administration of the pharmaceutical composition comprising the chemically modified sgRNA to the subject results in reduction of LDL-C levels in the subject. In some embodiments, administration of the pharmaceutical composition to the subject results in a reduction in serum levels of at least one hepatic enzyme chosen from alanine aminotransferase, aspartate aminotransferase, γ-glutamyltransferase, and alkaline phosphatase. In some embodiments, administration of the pharmaceutical composition to the subject results in a decrease in hepatic lipid deposits.

Suitable methods of administering the pharmaceutical composition to the subject may include oral administration, parenteral delivery, including intramuscular, subcutaneous, intramedullary injections, as well as intrathecal, direct intraventricular, intravenous, or intraperitoneal injections. In a particular embodiment, the pharmaceutical composition is administered by injection. In some embodiments, the pharmaceutical composition is administered by intravenous infusion.

### Lipid Nanoparticles

Intracellular delivery of messenger RNA (mRNA) has the potential to induce protein production for many therapeutic applications. Although lipid nanoparticles have shown considerable promise for the delivery of small interfering RNAs (siRNA), their utility as agents for mRNA delivery has only recently been investigated. The most common siRNA formulations contain four components: an amine-containing lipid or lipid-like material, phospholipid, cholesterol, and lipid-anchored polyethylene glycol, the relative ratios of which can have profound effects on the formulation potency. Here, we develop a generalized strategy to optimize lipid nanoparticle formulations for mRNA delivery to the liver in vivo using Design of Experiment (DOE) methodologies including Definitive Screening and Fractional Factorial Designs. By simultaneously varying lipid ratios and structures, we developed an optimized formulation which increased the potency of erythropoietin-mRNA-loaded C12-200 lipid nanoparticles 7-fold relative to formulations previously used for siRNA delivery. Key features of this optimized formulation were the incorporation of 1,2-dioleoyl-sn-glycero-3-phosphoethanolamine (DOPE) and increased ionizable lipid:mRNA weight ratios. Interestingly, the optimized lipid nanoparticle formulation did not improve siRNA delivery, indicating differences in optimized formulation parameter design spaces for siRNA and mRNA. We believe the general method described here can accelerate in vivo screening and optimization of nanoparticle formulations with large multidimensional design spaces.

### EXAMPLES

### Example 1 DNA-RNA Chimeric Guides Enable Efficient Genome Editing

To accelerate the process of guide sequence evaluation, we made use of a cell reporter system that measures the editing efficiency of various modified crRNAs. HEK293T cells were infected by lentiviruses to constitutively express GFP and Streptococcus pyogenes Cas9 (SpCas9) (FIGS. 1a and b)(24). Transfecting the cells with a functional 42 nt crRNA targeting GFP and a 75 nt tracrRNA induces frame shifting indel formations at the GFP site and abrogates the expression of GFP (FIGS. 1a and 1b). Thus, if partial replacement with DNA in the guide sequence is not well tolerated, it will be less efficient at reducing GFP expression in cells than the unmodified RNA.

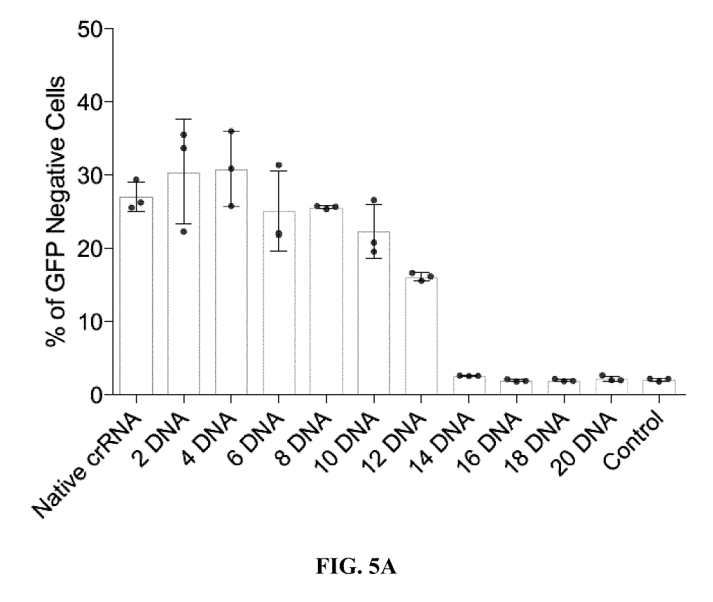

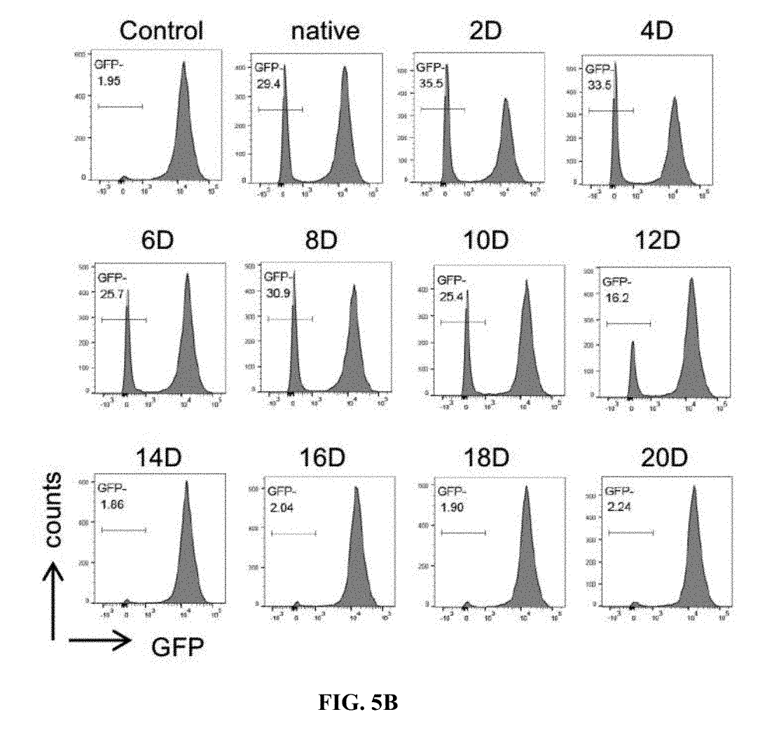

The crystal structure of Cas9-sgRNA indicates that RNA at the seed region (10 nucleotides at the 3′ end of the guide sequence) is essential for Cas9-sgRNA binding and recognition of targeted DNA (25,26). In contrast, the tail region (10 nucleotides at the 5′ end) of the guide sequence interacts with Cas9 less than the seed region (25,26). We hypothesized that the guide sequence may tolerate partial DNA replacement based on the structure of Cas9 and guide RNA (25,26). To test our hypothesis, we first synthesized unmodified crRNAs (native crRNA) based on a previously identified guide sequence targeting GFP²⁷. A number of crRNAs with the same guide sequence but varying degrees of DNA substitutions at the 5′ end were synthesized (FIG. 1c). When evaluated in HEK293T cells, native crRNA targeting GFP generated 27%±2% GFP negative cells (FIGS. 5A and 5B). We found that replacing 2, 4, 6, 8 or 10 RNA nucleotides with DNA nucleotides starting from the 5′ end of the guide sequences also generated similar levels of GFP negative cells (ranging from 22% to 31%) (FIGS. 5A and 5B), indicating that partial replacement of up to 10 nt DNA at the 5′ end of the guide sequence was tolerated. However, replacement with 12 DNA nucleotides generated significantly fewer GFP negative cells than the native crRNA, and replacement with 14 or more DNA nucleotides reduced GFP negative cells to background levels (˜2%) (FIGS. 5A and 5B). The efficiency of genome editing after DNA nucleotides replacement at the guide sequence was confirmed by Tracking of Indels by Decomposition (TIDE) analysis (28) of the PCR amplicons from the GFP genomic locus (FIG. 1d).

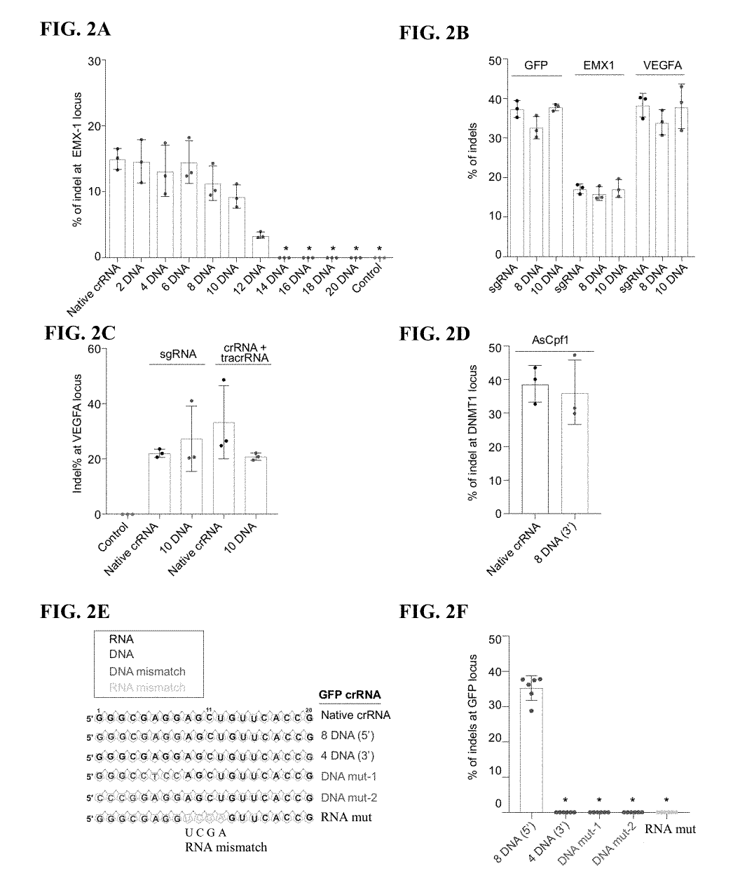

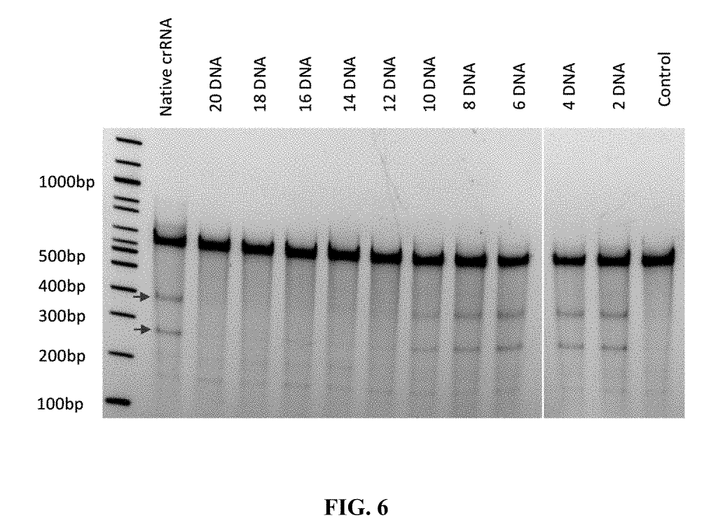

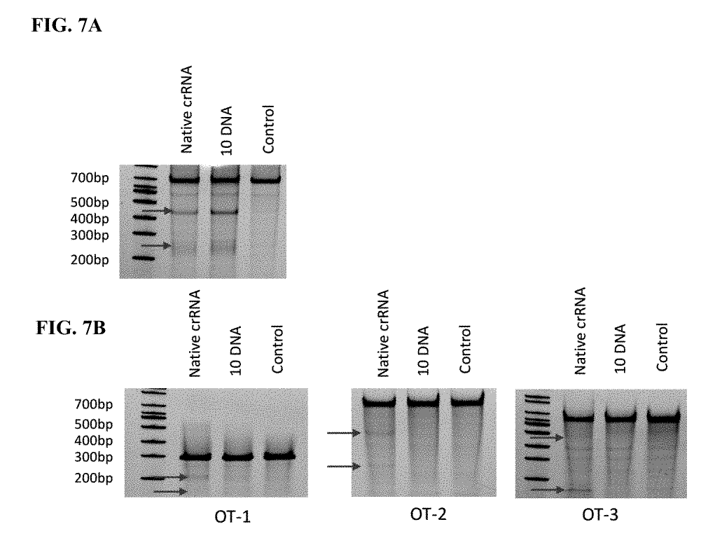

To study the effects of DNA nucleotide replacement on an endogenous gene, we synthesized crRNAs targeting the human gene EMX1 (Empty spiracles homeobox 1) (29), with various levels of DNA replacement at the 5′ end. After transfection of a crRNA targeting EMX1 and the tracrRNA into HEK293T stably expressing SpCas9, we found that replacing 2, 4, 6, 8 or 10 RNA nucleotides with DNA nucleotides induced comparable frequencies of indel formation as the native crRNA (FIG. 2a). In contrast, replacing 12 or more RNA nucleotides with DNA significantly decreased or abolished the editing activity of EMX1 crRNA (FIG. 2a). The efficiency of genome editing at the EMX1 locus was further examined by Surveyor nuclease assay (30), indicating that replacing 2 to 10 RNA nucleotides with DNA induced indels (FIG. 6).

To investigate whether partial DNA replacement can be tolerated by sgRNA, we synthesized sgRNAs targeting GFP, EMX1 and Vascular endothelial growth factor A (VEGFA) with and without DNA replacement (24). After transfecting the sgRNAs in HEK293T reporter cells, we found that the native sgRNAs and sgRNAs with substitution of 8 DNA and 10 DNA nucleotides in the 5′ end generated similar levels of indels in cells for all three guide sequences (FIG. 2b). These data show that partial DNA replacement of both crRNA and sgRNA retain on-target genome editing.

To investigate whether DNA-RNA chimeric crRNAs or sgRNA can be applied in therapeutic delivery, we incubated VEGFA 10DNA crRNA and a tracrRNA with Cas9 protein, or VEGFA 10DNA sgRNA with Cas9 protein to form ribonucleoprotein (RNP). We found that they induced similar levels of indels as native crRNA or sgRNA after RNP electroporation in Jurkat T cells (FIG. 2c).

To test whether DNA replacement works with other Cas enzymes, we synthesized AsCpf1 native crRNA targeting DNMT1 (31) or crRNA with 8 nt DNA replacement at 3′ end (as the guide sequence of Cpf1 crRNA is at 3′ end). HEK293T cells were transfected with a plasmid expressing AsCpf1 protein and then the crRNAs after 24 hrs. The Cpf1 crRNA with partial DNA replacement fully maintains its activity in human cells (FIG. 2d).

To investigate whether the DNA replacement could be introduced at the 3′ end of the guide sequence, 4 RNA nucleotides were replaced with DNA at the 3′ end of the guide sequences (FIGS. 2e and f). This modified crRNA did not induce indels of GFP (FIG. 2f), indicating replacement with DNA nucleotides at 3′ of guide sequence is not well-tolerated.

To further evaluate the potential of DNA nucleotide substitution in the guide sequence, we synthesized GFP crRNAs with 8 DNA nucleotides at the 5′ end (GFP-8DNA), but we mutated 4 nt of either DNA or RNA sequence (FIG. 2e). As expected, mutation of 4 RNA nucleotides (position 9-12 from 5′ end) fully abolished its genome editing activity (FIG. 20. We also found that mutations of 4 DNA nucleotides (position 1-4 or 5-8 from 5′ end) eliminated the activity of chimera crRNA, indicating that complementarity of DNA sequences in the guide region play an essential role in recognizing genomic DNA sequences.

### Example 2. DNA-RNA Chimeric Guides Reduce Off-Target Editing

Previous studies have reported that truncation of the RNA guide sequence to 17 RNA nucleotides instead of 20 RNA nucleotides decreases off-target activity of CRISPR/Cas9 (21). It was also reported that some truncated guide sequences reduce on-target editing (21). Because duplexes of DNA-DNA are less thermodynamically stable than the binding of DNA-RNA duplexes (32, 33), a partially DNA substituted crRNA guide sequence is less likely to tolerate mismatches when base pairing with genomic DNA. We therefore hypothesized that DNA-DNA binding between the crRNA with 5′ DNA replacement and target genomic DNA strand might reduce off-target editing. We synthesized crRNAs targeting a site within VEGFA (FIG. 3a) that is known to have high off-target frequencies in the human genome (24).

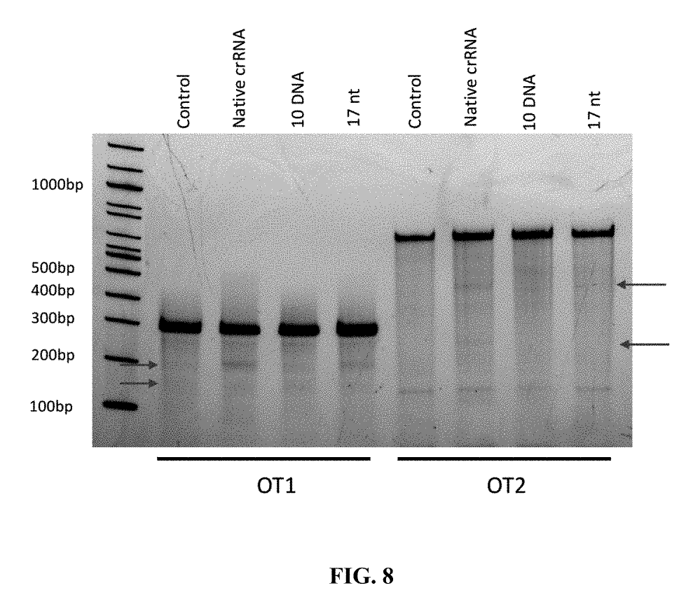

We found that the native VEGFA crRNA and the crRNA with 10 DNA nucleotides at the 5′ end (10DNA) generated similar levels of indels at the on-target genomic locus (FIG. 3b), further demonstrating that replacement of RNA with DNA was tolerated by various guide sequences. Interestingly, at the top 3 off-target sites, VEGFA 10DNA crRNA generated no detectable indels at 2 of 3 off-target sites and less than 4% indels at another site, compared with 10-15% off-target indels generated by native crRNA (FIG. 3c and FIG. 6). To compare off-target effects of a crRNA with partial DNA replacement and a crRNA with truncated 17 nt guide sequence, which has been shown to reduce off-target effects (21), a VEGFA crRNA with 17 nt guide sequence was synthesized. We found that the crRNA with 10 DNA nucleotides replaced showed comparable levels of off-target site indel formation as the truncated guide RNA (FIG. 8).

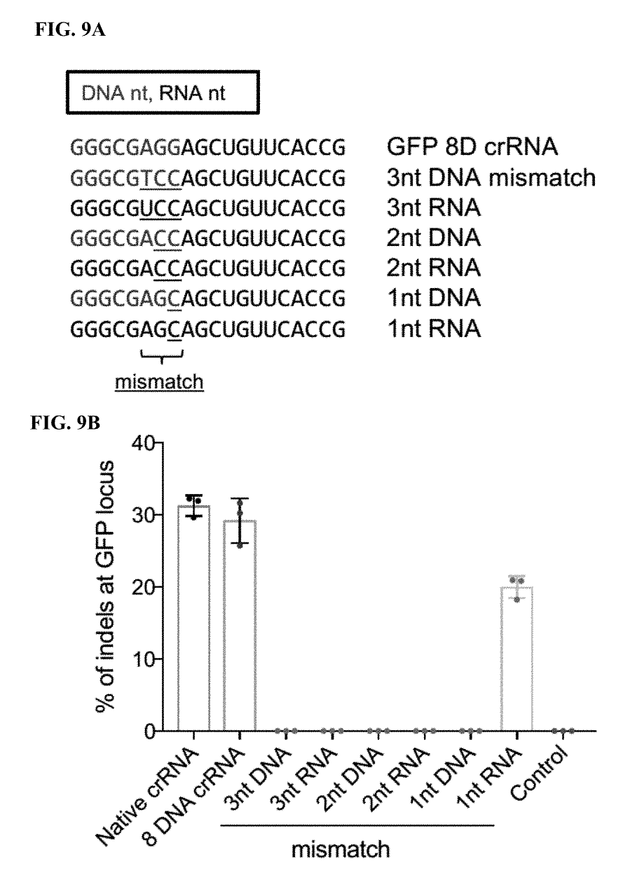

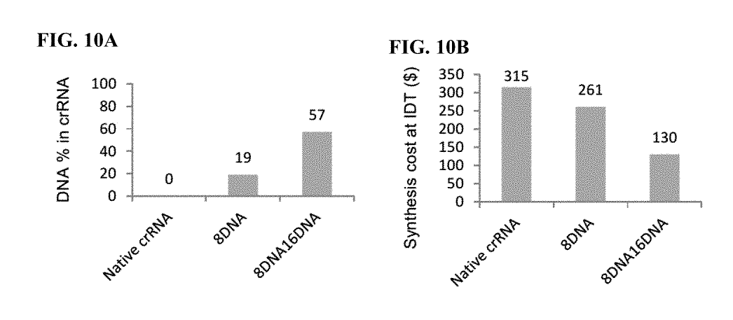

We hypothesized that if DNA replacement at the guide sequences provides higher specificity than native crRNA, the chimeric crRNAs would be less tolerant for mismatches than native crRNA. When both native GFP crRNA and GFP-8 DNA were mutated 2-4 nucleotides at the tail region of guide sequences, their activities of genome editing were abolished (FIGS. 2e and f, FIG. 9). Importantly, native GFP crRNA, but not GFP-8 DNA, tolerated single nucleotide mismatch in the tail region, as demonstrated by slight reduction of indels by native GFP crRNA with one nucleotide mutation in the tail region of crRNA, compared to no indels produced by GFP-8 DNA with one nucleotide mutation (FIG. 9). To investigate whether DNA replacement could enhance specificity of other guide sequences, we studied another GFP guide sequence (named GFP2) (FIG. 3d) (24). One nt mismatch in the GFP2 guide sequence has also been shown to mediate off-target genome editing (24). GFP2 native crRNA and crRNA replaced with 10 DNA nucleotides at the 5′ end (GFP2-10DNA) generated similar levels of indels at the GFP site (FIG. 3d-e). To compare “simulating” off-target editing of GFP2 native crRNA and GFP2-10DNA, we introduced a point mutation either at the seed or tail region of GFP2 crRNA and GFP2-10DNA to create a mismatch between the guide sequence and the target GFP sequence (FIG. 3d). If DNA replacement can enhance specificity and reduce off-target editing, then GFP2-10DNA will be less tolerant of mismatches than the native GFP crRNA. We found that mutations on GFP2 native crRNAs induced substantial indels of GFP in human cells (FIG. 3e). In contrast, mutated GFP2-10DNA showed reduced levels of indels (FIG. 3e), further indicating that partial DNA replacement can reduce off-target effects.

We performed GUIDE-Seq¹⁰ to systematically compare the genome-wide off-target activity of native and 10DNA crRNA. Three guide sequences were chosen: mouse Pcsk9³⁴, human EMX1, and human 293 site 4 (10). Mouse Hepa1-6 liver cells or human HEK293 cells stably expressing SpCas9 were transfected with Guide-Seq oligos, tracrRNA, and: (1) native crRNA or (2) 10DNA crRNA. Analysis of the off-target peaks revealed that all three 10DNA crRNAs had no detectable off-target sites using the threshold of 6 or fewer total mismatches between the guide and PAM (FIGS. 3f and 3g), although the depth of the GUIDE-Seq data for the EMX1 target site is suboptimal for definitive conclusions for this sequence. Consistent with the VEGFA off-target analysis, these data collected via an alternate approach indicate that 10DNA crRNA substantially reduces off-target editing.

### Example 3: Structure-Guided DNA Substitution at the 3′ of crRNA

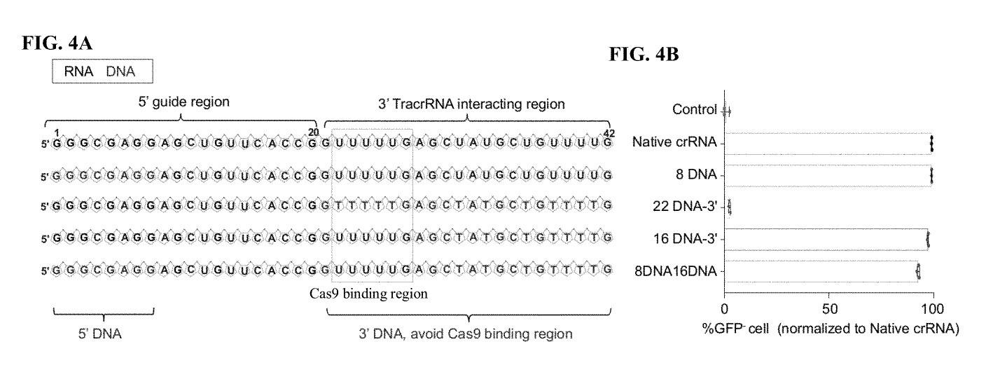

To explore whether the 3′ tracrRNA interacting region of crRNA can tolerate DNA replacement, we synthesized series of chimeric crRNAs targeting GFP (FIG. 4a). U2OS-GFP-PEST cells (21) stably expressing Cas9 were transfected with crRNAs and tracrRNA. GFP negative cells caused by Cas9-mediated frameshift NHEJ were measured by FACS at day 3 to report genome editing efficiency. As shown in FIG. 4b, replacing all 22 RNA nucleotides in the 3′ region with DNA (22 DNA) abolished genome editing. These data are consistent with our recent finding that 2-O-methyl chemical RNA modification of the entire 3′ region abolished sgRNA activity (34). Within the structure of Cas9-sgRNA complex (25,25), 6 nucleotides in the 3′ of crRNA/sgRNA have been shown to interact with the Cas9 protein. We hypothesized that avoiding substitution of RNA bases with DNA bases in those 6 nucleotides would help preserve crRNA activity. As expected, this 16DNA design (16 nt DNA substitution in 3′ region, avoiding Cas9 binding region) efficiently generated GFP⁻ cells comparable to native crRNA or 8DNA crRNA (FIG. 4b). We subsequently tested whether crRNA functionality can be maintained with 5′ and 3′ DNA substitution within one crRNA. crRNA with both 8DNA and 16DNA design (8DNA16DNA, 8 nt DNA in 5′ and 16 nt DNA in 3′) induced efficient genome editing (FIG. 4b). Of note, the majority of the 8DNA16DNA crRNA is comprised of DNA bases (57%) (FIG. 10a). Because DNA bases are more than 10-fold cheaper than RNA bases in oligo synthesis, the cost of 8DNA16DNA crRNA is ˜60% less than the cost of synthesis for native crRNA (FIG. 10b). Together, these data present an optimized DNA-RNA chimeric crRNA design that enables efficient genome editing in human cells and has potential to significantly reduce cost for certain CRISPR-Cas9 applications.

### Discussion

In this study, we demonstrate that partial DNA replacement of the guide RNA can retain on-target genome editing by CRISPR/Cas9. Importantly, replacement of RNA with DNA can significantly reduce off-target activity. Our study indicates that partial DNA guided genome editing of Cas9 is feasible in mammalian cells, extending the toolkit of CRISPR from a RNA-guided nuclease platform to a partial DNA-guided nuclease system. Moreover, our study indicates a simple and effective way to decrease off-target effects, which may be combined with modified Cas9 protein or improved delivery technologies to further minimize off-target activity (14-20, 35).

Our study indicates that partial DNA replacement is feasible for crRNA of spCas9 and Cpf1 (FIG. 2d). It is possible that this approach has utility for guide sequences of other Cas9 proteins, such as Staphylococcus aureus (Sa) Cas9 (36) or Neisseria meningitidis (Nm) Cas9 (37). Future work is required to study other types of chemical modifications or incorporation of DNA aptamers at the 5′ end of crRNA/sgRNA (38). In addition, whether DNA Cpf1 crRNA can decrease off-target specificity is to be tested. Because crRNA alone is sufficient to mediate genome editing by Cpf1, DNA-RNA chimera crRNA may be particular attractive for generating array or plate of crRNA libraries with reduced cost.

Our study indicates that the tail region of guide sequence is more amenable to DNA replacement than the seed region. Replacement of the 5′ end of the guide sequence maintains the activity of genome editing. However, Cas9 loses its genome editing capability in cells with crRNA harboring >12 nt DNA nucleotides at the 5′ end or 4 nt DNA at the 3′ end of the guide region. The crystal structure of Cas9-sgRNA complex indicates that the seed region of the guide sequence is essential for Cas9-sgRNA binding and recognition of targeted DNA (25,26). In contrast, the tail region is less interactive with Cas9 than the seed region (25,26). This likely explains why the tail, but not the seed region, can tolerate DNA replacement (FIGS. 1 and 2). This is consistent with previously studies that reported that the seed region is sensitive to chemical modifications (23). In our recent study, we demonstrated that using structure of Cas9-sgRNA as a guide, sgRNA and crRNA can be chemically modified to enhance their activity in cells and animals (34). We identified regions of sgRNA and crRNA that can tolerate modifications of the 2′OH, and we avoided modifying the 2′OH that interact with Cas9 protein (34). We also showed that modification of even one “interacting” 2′OH with 2′ 0-Methyl RNA (2′ OMe) at the guide region of crRNA abolishes its activity in mammalian cells (34). This finding is consistent with the data that replacing RNA with DNA at the tail region, but not seed could maintain the activity of crRNA (FIGS. 1 and 2). Furthermore, the helix geometry of DNA and RNA are different (32,33). It is possible that the variation of helix geometry of DNA-RNA chimeras may in part explain their activities in cells. We noticed that in the GUIDE-seq the number of sequencing reads of crRNAs targeting the human EMX1 and 293 site 4 in HEK293T cells were lower than the crRNA targeting mouse Pcsk9 in Hepa1-6 cells (FIGS. 3f and 3g). The numbers of our GUIDE-seq reads for the on-target locus are similar as the published study (10). Furthermore, the different form of guides and the delivery methods applied may also affect the outcome of the GUIDE-seq (10, 34). The higher reads in Hepa1-6 cells than HEK293T may be explained by higher concentration of GUIDE-seq oligonucleotide used in Hepa1-6 cells, because those cells are more tolerated for DNA oligo transfection. We noticed that in the GUIDE-seq of EMX1, three off-target sites have more than 10% reads as the designed for the DNA-substituted crRNA. However, to rule out unspecific reads, GUIDE-seq applied the criteria of using the threshold of <6 mismatches between the guide and the PAM sequence in total (10). These three off-target sites contain more than 6 mismatches, so they are not likely to be bona fide off-target cleavage sites.

Here we demonstrated for the first time of using chemical modification to significantly reduce off-target activities of CRISPR/Cas9 (FIG. 3). Learned from RNA therapeutics, in particular siRNA therapy, other chemical modifications also hold the potential to reduce off-target activities (39). For example, nucleobase modifications of siRNA have been shown to reduce miRNA-like off-target effects (40). Position-dependent 2′OMe modification in the guide strand of siRNAs decreases “off-target” transcript silencing (41). It is worthy to investigate other chemical modifications to reduce off-target activities of CRISPR systems.

High-fidelity and enhanced specificity Cas9 variants demonstrated reduced off-target activities in mammalian cells (18,19). A recent study indicates that tuning the natural conformational threshold of Cas9-sgRNA-substrate DNA complex can improve the specificity of Cas9, and a new hyper-accurate Cas9 variant was designed and developed accordingly (42). It is possible that the DNA-RNA chimera guides, along with truncate guides and Cas9 variants share the similar principles of reducing off-target activities. It is worthy to study further the underlining mechanisms that how chemically modified guides are able to reduce off-target cleavages.

Our data suggest that the DNA nucleotides play a functional role in the recognition of the genomic DNA, and that they support the endonuclease activity of Cas9. Interestingly, truncation of the guide sequence to 14 RNA nucleotides, combined with catalytically active Cas9 fused with a functional domain such as transcription activator or inhibitor can modulate gene expression without inducing double-stranded breaks (43,44). Our data indicates that guide sequences with 14 RNA nucleotides and 6 DNA nucleotides are fully capable of inducing double-stranded breaks in the human genome (FIG. 1b, 1c, FIG. 2a). Indeed, mutation of 1-4 DNA nt in the guide region abolishes the editing effect (FIG. 2e-2f, FIG. 9), demonstrating that DNA sequence substitutions can play a role in recognizing the complementary genomic sequences. Considering the weaker binding of DNA-DNA than DNA-RNA (32,33), it will be interesting to test the possibility that some DNA-RNA chimeras might guide catalytically active Cas9 for binding of genomic locus and modulating of gene expression but not causing indels.

Due to the high cost of synthesis of sgRNA, a recent study conjugated a 65 nt 5′-hexyne tracrRNA and a 34 nt 3′-azide crRNA component (45). The synthetic conjugated sgRNA showed efficiently genome editing activity in cells (45). It is feasible to replace about half of the RNA with DNA nucleotides in the 3′-azide crRNA component which have the guide sequence. Such strategy may further reduce the cost of synthesis and increase specificity of guide sequences. It allows generating of single and libraries of synthetic sgRNA more practical for research and development purposes.

Our data showed the DNA-RNA chimeric guides can induce efficient genome editing in human cells with reduced off-target effects, highlighting its possible usage for biomedical research and therapeutic genome editing. Such partial DNA crRNAs or sgRNA can be easily synthesized for genome editing in cells at reduced cost and can be potentially delivered to animals using lipid or polymer nanoparticles for research and therapeutic applications (34,35). It is worth investigating whether DNA-RNA chimeric guides can reduce off-target in living animals, and to understand the associated immune responses. Our data suggests that incorporation of other nucleotides or chemical modifications into guide RNA sequences may have the potential to further decrease off-target effects of CRISPR-Cas systems.

### REFERENCES

1. Cong, L. et al. Multiplex genome engineering using CRISPR/Cas systems. Science (New York, N.Y.) 339, 819-823 (2013).
2. Mali, P. et al. RNA-guided human genome engineering via Cas9. Science (New York, N.Y.) 339, 823-826 (2013).
3. Doudna, J. A. & Charpentier, E. Genome editing. The new frontier of genome engineering with CRISPR-Cas9. Science (New York, N.Y.) 346, 1258096 (2014).
4. Jinek, M. et al. A programmable dual-RNA-guided DNA endonuclease in adaptive bacterial immunity. Science (New York, N.Y.) 337, 816-821 (2012).
5. Cox, D. B., Platt, R. J. & Zhang, F. Therapeutic genome editing: prospects and challenges. Nature medicine 21, 121-131 (2015).
6. Swarts, D. C. et al. DNA-guided DNA interference by a prokaryotic Argonaute. Nature 507, 258-261 (2014).
7. Yuan, Y. R. et al. Crystal structure of A. aeolicus argonaute, a site-specific DNA-guided endoribonuclease, provides insights into RISC-mediated mRNA cleavage. Molecular cell 19, 405-419 (2005).
8. Gabriel, R. et al. An unbiased genome-wide analysis of zinc-finger nuclease specificity. Nature biotechnology 29, 816-823 (2011).
9. Sander, J. D. et al. In silico abstraction of zinc finger nuclease cleavage profiles reveals an expanded landscape of off-target sites. Nucleic acids research 41, e181 (2013).
10. Tsai, S. Q. et al. GUIDE-seq enables genome-wide profiling of off-target cleavage by CRISPR-Cas nucleases. Nature biotechnology 33, 187-197 (2015).
11. Frock, R. L. et al. Genome-wide detection of DNA double-stranded breaks induced by engineered nucleases. Nature biotechnology 33, 179-186 (2015).
12. Kim, D. et al. Digenome-seq: genome-wide profiling of CRISPR-Cas9 off-target effects in human cells. Nature methods 12, 237-243, 231 p following 243 (2015).
13. Wang, X. et al. Unbiased detection of off-target cleavage by CRISPR-Cas9 and TALENs using integrase-defective lentiviral vectors. Nature biotechnology 33, 175-178 (2015).
14. Ran, F. A. et al. Double nicking by RNA-guided CRISPR Cas9 for enhanced genome editing specificity. Cell 154, 1380-1389 (2013).
15. Mali, P. et al. CAS9 transcriptional activators for target specificity screening and paired nickases for cooperative genome engineering. Nature biotechnology 31, 833-838 (2013).
16. Tsai, S. Q. et al. Dimeric CRISPR RNA-guided Fold nucleases for highly specific genome editing. Nature biotechnology 32, 569-576 (2014).
17. Guilinger, J. P., Thompson, D. B. & Liu, D. R. Fusion of catalytically inactive Cas9 to Fold nuclease improves the specificity of genome modification. Nature biotechnology 32, 577-582 (2014).
18. Slaymaker, I. M. et al. Rationally engineered Cas9 nucleases with improved specificity. Science (New York, N.Y.) 351, 84-88 (2016).
19. Kleinstiver, B. P. et al. High-fidelity CRISPR-Cas9 nucleases with no detectable genome-wide off-target effects. Nature 529, 490-495 (2016).
20. Bolukbasi, M. F. et al. DNA-binding-domain fusions enhance the targeting range and precision of Cas9. Nat Meth 12, 1150-1156 (2015).
21. Fu, Y., Sander, J. D., Reyon, D., Cascio, V. M. & Joung, J. K. Improving CRISPR-Cas nuclease specificity using truncated guide RNAs. Nature biotechnology 32, 279-284 (2013).
22. Hendel, A. et al. Chemically modified guide RNAs enhance CRISPR-Cas genome editing in human primary cells. Nature biotechnology 33, 985-989 (2015).
23. Randar, M. et al. Synthetic CRISPR RNA-Cas9-guided genome editing in human cells. Proc Natl Acad Sci USA 112, E7110-7117 (2015).
24. Fu, Y. et al. High-frequency off-target mutagenesis induced by CRISPR-Cas nucleases in human cells. Nature biotechnology 31, 822-826 (2013).
25. Jiang, F., Zhou, K., Ma, L., Gressel, S. & Doudna, J. A. STRUCTURAL BIOLOGY. A Cas9-guide RNA complex preorganized for target DNA recognition. Science (New York, N.Y.) 348, 1477-1481 (2015).
26. Nishimasu, H. et al. Crystal Structure of Cas9 in Complex with Guide RNA and Target DNA. Cell 156, 935-949.
27. Gilbert, L. A. et al. CRISPR-mediated modular RNA-guided regulation of transcription in eukaryotes. Cell 154, 442-451 (2013).
28. Brinkman, E. K., Chen, T., Amendola, M. & van Steensel, B. Easy quantitative assessment of genome editing by sequence trace decomposition. Nucleic Acids Res 42, e168 (2014).
29. Hsu, P. D. et al. DNA targeting specificity of RNA-guided Cas9 nucleases. Nature biotechnology 31, 827-832 (2013).
30. Yin, H. et al. Genome editing with Cas9 in adult mice corrects a disease mutation and phenotype. Nature biotechnology 32, 551-553 (2014).
31. Zetsche, B. et al. Cpf1 is a single RNA-guided endonuclease of a class 2 CRISPR-Cas system. Cell 163, 759-771 (2015).
32. Lesnik, E. A. & Freier, S. M. Relative thermodynamic stability of DNA, RNA, and DNA:RNA hybrid duplexes: relationship with base composition and structure. Biochemistry 34, 10807-10815 (1995).
33. Gyi, J. I., Lane, A. N., Conn, G. L. & Brown, T. The orientation and dynamics of the CT-OH and hydration of RNA and DNA.RNA hybrids. Nucleic acids research 26, 3104-3110 (1998).
34. Yin, H. et al. Structure-guided chemical modification of guide RNA enables potent non-viral in vivo genome editing. Nature biotechnology (2017).
35. Yin, H. et al. Therapeutic genome editing by combined viral and non-viral delivery of CRISPR system components in vivo. Nature biotechnology 34, 328-333 (2016).
36. Ran, F. A. et al. In vivo genome editing using Staphylococcus aureus Cas9. Nature 520, 186-191 (2015).
37. Hou, Z. et al. Efficient genome engineering in human pluripotent stem cells using Cas9 from Neisseria meningitidis. Proceedings of the National Academy of Sciences of the United States of America 110, 15644-15649 (2013).
38. Lee, K. et al. Synthetically modified guide RNA and donor DNA are a versatile platform for CRISPR-Cas9 engineering. eLife 6 (2017).
39. Deleavey, G. F. & Damha, M. J. Designing chemically modified oligonucleotides for targeted gene silencing. Chemistry & biology 19, 937-954 (2012).
40. Suter, S. R. et al. Controlling miRNA-like off-target effects of an siRNA with nucleobase modifications. Organic & biomolecular chemistry (2017).
41. Jackson, A. L. et al. Position-specific chemical modification of siRNAs reduces “off-target” transcript silencing. RNA 12, 1197-1205 (2006).
42. Chen, J. S. et al. Enhanced proofreading governs CRISPR-Cas9 targeting accuracy. Nature 550, 407 (2017).
43. Kiani, S. et al. Cas9 gRNA engineering for genome editing, activation and repression. Nature methods 12, 1051-1054 (2015).
44. Dahlman, J. E. et al. Orthogonal gene knockout and activation with a catalytically active Cas9 nuclease. Nature biotechnology 33, 1159-1161 (2015).
45. He, K., Chou, E. T., Begay, S., Anderson, E. M. & van Brabant Smith, A. Conjugation and Evaluation of Triazole-Linked Single Guide RNA for CRISPR-Cas9 Gene Editing. Chembiochem 17, 1809-1812 (2016).

### Methods

### Oligonucleotides Synthesis

The oligonucleotides were synthesized by Integrated DNA Technologies (IDT) using the solid phase synthesis and phosphoroamidite chemistry²². The sequences of all oligonucleotide are shown in Table 10 below. The guide sequences were published elsewhere^{22, 24, 27.} The oligonucleotides were dissolved in sodium citrate buffer (pH 4.5), aliquoted and stored at −80° C.

TABLE 10
Oligonucleotides sequences used in this study.
DNA nucleotides are in uppercase (A, T, C, G).
RNA nucleotides are shown as r plus uppercase (rA, rU, rC, rG).

| Name | Sequence (5′ to 3′) | Note |
|---|---|---|
| GFP-Native | rGrGrGrCrGrArGrGrArGrCrUrGrUrUrCrAr | crRNA without any |
| Strand | CrCrGrGrUrUrUrUrArGrArGrCrUrA | modifications |
| | (SEQ ID NO: 170) | |
| GFP-2DNA | GGrGrCrGrArGrGrArGrCrUrGrUrUrCrArC | Substitutes 2 RNA |
| | rCrGrGrUrUrUrUrArGrArGrCrUrArUrGrCr | nucleotides with DNA |
| | UrGrUrUrUrUrG (SEQ ID NO: 171) | nucleotides at 5′ end of |
| | | guide sequence |
| GFP-4DNA | GGGCrGrArGrGrArGrCrUrGrUrUrCrArCr | Substitutes 4 RNA |
| | CrGrGrUrUrUrUrArGrArGrCrUrArUrGrCr | nucleotides with DNA |
| | UrGrUrUrUrUrG (SEQ ID NO: 172) | nucleotides at 5′ end of |
| | | guide sequence |
| GFP-6DNA | GGGCGArGrGrArGrCrUrGrUrUrCrArCrC | Substitutes 6 RNA |
| | rGrGrUrUrUrUrArGrArGrCrUrArUrGrCrUr | nucleotides with DNA |
| | GrUrUrUrUrG (SEQ ID NO: 173) | nucleotides at 5′ end of |
| | | guide sequence |
| GFP-8DNA | GGGCGAGGrArGrCrUrGrUrUrCrArCrCr | Substitutes 8 RNA |
| | GrGrUrUrUrUrArGrArGrCrUrArUrGrCrUr | nucleotides with DNA |
| | GrUrUrUrUrG (SEQ ID NO: 174) | nucleotides at 5′ end of |
| | | guide sequence |
| GFP-10DNA | GGGCGAGGAGrCrUrGrUrUrCrArCrCrGr | Substitutes 10 RNA |
| | GrUrUrUrUrArGrArGrCrUrArUrGrCrUrGr | nucleotides with DNA |
| | UrUrUrUrG (SEQ ID NO: 175) | nucleotides at 5′ end |
| | | of guide sequence |
| GFP-12DNA | GGGCGAGGAGCTrGrUrUrCrArCrCrGrG | Substitutes 12 RNA |
| | rUrUrUrUrArGrArGrCrUrArUrGrCrUrGrUr | nucleotides with DNA |
| | UrUrUrG | nucleotides at 5′ end |
| | (SEQ ID NO: 176) | of guide sequence |
| GFP-14DNA | GGGCGAGGAGCTGTrUrCrArCrCrGrGr | Substitutes 14 RNA |
| | UrUrUrUrArGrArGrCrUrArUrGrCrUrGrUr | nucleotides with DNA |
| | UrUrUrG | nucleotides at 5′ end |
| | (SEQ ID NO: 177) | of guide sequence |
| GFP-16DNA | GGGCGAGGAGCTGTTCrArCrCrGrGrUr | Substitutes 16 RNA |
| | UrUrUrArGrArGrCrUrArUrGrCrUrGrUrUr | nucleotides with DNA |
| | UrUrG | nucleotides at 5′ end |
| | (SEQ ID NO: 178) | of guide sequence |
| GFP-18DNA | GGGCGAGGAGCTGTTCACrCrGrGrUrU | Substitutes 18 RNA |
| | rUrUrArGrArGrCrUrArUrGrCrUrGrUrUrUr | nucleotides with DNA |
| | UrG | nucleotides at 5′ end |
| | (SEQ ID NO: 179) | of guide sequence |
| GFP-20DNA | GGGCGAGGAGCTGTTCACCGrGrUrUr | Substitutes 20 RNA |
| | UrUrArGrArGrCrUrArUrGrCrUrGrUrUrUr | nucleotides with DNA |
| | UrG | nucleotides at 5′ end |
| | (SEQ ID NO: 180) | of guide sequence |
| EMX1-Native | rGrArGrUrCrCrGrArGrCrArGrArArGrArAr | crRNA without any |
| Strand | GrArArGrUrUrUrUrArGrArGrCrUrArUrGr | modifications |
| | CrUrGrUrUrUrUrG | |
| | (SEQ ID NO: 181) | |
| EMX1-2DNA | GArGrUrCrCrGrArGrCrArGrArArGrArArG | Substitutes 2 RNA |
| | rArArGrUrUrUrUrArGrArGrCrUrArUrGrCr | nucleotides with DNA |
| | UrGrUrUrUrUrG (SEQ ID NO: 182) | nucleotides at 5′ end of |
| | | guide sequence |
| EMX1-4DNA | GAGTrCrCrGrArGrCrArGrArArGrArArGrA | Substitutes 4 RNA |
| | rArGrUrUrUrUrArGrArGrCrUrArUrGrCrUr | nucleotides with DNA |
| | GrUrUrUrUrG (SEQ ID NO: 183) | nucleotides at 5′ end of |
| | | guide sequence |
| EMX1-6DNA | GAGTCCrGrArGrCrArGrArArGrArArGrAr | Substitutes 6 RNA |
| | ArGrUrUrUrUrArGrArGrCrUrArUrGrCrUr | nucleotides with DNA |
| | GrUrUrUrUrG (SEQ ID NO: 184) | nucleotides at 5′ end of |
| | | guide sequence |
| EMX1-8DNA | GAGTCCGArGrCrArGrArArGrArArGrArAr | Substitutes 8 RNA |
| | GrUrUrUrUrArGrArGrCrUrArUrGrCrUrGr | nucleotides with DNA |
| | UrUrUrUrG | nucleotides at 5′ end of |
| | (SEQ ID NO: 185) | guide sequence |
| EMX1- | GAGTCCGAGCrArGrArArGrArArGrArAr | Substitutes 10 RNA |
| 10DNA | GrUrUrUrUrArGrArGrCrUrArUrGrCrUrGr | nucleotides with DNA |
| | UrUrUrUrG | nucleotides at 5′ end |
| | (SEQ ID NO: 186) | of guide sequence |
| EMX1- | GAGTCCGAGCAGrArArGrArArGrArArGr | Substitutes 12 RNA |
| 12DNA | UrUrUrUrArGrArGrCrUrArUrGrCrUrGrUr | nucleotides with DNA |
| | UrUrUrG | nucleotides at 5′ end |
| | (SEQ ID NO: 187) | of guide sequence |
| EMX1- | GAGTCCGAGCAGAArGrArArGrArArGrU | Substitutes 14 RNA |
| 14DNA | rUrUrUrArGrArGrCrUrArUrGrCrUrGrUrUr | nucleotides with DNA |
| | UrUrG | nucleotides at 5′ end |
| | (SEQ ID NO: 188) | of guide sequence |
| EMX1- | GAGTCCGAGCAGAAGArArGrArArGrUr | Substitutes 16 RNA |
| 16DNA | UrUrUrArGrArGrCrUrArUrGrCrUrGrUrUr | nucleotides with DNA |
| | UrUrG | nucleotides at 5′ end |
| | (SEQ ID NO: 189) | of guide sequence |
| EMX1- | GAGTCCGAGCAGAAGAAGrArArGrUrUr | Substitutes 18 RNA |
| 18DNA | UrUrArGrArGrCrUrArUrGrCrUrGrUrUrUr | nucleotides with DNA |
| | UrG | nucleotides at 5′ end |
| | (SEQ ID NO: 190) | of guide sequence |
| EMX1- | GAGTCCGAGCAGAAGAAGAArGrUrUrU | Substitutes 20 RNA |
| 20DNA | rUrArGrArGrCrUrArUrGrCrUrGrUrUrUrUrG | nucleotides with DNA |
| | (SEQ ID NO: 191) | nucleotides at 5′ end |
| | | of guide sequence |
| tracrRNA | rArArArCrArGrCrArUrArGrCrArArGrUrUr | tracrRNA without any |
| | ArArArArUrArArGrGrCrUrArGrUrCrCrGrU | modification |
| | rUrArUrCrArArCrUrUrGrArArArArArGrUr | |
| | GrGrCrArCrCrGrArGrUrCrGrGrUrGrCrUr | |
| | UrUrUrUrUrU (SEQ ID NO: 192) | |
| GFP-4DNA | rGrGrGrCrGrArGrGrArGrCrUrGrUrUrCAC | Substitutes 4 RNA |
| at 3′ end | CGrGrUrUrUrUrArGrArGrCrUrArUrGrCrU | nucleotides with DNA |
| | rGrUrUrUrUrG (SEQ ID NO: 193) | nucleotides at 3′ end of |
| | | guide sequence |
| VEGFA- | rGrArCrCrCrCrCrUrCrCrArCrCrCrCrGrCr | crRNA without any |
| Native Strand | CrUrCrGrUrUrUrUrArGrArGrCrUrArUrGr | modifications |
| | CrUrGrUrUrUrUrG | |
| | (SEQ ID NO: 194) | |
| VEGFA- | GACCCCCTCCrArCrCrCrCrGrCrCrUrCr | Substitutes 10 RNA |
| 10DNA | GrUrUrUrUrArGrArGrCrUrArUrGrCrUrGr | nucleotides with DNA |
| | UrUrUrUrG | nucleotides at 5′ end |
| | (SEQ ID NO: 195) | of guide sequence |
| GFP sgRNA | rGrGrG rCrGrA rGrGrA rGrCrU rGrUrU | GFP-targeting sgRNA |
| | rCrArC rCrGrG rUrUrU rUrArG rArGrC | without any modifications |
| | rUrArG rArArA rUrArG rCrArA rGrUrU | |
| | rArArA rArUrA rArGrG rCrUrA rGrUrC | |
| | rCrGrU rUrArU rCrArA rCrUrU rGrArA | |
| | rArArA rGrUrG rGrCrA rCrCrG rArGrU | |
| | rCrGrG rUrGrC rUrUrU rUrU | |
| | (SEQ ID NO: 196) | |
| GFP | GGG CGA GGrA rGrCrU rGrUrU rCrArC | Substitutes 8 RNA |
| sgRNA- | rCrGrG rUrUrU rUrArG rArGrC rUrArG | nucleotides with DNA |
| 8D | rArArA rUrArG rCrArA rGrUrU rArArA | nucleotides at 5′ end of |
| | rArUrA rArGrG rCrUrA rGrUrC rCrGrU | guide sequence |
| | rUrArU rCrArA rCrUrU rGrArA rArArA | |
| | rGrUrG rGrCrA rCrCrG rArGrU rCrGrG | |
| | rUrGrC rUrUrU rUrU | |
| | (SEQ ID NO: 197) | |
| EMX1 | rGrArGrUrCrCrGrArGrCrArGrArArGrArAr | EMX1-targeting sgRNA |
| sgRNA | GrArArG rUrUrU rUrArG rArGrC rUrArG | without any modifications |
| | rArArA rUrArG rCrArA rGrUrU rArArA | |
| | rArUrA rArGrG rCrUrA rGrUrC rCrGrU | |
| | rUrArU rCrArA rCrUrU rGrArA rArArA | |
| | rGrUrG rGrCrA rCrCrG rArGrU rCrGrG | |
| | rUrGrC rUrUrU rUrU | |
| | (SEQ ID NO: 198) | |
| EMX1 | GAGTCCGArGrCrArGrArArGrArArGrArAr | Substitutes 8 RNA |
| sgRNA-8D | G rUrUrU rUrArG rArGrC rUrArG rArArA | nucleotides with DNA |
| | rUrArG rCrArA rGrUrU rArArA rArUrA | nucleotides at 5′ end of |
| | rArGrG rCrUrA rGrUrC rCrGrU rUrArU | guide sequence |
| | rCrArA rCrUrU rGrArA rArArA rGrUrG | |
| | rGrCrA rCrCrG rArGrU rCrGrG rUrGrC | |
| | rUrUrU rUrU | |
| | (SEQ ID NO: 199) | |
| VEGFA | rGrArCrCrCrCrCrUrCrCrArCrCrCrCrGrCr | VEGFA-targeting sgRNA |
| sgRNA | CrUrCrG rUrUrU rUrArG rArGrC rUrArG | without modifications |
| | rArArA rUrArG rCrArA rGrUrU rArArA | |
| | rArUrA rArGrG rCrUrA rGrUrC rCrGrU | |
| | rUrArU rCrArA rCrUrU rGrArA rArArA | |
| | rGrUrG rGrCrA rCrCrG rArGrU rCrGrG | |
| | rUrGrC rUrUrU rUrU (SEQ ID NO: 200) | |
| VEGFA | GACCCCCTrCrCrArCrCrCrCrGrCrCrUrC | Substitutes 8 RNA |
| | (SEQ ID NO: 201) | nucleotides with |
| sgRNA-8D | rG rUrUrU rUrArG rArGrC rUrArG rArArA | DNA nucleotides at 5′ |
| | rUrArG rCrArA rGrUrU rArArA rArUrA | end of guide sequence |
| | rArGrG rCrUrA rGrUrC rCrGrU rUrArU | |
| | rCrArA rCrUrU rGrArA rArArA rGrUrG | |
| | rGrCrA rCrCrG rArGrU rCrGrG rUrGrC | |
| | rUrUrU rUrU (SEQ ID NO: 202) | |
| RNA mut | GGGCGAGGrUrCrGrArGrUrUrCrArCrCr | Substitutes 8 RNA |
| | GrGrUrUrUrUrArGrArGrCrUrArUrGrCrUr | nucleotides with DNA |
| | GrUrUrUrUrG (SEQ ID NO: 203) | nucleotides at 5′ end of |
| | | guide sequence, and |
| | | mutates 4 RNA |
| | | nucleotides (position 9-12 |
| | | from 5′ end). |
| DNA mut-1 | GGGCCTCCrArGrCrUrGrUrUrCrArCrCrG | Substitutes 8 RNA |
| | rGrUrUrUrUrArGrArGrCrUrArUrGrCrUrGr | nucleotides with DNA |
| | UrUrUrUrG (SEQ ID NO: 204) | nucleotides at 5′ end of |
| | | guide sequence, and |
| | | mutates 4 DNA |
| | | nucleotides (position 5-8 |
| | | from 5′ end). |
| DNA mut-2 | CCCGGAGGrArGrCrUrGrUrUrCrArCrCr | Substitutes 8 RNA |
| | GrGrUrUrUrUrArGrArGrCrUrArUrGrCrUr | nucleotides with DNA |
| | GrUrUrUrUrG (SEQ ID NO: 205) | nucleotides at 5′ end of |
| | | guide sequence, and |
| | | mutates 4 DNA |
| | | nucleotides (position 1-4 |
| | | from 5′ end). |
| GFP2 Native | rGrGrGrCrArCrGrGrGrCrArGrCrUrUrGrCr | crRNA without any |
| crRNA | CrGrGrGrUrUrUrUrArGrArGrCrUrArUrGr | modifications |
| | CrUrGrUrUrUrUrG (SEQ ID NO: 206) | |
| GFP2 RNA- | rGrGrGrCrArCrGrGrGrCrArGrCrUrUrGrG | crRNA without any |
| mut 1 | rCrGrGrGrUrUrUrUrArGrArGrCrUrArUrGr | modifications and with |
| | CrUrGrUrUrUrUrG (SEQ ID NO: 207) | 1 nt mismatch mutation |
| | | at the seed region |
| GFP2 RNA- | rGrGrGrCrArCrGrGrCrCrArGrCrUrUrGrCr | crRNA without |
| mut 2 | CrGrGrGrUrUrUrUrArGrArGrCrUrArUrGr | modifications and with |
| | CrUrGrUrUrUrUrG (SEQ ID NO: 208) | 1 nt mismatch mutation |
| | | at the tail region |
| GFP2 10DNA | GGGCACGGGCrArGrCrUrUrGrCrCrGrG | Substitutes 10 RNA |
| | rGrUrUrUrUrArGrArGrCrUrArUrGrCrUrGr | nucleotides with DNA |
| | UrUrUrUrG (SEQ ID NO: 209) | nucleotides at 5′ end |
| | | of guide sequence |
| GFP2 | GGGCACGGGCrArGrCrUrUrGrGrCrGrG | Substitutes 10 RNA |
| 10DNA-mut 1 | rGrUrUrUrUrArGrArGrCrUrArUrGrCrUrGr | nucleotides with DNA |
| | UrUrUrUrG (SEQ ID NO: 210) | nucleotides at 5′ end |
| | | of guide sequence; |
| | | with 1 nt mismatch |
| | | mutation at the seed |
| | | region |
| GFP2 | GGGCACGGCCrArGrCrUrUrGrCrCrGrG | Substitutes 10 RNA |
| 10DNA-mut 2 | rGrUrUrUrUrArGrArGrCrUrArUrGrCrUrGr | nucleotides with DNA |
| | UrUrUrUrG | nucleotides at 5′ end |
| | (SEQ ID NO: 211) | of guide sequence; |
| | | with 1 nt mismatch |
| | | mutation at the tail |
| | | region |
| Mus Pcsk9-2- | rGrCrU rCrGrC rCrCrU rCrCrC rGrUrC | crRNA without any |
| crRNA | rCrCrA rGrG rG rUrUrU rUrArG rArGrC | modifications |
| | rUrArU rGrCrU rGrUrU rUrUrG (SEQ ID NO: | |
| | 212) | |
| Mus Pcsk9-2- | GCT CGC CCT CrCrC rGrUrC rCrCrA | Substitutes 10 RNA |
| 10DNA | rGrG rG rUrUrU rUrArG rArGrC rUrArU | nucleotides with DNA |
| | rGrCrU rGrUrU rUrUrG (SEQ ID NO: 213) | nucleotides at 5′ end |
| | | of guide sequence |
| 293 site 4- | rGrGrC rArCrU rGrCrG rGrCrU rGrGrA | crRNA without any |
| crRNA | rGrGrU rGrG rG rUrUrU rUrArG rArGrC | modifications |
| | rUrArU rGrCrU rGrUrU rUrUrG (SEQ ID NO: | |
| | 214) | |
| 293 site 4- | GGC ACT GCG GrCrU rGrGrA rGrGrU rGrG | Substitutes 10 RNA |
| 10DNA | rG rUrUrU rUrArG rArGrC rUrArU rGrCrU | nucleotides with DNA |
| | rGrUrU rUrUrG (SEQ ID NO: 215) | nucleotides at 5′ end |
| | | of guide sequence |
| GFP 3 nt DNA | GGGC GTCC | Substitutes 8 RNA |
| mismatch | rArGrCrUrGrUrUrCrArCrCrGrGrUrUrUrUr | nucleotides with DNA |
| | ArGrArGrCrUrArUrGrCrUrGrUrUrUrUrG | nucleotides at 5′ end of |
| | (SEQ ID NO: 216) | guide sequence; with 3 nt |
| | | mismatch mutation at the |
| | | seed region |
| GFP 3 nt RNA | rGrGrGrC rGrUrCrC | crRNA without |
| mismatch | rArGrCrUrGrUrUrCrArCrCrGrGrUrUrUrUr | modifications and with |
| | ArGrArGrCrUrArUrGrCrUrGrUrUrUrUrG | 3 nt mismatch mutation |
| | (SEQ ID NO: 217) | at the tail region |
| GFP 2 nt DNA | GGGCGACbCrArGrCrUrGrUrUrCrArCrCrGr | Substitutes 8 RNA |
| mismatch | GrUrUrUrUrArGrArGrCrUrArUrGrCrUrGrUr | nucleotides with DNA |
| | Ur UrUrG | nucleotides at 5′ end of |
| | (SEQ ID NO: 218) | guide sequence; with 2 nt |
| | | mismatch mutation at the |
| | | seed region |
| GFP 2 nt RNA | rGrGrGrC rGrArCrC | crRNA without |
| mismatch | rArGrCrUrGrUrUrCrArCrCrGrGrUrUrUrUr | modifications and with |
| | ArGrArGrCrUrArUrGrCrUrGrUrUrUrUrG | 2 nt mismatch mutation |
| | (SEQ ID NO: 219) | at the tail region |
| GFP 1 nt | GGGCGAGCrArGrCrUrGrUrUrCrArCrCrGrG | Substitutes 8 RNA |
| DNA | rUrUrUrUr | nucleotides with DNA |
| mismatch | ArGrArGrCrUrArUrGrCrUrGrUrUrUrUrG | nucleotides at 5′ end of |
| | (SEQ ID NO: 220) | guide sequence; with 1 nt |
| | | mismatch mutation at the |
| | | seed region |
| GFP 1 nt | rGrGrGrC rGrArGrC | crRNA without |
| RNA | rArGrCrUrGrUrUrCrArCrCrGrGrUrUrUrUr | modifications and with |
| mismatch | ArGrArGrCrUrArUrGrCrUrGrUrUrUrUrGr | 1 nt mismatch mutation |
| | UrG | at the tail region |
| | (SEQ ID NO: 221) | |
| GFP-sg 10D | GGG CGA GGA GrCrU rGrUrU rCrArC | Substitutes 10 RNA |
| | rCrGrG rUrUrU rUrArG rArGrC rUrArG | nucleotides with DNA |
| | rArArA rUrArG rCrArA rGrUrU rArArA | nucleotides at 5′ end |
| | rArUrA rArGrG rCrUrA rGrUrC | of guide sequence of |
| | rCrGrUrUrArU rCrArA rCrUrU rGrArA | sgRNA |
| | rArArA rGrUrG rGrCrA rCrCrG rArGrU | |
| | rCrGrG rUrGrC rUrArU rUrU (SEQ ID NO: | |
| | 222) | |
| EMX-sg10D | GAGTCCGAGCrArGrArArGrArArGrArAr | Substitutes 10 RNA |
| | G rUrUrU rUrArG rArGrC rUrArG rArArA | nucleotides with DNA |
| | rUrArG rCrArA rGrUrU rArArA rArUrA | nucleotides at 5′ end |
| | rArGrG rCrUrA rGrUrC rCrGrU rUrArU | of guide sequence of |
| | rCrArA rCrUrU rGrArA rArArA rGrUrG | sgRNA |
| | rGrCrA rCrCrG rArGrU rCrGrG rUrGrC | |
| | rUrUrU rUrU (SEQ ID NO: 223) | |
| VEGFA- | GACCCCCTCCrArCrCrCrCrGrCrCrUrCr G | Substitutes 10 RNA |
| sg10D | rUrUrU rUrArG rArGrC rUrArG rArArA | nucleotides with DNA |
| | rUrArG rCrArA rGrUrU rArArA rArUrA | nucleotides at 5′ end of |
| | rArGrG rCrUrA rGrUrC rCrGrU rUrArU | guide sequence of |
| | rCrArA rCrUrU rGrArA rArArA rGrUrG | sgRNA |
| | rGrCrA rCrCrG rArGrU rCrGrG rUrGrC | |
| | rUrUrU rUrU (SEQ ID NO: 224) | |
| 22 DNA-3′ | rGrGrGrCrGrArGrGrArGrCrUrGrUrUrCrAr | Substitutes 22 RNA |
| | CrCrGGTTTTAGAGCTATGCTGTTTTG | nucleotides with DNA |
| | (SEQ ID NO: 225) | nucleotides at 3′ end |
| | | of crRNA |
| 16 DNA-3′ | rGrGrGrCrGrArGrGrArGrCrUrGrUrUrCrAr | Substitutes 16 RNA |
| | CrCrGGrUrUrUrUrArGAGCTATGCTGTT | nucleotides with DNA |
| | TTG (SEQ ID NO: 226) | nucleotides at 3′ end |
| | | of guide sequence of |
| | | crRNA |
| 8DNA16DNA | GGGCGAGGrArGrCrUrGrUrUrCrArCrCr | Substitutes 8 RNA |
| | GGrUrUrUrUrArGAGCTATGCTGTTTTG | nucleotides with DNA |
| | (SEQ ID NO: 227) | nucleotides at 5′ end 16 |
| | | RNA nucleotides with |
| | | DNA nucleotides at 3′ |
| | | end of crRNA |
| Ascpf1 | rUrArArUrUrUrCrUrArCrUrCrUrUrGrUrAr | Ascpf1 crRNA without |
| | GrArUrCrUrGrArUrGrGrUrCrCrArUrGrUr | modifications |
| | CrUrGrUrUrArCrUC | |
| | (SEQ ID NO: 228) | |
| 8DNA- | rUrArArUrUrUrCrUrArCrUrCrUrUrGrUrAr | Substitutes 8 RNA |
| Ascpf1 | GrArUrCrUrGrArUrGrGrUrCrCrArUrGrUr | nucleotides with DNA |
| | CTGTTACTC | nucleotides at 3′ end of |
| | (SEQ ID NO: 229) | guide sequence of Ascpf1 |
| | | crRNA |

### Cell Culture and FACS

HEK293T cells were infected by lentiviral particles to stably express EF1a-GFP-PGK-Puro (addgene 26777)⁴⁶ and EFs-spCas9-Blast (addgene 52962)⁴⁷. Functional titer was used to ensure low MOI. Cells were infected with limiting dilution of lentivirus and wells with <40% GFP signal or cells surviving Blast selection (MOI<1) were chosen as described³⁴. U2OS-GFP-PEST cells (kindly provided by Keith Joung lab²¹) were transfected with lentivirus to allow stable expressing Cas9. HEK293T Cells were transfected with a crRNA targeting GFP and the tracrRNA (26 nM each, final concentration) using lipofectamine (Thermo Fisher Scientific). U2OS-GFP-PEST cells were transfected with the same concentration of the crRNA and the tracrRNA by electroporation (Neon Transfection System, Thermo Fisher Scientific, see below section for details of electroporation). GFP negative cells were counted by FACS 7 days for HEK293T cells and 3 days for U2OS-GFP-PEST after transfection. FACS was performed using MoFlo cell sorter (Beckman) or LSR (BD Biosciences) as described³⁵. Flowjo was used to perform data analysis. The axis labels indicates the fluorochrome used (FIG. 5B). The GFP negative cells were gated according to untreated GFP positive cells.

### Determining Allele Modification Frequencies Using TIDE Analysis and Surveyor Assay

Genomic DNA was extracted using QuickExtract DNA Extraction Solution (Epicentre). PCR (initiate heating and 25-30 cycles of 15 s at 94° C., 15 s at 55-62° C. and 1 min at 72° C.) was performed with 50 ng genomic DNA to yield the amplicons of the CRISPR target sites. The sequences of primer pairs are presented in Table 11 below.

TABLE 11
Primer Sequences

| ID | Sequence (5′->3′) | Notes |
|---|---|---|
| EF1a-F | TCAAGCCTCAGACAGTGGTTC | GFP PCR primers |
| | (SEQ ID NO: 121) | |
| GFP_R | TCCTTGAAGTCGATGCCCTT | |
| | (SEQ ID NO: 122) | |
| VEGFA_For | AGAGAAGTCGAGGAAGAGAGAG | VEGFA PCR Primers |
| | (SEQ ID NO: 230) | |
| VEGFA_Rev | CAGCAGAAAGTTCATGGTTTCG | |
| | (SEQ ID NO: 231) | |
| VEGFA_OFF_1_For | CAAGATGTGCACTTGGGCTA | VEGFA Off target site 1 |
| | (SEQ ID NO: 232) | |
| VEGFA_OFF_1_Rev | GCAGCCTATTGTCTCCTGGT | |
| | (SEQ ID NO: 233) | |
| VEGFA_OFF_2_For | CCAGGTGGTGTCAGCGGAGG | VEGFA Off target site 2 |
| | (SEQ ID NO: 234) | |
| VEGFA_OFF_2_Rev | TGCCTGGCCCTCTCTGAGTCT | |
| | (SEQ ID NO: 235) | |
| VEGFA_OFF_3_For | TGCTGCAGGTGGTTCCGCAG | VEGFA Off target site 3 |
| | (SEQ ID NO: 236) | |
| VEGFA_OFF_3_Rev | CTGGAACCGCATCCTCCGCA | |
| | (SEQ ID NO: 237) | |
| Sp-EMX1-F2 | CCATCCCCTTCTGTGAATGT | EMX1, PCR Primers |
| | (SEQ ID NO: 238) | |
| Sp-FMX1-R2 | GGAGATTGGAGACACGGAGA | |
| | (SEQ ID NO: 239) | |
| 293-site4F | GAGACCTGCTGAGGGCGGCTTCT | 293 site4 PCR primers |
| | CCC | |
| | (SEQ ID NO: 240) | |
| 293-site4 seq | AGTCAGTCCATGCCTGCAGGGTC | |
| | (SEQ ID NO: 241) | |
| 293-site4R | CCCACTGTAGTCACACAGCACCA | |
| | GAG | |
| | (SEQ ID NO: 242) | |
| Pcsk9_For | ATCACCCCAACCCCAAACCA | Pcsk9 PCR primers |
| | (SEQ ID NO: 243) | |
| Pcsk9_Rev | CCAGGCGTCCATGTCCTTCC | |
| | (SEQ ID NO: 244) | |

For the Tracking of Indels by Decomposition (TIDE) (28), the PCR products were purified using PCR purification kit (Qiagen), sequenced by Sanger method (Quintarabio) and analyzed by the online software (http://tide.nki.nl). For the surveyor assay, purified PCR amplicons were denatured, re-annealed and subsequently digested with Surveyor nuclease (IDT). Digested DNA was resolved by electrophoresis in a 4-20% TBE gel (Thermo Fisher Scientific), stained briefly with ethidium bromide, and visualized by UV light. The gels are representative of three experiments. Off-target sites of VEGFA were published elsewhere²⁴. Indel percentage was measured as 100×(1−(1−(b+c)/(a+b+c))½). a is the intensity of the uncut PCR product, and b and c are the intensities of cleavage PCR product²⁹.

### GUIDE-Seq Off-Target Analysis

We performed GUIDE-seq¹⁰ with some modifications as described previously (20). Briefly, Hepa1-6 or HEK293T cells stably expressing SpCas9 were transfected using Lipofectamine 3000 transfection reagent (Invitrogen) according to the manufacturer's suggested protocol. 26 nM of crRNA/tracrRNA and annealed GUIDE-seq oligonucleotide (7.5 pmol for HEK293T cells and 100 pmol for Hepa1-6 cells) were transfected into cells into each well of a 24 well plate. 48 h after transfection, genomic DNA was extracted with a PureLink Genomic DNA Mini Kit (invitrogen) according to the manufacturer's suggested protocol. Library preparations were done with original adaptors according to protocols described by Joung and colleagues¹⁰. Each library was barcoded within the P5 and P7 adaptors for pooled sequencing. The barcoded, purified libraries were deep-sequenced as a pool using a paired-end 150-bp Illumina MiSeq run. Data were analyzed using the GUIDEseq Bioconductor package (48) using the default settings except that min.reads is set to 2 and distance. Threshold to 70 with the identified sites filtered to those sequences with six or fewer mismatches within the input guide and PAM (NGG) sequences.

### Neon Transfection for Jurkat T Cells

We prepared a 5 μl mixture containing crRNA, tracrRNA and spCas9 protein (2 μM final for each) and kept them at room temperature for 15 minutes to form Cas9 RNP complex. Jurkat T cells were harvested, washed and resuspended in Resuspension Buffer (included in Neon Kits) at a final density of 2×10\hat{ }7 cells/ml. Cells were mixed with the 5 μl RNP complex. Transfection was performed according to the manufacturer's suggested protocol in a 10 μl tip with the parameters: 1325 v/10 ms/4 pulses. After transfection, cells were transferred into 500 μl RPMI 1640 media with L-glutamine and 10% FBS but without antibiotics and incubated at 37° C. in a humidified CO₂ incubator. Three days after transfection, genomic DNA was harvested for the analysis of indel % by TIDE.

### Statistics

Student's t-tests or One-Way ANOVA with Tukey post hoc test were used to calculate P values by Prism 7 (GraphPad). No adjustments were made for multiple comparisons. P<0.05 is considered significant. The definition of error bars is s.d. N indicates biological replicates as number of cell cultures. No inclusion and exclusion criteria of samples were used.

### REFERENCES

10. Tsai, S. Q. et al. GUIDE-seq enables genome-wide profiling of off-target cleavage by CRISPR-Cas nucleases. Nature biotechnology 33, 187-197 (2015).
20. Bolukbasi, M. F. et al. DNA-binding-domain fusions enhance the targeting range and precision of Cas9. Nat Meth 12, 1150-1156 (2015).
21. Fu, Y., Sander, J. D., Reyon, D., Cascio, V. M. & Joung, J. K. Improving CRISPR-Cas nuclease specificity using truncated guide RNAs. Nature biotechnology 32, 279-284 (2013).
22. Hendel, A. et al. Chemically modified guide RNAs enhance CRISPR-Cas genome editing in human primary cells. Nature biotechnology 33, 985-989 (2015).
24. Fu, Y. et al. High-frequency off-target mutagenesis induced by CRISPR-Cas nucleases in human cells. Nature biotechnology 31, 822-826 (2013).
27. Gilbert, L. A. et al. CRISPR-mediated modular RNA-guided regulation of transcription in eukaryotes. Cell 154, 442-451 (2013).
28. Brinkman, E. K., Chen, T., Amendola, M. & van Steensel, B. Easy quantitative assessment of genome editing by sequence trace decomposition. Nucleic Acids Res 42, e168 (2014).
29. Hsu, P. D. et al. DNA targeting specificity of RNA-guided Cas9 nucleases. Nature biotechnology 31, 827-832 (2013).
34. Yin, H. et al. Structure-guided chemical modification of guide RNA enables potent non-viral in vivo genome editing. Nature biotechnology (2017).
35. Yin, H. et al. Therapeutic genome editing by combined viral and non-viral delivery of CRISPR system components in vivo. Nature biotechnology 34, 328-333 (2016).
46. Zou, J. et al. Gene targeting of a disease-related gene in human induced pluripotent stem and embryonic stem cells. Cell stem cell 5, 97-110 (2009).
47. Sanjana, N. E., Shalem, O. & Zhang, F. Improved vectors and genome-wide libraries for CRISPR screening. Nature methods 11, 783-784 (2014).
48. Zhu, L. J. et al. GUIDEseq: a bioconductor package to analyze GUIDE-Seq datasets for CRISPR-Cas nucleases. BMC genomics 18, 379 (2017).

### Example 5

In some embodiments, sgRNA or crRNA:tracrRNA duplexes disclosed herein comprise Cas-binding domains and transcription terminator domains that are capable of binding one or more peptides with Cas9-like activity when exposed to a gene editing enzyme and a target DNA at a concentration sufficient to catalyze the reaction between the gene editing enzyme and the target DNA sequence. Below is a representative sample of amino acid sequences corresponding to gene editing enzymes which may be components in the disclosed CRISPR complexes. Vectors comprising nucleic acid sequences encoding such editing amino acid sequences are also contemplated by the disclosure.

An example of a crRNA sequence that may bind a peptide with Cas9-like activity at a biophysically acceptable concentration is

crRNA sequence:

(SEQ ID NO: 245)

AAUUUCUACUGUUGUAGAUGAGAGAUUUAGGAGAGAAUGAC

Underline: CFP1 binding region no underline: DNA binding region This sequence should be bound to a type V CRISPR-associated protein Cpf1 [Acidaminococcus sp. BV3L6] NCBI Reference Sequence: WP_021736722.1. FnCpf1 locus sequences (permanent sequence links)
Other DNA-binding domains can be found at:
pFnCpf1 (pY001) benchling.com/s/Yz1hC8BN/edit
pFnCpf1_min (pY002) benchling.com/s/UZ2wCOF2/edit
pFnCpf1_ΔCas (pY003) benchling.com/s/ctaThKG6/edit
which are incorporated by reference in their entireties.
type II CRISPR RNA-guided endonuclease Cas9 [Staphylococcus aureus] NCBI Reference Sequence: WP_001573634.1
type II CRISPR RNA-guided endonuclease Cas9 [Streptococcus pyogenes] GenBank: AMA70685.1
The C2c2 CRISPR locus was amplified from DNA from Leptotrichia shahii DSM 19757
Francisella novicida (FnCas9) type II-B CRISPR-associated RNA-guided endonuclease Cas9/Csx12 [Francisella tularensis] NCBI Reference Sequence: WP_032729892.1
Cas 3 CRISPR-associated helicase Cas3 [Escherichia coli] GenBank: CCQ30152.2

TABLE 12
Cas9 interacts with sgRNA at phosphodiester bond interaction:
Numbers below correspond to nucleotide number within the nucleotide sequence assuming a
guide sequence of at least 92 contiguous nucleotides in a molecule comprising the following
contiguous nucleotide domains in a 5′ to 3′ orientation: a DNA-Binding Domain, a Cas-
Binding Domain, and Transcription Terminator Domain.
Nucleotides numbers below indicate which nucleoside positions flank a phosphorothioate
bond that interacts with a Cas protein or peptide with Cas-like activity. For purposes of the
disclosure, a nucleotide position comprises a modification if the 3′Carbon of the sugar is
bound to the next contiguous nucleoside downstream by a bond other than a natural
phosphodiester bond. For example, “1 = 2” is a modification of position 1 of the nucleic acid
sequence disclosed below, whereas position 2 is considered unmodified because there is a
phosphodiester bond between the nucleosides at positions 2 and 3.
Within the DNA-binding domain:
1 = 2 3 = 4 4 = 5 5 = 6 11 = 12 12 = 13 13 = 14 14 = 15 15 = 16 16 = 17 17 = 18 18 = 19 19 = 20
Within the Fixed Region (the Cas-binding Domain and the Transcription Terminator
Domain)

| | | | | | | | | | |
|---|---|---|---|---|---|---|---|---|---|
| 21 = 22 | 23 = 24 | 25 = 26 | 26 = 27 | 27 = 28 | 40 = 41 | 43 = 44 | 44 = 45 | 45 = 46 | 47 = 48 |
| G = U | U = U | U = A | A = G | G = A | C = A | G = U | U = U | U = A | A = A |
| 48 = 49 | 49 = 50 | 51 = 52 | 52 = 53 | 58 = 59 | 59 = 60 | 60 = 61 | 61 = 62 | 62 = 63 | 63 = 64 |
| A = A | A = U | A = A | A = G | G = U | U = C | C = C | C = G | G = U | U = U |
| 64 = 65 | 65 = 66 | 66 = 67 | 67 = 68 | 79 = 80 | 80 = 81 | 81 = 82 | 83 = 84 | 91 = 92 | |
| U = A | A = U | U = C | C = A | G = U | U = G | G = U | C = A | C = G | |

TABLE 13
Accession Numbers of Cas proteins (or those related with Cas-like function)
and Nucleic Acids encoding the same.

All amino acid and nucleic acid sequences associated with the Accession Numbers below as
of Jun. 29, 2016, are incorporated by reference in their entireties. Any mutants or variants
that are at least 70, 75, 80, 85, 90, 95, 96, 97, 98, 99% homologous to the encoded nucleic
acids or amino acids set forth in the Accession Numbers below are also incorporated by
reference in their entireties.
NC_014644.1; NC_002967.9; NC_007929.1; NC_000913.3; NC_004547.2; NC_009380.1;
NC_011661.1; NC_010175.1; NC_010175.1; NC_010175.1; NC_003413.1; NC_000917.1;
NC_002939.5; NC_018227.2; NC_004829.2; NC_021921.1; NC_014160.1; NC_011766.1;
NC_007681.1; NC_021592.1; NC_021592.1; NC_021169.1; NC_020517.1; NC_018656.1;
NC_018015.1; NC_018015.1; NC_017946.1; NC_017576.1; NC_017576.1; NC_015865.1;
NC_015865.1; NC_015680.1; NC_015680.1; NC_015474.1; NC_015435.1; NC_013790.1;
NC_013790.1; NC_012883.1; NC_012470.1; NC_016051.1; NC_010610.1; NC_009515.1;
NC_008942.1; NC_007181.1; NC_007181.1; NC_006624.1; NC_006448.1; NC_002935.2;
NC_002935.2; NC_002950.2; NC_002950.2; NC_002663.1; NC_002663.1; NC_004557.1;
NC_004557.1; NC_019943.1; NC_019943.1; NC_019943.1; NC_017459.1; NC_017459.1;
NC_015518.1; NC_015460.1; NC_015416.1; NC_014933.1; NC_013961.1; NC_013202.1;
NC_013158.1; NC_009464.1; NC_008508.1; NC_007426.1; NC_000917.1; NC_003901.1;
NC_003901.1; NC_003106.2; NC_009434.1; NC_005085.1; NC_005085.1; NC_020247.1;
NC_020247.1; NC_020246.1; NC_020246.1; NC_018224.1; NC_015943.1; NC_011138.3;
NC_009778.1; NC_006834.1; NC_014228.1; NC_010002.1; NC_013892.1; NC_010296.1;
NC_009615.1; NC_012632.1; NC_012632.1; NC_012588.1; NC_012588.1; NC_007643.1;
NC_002939.5; NC_011296.1; NC_011296.1; NC_018609.1; NC_021355.1; NC_021355.1;
NC_020800.1; NC_019942.1; NC_019792.1; NC_015958.1; NC_015678.1; NC_015636.1;
NC_015562.1; NC_014222.1; NC_014222.1; NC_014002.1; NC_013887.1; NC_013156.1;
NC_011832.1; NC_009953.1; NC_009635.1; NC_009634.1; NC_008618.1; NC_007955.1;
NC_007955.1; NC_007955.1; NC_007955.1; NC_007955.1; NC_007796.1; NC_002754.1;
NC_002754.1; NC_011835.1; NC_013198.1; NC_000962.3; NC_002163.1; NC_017034.1;
NC_009089.1; NC_008698.1; NC_020419.1; NC_020419.1; NC_020419.1; NC_015847.1;
NC_014374.1; NC_013520.1; NC_010482.1; NC_009776.1; NC_009776.1; NC_009033.1;
NC_000916.1; NC_018015.1; NC_015518.1; NC_014537.1; NC_009440.1; NC_007644.1;
NC_007644.1; NC_022246.1; NC_019943.1; NC_016023.1; NC_016023.1; NC_015416.1;
NC_013722.1; NC_013722.1; NC_009464.1; NC_007643.1; NC_007643.1; NC_007643.1;
NC_003106.2; NC_004342.2; NC_018658.1; NC_017276.1; NC_017275.1; NC_016112.1;
NC_016112.1; NC_003552.1; NC_003197.1; NC_003198.1; NC_012726.1; NC_012623.1;
NC_015964.1; NC_023069.1; NC_023044.1; NC_022777.1; NC_022777.1; NC_022777.1;
NC_013769.1; NC_013769.1; NC_011832.1; NC_011296.1; NC_009712.1; NC_009634.1;
NC_009439.1; NC_009135.1; NC_008599.1; NC_007796.1; NC_007796.1; NC_007796.1;
NC_007355.1; NC_021082.1; NC_018001.1; NC_009785.1; NC_022084.1; NC_018092.1;
NC_014804.1; NC_014147.1; NC_009053.1; NC_000961.1; NC_000961.1; NC_021058.1;
NC_018876.1; NC_018876.1; NC_018081.1; NC_011567.1; NC_016901.1; NC_014500.1;
NC_013715.1; NC_019977.1; NC_019042.1; NC_017274.1; NC_015954.1; NC_015676.1;
NC_015320.1; NC_014122.1; NC_014122.1; NC_013407.1; NC_014961.1; NC_013926.1;
NC_013926.1; NC_021353.1; NC_008818.1; NC_021058.1; NC_015151.1; NC_013849.1;
NC_009051.1; NC_018876.1; NC_018876.1; NC_014507.1; NC_015574.1; NC_014500.1;
NC_012622.1; NC_012589.1; NC_009515.1; NC_017275.1; NC_000913.3; NC_017527.1;
NC_018227.2; NC_007355.1; NC_014106.1; NC_010610.1; NC_008054.1; NC_007164.1;
NC_015760.1; NC_009953.1; NC_010572.1; NC_009613.3; NC_014334.1; NC_008526.1;
NC_026150.1; NC_015776.1; NC_007116.6; NC_012779.2; NC_003901.1; NC_020892.1;
NC_011832.1; NC_003143.1; NC_003143.1; NC_008800.1; NC_011308.1; NC_008942.1;
NC_007297.1; NC_005877.1; NC_005877.1; NC_002689.2; NC_006085.1; NC_004116.1;
NC_010397.1; NC_009917.1; NC_012490.1; NC_006067.1; NW_004197518.1; NC_022777.1;
NC_019042.1; NC_004547.2; NC_002695.1; NC_017634.1; NC_003143.1; NC_002737.2;
NC_002737.2; NC_000918.1; NC_020913.1; NC_006448.1; NC_022093.1; NC_022093.1;
NC_015680.1; NC_007297.1; NC_004350.2; NC_004350.2; NC_004350.2; NC_004350.2;
NC_003454.1; NC_000853.1; NC_018876.1; NC_009440.1; NC_009009.1; NC_009009.1;
NC_002932.3; NC_002932.3; NC_026150.1; NC_003552.1; NC_025263.1; NC_016112.1;
NC_011098.1; NC_007643.1; NC_007643.1; NC_007643.1; NC_006347.1; NC_005140.1;
NC_004342.2; NC_002945.3; NW_007382731.1; NW_007381138.1; NC_024320.1;
NW_005756335.1; NW_003384463.1; NC_019977.1; NC_011296.1; NC_007929.1; NC_000913.3;
NC_003413.1; NC_002754.1; NC_010175.1; NC_010175.1; NC_010175.1; NC_011661.1;
NC_014537.1; NC_012470.1; NC_004829.2; NC_015516.1; NC_014374.1; NC_009033.1;
NC_007681.1; NC_002689.2; NC_006085.1; NC_021592.1; NC_021592.1; NC_021169.1;
NC_020517.1; NC_018015.1; NC_018015.1; NC_018015.1; NC_017946.1; NC_017946.1;
NC_017576.1; NC_017576.1; NC_015865.1; NC_015865.1; NC_015847.1; NC_015680.1;
NC_015680.1; NC_015474.1; NC_015435.1; NC_014106.1; NC_013790.1; NC_012883.1;
NC_012804.1; NC_016051.1; NC_011529.1; NC_010482.1; NC_009515.1; NC_009440.1;
NC_008942.1; NC_008054.1; NC_007181.1; NC_006624.1; NC_006448.1; NC_006448.1;
NC_002935.2; NC_002935.2; NC_002950.2; NC_002663.1; NC_019943.1; NC_019943.1;
NC_017459.1; NC_017459.1; NC_016023.1; NC_015518.1; NC_015460.1; NC_015460.1;
NC_015416.1; NC_014933.1; NC_013202.1; NC_013158.1; NC_009464.1; NC_008508.1;
NC_003901.1; NC_009434.1; NC_005085.1; NC_020247.1; NC_020246.1; NC_018224.1;
NC_015943.1; NC_009380.1; NC_006834.1; NC_003552.1; NC_017276.1; NC_017275.1;
NC_010296.1; NC_009615.1; NC_012632.1; NC_012632.1; NC_012623.1; NC_012588.1;
NC_012588.1; NC_007181.1; NC_002939.5; NC_020247.1; NC_020246.1; NC_011296.1;
NC_011296.1; NC_011296.1; NC_018609.1; NC_015964.1; NC_021355.1; NC_020800.1;
NC_019942.1; NC_019792.1; NC_015958.1; NC_015760.1; NC_015678.1; NC_015636.1;
NC_015562.1; NC_014222.1; NC_014222.1; NC_013887.1; NC_013769.1; NC_013156.1;
NC_009953.1; NC_009635.1; NC_009634.1; NC_009135.1; NC_008618.1; NC_008599.1;
NC_007955.1; NC_007796.1; NC_007355.1; NC_002754.1; NC_010572.1; NC_015151.1;
NC_000962.3; NC_021921.1; NC_002163.1; NC_017034.1; NC_009089.1; NC_008698.1;
NC_020419.1; NC_020419.1; NC_020419.1; NC_014160.1; NC_011766.1; NC_007681.1;
NC_000916.1; NC_017527.1; NC_013790.1; NC_013790.1; NC_000917.1; NC_000917.1;
NC_004557.1; NC_004557.1; NC_022246.1; NC_017384.1; NC_013722.1; NC_007643.1;
NC_007643.1; NC_007643.1; NC_007643.1; NC_007643.1; NC_002967.9; NC_004342.2;
NC_016112.1; NC_016112.1; NC_005140.1; NC_005140.1; NC_012726.1; NC_023069.1;
NC_023044.1; NC_022777.1; NC_022777.1; NC_011296.1; NC_021355.1; NC_009634.1;
NC_007796.1; NC_007355.1; NC_021082.1; NC_013926.1; NC_020913.1; NC_014961.1;
NC_014658.1; NC_013198.1; NC_005877.1; NC_009785.1; NC_022084.1; NC_018092.1;
NC_014804.1; NC_000961.1; NC_021058.1; NC_018081.1; NC_013849.1; NC_011567.1;
NC_015574.1; NC_014500.1; NC_012622.1; NC_012589.1; NC_012589.1; NC_019977.1;
NC_019042.1; NC_017274.1; NC_017274.1; NC_015954.1; NC_015676.1; NC_015320.1;
NC_014122.1; NC_014122.1; NC_013407.1; NC_011835.1; NC_021353.1; NC_018001.1;
NC_008818.1; NC_000961.1; NC_015931.1; NC_019042.1; NC_013961.1; NC_011138.3;
NC_009778.1; NC_014228.1; NC_013892.1; NC_011832.1; NC_009439.1; NC_007955.1;
NC_007796.1; NC_013520.1; NC_016070.1; NC_007426.1; NC_003106.2; NC_003106.2;
NC_018227.2; NC_000913.3; NC_005085.1; NC_009613.3; NC_014334.1; NW_006726754.1;
NC_002663.1; NC_003143.1; NC_003076.8; NC_015666.1; NC_014644.1; NC_004116.1;
NC_003454.1; NC_011567.1; NC_024905.1; NC_003295.1; NC_008526.1; NC_012871.1;
NC_012871.1; NC_010682.1; NC_002737.2; NC_002737.2; NC_017954.1; NC_009515.1;
NC_007297.1; NC_007297.1; NC_004350.2; NC_004350.2; NC_000853.1; NC_009009.1;
NC_007644.1; NC_007644.1; NC_002967.9; NC_002932.3; NC_002932.3; NC_007643.1;
NC_007606.1; NC_006347.1; NC_002945.3; NW_006804726.1; NW_006383769.1; NC_013769.1;
NC_014644.1; NC_000913.3; NC_019943.1; NC_019943.1; NC_011661.1; NC_010175.1;
NC_002950.2; NC_004547.2; NC_013887.1; NC_013156.1; NC_007426.1; NC_002939.5;
NC_021169.1; NC_020517.1; NC_018015.1; NC_017946.1; NC_015865.1; NC_015680.1;
NC_009515.1; NC_004557.1; NC_005085.1; NC_006834.1; NC_011296.1; NC_010175.1;
NC_020800.1; NC_015958.1; NC_009635.1; NC_008618.1; NC_007355.1; NC_009089.1;
NC_020419.1; NC_021592.1; NC_021592.1; NC_015847.1; NC_013790.1; NC_016051.1;
NC_007644.1; NC_007644.1; NC_017459.1; NC_015416.1; NC_013722.1; NC_007643.1;
NC_007643.1; NC_007643.1; NC_009434.1; NC_005085.1; NC_003552.1; NC_014318.1;
NC_021355.1; NC_014222.1; NC_014222.1; NC_011832.1; NC_009634.1; NC_009135.1;
NC_021082.1; NC_000961.1; NC_015574.1; NC_014228.1; NC_014122.1; NC_009439.1;
NC_017459.1; NC_015460.1; NC_011138.3; NC_009380.1; NC_017275.1; NC_013892.1;
NC_021353.1; NC_015676.1; NC_011296.1; NC_007955.1; NC_009953.1; NC_009953.1;
NC_021921.1; NC_014160.1; NC_010482.1; NC_009776.1; NC_009033.1; NC_016070.1;
NC_016070.1; NC_015435.1; NC_009440.1; NC_017384.1; NC_013722.1; NC_016112.1;
NC_012726.1; NC_022777.1; NC_008698.1; NC_008599.1; NC_007955.1; NC_007355.1;
NC_014147.1; NC_021058.1; NC_021058.1; NC_016901.1; NC_014500.1; NC_014500.1;
NC_014961.1; NC_018001.1; NC_015931.1; NC_015151.1; NC_013849.1; NC_013715.1;
NC_011766.1; NC_018001.1; NC_014644.1; NC_017034.1; NC_009033.1; NC_002754.1;
NC_009089.1; NC_002939.5; NC_014106.1; NC_010610.1; NC_008054.1; NC_003413.1;
NC_009464.1; NC_008526.1; NC_015474.1; NC_012804.1; NC_015518.1; NC_017276.1;
NC_017275.1; NC_012632.1; NC_012623.1; NC_012588.1; NC_015636.1; NC_015562.1;
NC_013769.1; NC_002754.1; NC_017634.1; NC_014160.1; NC_011766.1; NC_016070.1;
NC_015435.1; NC_009440.1; NC_009440.1; NC_012726.1; NC_012632.1; NC_012588.1;
NC_013887.1; NC_013156.1; NC_011296.1; NC_002754.1; NC_011835.1; NC_018092.1;
NC_021058.1; NC_012622.1; NC_012589.1; NC_015954.1; NC_013407.1; NC_018001.1;
NC_013849.1; NC_017274.1; NC_000913.3; NC_003413.1; NC_018092.1; NC_000961.1;
NC_000918.1; NC_007796.1; NC_000868.1; NC_022084.1; NC_018015.1; NC_015865.1;
NC_015680.1; NC_015474.1; NC_014804.1; NC_012470.1; NC_006624.1; NC_002663.1;
NC_016023.1; NC_013202.1; NC_013158.1; NC_008508.1; NC_000917.1; NC_015943.1;
NC_019792.1; NC_019042.1; NC_015760.1; NC_015678.1; NC_014122.1; NC_004119.1;
NC_007681.1; NC_007681.1; NC_007297.1; NC_002935.2; NC_002932.3; NC_003454.1;
NC_014933.1; NC_011567.1; NC_004342.2; NC_016112.1; NC_003197.1; NC_022777.1;
NC_015320.1; NC_002695.1; NC_003143.1; NC_002737.2; NC_012883.1; NC_010610.1;
NC_000916.1; NC_004350.2; NC_000853.1; NC_000917.1; NC_006347.1; NC_018658.1;
NC_015870.2; NC_011751.1; NC_013961.1; NC_009778.1; NC_020990.1; NC_016112.1;
NC_000868.1; NC_003413.1; NC_022084.1; NC_018092.1; NC_017946.1; NC_015680.1;
NC_015680.1; NC_015474.1; NC_014106.1; NC_012804.1; NC_009053.1; NC_008054.1;
NC_006624.1; NC_000961.1; NC_021058.1; NC_015518.1; NC_018224.1; NC_017276.1;
NC_017276.1; NC_017275.1; NC_017275.1; NC_010296.1; NC_010296.1; NC_009615.1;
NC_012632.1; NC_012632.1; NC_012632.1; NC_012623.1; NC_012623.1; NC_012622.1;
NC_012622.1; NC_012589.1; NC_012589.1; NC_012588.1; NC_012588.1; NC_012588.1;
NC_020892.1; NC_019792.1; NC_017970.1; NC_017274.1; NC_017274.1; NC_016159.1;
NC_013887.1; NC_013769.1; NC_013156.1; NC_002754.1; NC_002754.1; NC_002754.1;
NC_003687.1; NC_006814.3; NC_006814.3; NC_014418.1; NC_010152.1; NC_017946.1;
NC_017954.1; NC_009776.1; NC_008818.1; NC_008818.1; NC_000961.1; NC_000918.1;
NC_015931.1; NC_015931.1; NC_014537.1; NC_007181.1; NC_006624.1; NC_003106.2;
NC_004342.2; NC_020247.1; NC_020246.1; NC_018472.1; NC_012623.1; NC_012589.1;
NC_006045.2; NC_023069.1; NC_022777.1; NC_019942.1; NC_017274.1; NC_013769.1;
NC_009953.1; NC_008698.1; NC_007493.2; NC_002754.1; NC_002754.1; NC_005125.1;
NC_021347.1; NC_022093.1; NC_022093.1; NC_015931.1; NC_007164.1; NC_015416.1;
NC_015151.1; NC_000917.1; NC_003106.2; NC_002932.3; NC_014500.1; NC_004337.2;
NC_007087.3; NC_012726.1; NC_024314.1; NW_003120284.1; NW_003120529.1;
NW_003126883.1; NW_003384275.1; NC_023069.1; NC_016567.1; NC_009954.1; NC_000913.3;
NC_000913.3; NC_000913.3; NC_000913.3; NC_027204.1; NC_002754.1; NC_010175.1;
NC_016070.1; NC_000868.1; NC_003413.1; NC_017527.1; NC_002939.5; NC_018227.2;
NC_007355.1; NC_014205.1; NC_014160.1; NC_009033.1; NC_007681.1; NC_020517.1;
NC_018015.1; NC_016070.1; NC_015865.1; NC_015847.1; NC_015680.1; NC_015474.1;
NC_015315.1; NC_013790.1; NC_012883.1; NC_012470.1; NC_016051.1; NC_011529.1;
NC_010610.1; NC_009515.1; NC_009440.1; NC_007181.1; NC_006624.1; NC_019943.1;
NC_019943.1; NC_017459.1; NC_015518.1; NC_014933.1; NC_007426.1; NC_003901.1;
NC_003106.2; NC_003106.2; NC_009434.1; NC_005085.1; NC_020247.1; NC_020246.1;
NC_006834.1; NC_017276.1; NC_017276.1; NC_017276.1; NC_017275.1; NC_017275.1;
NC_010296.1; NC_009615.1; NC_012632.1; NC_012632.1; NC_012623.1; NC_012623.1;
NC_012588.1; NC_012588.1; NC_007643.1; NC_021355.1; NC_020800.1; NC_015958.1;
NC_015678.1; NC_014222.1; NC_013769.1; NC_013769.1; NC_009635.1; NC_009634.1;
NC_009135.1; NC_008618.1; NC_007796.1; NC_002754.1; NC_002754.1; NC_017034.1;
NC_019413.1; NC_020419.1; NC_017946.1; NC_014374.1; NC_014374.1; NC_011766.1;
NC_011766.1; NC_010482.1; NC_009776.1; NC_009776.1; NC_009033.1; NC_008942.1;
NC_021169.1; NC_015518.1; NC_014537.1; NC_012804.1; NC_007644.1; NC_015416.1;
NC_013722.1; NC_007643.1; NC_016112.1; NC_016112.1; NC_012726.1; NC_012726.1;
NC_023069.1; NC_023069.1; NC_021355.1; NC_019942.1; NC_014002.1; NC_021082.1;
NC_005830.1; NC_018001.1; NC_017954.1; NC_014961.1; NC_022084.1; NC_018092.1;
NC_014804.1; NC_021058.1; NC_021058.1; NC_021058.1; NC_015574.1; NC_015216.1;
NC_014500.1; NC_012622.1; NC_012622.1; NC_012589.1; NC_012589.1; NC_012589.1;
NC_005265.1; NC_011661.1; NC_019977.1; NC_017274.1; NC_017274.1; NC_017274.1;
NC_015954.1; NC_008701.1; NC_009965.1; NC_014961.1; NC_013926.1; NC_018001.1;
NC_013741.1; NC_013741.1; NC_008818.1; NC_000961.1; NC_015931.1; NC_015931.1;
NC_015151.1; NC_015151.1; NC_013849.1; NC_015320.1; NC_014122.1; NC_000913.3;
NC_011217.1; NC_003413.1; NC_005360.1; NC_005361.1; NC_009986.1; NC_021921.1;
NC_007681.1; NC_021169.1; NC_017946.1; NC_015865.1; NC_015680.1; NC_015474.1;
NC_012804.1; NC_002950.2; NC_004557.1; NC_017459.1; NC_016023.1; NC_013202.1;
NC_013158.1; NC_018224.1; NC_015943.1; NC_003552.1; NC_012632.1; NC_012588.1;
NC_011296.1; NC_011296.1; NC_021355.1; NC_019942.1; NC_019792.1; NC_015958.1;
NC_007955.1; NC_009089.1; NC_018015.1; NC_018015.1; NC_007644.1; NC_004557.1;
NC_022777.1; NC_022777.1; NC_015562.1; NC_008599.1; NC_007355.1; NC_018092.1;
NC_000961.1; NC_011567.1; NC_013926.1; NC_021353.1; NC_022084.1; NC_015574.1;
NC_003106.2; NC_015320.1; NC_016070.1; NC_013588.1; NC_011835.1; NC_011766.1;
NC_009776.1; NC_009033.1; NC_010482.1; NC_009440.1; NC_015518.1; NC_003106.2;
NC_017276.1; NC_017275.1; NC_012632.1; NC_012623.1; NC_012588.1; NC_013769.1;
NC_002754.1; NC_017034.1; NC_014374.1; NC_015416.1; NC_003106.2; NC_012726.1;
NC_013715.1; NC_025439.1; NC_017954.1; NC_014961.1; NC_013849.1; NC_012622.1;
NC_012589.1; NC_017274.1; NC_018001.1; NC_015931.1; NC_009380.1; NC_000010.11;
NC_006477.3; AC_000185.1; NC_013907.1; NC_025687.1; NC_012021.3; NC_023046.1;
NC_023650.1; NW_006159565.1; NC_018160.1; NC_027878.1; NC_024803.1; NW_007540324.1;
NC_020497.1; NC_026657.1; NC_003280.10; NC_004354.4; NC_001140.6; NC_003070.9;
NT_078265.2; NW_006890056.1; NW_003613826.1; NC_026506.1; NC_023616.1; NC_025973.2;
NC_024333.1; NW_006717075.1; NW_006438113.1; NW_006399752.1; NW_005785394.1;
NC_019873.1; NC_019482.1; NC_018902.1; NC_022199.1; NC_008801.1; NC_026145.1;
NC_015446.2; NC_026940.1; NC_025695.1; NC_025207.1; NW_006749282.1; NC_015438.2;
NC_016093.1; NC_007418.2; NC_000076.6; NC_003423.3; NC_003423.3; NC_001134.8;
NC_015474.1; NC_005104.4; NC_007126.6; NC_007124.6; NC_006093.3; NC_024464.1;
NC_007124.6; NC_008397.2; NC_010572.1; NC_010572.1; NC_017301.1; NC_017941.2;
NC_016407.2; NC_016410.2; NC_012026.1; NC_021921.1; NC_018092.1; NC_017946.1;
NC_015865.1; NC_015680.1; NC_015435.1; NC_012804.1; NC_016051.1; NC_011529.1;
NC_009515.1; NC_009515.1; NC_009440.1; NC_007164.1; NC_006624.1; NC_002935.2;
NC_000916.1; NC_017459.1; NC_015151.1; NC_014933.1; NC_014729.1; NC_014537.1;
NC_013209.1; NC_013202.1; NC_013158.1; NC_011567.1; NC_010364.1; NC_008212.1;
NC_007426.1; NC_002607.1; NC_003366.1; NC_023013.1; NC_018224.1; NC_016946.1;
NC_016946.1; NC_015948.1; NC_015666.1; NC_014297.1; NC_013967.1; NC_013922.1;
NC_013743.1; NC_009783.1; NC_009380.1; NC_005786.2; NC_005785.6; NC_006396.1;
NC_002944.2; NC_010002.1; NC_020990.1; NC_009615.1; NC_009614.1; NC_008313.1;
NC_013929.1; NC_017765.1; NC_017844.1; NC_014323.1; NC_007973.1; NC_019962.1;
NC_019792.1; NC_019042.1; NC_016114.1; NC_016114.1; NC_015954.1; NC_011753.2;
NC_010505.1; NC_009953.1; NC_009953.1; NC_008268.1; NC_007866.1; NC_008596.1;
NC_008596.1; NC_000868.1; NC_004668.1; NC_009760.1; NC_006820.1; NC_003888.3;
NC_025463.1; NC_021921.1; NC_021921.1; NC_006883.2; NC_017034.1; NC_014423.1;
NC_017941.2; NC_004603.1; NC_007088.5; NC_008367.1; NC_021776.1; NT_078267.5;
NC_002656.1; NC_022774.1; NC_011091.1; NC_005881.2; NC_011183.1; NC_015937.1;
NC_008584.1; NC_024122.1; NC_022768.1; NC_022772.1; NC_013085.1; NC_010154.1;
NC_010152.1; NC_010155.1; NC_009804.1; NC_009803.1; NC_005342.2; NC_004333.2;
NC_023735.1; NC_023694.1; NC_027364.1; NC_019526.1; NC_023607.1; NC_021353.1;
NC_021592.1; NC_012039.1; NC_008942.1; NC_002936.3; NC_005877.1; NC_021169.1;
NC_021058.1; NC_020517.1; NC_020388.1; NC_020388.1; NC_018656.1; NC_015435.1;
NC_014804.1; NC_013790.1; NC_013790.1; NC_009440.1; NC_009051.1; NC_007929.1;
NC_007929.1; NC_005042.1; NC_003454.1; NC_003238.2; NC_021313.1; NC_019943.1;
NC_019943.1; NC_017459.1; NC_017384.1; NC_015288.1; NC_015287.1; NC_015284.1;
NC_015281.1; NC_015280.1; NC_014334.1; NC_014297.1; NC_013967.1; NC_013202.1;
NC_011129.1; NC_007426.1; NC_007426.1; NC_003901.1; NC_003901.1; NC_004342.2;
NC_014622.2; NC_023731.1; NC_023729.1; NC_023716.1; NC_017275.1; NC_015574.1;
NC_015216.1; NC_015216.1; NC_013922.1; NC_013922.1; NC_013743.1; NC_012966.1;
NC_012966.1; NC_011913.1; NC_010397.1; NC_010296.1; NC_009380.1; NC_006396.1;
NC_006347.1; NC_002944.2; NC_003552.1; NC_004663.1; NW_006890135.1; NC_026750.1;
NC_021285.1; NC_017765.1; NC_014942.1; NC_014435.1; NC_014318.1; NC_008526.1;
NC_007197.1; NC_006687.1; NC_022543.1; NC_022528.1; NC_022528.1; NC_012623.1;
NC_012622.1; NC_012589.1; NC_023617.1; NC_006049.2; NC_006048.2; NC_013486.1;
NC_006071.1; NC_006038.1; NC_006037.1; NC_012878.1; NC_012875.1; NC_008480.2;
NC_011089.1; NW_007907148.1; NW_008246060.1; NW_007726404.1; NW_007677829.1;
NW_007620716.1; NC_024318.1; NC_024311.1; NW_007578198.1; NW_007370681.1;
NW_007249782.1; NC_023643.1; NW_006921657.1; NW_006799972.1; NW_006804151.1;
NW_006784004.1; NW_006532274.1; NW_006725466.1; NW_006712311.1; NW_006610005.1;
NW_006501061.1; NW_006399824.1; NW_006408538.1; NW_006288853.1; NW_006383157.1;
NW_006224511.1; NW_005882862.1; NW_006210709.1; NW_006159730.1; NW_005859001.1;
NW_005855688.1; NW_005871110.1; NW_005843034.1; NW_005819063.1; NW_005815230.1;
NW_005395486.1; NW_005369027.1; NC_022320.1; NC_022280.1; NC_022028.1;
NW_007359849.1; NW_007359875.1; NC_021678.1; NW_004454176.1; NW_004205048.1;
NC_019861.1; NC_018434.1; NC_018733.2; NC_018893.1; NC_016133.1; NC_016135.1;
NC_016098.1; NC_010459.4; NC_022211.1; NC_015018.2; NC_013686.1; NC_011470.1;
NC_009144.2; NC_007796.1; NC_018609.1; NC_027340.1; NC_027218.1; NC_027132.1;
NC_026928.1; NC_026583.1; NC_026440.1; NC_025422.1; NC_025447.1; NC_023862.1;
NC_023553.1; NC_023587.1; NC_023564.1; NC_022973.1; NC_022984.1; NC_022086.1;
NC_021307.1; NC_021321.1; NC_021335.1; NC_021320.1; NC_020159.1; NC_019974.1;
NC_019974.1; NC_019974.1; NC_019974.1; NC_019962.1; NC_019962.1; NC_019942.1;
NC_018283.1; NC_018227.2; NC_016653.1; NC_016570.1; NC_013600.1; NC_015954.1;
NC_015958.1; NC_015676.1; NC_015274.1; NC_015296.1; NC_014371.1; NC_014253.1;
NC_013769.1; NC_013084.1; NC_013081.1; NC_013079.1; NC_012865.1; NC_012029.1;
NC_011023.1; NC_011019.1; NC_010537.1; NC_009954.1; NC_007796.1; NC_007493.2;
NC_007355.1; NC_002946.2; NC_002946.2; NC_002519.1; NC_006089.3; NC_006089.3;
NC_003214.2; NC_010356.1; NC_004463.1; NC_007797.1; NC_002737.2; NC_025375.1;
NC_005125.1; NC_027120.1; NC_018280.1; NC_016566.1; NC_007077.3; NC_019419.2;
NC_012223.2; NC_021783.1; NC_021342.2; NC_021315.1; NC_020204.1; NC_025443.1;
NC_023549.1; NC_019924.1; NC_019550.1; NC_019549.1; NC_014457.1; NC_023566.1;
NC_004927.1; NC_003345.1; NC_014322.1; NC_027119.1; NC_019452.1; NC_010153.1;
NC_021780.1; NC_021775.1; NC_020419.1; NC_020419.1; NC_013926.1; NC_005364.2;
NC_017954.1; NC_012039.1; NC_000918.1; NC_013790.1; NC_022093.1; NC_022093.1;
NC_022093.1; NC_022093.1; NC_021592.1; NC_021169.1; NC_018015.1; NC_015931.1;
NC_015435.1; NC_014408.1; NC_013790.1; NC_012883.1; NC_008818.1; NC_008818.1;
NC_008818.1; NC_008818.1; NC_007297.1; NC_006570.2; NC_000916.1; NC_004350.2;
NC_003454.1; NC_000853.1; NC_018876.1; NC_017527.1; NC_021313.1; NC_020388.1;
NC_015518.1; NC_015416.1; NC_015416.1; NC_015151.1; NC_015151.1; NC_014537.1;
NC_014537.1; NC_014537.1; NC_014537.1; NC_014507.1; NC_013849.1; NC_013849.1;
NC_009464.1; NC_000917.1; NC_000917.1; NC_000917.1; NC_000917.1; NC_002932.3;
NC_026153.1; NC_020177.2; NC_027207.1; NC_025260.1; NC_018224.1; NC_016449.1;
NC_016453.1; NC_015574.1; NC_007643.1; NC_007643.1; NC_026756.1; NC_026492.1;
NC_026490.1; NC_021366.1; NC_014946.1; NC_014950.1; NC_009667.1; NC_009048.1;
NC_006682.1; NW_001820527.3; NC_018213.1; NC_009364.1; NC_026477.1; NC_017844.1;
NC_016494.1; NC_016492.1; NC_016457.1; NC_016475.1; NC_016507.1; NC_016502.1;
NC_012726.1; NC_011672.1; NC_011687.1; NC_006036.2; NC_006032.2; NC_026933.1;
NC_026006.1; NC_026003.1; NC_015018.2; NC_025820.1; NC_025701.1; NC_024796.1;
NC_024460.1; NW_007378989.1; NW_007377463.1; NW_007546349.1; NW_007546273.1;
NC_024245.1; NC_024241.1; NC_024130.1; NC_024126.1; NW_006921822.1; NW_006534794.1;
NW_006749448.1; NW_006601119.1; NW_003140815.1; NW_006272001.1; NW_006280498.1;
NC_023166.1; NC_023167.1; NC_023200.1; NC_023183.1; NW_006263308.1; NC_016107.1;
NC_023053.1; NW_006238943.1; NW_006239104.1; NW_006235748.1; NW_006190770.1;
NW_006188714.1; NW_005871025.1; NW_005872046.1; NW_005841850.1; NW_005372290.1;
NC_010456.4; NC_021165.1; NC_021163.1; NC_020492.1; NC_020496.1; NC_015448.2;
NC_026660.1; NW_003803976.1; NC_019833.1; NC_012601.1; NW_003139842.1; NC_012012.3;
NW_003384299.1; NW_001760161.1; NC_008802.1; NC_027383.1; NC_023044.1; NC_026591.1;
NC_025446.1; NC_025442.1; NC_025428.1; NC_024783.1; NC_023856.1; NC_023755.1;
NC_023753.1; NC_023751.1; NC_023749.1; NC_023749.1; NC_020853.1; NC_020851.1;
NC_020845.1; NC_020844.1; NC_020837.1; NC_020082.1; NC_019977.1; NC_019942.1;
NC_019791.1; NC_019724.1; NC_019530.1; NC_019524.1; NC_019519.1; NC_019518.1;
NC_019457.1; NC_019417.1; NC_019401.1; NC_018396.1; NC_016520.1; NC_016531.1;
NC_016073.1; NC_015676.1; NC_015569.1; NC_015466.1; NC_015320.1; NC_012995.1;
NC_012992.1; NC_012867.1; NC_011832.1; NC_010175.1; NC_009954.1; NC_009712.1;
NC_007710.1; NC_009189.1; NC_009181.1; NC_008698.1; NC_008698.1; NC_007955.1;
NC_007796.1; NC_012589.1; NC_012588.1; NC_004086.1; NC_004342.2; NC_022096.1;
NC_014644.1; NC_011661.1; NC_010175.1; NC_002754.1; NC_002754.1; NC_003413.1;
NC_021921.1; NC_002754.1; NC_018015.1; NC_018015.1; NC_015865.1; NC_015847.1;
NC_015680.1; NC_015680.1; NC_015680.1; NC_015474.1; NC_015474.1; NC_015435.1;
NC_012804.1; NC_012470.1; NC_009515.1; NC_009440.1; NC_006624.1; NC_006624.1;
NC_004557.1; NC_004557.1; NC_016023.1; NC_015518.1; NC_013202.1; NC_013158.1;
NC_003106.2; NC_003106.2; NC_015943.1; NC_011138.3; NC_009778.1; NC_009380.1;
NC_003552.1; NC_014228.1; NC_017276.1; NC_017275.1; NC_013892.1; NC_012632.1;
NC_012632.1; NC_012623.1; NC_012588.1; NC_012588.1; NC_011296.1; NC_011296.1;
NC_019792.1; NC_015678.1; NC_014222.1; NC_013887.1; NC_013887.1; NC_013769.1;
NC_013156.1; NC_013156.1; NC_011832.1; NC_009953.1; NC_009635.1; NC_009634.1;
NC_009439.1; NC_008618.1; NC_007955.1; NC_007355.1; NC_010572.1; NC_016070.1;
NC_000868.1; NC_009089.1; NC_020419.1; NC_021592.1; NC_017946.1; NC_017946.1;
NC_011766.1; NC_010482.1; NC_009776.1; NC_009033.1; NC_021169.1; NC_014537.1;
NC_013790.1; NC_016051.1; NC_000917.1; NC_003454.1; NC_018224.1; NC_017459.1;
NC_015416.1; NC_014933.1; NC_016112.1; NC_003198.1; NC_012726.1; NC_014222.1;
NC_009135.1; NC_023069.1; NC_023044.1; NC_022777.1; NC_021355.1; NC_019942.1;
NC_015958.1; NC_015760.1; NC_009953.1; NC_008599.1; NC_022084.1; NC_018092.1;
NC_018092.1; NC_000961.1; NC_021058.1; NC_011567.1; NC_015574.1; NC_012622.1;
NC_012589.1; NC_002950.2; NC_000913.3; NC_017274.1; NC_015676.1; NC_015320.1;
NC_014122.1; NC_014122.1; NC_017301.1; NC_021353.1; NC_018001.1; NC_018001.1;
NC_000961.1; NC_000961.1; NC_015931.1; NC_015931.1; NC_013849.1; NC_002950.2;
NC_002663.1; NC_015460.1; NC_005085.1; NC_010002.1; NC_013722.1; NC_003106.2;
NC_007796.1; NC_014147.1; NC_009053.1; NC_016901.1; NC_014500.1; NC_003413.1;
NC_016070.1; NC_022777.1; NC_015516.1; NC_020517.1; NC_014106.1; NC_010610.1;
NC_008942.1; NC_008054.1; NC_007164.1; NC_002935.2; NC_013961.1; NC_009434.1;
NC_006834.1; NC_020990.1; NC_009615.1; NC_020800.1; NC_021082.1; NC_009089.1;
NC_014160.1; NC_007644.1; NC_019943.1; NC_017384.1; NC_013722.1; NC_007643.1;
NC_007643.1; NC_007643.1; NC_002939.5; NC_004342.2; NC_005085.1; NC_008526.1;
NC_014500.1; NC_009515.1; NC_003280.10; NC_008508.1; NC_008508.1; NC_004342.2;
NC_010296.1; NC_003197.1; NC_007090.3; NW_006623966.1; NC_015020.2; NC_019042.1;
NC_011273.1; NC_002695.1; NC_002737.2; NC_007581.1; NC_017954.1; NC_014961.1;
NC_014658.1; NC_007681.1; NC_007681.1; NC_000918.1; NC_000916.1; NC_022093.1;
NC_021592.1; NC_015865.1; NC_014804.1; NC_012883.1; NC_010610.1; NC_008818.1;
NC_007297.1; NC_004350.2; NC_000853.1; NC_015151.1; NC_007644.1; NC_000917.1;
NC_004113.1; NC_002932.3; NC_002932.3; NC_022239.1; NC_016112.1; NC_016112.1;
NC_003901.1; NC_018658.1; NC_014431.1; NC_014318.1; NC_011751.1; NC_009359.1;
NC_026939.1; NC_024803.1; NC_024313.1; NW_005890115.1; NC_026659.1; NC_023752.1;
NC_009954.1; NC_007955.1; NC_007796.1; NC_025443.1; NC_025687.1; NC_009698.1;
NC_022777.1; NC_002754.1; NC_000961.1; NC_011661.1; NC_000868.1; NC_000868.1;
NC_003413.1; NC_003413.1; NC_021921.1; NC_000916.1; NC_021169.1; NC_018015.1;
NC_018015.1; NC_018015.1; NC_017946.1; NC_017946.1; NC_015865.1; NC_015865.1;
NC_015865.1; NC_015680.1; NC_015680.1; NC_015680.1; NC_015474.1; NC_015474.1;
NC_012883.1; NC_012804.1; NC_012804.1; NC_016051.1; NC_016051.1; NC_011529.1;
NC_011529.1; NC_007181.1; NC_006624.1; NC_006624.1; NC_006624.1; NC_006448.1;
NC_004557.1; NC_004557.1; NC_017459.1; NC_015518.1; NC_015518.1; NC_015460.1;
NC_013202.1; NC_013158.1; NC_000917.1; NC_020247.1; NC_020246.1; NC_003552.1;
NC_017275.1; NC_010296.1; NC_012632.1; NC_012632.1; NC_012588.1; NC_012588.1;
NC_011296.1; NC_011296.1; NC_021355.1; NC_019792.1; NC_015958.1; NC_015636.1;
NC_015562.1; NC_013887.1; NC_013156.1; NC_007955.1; NC_007355.1; NC_002754.1;
NC_002754.1; NC_009778.1; NC_000962.3; NC_009089.1; NC_021592.1; NC_017946.1;
NC_015680.1; NC_018015.1; NC_014537.1; NC_014537.1; NC_012883.1; NC_012804.1;
NC_018224.1; NC_017459.1; NC_016023.1; NC_015943.1; NC_023069.1; NC_023044.1;
NC_019942.1; NC_014222.1; NC_008599.1; NC_002754.1; NC_022084.1; NC_022084.1;
NC_022084.1; NC_018092.1; NC_014804.1; NC_014804.1; NC_021058.1; NC_017274.1;
NC_015320.1; NC_014122.1; NC_013407.1; NC_014658.1; NC_000961.1; NC_000961.1;
NC_018092.1; NC_015151.1; NC_013849.1; NC_011567.1; NC_013926.1; NC_002754.1;
NC_013520.1; NC_013520.1; NC_007181.1; NC_007426.1; NC_003106.2; NC_020247.1;
NC_020246.1; NC_017276.1; NC_017275.1; NC_012632.1; NC_012623.1; NC_012588.1;
NC_011296.1; NC_011296.1; NC_013769.1; NC_013769.1; NC_012726.1; NC_021058.1;
NC_012622.1; NC_012589.1; NC_015151.1; NC_015954.1; NC_004547.2; NC_000913.3;
NC_010175.1; NC_016070.1; NC_002950.2; NC_009380.1; NC_009089.1; NC_009495.1;
NC_022777.1; NC_007796.1; NC_014106.1; NC_008054.1; NC_002663.1; NC_013961.1;
NC_011138.3; NC_014228.1; NC_020990.1; NC_013892.1; NC_015760.1; NC_009953.1;
NC_009953.1; NC_009439.1; NC_010572.1; NC_002971.3; NC_021353.1; NC_014644.1;
NC_010610.1; NC_002935.2; NC_013722.1; NC_009464.1; NC_007643.1; NC_002939.5;
NC_010002.1; NC_003198.1; NC_014318.1; NC_008526.1; NC_011832.1; NC_008701.1;
NC_007955.1; NC_007796.1; NC_014147.1; NC_009053.1; NC_014500.1; NC_016901.1;
NC_015676.1; NC_020418.1; NW_003613864.1; NC_000011.10; NW_006800487.1; NC_006603.3;
NW_003614246.1; NC_012602.1; NC_013790.1; NC_009515.1; NC_015574.1; NC_007871.1;
NC_006478.3; NC_004347.2; NC_009006.2; NT_078266.2; NC_003454.1; NW_006212882.1;
NC_011913.1; NC_009917.1; NW_007675828.1; NW_007370782.1; NW_007248774.1;
NC_023642.1; NW_006775074.1; NW_006730123.1; NW_006718075.1; NW_006711808.1;
NW_006400147.1; NW_006408681.1; NW_006384369.1; NW_005882764.1; NW_006200097.1;
NC_022285.1; NW_004209914.1; NC_018435.1; NC_018732.2; NC_018165.1; NC_027879.1;
NC_019830.1; NC_013906.1; NC_009150.2; NC_000964.3; NC_002696.2; NC_017034.1;
NC_007581.1; NC_018001.1; NC_017954.1; NC_014961.1; NC_014961.1; NC_011766.1;
NC_009776.1; NC_007681.1; NC_007681.1; NC_005877.1; NC_005877.1; NC_005877.1;
NC_002689.2; NC_000918.1; NC_000918.1; NC_021592.1; NC_015931.1; NC_015931.1;
NC_010482.1; NC_009033.1; NC_000853.1; NC_015518.1; NC_015416.1; NC_013849.1;
NC_009440.1; NC_009440.1; NC_009009.1; NC_007644.1; NC_000917.1; NC_003106.2;
NC_011916.1; NC_007643.1; NC_006347.1; NC_004342.2; NC_002945.3; NC_012589.1;
NC_012623.1; NC_011672.1; NC_016131.1; NW_004454187.1; NC_019862.1; NC_010451.3;
NC_015768.1; NC_020173.2; NC_017972.1; NC_015320.1; NC_011832.1; NC_010175.1;
NC_010175.1; NC_008599.1; NC_006461.1; NC_015637.1; NC_009784.1; NC_016114.1;
NC_001493.2; NC_008508.1; NC_003197.1; NC_017844.1; NW_006399893.1; NC_002695.1;
NC_017634.1; NC_003143.1; NC_017941.2; NC_004605.1; NC_004605.1; NC_019411.1;
NC_007164.1; NC_002932.3; NC_005085.1; NC_027207.1; NC_016452.1; NC_016112.1;
NC_009784.1; NC_007606.1; NC_002506.1; NC_018658.1; NC_011751.1; NC_016497.1;
NC_011678.1; NC_006035.2; NC_026147.1; NC_013913.1; NC_024132.1; NC_006461.1;
NC_000913.3; NC_014106.1; NC_008054.1; NC_013961.1; NC_011138.3; NC_009778.1;
NC_014228.1; NC_020990.1; NC_013892.1; NC_015760.1; NC_009953.1; NC_009953.1;
NC_009439.1; NC_010572.1; NC_014644.1; NC_010610.1; NC_002935.2; NC_009464.1;
NC_007643.1; NC_002939.5; NC_003198.1; NC_014318.1; NC_008526.1; NC_011832.1;
NC_007955.1; NC_007796.1; NC_015676.1; NC_015637.1; NC_009784.1; NC_016114.1;
NC_001493.2; NC_008508.1; NC_003197.1; NC_017844.1; NC_002695.1; NC_017941.2;
NC_004605.1; NC_007164.1; NC_002932.3; NC_016452.1; NC_016112.1; NC_007606.1;
NC_002506.1; NC_018658.1; NC_011751.1; NC_026147.1; NC_024132.1; NC_006461.1;
NC_004547.2; NC_002663.1; NC_002971.3; NC_013722.1; NC_010002.1; NC_014147.1;
NC_009053.1; NC_014500.1; NC_016901.1; NC_020418.1; NW_006399893.1; NC_017634.1;
NC_003143.1; NC_004605.1; NC_019411.1; NC_005085.1; NC_027207.1; NC_009784.1;
NC_016497.1; NC_011678.1; NC_006035.2; NC_013913.1; NC_000913.3; NC_002950.2;
NC_019943.1; NC_021355.1; NC_019942.1; NC_021058.1; NC_017576.1; NC_009785.1;
NC_019943.1; NC_010175.1; NC_002754.1; NC_016070.1; NC_003413.1; NC_003413.1;
NC_021921.1; NC_015516.1; NC_007681.1; NC_021169.1; NC_020517.1; NC_018015.1;
NC_017946.1; NC_017946.1; NC_015865.1; NC_015847.1; NC_015680.1; NC_015680.1;
NC_015680.1; NC_015474.1; NC_015435.1; NC_014106.1; NC_013790.1; NC_012470.1;
NC_010610.1; NC_010610.1; NC_010482.1; NC_009515.1; NC_009440.1; NC_008942.1;
NC_008054.1; NC_006624.1; NC_017459.1; NC_015518.1; NC_014933.1; NC_013961.1;
NC_013202.1; NC_013158.1; NC_000917.1; NC_003901.1; NC_003106.2; NC_009434.1;
NC_005085.1; NC_018224.1; NC_015943.1; NC_009380.1; NC_006834.1; NC_003552.1;
NC_020990.1; NC_017276.1; NC_017275.1; NC_009615.1; NC_012632.1; NC_012632.1;
NC_012623.1; NC_012588.1; NC_012588.1; NC_020800.1; NC_019792.1; NC_015760.1;
NC_015678.1; NC_014222.1; NC_013887.1; NC_013887.1; NC_013156.1; NC_013156.1;
NC_009953.1; NC_009634.1; NC_008618.1; NC_008599.1; NC_007355.1; NC_002754.1;
NC_002754.1; NC_021082.1; NC_000868.1; NC_009089.1; NC_009089.1; NC_020419.1;
NC_021592.1; NC_014644.1; NC_000916.1; NC_015474.1; NC_014537.1; NC_012804.1;
NC_016051.1; NC_007644.1; NC_006624.1; NC_004557.1; NC_004557.1; NC_017384.1;
NC_016023.1; NC_013722.1; NC_007643.1; NC_007643.1; NC_016112.1; NC_016112.1;
NC_014318.1; NC_008526.1; NC_012726.1; NC_023044.1; NC_022777.1; NC_014222.1;
NC_011296.1; NC_009953.1; NC_018001.1; NC_017954.1; NC_022084.1; NC_018092.1;
NC_018092.1; NC_018015.1; NC_000961.1; NC_011567.1; NC_000917.1; NC_015574.1;
NC_014500.1; NC_012622.1; NC_012589.1; NC_017274.1; NC_015320.1; NC_014122.1;
NC_021353.1; NC_000916.1; NC_000961.1; NC_000961.1; NC_015931.1; NC_013849.1;
NC_011296.1; NC_002754.1; NC_022084.1; NC_006590.3; NW_003613770.1; NW_006770037.1;
NW_005882719.1; NW_006717982.1; NW_003159449.1; NW_007676006.1; NW_004454173.1;
NC_009167.2; NC_021161.1; NC_007131.6; NC_010443.4; NC_013929.1; NC_016114.1;
NC_020416.1; NC_014160.1; NC_004342.2; NC_021366.1; NC_014430.1; NW_007727498.1;
NW_006777583.1; NW_006732012.1; NW_006399890.1; NW_005882776.1; NC_019874.1;
NC_013918.1; NC_026607.2; NC_022777.1; NC_019042.1; NC_011421.1; NC_009784.1;
NC_002695.1; NC_002737.2; NC_021315.1; NC_007581.1; NC_022761.1; NC_000918.1;
NC_012883.1; NC_007297.1; NC_004350.2; NC_003454.1; NC_000853.1; NC_009464.1;
NC_002932.3; NC_002932.3; NC_016112.1; NC_006347.1; NC_004342.2; NC_018658.1;
NC_010397.1; NC_002678.2; NC_011751.1; NC_011695.1; NC_025697.1; NC_025205.1;
NW_006239047.1; NW_005372402.1; NC_020494.1; NW_003804572.1; NC_016132.1;
NC_027344.1; NC_025442.1; NC_015320.1; NC_014923.1; NC_013046.1; NC_011167.1;
NC_007955.1; NC_002754.1; NC_008818.1; NC_000918.1; NC_014222.1; NC_011661.1;
NC_003413.1; NC_010175.1; NC_015474.1; NC_013202.1; NC_018224.1; NC_003552.1;
NC_009635.1; NC_007355.1; NC_009089.1; NC_021592.1; NC_015847.1; NC_009515.1;
NC_018015.1; NC_006624.1; NC_019943.1; NC_017459.1; NC_015943.1; NC_015416.1;
NC_022777.1; NC_022777.1; NC_014222.1; NC_011296.1; NC_009634.1; NC_022084.1;
NC_018092.1; NC_000961.1; NC_011567.1; NC_015574.1; NC_014122.1; NC_021353.1;
NC_015320.1; NC_014122.1; NC_019471.1; NC_026149.1; NW_006711753.1; NC_025697.1;
NW_007540505.1; NW_006749413.1; NC_008809.1; NT_033779.5; NC_000961.1; NC_015562.1;
NC_011645.1; NC_017034.1; NC_019912.1; NC_011318.1; NT_167064.1; NC_002939.5;
NC_011904.1; NC_008314.1; NC_024342.1; NW_007370677.1; NW_006804181.1;
NW_006711988.1; NW_006399752.1; NW_006299715.1; NW_005785016.1; NW_005805521.1;
NC_018726.2; NC_018726.2; NC_008801.1; NC_013081.1; NC_006840.2; NC_023007.1;
NC_014649.1; NC_007681.1; NC_007681.1; NC_007681.1; NC_000918.1; NC_021592.1;
NC_013790.1; NC_003454.1; NC_000853.1; NC_004557.1; NC_007644.1; NW_007544598.1;
NC_027207.1; NC_003901.1; NC_009908.2; NC_016506.1; NC_006041.1; NC_006029.1;
NC_025997.1; NW_007733735.1; NW_006799945.1; NW_006749276.1; NC_016088.1;
NW_005871062.1; NW_005369870.1; NW_005372669.1; NW_004204575.1; NC_021355.1;
NC_014222.1; NC_009135.1; NC_000868.1; NC_003413.1; NC_017946.1; NC_015680.1;
NC_015474.1; NC_012804.1; NC_006624.1; NC_016070.1; NC_013887.1; NC_013156.1;
NC_022084.1; NC_018092.1; NC_018001.1; NC_000961.1; NC_013849.1; NC_016091.1;
NC_013929.1; NC_018734.2; NC_018609.1; NC_003413.1; NC_014961.1; NC_018092.1;
NC_021313.1; NC_013488.1; NC_023186.1; NC_022777.1; NC_021921.1; NC_013158.1;
NC_019792.1; NC_004557.1; NC_019943.1; NC_015516.1; NC_012470.1; NC_016023.1;
NC_009434.1; NC_005085.1; NC_006834.1; NC_015678.1; NC_020419.1; NC_020517.1;
NC_007644.1; NC_017384.1; NC_014933.1; NC_013722.1; NC_007643.1; NC_016112.1;
NC_009615.1; NC_002950.2; NC_021082.1; NC_014500.1; NC_008804.1; NC_007873.1;
NC_004342.2; NC_018015.1; NC_016901.1; NC_007796.1; NC_008195.1; NC_013158.1;
NC_014371.1; NC_019042.1; NC_011044.1; NC_002946.2; NC_002946.2; NC_002737.2;
NC_018859.1; NC_026932.1; NC_010610.1; NC_008942.1; NC_007297.1; NC_004350.2;
NC_004350.2; NC_002932.3; NC_016112.1; NC_015574.1; NC_004342.2; NC_014942.1;
NC_026148.1; NC_025703.1; NW_007680270.1; NC_020493.1; NC_023554.1; NC_022327.1;
NC_022331.1; NC_022328.1; NC_016522.1; NC_002663.1; NC_018609.1; NC_006448.1;
NC_017576.1; NC_015964.1; NC_002163.1; NC_002967.9; NC_009785.1; NC_004829.2;
NC_017576.1; NC_002935.2; NC_013198.1; NC_020419.1; NC_009613.3; NC_022246.1;
NC_007643.1; NC_018081.1; NC_007117.6; NC_023622.1; NC_007297.1; NC_024462.1;
NC_003076.8; NC_019042.1; NC_002737.2; NC_004350.2; NC_004116.1; NC_017946.1;
NC_000961.1; NT_167064.1; NC_002967.9; NC_012963.1; NC_023658.1; NW_006725355.1;
NW_006400033.1; NW_006385373.1; NW_005815240.1; NW_004197941.1; NC_009094.1;
NC_019516.1; NC_008599.1; NC_004740.1; NC_007929.1; NC_006570.2; NC_014429.1;
NC_026477.1; NC_016462.1; NC_025688.1; NC_025693.1; NC_024800.1; NW_006399825.1;
NC_019403.1; NC_016524.1; NC_007709.1; NC_000007.14; NC_000962.3; NC_021058.1;
NC_021058.1; NC_002754.1; NC_017946.1; NC_015680.1; NC_011529.1; NC_017276.1;
NC_012622.1; NC_012622.1; NC_000961.1; NC_013520.1; NC_006448.1; NC_002950.2;
NC_017276.1; NC_017275.1; NC_012632.1; NC_012588.1; NC_015636.1; NC_013887.1;
NC_013769.1; NC_013156.1; NC_009635.1; NC_015680.1; NC_010482.1; NC_009698.1;
NC_012726.1; NC_015562.1; NC_011296.1; NC_011296.1; NC_009634.1; NC_012589.1;
NC_012589.1; NC_013407.1; NC_013926.1; NC_012622.1; NC_013156.1; NC_011296.1;
NC_013790.1; NC_009033.1; NC_016070.1; NC_013849.1; NC_019977.1; NC_013407.1;
NC_015869.2; NC_011661.1; NC_000853.1; NC_000917.1; NC_002945.3; NC_002754.1;
NC_007114.6; NC_019473.1; NC_023623.1; NW_007577872.1; NC_013900.1; NW_003155240.1;
NC_000005.10; NC_002689.2; NC_009009.1; NC_014222.1; NC_003413.1; NC_014374.1;
NC_011766.1; NC_000918.1; NC_022093.1; NC_018092.1; NC_000853.1; NC_017527.1;
NC_015151.1; NC_009495.1; NC_015562.1; NC_014222.1; NC_015460.1; NC_015460.1;
NC_015460.1; NC_009440.1; NC_007643.1; NC_003552.1; NW_006263392.1; AC_000177.1;
NC_004316.3; NC_016411.2; NC_012225.1; NC_005788.4; NC_014323.1; NC_008818.1;
NC_017960.1; NW_006436284.1; NW_006211808.1; NC_013743.1; NC_009380.1;
NW_005393620.1; NC_011089.1; NW_007727769.1; NW_007673341.1; NC_024350.1;
NW_006921745.1; NW_006533007.1; NW_006532497.1; NW_006712293.1; NW_006501110.1;
NW_006400054.1; NW_006408600.1; NW_005882775.1; NW_005785850.1; NW_005812630.1;
NC_022312.1; NW_004454213.1; NC_018441.1; NC_018723.2; NC_016133.1; NC_010446.4;
NC_009164.2; NC_003047.1; NC_007088.5; NC_009776.1; NC_015931.1; NC_000916.1;
NC_015518.1; NC_007643.1; NC_003901.1; NC_016491.1; NC_026938.1; NW_007379404.1;
NW_007377898.1; NC_008809.1; NC_016103.1; NC_016099.1; NW_005372401.1;
NW_003140313.1; NW_003383754.1; NC_008599.1; NC_016522.1; NC_014122.1; NC_011167.1;
NC_010175.1; NC_008698.1; NC_007355.1; NC_005877.1; NC_003106.2; NC_012623.1;
NC_012589.1; NC_023069.1; NC_008701.1; NC_018015.1; NC_007181.1; NC_020247.1;
NW_004218575.1; NC_022973.1; NC_022984.1; NC_017459.1; NC_007426.1; NC_002663.1;
NC_016023.1; NW_006433950.1; NW_006383296.1; NC_015636.1; NC_015562.1; NC_015562.1;
NC_015954.1; NC_011832.1; NC_007796.1; NC_004547.2; NC_002663.1; NC_010002.1;
NC_015460.1; NC_013722.1; NC_014147.1; NC_009053.1; NC_016901.1; NC_014500.1;
NC_020418.1; NW_006890166.1; NC_012019.3; NC_017634.1; NC_003143.1; NC_002971.3;
NC_016477.1; NC_025693.1; NC_004547.2; NC_002663.1; NC_005085.1; NC_010002.1;
NC_015460.1; NC_020418.1; NC_013722.1; NC_014147.1; NC_009053.1; NC_016901.1;
NC_014500.1; NC_000919.1; NC_009784.1; NW_006804308.1; NC_017634.1; NC_003143.1;
NC_004605.1; NC_007284.2; NC_019472.1; NC_004547.2; NC_009784.1; NC_020418.1;
NC_002663.1; NC_015460.1; NC_005085.1; NC_010002.1; NC_002971.3; NC_013722.1;
NC_014147.1; NC_009053.1; NC_016901.1; NC_014500.1; NC_017634.1; NC_003143.1;
NC_004605.1; NC_016457.1; NW_007546277.1; NC_014644.1; NC_002939.5; NC_000913.3;
NC_011138.3; NC_014228.1; NC_013892.1; NC_009953.1; NC_003198.1; NC_014106.1;
NC_007643.1; NC_007643.1; NC_016112.1; NC_014318.1; NC_011832.1; NC_009953.1;
NC_009439.1; NC_007955.1; NC_014500.1; NC_015676.1; NC_014106.1; NC_010610.1;
NC_009440.1; NC_008054.1; NC_007164.1; NC_015518.1; NC_017276.1; NC_017275.1;
NC_012632.1; NC_012623.1; NC_012588.1; NC_015760.1; NC_013769.1; NC_009953.1;
NC_002754.1; NC_010572.1; NC_012622.1; NC_012589.1; NC_017274.1; NC_008054.1;
NC_020990.1; NC_015760.1; NC_009778.1; NC_008526.1; NC_008481.2; AC_000180.1;
NW_005842445.1; NC_026935.1; NW_005784896.1; NW_007359860.1; NC_013961.1;
NC_010610.1; NC_009440.1; NC_007164.1; NC_008508.1; NC_010296.1; NC_003197.1;
NW_008237220.1; NW_005882971.1; NW_005853887.1; NW_005394383.1; NC_018890.1;
NC_018390.1; NC_013051.1; NC_002695.1; NC_002677.1; NC_009464.1; NC_002932.3;
NC_018658.1; NC_011751.1; NC_026504.1; NC_013486.1; NW_005372181.1; NC_007796.1;
NC_014644.1; NC_002939.5; NC_000913.3; NC_011138.3; NC_020990.1; NC_011832.1;
NC_009953.1; NC_009439.1; NC_014106.1; NC_007643.1; NC_016112.1; NC_003198.1;
NC_014318.1; NC_015676.1; NC_007955.1; NC_013961.1; NC_015760.1; NC_002935.2;
NC_008526.1; NC_014644.1; NW_005819431.1; NC_016105.1; NC_009778.1; NC_008508.1;
NC_003197.1; NC_014228.1; NC_013892.1; NW_005785758.1; NC_002695.1; NC_010572.1;
NC_010610.1; NC_007164.1; NC_009464.1; NC_002932.3; NC_007643.1; NC_018658.1;
NC_011751.1; NC_025998.1; NC_009953.1; NC_007796.1; NC_017459.1; NC_007426.1;
NC_010296.1; NC_015562.1; NC_021058.1; NC_013407.1; NC_007181.1; NC_020247.1;
NC_020246.1; NC_013769.1; NC_011832.1; NC_007796.1; NC_015954.1; NC_005877.1;
NW_007377947.1; NC_008698.1; NC_017459.1; NC_007426.1; NC_010296.1; NC_015636.1;
NC_015562.1; NC_011832.1; NC_007796.1; NC_021058.1; NC_015954.1; NC_013407.1;
NC_011832.1; NC_007796.1; NC_007181.1; NC_020247.1; NC_020246.1; NC_017275.1;
NC_013769.1; NC_000016.10; NC_005877.1; NC_008698.1; NC_011296.1; NC_002754.1;
NC_009033.1; NC_017946.1; NC_015680.1; NC_015518.1; NC_003106.2; NC_017276.1;
NC_017275.1; NC_012632.1; NC_012588.1; NC_013887.1; NC_013769.1; NC_013156.1;
NC_002754.1; NC_015435.1; NC_009440.1; NC_006624.1; NC_012726.1; NC_012623.1;
NC_022084.1; NC_021058.1; NC_012622.1; NC_012589.1; NC_000961.1; NC_011766.1;
NC_010482.1; NC_000917.1; NC_011913.1; NW_006715916.1; NC_021792.1; NC_009776.1;
NC_022093.1; NC_015931.1; NC_008818.1; NC_026477.1; NC_023069.1; NC_024142.1;
NC_017576.1; NC_007929.1; NC_002967.9; NC_018081.1; NC_017576.1; NC_013198.1;
NC_009785.1; NC_022279.1; NC_000003.12; NC_025981.2; NC_018153.1; NC_006448.1;
NC_008482.2; NC_007865.1; NW_006890062.1; NC_012871.1; NC_008476.2; NW_006204462.1;
NW_005819170.1; NC_019042.1; NC_002737.2; NC_007297.1; NC_004350.2; NC_004116.1;
NC_014334.1; NC_022246.1; NC_011661.1; NC_010175.1; NC_015680.1; NC_013520.1;
NC_011529.1; NC_006448.1; NC_015460.1; NC_011296.1; NC_011296.1; NC_013887.1;
NC_009635.1; NC_000962.3; NC_017946.1; NC_018015.1; NC_013790.1; NC_015562.1;
NC_013156.1; NC_013407.1; NC_013926.1; NC_013407.1; NC_015518.1; NC_015636.1;
NC_015562.1; NC_009387.2; NC_007268.2; NC_007244.2; NC_000916.1; NC_021058.1;
NC_017276.1; NC_017275.1; NC_018229.1; NC_012632.1; NC_012623.1; NC_012622.1;
NC_012589.1; NC_012588.1; NC_013769.1; NC_002754.1; NC_005027.1; NC_005877.1;
NC_000961.1; NC_000853.1; NC_014537.1; NC_009009.1; NC_003552.1; NC_002945.3;
NC_012726.1; NW_007547664.1; NC_010175.1; NC_010175.1; NC_000962.3; NC_011296.1;
NC_000961.1; NC_011661.1; NC_017946.1; NC_016070.1; NC_015680.1; NC_013520.1;
NC_011529.1; NC_014537.1; NC_011296.1; NC_015636.1; NC_015562.1; NC_013887.1;
NC_009635.1; NC_008698.1; NC_008698.1; NC_014205.1; NC_009033.1; NC_018015.1;
NC_013790.1; NC_015562.1; NC_013156.1; NC_015151.1; NC_013407.1; NC_013407.1;
NC_013926.1; NC_008701.1; NC_003106.2; NC_003106.2; NC_012589.1; NC_000916.1;
NC_003552.1; NC_002754.1; NC_002754.1; NC_002689.2; NC_000853.1; NC_002945.3;
NC_001140.6; NC_011296.1; NC_000962.3; NC_011661.1; NC_010175.1; NC_000961.1;
NC_015680.1; NC_013520.1; NC_011529.1; NC_006448.1; NC_015460.1; NC_015636.1;
NC_013156.1; NC_009635.1; NC_017946.1; NC_015562.1; NC_013887.1; NC_011296.1;
NC_013407.1; NC_013926.1; NC_013407.1; NC_002689.2; NC_000916.1; NC_000853.1;
NC_003552.1; NC_002945.3; NC_000962.3; NC_000961.1; NC_011661.1; NC_010175.1;
NC_017946.1; NC_015680.1; NC_013520.1; NC_011529.1; NC_006448.1; NC_011296.1;
NC_011296.1; NC_015636.1; NC_015562.1; NC_013887.1; NC_013156.1; NC_009635.1;
NC_014205.1; NC_009033.1; NC_014537.1; NC_013790.1; NC_015562.1; NC_008698.1;
NC_013407.1; NC_013926.1; NC_013407.1; NC_008701.1; NC_002689.2; NC_000853.1;
NC_003552.1; NC_002945.3; NC_000962.3; NC_003106.2; NC_017276.1; NC_017275.1;
NC_012632.1; NC_012588.1; NC_002754.1; NC_016070.1; NC_014537.1; NC_014537.1;
NC_012726.1; NC_009954.1; NC_009954.1; NC_017274.1; NC_021058.1; NC_015151.1;
NC_015151.1; NC_009135.1; NC_009785.1; NC_009615.1; NC_015678.1; NC_002737.2;
NT_078268.4; NC_012470.1; NC_007297.1; NC_021058.1; NC_017576.1; NC_022246.1;
NC_022246.1; NC_009668.1; NW_006921704.1; NC_012004.1; NC_019042.1; NC_025414.1;
NC_014374.1; NC_008818.1; NC_022093.1; NC_012804.1; NC_004350.2; NC_004116.1;
NC_009009.1; NC_009009.1; NC_002662.1; NC_022239.1; NC_016112.1; NC_002945.3;
NC_025689.1; NC_024243.1; NC_018227.2; NC_002754.1; NC_012589.1; NC_017276.1;
NC_012726.1; NC_002754.1; NC_012622.1; NC_012622.1; NC_017274.1; NC_012632.1;
NC_012588.1; NC_010482.1; NC_017527.1; NC_017275.1; NC_011296.1; NC_012622.1;
NC_012589.1; NC_021058.1; NC_013849.1; NC_019977.1; NC_013156.1; NC_021058.1;
NC_013407.1; NC_003413.1; NC_003413.1; NC_000918.1; NC_018092.1; NC_018092.1;
NC_015680.1; NC_000916.1; NC_000853.1; NC_000917.1; NC_003106.2; NC_003901.1;
NC_013769.1; NC_007355.1; NC_001134.8; NC_009698.1; NC_002950.2; NC_003106.2;
NC_017276.1; NC_002754.1; NC_015680.1; NC_017527.1; NC_009634.1; NC_012622.1;
NC_017276.1; NC_017275.1; NC_012632.1; NC_012588.1; NC_013769.1; NC_014222.1;
NC_021058.1; NC_012622.1; NC_012589.1; NC_021058.1; NC_012622.1; NC_012589.1;
NC_003413.1; NC_000918.1; NC_018092.1; NC_000853.1; NC_000917.1; NC_002754.1;
NC_009698.1; NC_011296.1; NC_015680.1; NC_002950.2; NC_000917.1; NC_003106.2;
NC_002754.1; NC_000916.1; NC_002754.1; NC_017274.1; NC_006448.1; NC_017527.1;
NC_017276.1; NC_012632.1; NC_012588.1; NC_014374.1; NC_010482.1; NC_009776.1;
NC_009776.1; NC_017276.1; NC_012726.1; NC_013769.1; NC_012622.1; NC_012622.1;
NC_012622.1; NC_012589.1; NC_012589.1; NC_021058.1; NC_021058.1; NC_015931.1;
NC_013849.1; NC_017275.1; NC_003413.1; NC_008818.1; NC_000918.1; NC_000918.1;
NC_018092.1; NC_000853.1; NC_014222.1; NC_002754.1; NC_000917.1; NC_002754.1;
NC_015680.1; NC_003106.2; NC_017276.1; NC_017276.1; NC_012632.1; NC_012588.1;
NC_002754.1; NC_017527.1; NC_009634.1; NC_013769.1; NC_008698.1; NC_021058.1;
NC_012622.1; NC_012622.1; NC_012622.1; NC_012589.1; NC_012589.1; NC_017274.1;
NC_021058.1; NC_002950.2; NC_009698.1; NC_017275.1; NC_010482.1; NC_003413.1;
NC_011766.1; NC_000918.1; NC_022093.1; NC_018092.1; NC_008818.1; NC_014537.1;
NC_009698.1; NC_009495.1; NC_012726.1; NC_014222.1; NC_014222.1; NC_009954.1;
NC_002754.1; NC_002754.1; NC_015680.1; NC_017274.1; NC_002754.1; NC_017527.1;
NC_014537.1; NC_017276.1; NC_017276.1; NC_017275.1; NC_012632.1; NC_012588.1;
NC_009954.1; NC_014374.1; NC_010482.1; NC_012726.1; NC_013769.1; NC_009634.1;
NC_021058.1; NC_015151.1; NC_012622.1; NC_012622.1; NC_012622.1; NC_012589.1;
NC_021058.1; NC_012589.1; NC_003106.2; NC_002950.2; NC_003413.1; NC_000918.1;
NC_018092.1; NC_000853.1; NC_000917.1; NC_002939.5; NC_011835.1; NC_018227.2;
NC_013715.1; NC_016091.1; NC_016102.1; NC_012594.1; NC_009953.1; NC_007355.1;
NC_002939.5; NC_023646.1; NC_006111.3; NC_025815.1; NC_010449.4; NC_024467.1;
NC_024459.1; NC_016114.1; NC_006113.3; NC_013172.1; NW_006921737.1; NW_006159630.1;
NW_005785086.1; NC_013715.1; NC_009953.1; NC_007425.2; NC_017527.1; NC_007643.1;
NC_007643.1; NC_025202.1; NC_024803.1; NC_023179.1; NC_022205.1; NC_018227.2;
NC_007355.1; NC_009668.1; NC_013715.1; NW_005876445.1; NC_005115.4; NC_000080.6;
NW_003614076.1; NC_022058.1; NC_024797.1; NW_003805272.1; NC_018227.2; NC_009135.1;
NC_007355.1; NC_002939.5; NW_007359863.1; NC_012875.1; NC_010175.1; NC_010175.1;
NC_015460.1; NC_005140.1; NC_005140.1; NC_000916.1; NC_015518.1; NC_021058.1;
NC_017276.1; NC_017275.1; NC_012632.1; NC_012623.1; NC_012588.1; NC_013769.1;
NC_002754.1; NC_002754.1; NC_012622.1; NC_012589.1; NC_002935.2; NC_015435.1;
NC_009440.1; NC_012726.1; NW_003383698.1; NC_011661.1; NC_017527.1; NC_013849.1;
NC_010175.1; NC_015680.1; NC_018015.1; NC_000917.1; NC_000918.1; NC_011296.1;
NC_011661.1; NC_015518.1; NC_011661.1; NC_010175.1; NC_016070.1; NC_015680.1;
NC_015435.1; NC_011529.1; NC_003106.2; NC_013887.1; NC_013887.1; NC_013156.1;
NC_002754.1; NC_015680.1; NC_017946.1; NC_014205.1; NC_011766.1; NC_009033.1;
NC_016070.1; NC_015518.1; NC_015460.1; NC_015435.1; NC_014537.1; NC_013790.1;
NC_013790.1; NC_009440.1; NC_012623.1; NC_013156.1; NC_015562.1; NC_014222.1;
NC_011296.1; NC_011296.1; NC_008698.1; NC_007796.1; NC_007796.1; NC_007796.1;
NC_002754.1; NC_014147.1; NC_013407.1; NC_013407.1; NC_008701.1; NC_013926.1;
NC_015151.1; NC_015151.1; NC_017274.1; NC_017274.1; NC_013407.1; NC_011753.2;
NW_006494614.1; AC_000171.1; NW_005785783.1; NW_007546298.1; NC_026658.1;
NC_013926.1; NC_007624.1; NC_011910.1; NC_009668.1; NC_009914.1; NC_026504.1;
NC_015320.1; NC_007859.1; NC_006584.3; NC_003413.1; NC_002754.1; NC_013520.1;
NC_013520.1; NC_008818.1; NC_022084.1; NC_021058.1; NC_021058.1; NC_021058.1;
NC_018015.1; NC_018015.1; NC_015865.1; NC_015518.1; NC_015435.1; NC_014804.1;
NC_012883.1; NC_011529.1; NC_007181.1; NC_014729.1; NC_013158.1; NC_010575.1;
NC_009464.1; NC_005085.1; NC_020247.1; NC_020246.1; NC_017276.1; NC_017276.1;
NC_017276.1; NC_017275.1; NC_017275.1; NC_016901.1; NC_012724.2; NC_010296.1;
NC_007952.1; NC_004663.1; NC_013929.1; NC_012632.1; NC_012632.1; NC_012623.1;
NC_012623.1; NC_012622.1; NC_012589.1; NC_012589.1; NC_012588.1; NC_012588.1;
NW_007907215.1; NW_007729274.1; NW_006713510.1; NW_006384629.1; NW_005819424.1;
NW_005395962.1; NC_022273.1; NC_019466.1; NC_018739.2; NC_016132.1; NC_012593.1;
NC_026744.1; NC_026585.1; NC_026584.1; NC_022067.1; NC_017274.1; NC_017274.1;
NC_017274.1; NC_016563.1; NC_015562.1; NC_013769.1; NC_013769.1; NC_010175.1;
NC_002754.1; NC_002754.1; NC_009089.1; NC_014374.1; NC_009776.1; NC_005877.1;
NC_005877.1; NC_005877.1; NC_002689.2; NC_002689.2; NC_002689.2; NC_000918.1;
NC_022093.1; NC_022093.1; NC_022093.1; NC_018092.1; NC_015931.1; NC_015931.1;
NC_015931.1; NC_015865.1; NC_010482.1; NC_010482.1; NC_000916.1; NC_000961.1;
NC_000961.1; NC_000853.1; NC_000853.1; NC_021313.1; NC_020388.1; NC_018876.1;
NC_015151.1; NC_013849.1; NC_009440.1; NC_007426.1; NC_007181.1; NC_007181.1;
NC_007181.1; NC_003106.2; NC_027207.1; NC_027206.1; NC_020247.1; NC_020247.1;
NC_020247.1; NC_020246.1; NC_020246.1; NC_020246.1; NC_006347.1; NC_005140.1;
NC_013486.1; NC_013486.1; NC_012726.1; NC_012632.1; NC_012589.1; NC_012588.1;
NC_006038.1; NC_012012.3; NW_007540743.1; NW_007546279.1; NC_024240.1; NC_024325.1;
NC_024128.1; NC_024128.1; NW_001814964.1; NW_006739654.1; NW_006272017.1;
NW_006272029.1; NC_006586.3; NC_015562.1; NC_015562.1; NC_023069.1; NC_023069.1;
NC_023069.1; NC_023069.1; NC_023752.1; NC_019792.1; NC_017274.1; NC_015636.1;
NC_013887.1; NC_011296.1; NC_010175.1; NC_010175.1; NC_010175.1; NC_009954.1;
NC_009635.1; NC_008698.1; NC_007796.1; NC_007796.1; NC_002754.1; NC_002754.1;
NC_007422.4; NC_010175.1.

1. A composition comprising:
(i) a nucleic acid sequence comprising at least one or a combination of domains from a 5′ to 3′ orientation: a DNA-binding domain and a Cas protein-binding domain; wherein the DNA-binding domain comprises from about 0% to about 100% modified nucleotides and/or wherein the Cas protein-binding domain comprises from about 0% to about 100% modified nucleotides, or a salt thereof; or
(ii) a first nucleic acid molecule and a second nucleic acid molecule; wherein the first nucleic and/or second nucleic acid molecule are sufficiently complementary to form a duplex and in combination comprise a series of contiguous domains from a 5′ to 3′ orientation: a DNA-binding domain and a Cas protein-binding domain; wherein from about 0% to about 100% of the nucleotides in the DNA-binding domain are modified nucleotides; and/or wherein from about 1% to about 100% of the nucleotides in the Cas protein-binding domain are modified nucleotides, or a salt thereof.

2. The composition of claim 1, wherein the DNA-binding domain is from about 25 to about 55 nucleotides in length, the Cas protein-binding domain is from about 30 to about 55 nucleotides in length and the transcription terminator domain is from about 17 to about 45 nucleotides in length; and wherein the DNA-binding domain comprises from about 40% to about 60% modified ribonucleotides at the 2′carbon position of a pentose sugar and/or the transcription terminator domain comprises from about 40% to about 60% modified ribonucleotides at the 2′carbon position of a pentose sugar.

3. The composition of claim 1, wherein the DNA-binding domain from the nucleic acid of (i) or the DNA-binding domain from the first or second nucleic acid molecule of (ii) comprises at least one fluorinated nucleic acid and is unmodified at positions 1, 2, 3, 4, and/or 5 from the 5′ end of the sequence.

4. The composition of claim 1, wherein the Cas-protein binding domain comprises a nucleotide sequence having at least 70% sequence identity to SEQ ID NO: 112, at least one fluorinated nucleic acid and an unmodified nucleotide at position 2, 3, 4, 23, 24, 25, 27, 31 and/or 42 of SEQ ID NO:112.

5. The composition of claim 1, wherein the transcription terminator domain comprises a nucleotide sequence having at least 70% sequence identity to SEQ ID NO: 114, at least one fluorinated nucleic acid, and a conserved nucleotide at position 1, 2, 3 and/or 6 of SEQ ID NO:114.

6. The composition of claim 1, wherein the DNA-binding domain consists of from about 15 to about 25 contiguous ribonucleotides; wherein the Cas protein-binding domain consists of from about 38 to about 42 contiguous ribonucleotides; and wherein the transcription terminator domain consists of from about 38 to about 42 contiguous ribonucleotides.

7. The composition of claim 1, wherein the Cas protein-binding domain is at least 70% homologous to SEQ ID NO:2 and binds a target sequence of a DNA molecule in the presence of a concentration of Cas9 protein or functional fragment thereof sufficient to cause hybridization of the DNA-binding domain to the target sequence; wherein the transcription terminator domain is at least 70% homologous to SEQ ID NO:3 and wherein the nucleic acid sequence binds a target sequence of a DNA molecule in the presence of a concentration of Cas protein or functional fragment thereof sufficient to cause hybridization of the DNA-binding domain to the target sequence.

8. The composition of claim 1, wherein the nucleic acid sequence is at least 70% homologous to SEQ ID NO:4 and wherein the nucleic acid sequence binds a target sequence of a DNA molecule in the presence of a concentration of Cas protein or functional fragment thereof sufficient to cause hybridization of the DNA-binding domain to the target sequence.

9-11. (canceled)

12. The composition of claim 1 wherein the Cas protein-binding domain comprises a nucleotide sequence at least 75% homologous to SEQ ID NO:11; wherein the transcription terminator domain comprises a nucleotide sequence at least 75% homologous to SEQ ID NO:12; and wherein the DNA-binding domain comprises a nucleotide sequence at least 75% homologous to any sequence of Table 4 or a sequence complementary to a nucleotide sequence at least 75% homologous to any sequence of Table 4.

13. The composition of claim 1, wherein the DNA-binding domain comprises from about 1% to about 100% modified nucleotides and/or the transcription terminator domain comprises from about 1% to about 100% modified nucleotides; wherein the DNA-binding domain comprises from about 1 to about 200 nucleotides substantially complementary to a target sequence; and wherein the nucleotides of the DNA-binding domain comprise a deoxyribonucleic acid content of an amount sufficient to increase the binding of the DNA-binding domain to the target sequence without abolishing the effect of a biologically active CRISPR complex.

14. The composition of claim 13, wherein the DNA-binding domain comprises from about 25 to about 45 nucleotides; wherein no more than about 50% of the DNA-binding domain comprises deoxyribonucleic acid or modified deoxyribonucleic acid.

15. The composition of claim 14, wherein from about 1% to about 100% of the modified nucleotides are modified ribonucleotides or modified deoxyribonucleic acids at the 2′carbon position of a pentose sugar.

16. (canceled)

17. The composition of claim 1, wherein the first nucleic acid molecule and second nucleic acid molecule are sufficiently complementary such that, in the presence of a Cas protein or a functional fragment thereof and a target nucleic acid sequence, the first and/or second nucleic acid molecules form a duplex capable of binding to the Cas protein or functional fragment thereof thereby rendering the Cas protein or functional fragment thereof enzymatically active upon the target nucleic acid sequence.

18. The composition of claim 17, wherein from about 1% to about 75% of the modified or unmodified nucleotides are deoxyribonucleotides or a derivative thereof.

19. The composition of claim 1, wherein one or more of the nucleotides at positions 1, 2, 3, 4, 5, 6, 7, 8, 9, and/or 10 from the 5′ end of the DNA-binding domain is a deoxyribonucleotide.

20. The composition of claim 1, wherein the nucleic acid sequence or the first nucleic acid molecule comprise about 1, 2, 3, 4, 5, 6, 7, 8, 9, 10, 11 or 12 deoxyribonucleotides in the DNA-binding domain.

21-30. (canceled)

31. A pharmaceutical composition comprising the composition of claim 1 and a pharmaceutically acceptable carrier.

32. A eukaryotic cell comprising a target sequence and the composition of claim 1, wherein the DNA-domain hybridizes with the target sequence, wherein the target sequence encodes and the cell expresses at least one gene product, is a regulatory sequence operably linked to an expressible coding sequence, or is a mitochondrial DNA.

33. (canceled)

34. A method of altering expression of at least one gene product in a cell comprising introducing into a cell an engineered, non-naturally occurring Clustered Regularly Interspaced Short Palindromic Repeats (CRISPR)-CRISPR associated (Cas) (CRISPR-Cas) system comprising: (a) a vector comprising a nucleotide sequence encoding a Cas protein or functional fragment thereof; and (b) the composition of claim 1, wherein components (a) and (b) are located on same or different vectors of the system; wherein the cell contains and expresses a DNA molecule having a target sequence and encoding the gene product; and wherein the guide RNA targets and hybridizes with a DNA target sequence and the Cas protein or functional fragment thereof cleaves the DNA molecule, whereby expression of the at least one gene product is altered.

35-38. (canceled)

39. A method of introducing a mutation in the genomic DNA of a eukaryotic cell comprising contacting said cell with the composition of claim 1.

40. (canceled)
